(19) United States
(12) Patent Application Publication
(10) Pub. No.: US 2026/0139016 A1
(43) Pub. Date: May 21, 2026

(54) METHODS AND COMPOSITIONS FOR TREATING METABOLIC DISORDERS

(71) Applicant: President and Fellows of Harvard College, Cambridge, MA (US)

(72) Inventors: Jose Rivera-Feliciano, Cambridge, MA (US); Douglas A. Melton, Cambridge, MA (US); Timothy Kunz, Cambridge, MA (US)

(21) Appl. No.: 19/392,118

(22) Filed: Nov. 17, 2025

Related U.S. Application Data
(60) Provisional application No. 63/721,263, filed on Nov. 15, 2024.
(60) Provisional application No. 63/805,694, filed on May 14, 2025.

(51) Int. Cl. C07K 14/435; A61K 45/06; A61P 3/08

(52) U.S. Cl. CPC C07K 14/435; A61K 45/06; A61P 3/08; C07K 2319/30

(57) ABSTRACT

Peptide agents are disclosed herein that increase blood glucose clearance independent of insulin action. Also described herein are methods of treating metabolic disorders such as diabetes or obesity by administering the disclosed peptide agents.

### CROSS-REFERENCE TO RELATED APPLICATIONS

This application claims the benefit of U.S. Provisional Application No. 63/721,263, filed on Nov. 15, 2024, and U.S. Provisional Application No. 63/805,694, filed on May 14, 2025. The entire teachings of the above applications are incorporated herein by reference.

### SEQUENCE LISTING

The Sequence Listing associated with this application is provided in .xml format in lieu of a paper copy and is hereby incorporated by reference into the specification. The name of the .xml file containing the Sequence Listing is HRVY-245-101X. The xml file is 75,058 bytes, was created on Dec. 4, 2025, and is being submitted electronically via Patent Center.

### BACKGROUND OF THE INVENTION

Glucose is a central metabolic substrate that serves as the primary energy source for most mammalian cells. Under normal physiological conditions, glucose homeostasis is tightly regulated through coordinated hormonal and cellular mechanisms. Dysregulation of glucose production, uptake, or utilization is a hallmark of numerous metabolic diseases, including diabetes and obesity. In these conditions, impaired insulin signaling, altered glucose transport, and abnormal hepatic glucose output contribute to chronic hyperglycemia and downstream metabolic complications. Accordingly, there remains a need for improved therapeutic strategies and diagnostic tools that target glucose metabolism and its associated regulatory pathways.

### SUMMARY OF THE INVENTION

New developments in cell-based treatments for diabetes allow the creation of functional insulin-producing beta cells from human pluripotent stem cells through multi-step in vitro protocols (Pagliuca et al.). Stem cell-derived beta cells can be used to investigate the proteins secreted during distinct stages of development, enabling the identification of non-canonical secreted proteins, growth factors, cell fate determinants, and hormones with important roles in beta cell differentiation or blood glucose regulation. An endoplasmic reticulum mRNA sequencing (ER-seq) technique was developed to identify secreted and transmembrane proteins expressed during differentiation of stem cell-derived pancreatic beta cells. Among the mRNAs identified was a novel transcript encoding a secreted protein, C1ORF127 (also referred to herein as “Atollin”). C1ORF127 is highly expressed in mouse and human beta cells and regulated by beta cell transcription factors. Mice overexpressing C1ORF127 exhibited decreased blood glucose levels without hypoglycemia in a glucose-dependent manner, and lowered glucose even with insulin receptor inhibition or beta cell ablation. Long-term overexpression models demonstrated enhanced peripheral insulin sensitivity as well as mitigated age-related weight gain and elevated cholesterol levels. This suggests that C1ORF127 is an important regulator of glucose homeostasis and metabolism, operating through novel insulin-independent mechanisms, and thus has significant therapeutic and diagnostic potential.

Disclosed herein is an agent comprising a functional portion of a C1ORF127 gene product, as well as pharmaceutical compositions comprising the agent. Also disclosed herein are methods of treating or preventing a disorder associated with elevated blood glucose levels in a subject, comprising administering to said subject an effective amount of an agent comprising a functional portion of a C1ORF127 gene product. Further disclosed herein are methods of treating or preventing a metabolic disorder in a subject, comprising administering to said subject an effective amount of an agent comprising a functional portion of a C1ORF127 gene product.

In some embodiments, the functional portion of a C1ORF127 gene product has a size of approximately 25 kDa. In some embodiments, the functional portion of a C1ORF127 gene product has a size of approximately 5 kDa. In some embodiments, the functional portion of a C1ORF127 gene product has a size of approximately 4.4 kDa.

In some embodiments, the functional portion of a C1ORF127 gene product has 15-50 amino acids, e.g., 40-45, 15-35, 20-30, 22-25 amino acids. In specific embodiments, the functional portion of a C1ORF127 gene product has 22, 23, 42, 43, 45 or 46 amino acids. In some embodiments, the functional portion of a C1ORF127 gene product is an N-terminal fragment of C1ORF127. In some embodiments, the functional portion of a C1ORF127 gene product comprises an amino acid sequence at least 85%, at least 90%, or at least 95% identical to the amino acid sequence of SEQ ID NO: 10, SEQ ID NO: 11, or SEQ ID NO: 26, or a functional variant thereof. In some embodiments, the functional portion of a C1ORF127 gene product comprises an amino acid sequence of SEQ ID NO: 10, SEQ ID NO: 11, or SEQ ID NO: 26, or a functional variant thereof.

In some embodiments, a functional fragment of the C1ORF127 gene product comprises an amino acid sequence at least 90% or at least 95% identical to an amino acid sequence of any one of SEQ ID NO: 27-44 or a functional variant thereof. In some embodiments, the functional portion of a C1ORF127 gene product comprises an amino acid sequence of SEQ ID NO: 27-44, or a functional variant thereof.

In some embodiments, the functional portion of a C1ORF127 gene product is formed into a cyclic or a linear peptide. In other embodiments, the functional portion of a C1ORF127 gene product is fused, directly or via a linker, to an Fc region of an antibody, e.g., IgG1Fc. In some embodiments, the agent is provided as a fusion protein, e.g., where the fusion protein comprises the amino acid sequence of SEQ ID NO: 12 or SEQ ID NO: 23 or a functional variant thereof.

In some embodiments, the functional portion or functional variant of a C1ORF127 gene product has different glycosylation, phosphorylation, or multimerization than a native or naturally occurring C1ORF127 gene product. In some embodiments, the functional portion or functional variant of a C1ORF127 gene product has at least one different post-translational modification than a native C1ORF127 gene product. The different post-translational modification may be selected from different glycosylation and different phosphorylation. In some embodiments, the functional portion or functional variant of a C1ORF127 gene product has at least one substituted, deleted, or added amino acid compared with a corresponding native C1ORF127 gene product. In some embodiments, the functional portion or functional variant of a C1ORF127 gene product has a different activity or activity level than a corresponding native C1ORF127 gene product.

In some embodiments, the agent improves blood glucose clearance upon administration to the subject. In some embodiments, the agent lowers blood glucose upon administration to the subject. In some embodiments, the agent enhances glucose uptake upon administration to the subject. In some embodiments, the agent enhances glucose disposal upon administration to the subject. In some embodiments, the agent directs glucose into adipose tissue upon administration to the subject. In some embodiments, the agent facilitates glucose burning in adipose tissue upon administration to the subject. In some embodiments, the agent reduces hepatic glucose production upon administration to the subject. The blood glucose clearance property of the agent is, generally, independent of insulin activity. In some embodiments, the agent facilitates weight loss upon administration to the subject. In some embodiments, the agent reduces age-related weight gain and/or elevated cholesterol levels upon administration to the subject. In some embodiments, the agent does not cause hypoglycemia when administered to the subject.

In some embodiments, the subject has a metabolic disorder, such as diabetes (e.g., Type I or Type II Diabetes), metabolic dysfunction-associated steatohepatitis, hepatosteatosis, or obesity. In some embodiments, the subject is insulin resistant. The subject may be a human or non-human, e.g., murine.

Also disclosed herein is an agent comprising a functional portion of a C1ORF127 gene product, wherein the C1ORF127 gene product comprises an amino acid sequence of SEQ ID NO: 10 or a functional variant or fragment thereof. Also disclosed herein is an agent comprising a functional portion of a C1ORF127 gene product, wherein the C1ORF127 gene product comprises an amino acid sequence of SEQ ID NO: 11 or a functional variant or fragment thereof. Also disclosed herein is an agent comprising a functional portion of a C1ORF127 gene product, wherein the C1ORF127 gene product comprises an amino acid sequence of SEQ ID NO: 26 or a functional variant or fragment thereof. Further disclosed herein is an agent comprising a C1ORF127 fusion protein having an amino acid sequence of SEQ ID NO: 12 or a functional variant thereof. Further disclosed herein is an agent comprising a C1ORF127 fusion protein having an amino acid sequence of SEQ ID NO: 23 or a functional variant thereof.

Also disclosed herein is a composition comprising an agent described herein and a pharmaceutically acceptable carrier. In some embodiments, the composition further comprises an additional therapeutic agent (e.g., an anti-diabetic and/or an anti-obesity therapeutic), such as insulin or analogues thereof, GLPR1 agonist, SGLT2 inhibitor, metformin, sulfonylureas, thiazolidinediones, CB1 receptor blocker, Apellin agonist, leptin sensitizer, myostatin agonist, mitochondrial uncoupler, and combinations thereof.

Also disclosed herein are methods for detecting a metabolic disease or condition in a subject comprising obtaining a sample from the subject; measuring the level of C1ORF127 in the sample; comparing the level of C1ORF127 in the sample with the level of C1ORF127 in a control sample; and identifying the subject as having a metabolic disease or condition if (1) the level of C1ORF127 in the sample from the subject is decreased as compared with the level of C1ORF127 in a normal control sample or (2) the level of C1ORF127 in the sample from the subject is similar to the level of C1ORF127 in a control sample from a standard for the metabolic disease or condition. Alternatively or additionally, the C1ORF127 in the sample can be assessed to determine whether or not it is functional, e.g., whether it comprises a mutation rendering its function decreased or absent. In some embodiments the methods of the invention may be combined with methods known in the art for testing for insulin levels. The methods disclosed herein can be used to direct or aid in the direction of therapeutic interventions. For example, if the subject is identified as having a metabolic disorder by virtue of having reduced levels of C1ORF127 or a mutation in C1ORF127 rendering its function reduced or absent, the subject can be treated by administration of an agent described herein in accordance with methods described herein.

Also disclosed herein are methods for processing a sample from a subject comprising obtaining a sample from the subject and processing the sample by measuring the level of C1ORF127 in the sample and comparing it with the level of C1ORF127 in a control sample. Other methods for processing a sample are disclosed in which a sample comprising C1ORF127 is assessed to determine whether the C1ORF127 is functional and/or to determine whether the C1ORF127 comprises a mutation rendering its function decreased or absent.

### BRIEF DESCRIPTION OF THE DRAWINGS

These and other characteristics of the present invention will be more fully understood by reference to the following detailed description in conjunction with the attached drawings. The patent or application file contains at least one drawing executed in color. Copies of this patent or patent application publication with color drawings will be provided by the Office upon request and payment of the necessary fee.

FIGS. 1A-IT demonstrate development, validation and application of ERSeq to determine the stage specific expression of secreted factors in the developmental trajectory of SC-beta cells. FIG. 1A shows a monomeric AcGFP tag (green) was engineered into the cytosolic domain of SEC61β to facilitate the isolation of ribosome (orange)/translocon (blue) complexes at the ER membrane (grey in inset). The translated mRNA is red. FIG. 1B provides a strategy for the TALEN-mediated knock-in of CAGGS::AcGFP-SEC61β transgene into the AAVS1 locus. FIG. 1C provides live imaging of transgenic hPSCs expressing AcGFP-Sec61β in ER. Multiple cells shown. FIG. 1D shows sequential biochemical fractionation approach for the step-wise isolation of cytosolic, ER and nuclear components. ER fraction was subjected to immunopurification of ribsome/translocon complexes and associated RNA. Magnetic nanobodies against GFP depicted as gray spheres with antigen epitope in red. FIG. 1E provides Western blot of Sec61β, GFP and ribosomal protein L13a in multiple subcellular fractions after biochemical fractionation demonstrating enrichment for ribosome-translocon complexes in the ER-microsome fraction. FIG. 1F shows a RNA gel, identification of 28S and 18S ribosomal RNA subunits confirms the presence of ribosomes and the integrity of the RNA extractions. T: total; Cy: cytosolic fraction; Nu: nuclear fraction; Un: unbound ER fraction after immunoprecipation; IP: immunopurified ER fraction. FIG. 1G provides a qPCR analysis of secreted and cytosolic/nuclear factors in immunopurified ER fraction (IP) and unfractionated total RNA. Values are presented as fold change relative to total unfractionated RNA expression level. Data represent mean values ±SEM, *p<0.05, *p<0.001 Bonferroni-Dunn's multiple comparison, n=4. H). FIG. 1H provides a scatterplot of log-normalized microarray signal intensities of genes defected in IP and unfractionated cell extracts. Each dot represents a gene. Differentially expressed genes are colored light grey. Candidate secreted or translocon-associated factors are labeled and colored red. FIG. 1I provides a pie chart of predicted localization of IP-enriched genes. FIG. 1J shows top gene ontology terms of IP-enriched genes. FIG. 1K provides a diagram of the transgenic hPSC cell line that expresses AcGFP-SEC61β in insulin-expressing B cells. FIG. 1L provides immunostaining analysis of Insulin and GFP transgene expression in SC-β cells, note the high concordance of staining (yellow in the merged image). FIG. 1M provides a directed differentiation protocol for the generation of SC-β cells using the INS::AcGFP-SEC61β cell line. FIG. 1N shows FACS sorting strategy for the isolation of GFP+TSQ+endocrine cells based on Zinc content. TSQ is Zinc-dye. FIG. 1O shows the insulin transgenic cell line shares a gene expression signature of bona fide SC-β cells. Nanostring gene expression analysis of endocrine and non-endocrine genes of sorted GFP+,TSQ+ and GFP+,TSQ-endocrine cells. Values are presented as log 2 fold change relative to GFP−,TSQ-non-endocrine cells. FIG. 1P provides a scatterplot of log 10-normalized expression of genes detected in IP and total unfractionated RNA. Candidate genes are labeled, and colored red. Differentially expressed genes are colored light grey. FIG. 1Q provides a pie chart of predicted localization of IP-enriched genes (fold change>2 relative to total). FIG. 1R shows enrichment of IP expression relative to total RNA. Log2 enrichment of genes predicted to be part of the secretome or cytosolic/nuclear relative to total unfractionated RNA. FIG. 1S shows top gene ontology terms (FDR<0.05) of IP-enriched genes. FIG. 1T provides a heatmap of differentially expressed genes identified by ER-seq across multiple stages of differentiation demonstrates stage-specific expression patterns of translocon-associated mRNAs. SR, self renewing; DE, Definitive endoderm; GTE, gut tube endoderm; PP, pancreatic progenitor; EN, endocrine progenitor; β, β cells.

FIGS. 2A-2P demonstrate identification of Atollin, an SC-beta ERSeq enriched transcript, as an essential gene with glucose lowering activity. FIG. 2A shows results from a screen designed to test the glucose handling ability of SC-beta ERSeq enriched transcripts. Intraperitoneal glucose tolerance test (IPGTT) curves are represented as area under the curve (AUC). Furin-cleavable Insulin is shown as a positive control (F-INS). The full-length cDNA of candidates or fluorescent control protein were used to transfect liver via hydrodynamics-based tail vein transfection (HTVi) followed by an IPGTT (FIGS. 2B-2C). IPGTT of Atollin, the cDNA with highest improvement in glucose tolerance. No changes in fed or fasting state hypoglycemia observed. Insulin (FIG. 2D) or Pyruvate (FIG. 2E) tolerance test (ITT. PTT) is shown on Atollin overexpression animals. FIG. 2F provides a diagram of the predicted 73 kDa full-length protein, which includes the domain of unknown function (DUF) and the intrinsically disorded region (IDR). FIG. 2G shows the molecular phylogeny of Atollin, a vertebrate exclusive transcript lost in reptiles and birds. FIG. 2H provides alignments of protein sequences from primate orthologues demonstrated the high conservation of the N-terminal DUF domain. DUF black elipsoids. FIGS. 2I-2N shows Atollin is coexpressed with insulin in beta cells and their progenitors. FIG. 2I provides single cell RNA (scRNA) sequencing of adult cadaveric human islets (left). scRNA sequencing: pseudotime developmental trajectory of SC-beta (right), and scRNA sequencing of mature SC-beta (right bottom). FIGS. 2J-2K provides immunofluorescence stainings (IF)—adult human cadaveric islet (FIG. 2J) and mouse pancreas sections (FIG. 2K). FIG. 2L shows a rat insulinoma cell line 1e (Ins1e). FIG. 2M shows Lattice-sim imaging (superresolution microscopy) of murine islets of Langerhans. FIG. 2N shows a para-sagittal section of embryonic pancreas. FIG. 2O shows Atollin, perinatal lethal allele. FIG. 2P shows elevated beta-hydroxybutyrate levels (ketones) in null animals.

FIGS. 3A-3M demonstrate Atollin is an endocrine modulator of metabolism. FIGS. 3A-3D shows Atollin improves glycemia in models of diabetes, FIGS. 3A-3F show IPGTT after HTVi cDNA overexpression. FIG. 3A shows Chronic-Streptozotocin beta-cell ablation model rescue (STZ). FIGS. 3B-3C show acute hyperglycemia-S961 mediated insulin receptor inhibition rescue at high glucose bolus (FIG. 3B) and moderate glucose bolus (FIG. 3C). FIG. 3D shows diet induced obese animals (DIO). FIG. 3E provides an Atollin tumor xenotransplantation model. Stably transfected HepG2 cells overexpressing Atollin were transplanted under the kidney capsule of SCID/Beige mice. FIG. 3E shows S961 IPGTT on tumor bearing mice. This demonstrates that Atollin is a secreted endocrine modulator. FIG. 3F-3K show adeno-associated viral overexpression of Atollin in muscle (Muscle creatine kinase, MCK, promoter driven cDNA overexpression) reduces ad libitum glycemia (FIG. 3F), curtails aged associated weight gain (FIG. 3G), and reduces cholesterol levels (FIG. 3H). FIG. 3I-3K show that animals maintain their ability to overcome S961 mediated inhibition of glucose uptake (FIG. 3I) and do not have an ITT phenotype albeit having lower circulating C-peptide levels (FIGS. 3J-3K). It is suggested that Atollin is a strong insulin sensitizer that can control glycemia through insulin independent mechanisms. FIGS. 3L-3M show I.P. injection of recombinant protein of truncated Atollin harboring the DUF domain has glucose lowering activity. N-terminal flag tagged Duf only rAtollin (Flag-rAtollinDUF, no Fc fusion) (FIG. 3L). C-terminal Fc-fusion (rAtollinDUF-Fc) (FIG. 3M). This is a first indication of therapeutic formulations.

FIGS. 4A-4Q demonstrate Atollin is endogenously processed into a ˜5 kDa fragment with glucose lowering activity. FIGS. 4A-4B show endogenous processing of Atollin using 176a antibody (white). Western blots from tissue lysates extracted from human cadaveric islets (non-diabetic, ND: T2D, type 2 diabetics) reveal processing at LVKR|G (SEQ ID NO: 52). FIGS. 4C-4D provide immunoprecipitation of Atollin with 176a antibody reveals a molecular interaction with PC1/3. FIG. 4E provides a mass spec analysis of co-IP partners demonstrating enrichment for molecules involved in vesicular secretion. FIGS. 4F-4H assess moxGFP-Atollin fusions. FIG. 4F provides a diagram of a fusion. FIG. 4G shows epifluorescence demonstrates perinuclear, vesicular like pattern as seen from IF data. FIG. 4H provides western blots from tissue lysates from moxGFP-Atollin fusions (stably transfected cells) blotted against anti-Atollin T27. Band of 33 kDa corresponds to a 5 kDa N-terminal processing event in the endogenous protein. FIGS. 41-4K show endogenous Atollin processing in beta cells. FIG. 4I provides a diagram of endogenous Atollin processing. FIG. 4J shows 15 kDa processing in Human islets stained with T27. FIG. 4K shows endogenous and specific processing of a 5 kDa fragment in tissues containing beta or beta-like cells. HUES8 (self renewing control), Alpha TC (alpha cell line). FIG. 4L provides a FLAG tag strategy, Flag tags reveal processing of the DUF domain in Liver. FIGS. 4M-4Q provide functional assays showing the 5 kDA fragment harbors the glucose lowering endocrine activity. FIG. 4M shows HTVi IPGTT with cDNA for the 45 amino acid (aa) region of interest. FIG. 4N shows IPGTT using cyclic peptide works with a good dose response. The cyclic peptide improves glucose tolerance in a dose dependent manner. FIG. 4O shows the cyclic peptide is able to improve glycemia in a beta cell ablation model, STZ animals. FIGS. 4P-4Q show a 42 amino acid N-terminal fusion of Fc to Atollin has glucose lowering activity (Atollin42-Fc) (FIG. 4P). Atollin42-Fc also works to lower glycemia by overcoming Insulin receptor inhibition (S961) (FIG. 4Q).

FIGS. 5A-5D provide a characterization of the C-terminus of C1ORF127 as an intrinsically disordered region (IDR). Computational analysis is provided using four different platforms: IUPred2a (FIG. 5A), PONDR (FIG. 5B), PRDOS (FIG. 5C), and Treacle (FIG. 5D) reveals the C-terminus as an IDR. The IUPred2A prediction shows disorder tendency of protein residues, where higher values correspond to a higher probability of disorder. FIG. 5D provides a comparison to a case study on Nopp140 as an example of a large, intrinsically disordered protein. A computational prediction of disordered regions in nucleolar phosphoprotein 140 (NOPP140) and Treacle using IUPred server. If the residue value of these proteins exceeds a threshold (dotted line), the residue is considered disordered.

FIGS. 6A-6G provides a characterization of C1ORF127. FIG. 6A provides a re-analysis of expression data from a public dataset, revealing that the mouse ortholog of C1ORF127 is expressed in Neurog3 cells at 15.5 dpc. FIG. 6B provides a characterization of the putative promoter and an enhancer of C1ORF127, blue bars. FIG. 6C provides other analysis indicating regions of active chromatin. Additionally, CHIPseq analysis to demonstrate putative binding by NeuroD1, Nkx6.1, PDX1 and NeuroD to the promoter region of C1ORF127. FIGS. 6D-6E shows expression time course in mouse (FIG. 6D) and human (FIG. 6E) demonstrating that C1ORF127 is maturity associated. FIG. 6F shows C1ORF127 does not localize in mouse islets. FIG. 6G provides a genetic strategy for the two loss of function mouse alleles. One causes a 13.5 kb deletion and the other a 1.7 kb deletion. The Tables show no nulls were recovered at weaning, indicating perinatal lethality.

FIG. 7 provides a schematic of C1ORF127 (Atollin), as well as sequence information for an Atollin-cyclic peptide and a recombinant Atollin-IgGFc-fusion.

FIG. 8 demonstrates that C1ORF127 has a primary structure that is characteristic of a peptide hormone. C1ORF127 may be divided into individual regions or fragments, including a ˜57 kDa fragment and a ˜15.2 kDa fragment. PC1/3 sites are conserved endopeptidase cleavage sites commonly present in peptide hormones (i.e., insulin, glucagon, and amylin).

FIG. 9 demonstrates injection of a 26 kDa N-terminal fragment of C1ORF127 fused to the Fc portion of IgG1 improves glucose tolerance in mice. rC1ORF127-IgG1-Fc was injected at 1000 ng per animal intraperitoneally 30 minutes before orally giving the mice a 20% glucose solution (Oral glucose tolerance test, OGTT). Experiment was repeated twice.

FIG. 10 shows a Western blot detecting a small fragment (predicted 4.4 kDa, in gel at ˜7.5 kDa) in human islets (HI), Stem Cell-derived beta-cells (SC-beta), and rat insulinoma cell line (INSIE). The fragment was not detected in self-renewing Human Embryonic Stem Cells (HUES) and alpha cell lines (alpha TC1-9). Antibody: rb 4415.

FIG. 11 provides a mass spectrometry analysis of pulldowns from human islets showing enrichment for proteins associated with the secretory pathway.

FIG. 12 provides a schematic of a preparative method for the isolation of Atollin from human serum.

FIG. 13 demonstrates that a 5 kDa fragment of Atollin is present in human serum.

FIGS. 14A-14B demonstrate results of 5 kDa N-terminal fragment of Atollin fused to the Fc portion of IgG1. FIG. 14A shows injection of the 5 kDa N-terminal fragment of Atollin fused to the Fc portion of IgG1 improves glucose tolerance in mice. FIG. 14B shows the 5 kDa N-terminal fragment of Atollin fused to the Fc portion is sufficient to bypass INSR inhibition (s961).

FIG. 15 shows that peptide mimics of the 5 kDa fragment exhibit activity.

FIGS. 16A-16B demonstrates Atollin enhances glucose uptake in myotubes. FIG. 16A shows Atollin enhances glucose uptake in mouse skeletal myotubes derived from the L6 cell line. FIG. 16B shows Atollin enhances glucose uptake in primary human skeletal myotubes in a cAMP independent manner.

FIG. 17 shows Atollin promotes glucose uptake through an Akt independent pathway.

FIG. 18 shows mAtollin mutants have elevated circulating ketones and deficits in gluconeogenic enzymes.

FIG. 19 provides sequences of endogenous Atollin (SEQ ID NO: 25), a 45 aa fragment of Atollin fused to Fc (SEQ ID NO: 23), a 42 aa fragment of Atollin fused to Fc (SEQ ID NO: 12), and a N-terminal 25 kDa fragment of Atollin fused to Fc (SEQ ID NO: 24).

FIGS. 20A-20F demonstrate that secretome profiling by ER-seq in beta cells uncovers C1ORF127 as a metabolic modulator. FIG. 20A shows a monomeric AcGFP tag (green) was engineered into the cytosolic domain of SEC61β to facilitate isolation of ribosome (orange)/translocon (blue) complexes at the ER membrane (grey in inset). The translated mRNA is red. FIG. 20B shows a sequential biochemical fractionation approach for the step-wise isolation of cytosolic, ER and nuclear components. ER fraction was subjected to immunopurification of ribosome/translocon complexes and associated RNA. Magnetic nanobodies against GFP are depicted as gray spheres with antigen epitope in red. DDM, dodecyl maltoside. FIG. 20C (top) shows TALEN-mediated knock-in of CAGGS::AcGFP-SEC61β transgene into the AAVS1 locus. Expression in an undifferentiated human ES cell. FIG. 20C (bottom) provides epifluorescence imaging of transgenic hPSCs expressing AcGFP-Sec61β in ER. A differentiated colony of SC-islets is shown. FIG. 20D (top) shows a strategy for the CRISPR/Cas9 mediated, 3′UTR knockin of AcGFP-SEC61β into the insulin locus. FIG. 20D (bottom) provides immunostaining of insulin and GFP transgene expression in SC-islets. A high concordance of B-cell staining is shown (yellow in the merged image). FIG. 20E (top) provides a schematic of the directed differentiation protocol for the generation of SC-islets using the two transgenic cell lines. FIG. 20E (bottom) provides a heatmap of differentially expressed genes identified by ER-seq across multiple stages of differentiation demonstrating stage-specific expression patterns of translocon-associated mRNAs. SR, self renewing; DE, Definitive endoderm; GTE, gut tube endoderm; PP, pancreatic progenitor; EN, insulin expressing endocrine progenitor: β, β cells. 11 genes of interest in SC-β cells. Top gene ontology terms (FDR<0.05) of IP-enriched genes. FIG. 20E (bottom) further provides a pie chart of predicted localization of 2,732 IP-enriched genes (fold change>2 relative to total). FIG. 20F provides a meta-analysis of a mouse genome-wide association study (GWAS) using the Diversity Outbred strain (DO) collection which revealed a strong Logarithm Off the Odds (LOD, left panel) score in chromosome 4, in the genetic interval were the mouse ortholog of C1ORF127, Gm572, resides (black horizontal bar). Right, linkage between Gm572 and an expression Quantitative Trait Loci (eQTL) arising from the difference in expression of Gm572 in A/J vs NZO strains. Amongst the DO strains, A/J animals (yellow), which are resistant to diabetes and obesity, have the highest levels of Gm572. NZO animals, which have impaired glucose tolerance, severe obesity, and diabetes have the lowest levels (light blue).

FIGS. 21A-21I demonstrate C1ORF127 is a beta cell proprotein encoding the circulating hormone Atollin. FIG. 21A provides single cell RNAseq analysis of adult human cadaveric islets. FIG. 21B (left) provides taxonomy and FIG. 21B (right) provides Primate Conservation. FIG. 21C provides a schematic diagram of the predominant isoform of C1ORF127 found in pancreatic beta cells (684aa, 73 kDa). Peptide epitopes used to make rabbit polyclonal antibodies are marked. Putative endopeptidase cleavage sites are depicted in blue. FIG. 21D shows immunofluorescent staining in mouse pancreas sections (top) and human cadaveric islets (bottom) revealing C1ORF127 is co-expressed with Insulin in beta-cells (rabbit anti-Epitope 4 used for staining). FIG. 21E shows double immunogold Transmission Electron Microscopy (TEM) reveals that C1ORF127 and Insulin are present in human beta-cell dense core granules. FIG. 21F shows immunoprecipitation mass spectrometry analysis identifies vesicular components associated with pro-protein processing. FIG. 21G shows Western Blot analysis of protein lysates using rabbit anti-Epitope 1 antibodies demonstrate the presence of a mature, 5 kDa species, in tissues that have beta-cells. FIG. 21H shows a mature 5 kDa peptide is present in human serum. FIG. 21I (top) shows prosperous cleavage analysis predicts that the pro-protein is processed by PCSK1/2 and PCSK2. CPE is predicted to polish the C-terminal R to generate a 45aa peptide (SEQ ID NO: 47). FIG. 21I (bottom) shows semi-quantitative LC-MS analysis identifies Atollin-45aa in human serum. Left, MS1 chromatogram; Right, Normalized MS1 Spiked 45aa to endogenous in DTT lipid enriched serum.

FIGS. 22A-22J demonstrate Atollin lowers blood glucose in mice independent of insulin action. Recombinant fragments of Atollin fused to the Fc region of human IgG (5 kDa-Fc) where injected into mice and glucose tolerance was assessed via IPGTT (intraperitoneal glucose tolerance test). Recombinant Fc-only is used as a control. Quantification of the effect is provided in the Area Under the Curve graphs on the right (iAUC, incremental AUC). FIGS. 22A-22C show injection of recombinant Atollin improves glucose tolerance in normoglycemic male and female mice (FIGS. 22A-22B) as well as in the Insulin resistant and obese strain (FIG. 22C, NZO). FIG. 22D shows Atollin-Fc improves glucose tolerance in an OGTT in wildtype mice. FIG. 22E shows injections of Atollin are sufficient to bypass pharmacological inhibition of the GLPIR (using Ex9-39). FIG. 22F shows Atollin-Fc lowers glycemia without an increase in insulin secretion. FIG. 22G shows Atollin-Fc bypasses pharmacological inhibition of the Insulin receptor (INSR) using the S961 peptide. FIG. 22H shows Atollin-Fc does not cause hypoglycemia at the doses tested. FIGS. 22I-22J show Atollin-Fc improves glycemia in beta-cell alblation (FIG. 22I, STZ) and Akita models of diabetes (FIG. 22J).

FIGS. 23A-23C′ demonstrates Atollin suppresses hepatic glucose output and redirects glucose to adipose glycolysis. FIG. 23A provides a schematic of the Hyperinsulinemic-euglycemic clamp with dual tracers (HIEC) study. FIGS. 23B-23C show blood glucose levels (FIG. 23B) and glucose infusion rate (FIG. 23C) during the clamp period. FIG. 23D provides quantification of the glucose infusion rate. FIGS. 23E-23G provide quantification of body composition metrics, including body weight (FIG. 23E), fat mass (FIG. 23F) and lean mass (FIG. 23G). FIGS. 23H-23I show Basal glucose levels (FIG. 23H) and Clamp glucose levels (FIG. 23I). FIGS. 23J-23K show Basal hepatic glucose production rates (FIG. 23J) and Clamp hepatic glucose production rates (FIG. 23K). FIG. 23L provides an estimation of Hepatic Insulin Action. FIGS. 23M-230 provide rates of glucose synthesis (FIG. 23M), glucose turnover (FIG. 23N) and whole body glycolysis (FIG. 23O). FIGS. 23P-23V show 2-Deoxy glucose tissue disposition in the quadricep (FIG. 23P), gastrocnemius (FIG. 23Q), heart (FIG. 23R), brain (FIG. 23S), liver (FIG. 23T), brown adipose (FIG. 23U), and white adipose (FIG. 23V). FIG. 23W provides a schematic of the De Novo Lipogenesis (DNL) study. FIGS. 23X-23Y show blood glucose and plasma ¹⁴C-levels. FIG. 23Z provides a Body composition analysis. FIG. 23A′ provides ¹⁴C-labeled triglyceride. FIG. 23B′ provides de novo lipogenesis rate. FIG. 23C′ shows increased blood lactate levels in Akita mice.

FIGS. 24A-24J demonstrate Atollin is essential for neonatal survival and regulation of lipid oxidation. FIG. 24A provides a schematic of the 13.5 KB deletion which results in a truncated product with a premature stop. FIG. 24B shows the 13.5 kb allele is perinatal lethal. FIG. 24C shows Atollin mutants have a slight reduction in mass and blood glucose relative to littermates. FIG. 24D (top) shows neonates move freely, can breathe and drink milk. FIG. 24D (bottom) shows Insulin and Glucagon content are not significantly changed between genotypes. FIG. 24E provides a histological analysis of the pancreas that reveals no difference in the number of Insulin or Glucagon expressing cells. FIG. 24F shows blood ketones were significantly elevated in the plasma of mutant animals. FIG. 24G provides a histological analysis of liver sections at PO. Top, H&E. Bottom, Oil-red-O. Note: perisinusoidal accumulation of nuclei (arrows in mutant H&E) and increased neutral lipids. FIG. 24H shows Bulk RNA-sequencing and biological enrichment analysis demonstrates derangements in genes involved in sterol synthesis. FIG. 24I shows levels of G6PC1 protein are reduced in mAtollin mutant livers. FIG. 24J provides a Western blot with alpha-tubulin control.

FIG. 25 provides percent identity and an alignment of Atollin (SEQ ID NO: 25) with incretins and glucagon (SEQ ID NOs: 48-51).

FIG. 26 demonstrates that a 25 kDa-Fc fusion of Atollin lowers blood glucose. An intraperitoneal glucose tolerance test was performed in C57B16 male mice.

### DETAILED DESCRIPTION OF THE INVENTION

The previously identified C1ORF127 gene (also referred to as ERseq08 or Atollin) has a molecular weight of 73 kDa and a well conserved N-terminal domain of 215 amino acids. Within this domain there are two predicted glycosylation sites, one predicted phosphorylation site, five conserved Cysteine residues, and putative endopeptidase cleavage site (pro-hormone convertase like). Heretofore unidentified peptide fragments with glucose-lowering activity (e.g., about 5 kDa and smaller) are disclosed herein as providing a new methodology for treatment of diseases and conditions including metabolic diseases and/or diseases involving blood glucose clearance, such as diabetes.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ^{˜}98% of the cells become exocrine, ductular, or matrix cells, and ^{˜}2% become endocrine cells.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods described herein can be performed both in vivo and in vitro.

As used herein, the term “adult cell” refers to a cell found throughout the body after embryonic development.

The term “endoderm cell” as used herein refers to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of respiratory and digestive tracts (e.g., the intestine), the liver and the pancreas.

The term “a cell of endoderm origin” as used herein refers to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “pancreatic progenitor” or “pancreatic precursor” are used interchangeably herein and refer to a stem cell which is capable of forming any of pancreatic endocrine cells, pancreatic exocrine cells, or pancreatic duct cells. The term “Pdx1-positive pancreatic progenitor” or “Pdx1+pancreatic progenitor” as used herein refers to a cell which is a pancreatic endoderm (PE) cell. A Pdx1-positive pancreatic progenitor expresses the marker Pdx1. Other markers include, but are not limited to Cdcp1, or Ptf1a, or HNF6 or NRx2.2. The expression of Pdx1 may be assessed by any method known by the skilled person such as immunochemistry using an anti-Pdx1 antibody or quantitative RT-PCR. The term “Pdx1-positive, NKX6-1-positive pancreatic progenitor” or “Pdx1+, NKX6-1+pancreatic progenitor” as used herein refers to a cell which is a pancreatic endoderm (PE) cell. A Pdx1-positive, NKX6-1-positive pancreatic progenitor expresses the markers Pdx1 and NKX6-1. Other markers include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR.

The terms “stem cell-derived β cell”, “SC-β cell”, and “mature SC-β cell” refer to cells (e.g., pancreatic B cells) that display at least one marker indicative of a pancreatic B cell, express insulin, and display a GSIS response characteristic of an endogenous mature β cell. In some embodiments, the “SC-β cell” comprises a mature pancreatic B cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner). Examples of SC-β cells, and methods of obtaining such SC-β cells, are described in WO 2015/002724 and WO 2014/201167, both of which are incorporated herein by reference in their entirety.

The term “exocrine cell” as used herein refers to a cell of an exocrine gland, i.e., a gland that discharges its secretion via a duct. In particular embodiments, an exocrine cell refers to a pancreatic exocrine cell, which is a pancreatic cell that produces enzymes that are secreted into the small intestine. These enzymes help digest food as it passes through the gastrointestinal tract. Pancreatic exocrine cells are also known as islets of Langerhans, that secrete two hormones, insulin and glucagon.

The term “phenotype” refers to one or a number of total biological characteristics that define the cell or organism under a particular set of environmental conditions and factors, regardless of the actual genotype.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the agent is selected from the group consisting of a nucleic acid, a small molecule, a polypeptide, and a peptide. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

“Small molecule” is defined as a molecule with a molecular weight that is less than 10 kD, typically less than 2 kD, and preferably less than 1 kD. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules.

As used herein, the term “polypeptide” or “protein” is used to designate a series of amino acid residues connected to the other by peptide bonds between the alpha-amino and carboxy groups of adjacent residues. The term “polypeptide” refers to a polymer of protein amino acids, including modified amino acids (e.g., phosphorylated, glycated, glycosylated, etc.) and amino acid analogs, regardless of its size or function. The term “peptide” is often used in reference to small polypeptides, but usage of this term in the art overlaps with “protein” or “polypeptide.” Exemplary polypeptides include gene products, naturally occurring proteins, homologs, orthologs, paralogs, fragments and other equivalents, as well as both naturally and non-naturally occurring variants, fragments, and analogs of the foregoing.

The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, ST (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929, 226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long.

The terms “decrease,” “reduce,” “reduced,” “reduction,” “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or more as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “contacting” is intended to include incubating an agent or cell with another agent, medium or cell together in vitro. In some embodiments, the term “contacting” is not intended to include the in vivo exposure of cells to compounds or agents as disclosed herein that may occur naturally in a subject (i.e., exposure that may occur as a result of a natural physiological process). A step of contacting can be conducted in any suitable manner.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a stem cell derived cell described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP. Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. ScL USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25:3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL world-wide web address of: “ncbi.nlm nih.gov” for these programs. In a specific embodiment, percent identity is calculated using BLAST2 with default parameters as provided by the NCBI.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term a “variant” in referring to a polypeptide could be, e.g., a polypeptide at least 80%, 85%, 90%, 95%, 98%, or 99% identical to full length polypeptide. The variant could be a fragment of a full-length polypeptide. The variant could be a naturally occurring splice variant. The variant could be a polypeptide at least 80%. 85%, 90%, 95%, 98%, or 99% identical to a fragment of the polypeptide, wherein the fragment is at least 50%, 60%, 70%, 80%, 85%, 90%, 95%, 98%, or 99% as long as the full-length wild type polypeptide or a domain thereof having an activity of interest. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full-length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein.

The term “functional fragments” as used herein is a polypeptide having an amino acid sequence which is smaller in size than, but substantially homologous to the polypeptide it is a fragment of, and where the functional fragment polypeptide sequence is about at least 50%, or 60% or 70% or 80% or 90% or 100% or greater than 100%, for example 1.5-fold, 2-fold, 3-fold, 4-fold or greater than 4-fold effective biological action as the polypeptide from which it is a fragment of. Functional fragment polypeptides may have additional functions that can include decreased antigenicity, increased DNA binding (as in transcription factors), or altered RNA binding (as in regulating RNA stability or degradation).

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g., promoters, enhancers, and termination elements) in an expression vector. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances, the promoter sequence is recognized by the synthetic machinery of the cell, or introduced synthetic machinery, required for initiating transcription of a specific gene.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

As used herein, “prevent” when used in reference to a disease, disorder or medical condition, refers to reducing or eliminating the likelihood of development of the disease, disorder or medical condition.

As used herein, the term “administering,” refers to the placement of the agent as disclosed herein into a subject by a method or route which results in delivery to a site of action. The agent can be administered by any appropriate route which results in an effective treatment in the subject. Thus, administration via the intravenous route is specifically contemplated. However, with appropriate formulation, other routes are contemplated, including, for example, intranasally, intraarterially; intra-coronary arterially; orally, by inhalation, intraperitoneally, intramuscularly, subcutaneously, intracavity, or by other means known by those skilled in the art. The agents are administered in a manner compatible with the dosage formulation, and in a therapeutically effective amount. The quantity to be administered and timing depends on the subject to be treated, capacity of the subject's system to utilize the active ingredient, and degree of therapeutic effect desired.

A “therapeutically effective amount” is an amount of an agent that is sufficient to produce a statistically significant, measurable change in, for example, blood glucose clearance. Such effective amounts can be gauged in clinical trials as well as animal studies. A treatment is considered “effective treatment,” as the term is used herein, if any one or all of the signs or symptoms are improved or ameliorated, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies-A Laboratory Manual, Cold Spring Harbor Laboratory Press, Cold Spring Harbor, 1988; Freshney, R.I., “Culture of Animal Cells, A Manual of Basic Technique”, 5th ed., John Wiley & Sons, Hoboken, NJ, 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, MD) and National Center for Biotechnology Information, National Library of Medicine (Bethesda, MD), as of May 1, 2010, ncbi.nlm.nih.gov/omim/and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### Agents and Compositions

Aspects of the disclosure relate to agents and pharmaceutical compositions comprising the same. Some aspects of the disclosure are related to agents that decrease, lower or reduce blood glucose when administered to a subject. In some embodiments, the agent lowers blood glucose in a glucose dependent manner. The agent may agent lower blood glucose in a glucose dependent manner without causing hypoglycemia. In some embodiments, the agent improves glucose tolerance in a subject. In some aspects, the agent acts independently of insulin action.

Some aspects of the disclosure are related to agents that facilitate the transport of glucose into adipose tissue, e.g., where the glucose is rapidly broken down through glycolysis. For example, the agent may exhibit fat burning capabilities. In some embodiments, the agent enhances or enables glucose disposal. In some aspects, the agent promotes glucose uptake in myotubes.

In some embodiments, the agent improves or increases blood glucose clearance. In some embodiments, the agent returns blood glucose levels in a subject from a pathological level to a non-pathological level (e.g., from a hyperglycemic state to a normal state). In some embodiments, the agent reduces blood glucose levels in a subject by about 5%, 10%, 15%, 20%, 25%, 30%, 35%, 40%, 45%, 50%, 55%, 60%, 65%, 70%, 75% or more. In some embodiments, the agent reduces hepatic glucose production in a subject. In some embodiments, the agent reduces hepatic glucose production in a subject by about 5%, 10%, 15%, 20%, 25%, 30%, 35%, 40%, 45%, 50%, 55%, 60%, 65%, 70%, 75% or more. In some embodiments, the agent does not cause hypoglycemia in the subject (e.g., a blood sugar of less than about 70 mg/dL, a blood sugar of less than about 60 mg/dL, or to a blood sugar level wherein the subject does not exhibit signs of hypoglycemia). In some embodiments, the blood glucose clearance property of the agent is independent of insulin activity. In some embodiments, the blood glucose clearance property of the agent is not independent of insulin activity.

In some embodiments, the agent comprises a small molecule, a protein, or a nucleic acid. In particular aspects, desirable agents (e.g., compounds) comprise a native C1ORF127 gene product (also referred to herein as ERseq08 or Atollin) or a functional portion, fragment or variant thereof. Suitable compounds/agents include, but are not limited to, chemical compounds and mixtures of chemical compounds, e.g., small organic or inorganic molecules; saccharides; oligosaccharides; polysaccharides; biological macromolecules, e.g., peptides, proteins, and peptide analogs and derivatives; peptidomimetics; nucleic acids; nucleic acid analogs and derivatives; extracts made from biological materials such as bacteria, plants, fungi, or animal cells or tissues; naturally occurring or synthetic compositions; peptides; aptamers; and antibodies, or fragments thereof.

A compound/agent can be a nucleic acid RNA or DNA and can be either single or double stranded. Example nucleic acid compounds include, but are not limited to, a nucleic acid encoding a protein activator or inhibitor (e.g., transcriptional activators or inhibitors), oligonucleotides, nucleic acid analogues (e.g., peptide-nucleic acid (PNA), pseudo-complementary PNA (pc-PNA), locked nucleic acid (LNA) etc.), antisense molecules, ribozymes, small inhibitory or activating nucleic acid sequences (e.g., RNAi, shRNAi, siRNA, micro RNAi (mRNAi), antisense oligonucleotides etc.).

A protein and/or peptide agent can be any protein that modulates gene expression or protein activity. Non-limiting examples include mutated proteins; therapeutic proteins and truncated proteins, e.g., wherein the protein is normally absent or expressed at lower levels in the target cell. Proteins can also be selected from genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. In some embodiments, a protein and/or peptide agent comprises a fusion protein (e.g., an Fc-fusion protein). In some embodiments, a protein and/or peptide agent comprises a linear peptide (e.g., linear synthetic peptide or linear lipidated synthetic peptide) or cyclic peptide (e.g., disulfide bridged peptide or cyclic lipidated peptide). In some embodiments, a protein or polypeptide agent may be a functional variant or functional fragment of native C1ORF127 gene product.

C1ORF127 is an approximately 73 kDa full-length protein comprising a domain of unknown function (DUF) and an intrinsically disordered region (IDR). C1ORF127 has a well conserved N-terminal domain of approximately 215 amino acids. Within this domain of unknown function, two predicted glycosylation sites, one predicted phosphorylation site, five conserved Cysteine residues, and a putative endopeptidase cleavage site (pro-hormone convertase 2 like) have been identified. C1ORF127 may be co-expressed with insulin in beta cells (e.g., stem cell-derived beta cells or endogenous beta cells) and their progenitors.

C1ORF127 has two isoforms—Isoform 1 and Isoform 2. Isoform 1 is predicted to encode a transcript of 2,798 bp with a coding sequence of 2,472 bp and a protein sequence of 823 aa. Isoform 2 is predicted to encode a transcript of 2,461 bp with a coding sequence of 2,055 bp and a protein sequence of 684 aa.

Isoform 1 of C1ORF127 has an mRNA sequence of SEQ ID NO: 19:
AAATTCTCCCAGGGCAGGAGAGAGCTGCTGGGCCTGAGGATGTGGGGGTC
CCCAGCACTTGCCTGGGCCGTCTGGCTTGCCTGTGTTCAGCCCACGGTGTT
CCCATGGAGCCTCTCCTTCAGGTCAGACACGGACAAGCCAAGCTCTGCAG
CAGAAGTTTTAACAGAAGCATCTTCTTGTTGGACAGACACAGTTGAGTGCT
TCTCAGACTACATGACCCTGTGGATCCCAAGGAGCCACGTGGAGGGGCTG
AGGCGGTGGCTGGCCAGGACCTTGCACTTGCCAGGCACCTGGAGGTCCCC
TGACCACCTGGATTCTTCTCTTGCTAAATGTGGCTACTTCCTGCATCTGGCT
TCAGACGGTGACTTCCTTTTTCGAGTTCAATACTCGGCCTGCTTTGTGCAG
AAAGAGAAAGCAAATTACAGGCTGGAAATCAGAATATTTCAGAAAGGGGT
GATGGGGTTGGAGCGGAGTGATCGCTACATAATGAAGTGTCCGATGCTAAG
GTCAAGGCTGGGCCAGGAAAGCGTCCACTGTGGGCCCATGTTCATCCAGG
TCTCCCGGCCCCTGCCCCTGTGGAGGGACAATAGACAGACTCCATGGCTGC
TGTCCCTTCGAGGGGAGCTGGTGGCTTCTCTTGAAGACGCCAGCCTGATGG
GACTGTATGTGGACATGAATGCCACCACTGTCACCGTCCAAAGCCCGAGAC
AAGGCCTTCTTCAGAGGTGGGAGGTGCTGAACACCTCTGCTGAGCTCCTG
CCACTATGGCTGGTGAGCGGTCACCATGCCTATTCTTTAGAAGCTGCTTGCC
CACCGGTGTCATTCCAGCCAGAGTCGGAGGTCTTAGTTCACATCCCCAAGC
AGAGACTGGGTCTAGTCAAAAGAGGTTCCTACATTGAGGAAACCCTGAGC
CTCAGATTCCTCCGAGTCCACCAGTCCAACATCTTTATGGTGACTGAGAAC
AAGGACTTTGTGGTGGTCAGCATTCCGGCGGCCGGGGTGCTCCAGGTCCA
GCGATGCCAAGAAGTCGGAGGAACCCCGGGAACACAAGCTTTCTATAGGG
TAGACCTGAGCCTGGAATTTGCCGAGATGGCTGCCCCGGTCCTCTGGACAG
TGGAGAGCTTCTTCCAGTGTGTGGGTTCAGGAACAGAGTCGCCTGCCTCA
ACTGCTGCACTGAGGACCACTCCCTCCCCACCATCCCCAGGACCAGAGAC
CCCTCCAGCGGGAGTGCCACCTGCTGCTTCCTCCCAGGTGTGGGCTGCAG
GACCAGCTGCCCAGGAATGGCTTTCTCGGGACCTCCTGCACCGGCCTTCCG
ACGCACTGGCCAAAAAGGGGCTTGGACCATTCCTGCAAACAGCCAAACCG
GCGAGAAGAGGCCAGACATCTGCCTCCATTCTCCCCAGAGTGGTGCAAGC
TCAGCGAGGTCCCCAGCCTCCCCCAGGGGAAGCAGGGATCCCTGGACACC
CCACACCTCCAGCCACGCTCCCCTCGGAGCCTGTAGAGGGTGTCCAGGCT
AGTCCCTGGCGGCCACGTCCAGTCTTGCCAACGCACCCGGCTCTGACCCT
GCCCGTGTCCTCAGATGCCTCCTCTCCTTCACCGCCAGCCCCGAGGCCTGA
ACGACCTGAATCACTTCTGGTCTCAGGACCATCTGTCACCCTGACTGAAGG
TCTAGGAACTGTGAGGCCTGAACAGGACCCCGCCAAGTCTCCAGGAAGTC
CCCTCCTGCTGAGAGGCTTGTCAAGCGGGGATGTGGCTGCACCTGAGCCC
ATCATGGGGGAGCCCGGCCAAGCCAGTGAGGAGTTCCAGCCATTGGCGAG
GCCCTGGCGGGCCACACTGGCTGCAGAGGAGCTGGTTTCTCACCGTTCTC
CCGGAGAGCCCCAGGAAACGTGCTCTGGAACGGAGGTGGAGAGGCCACG
CCAGACAGGGCCTGGTCTCCCCAGGGAGGGGGCCAGGGGGCACATGGAC
CTTTCATCCTCAGAACCAAGCCAGGACATAGAGGGGCCGGGACTCTCCATC
CTGCCAGCGAGGGATGCCACATTCTCCACCCCAAGTGTGAGGCAGCCAGA
CCCCAGTGCCTGGCTGAGTTCAGGACCTGAACTCACCGGGATGCCCAGGG
TGAGGCTGGCAGCGCCCCTGGCAGTTCTTCCTATGGAACCTCTGCCACCAG
AACCTGTTCGCCCAGCAGCTCTTCTGACACCCGAAGCCTCATCTGTGGGAG
GGCCAGACCAGGCCCGATACCTGGAGTCAGCCCCTGGCTGGCCTGTGGGC
CAGGAGGAGTGGGGGGTTGCACACACGTCCAGCCCTCCATCCACGCAAAC
CCTGAGCCTGTGGGCTCCCACAGGAGTGTTGCTACCCAGCCTGGTGGAGC
TTGAATACCCCTTCCAGGCTGGCCGGGGGGCCTCACTCCAGCAGGAGCTG
ACAGAGCCCACCTTGGCCCTCAGTGCTGAAAGCCACAGGCCTCCTGAGCT
TCAAGACAGTGTGGAGGGGCTTTCTGAGAGGCCCTCACGCTGAGCCTCCA
TGGAGACTCCAGGCAGCCGGTACCCCGCTGGCTTGGACAGCCCCAGTGAC
CCTCAGTGACCCAGCTGTCCCCAGGCTCCCTGGCTTAGATGCCCAGCTGTG
GCTGGGTACAGTGTTCTGGGCCGGAAGAGCACTCACTCAGTGCTTCCCTG
GAAAGGTGGACCCCCAGACGCCCACCTCCAGGCAGACCCATCACTGGAAG
TGGGATTTGAAATGCAGCCAAGAAGAGGAGACCGTCGGCAACGCCCGCCC
GGAACAAGAAATAAATAAATAAATAATCA (NM_001170754).
Isoform 1 has a coding sequence of SEQ ID NO: 20:
ATGTGGGGGTCCCCAGCACTTGCCTGGGCCGTCTGGCTTGCCTGTGTTCAG
CCCACGGTGTTCCCATGGAGCCTCTCCTTCAGGTCAGACACGGACAAGCC
AAGCTCTGCAGCAGAAGTTTTAACAGAAGCATCTTCTTGTTGGACAGACAC
AGTTGAGTGCTTCTCAGACTACATGACCCTGTGGATCCCAAGGAGCCACGT
GGAGGGGCTGAGGCGGTGGCTGGCCAGGACCTTGCACTTGCCAGGCACCT
GGAGGTCCCCTGACCACCTGGATTCTTCTCTTGCTAAATGTGGCTACTTCCT
GCATCTGGCTTCAGACGGTGACTTCCTTTTTCGAGTTCAATACTCGGCCTGC
TTTGTGCAGAAAGAGAAAGCAAATTACAGGCTGGAAATCAGAATATTTCAG
AAAGGGGTGATGGGGTTGGAGCGGAGTGATCGCTACATAATGAAGTGTCC
GATGCTAAGGTCAAGGCTGGGCCAGGAAAGCGTCCACTGTGGGCCCATGT
TCATCCAGGTCTCCCGGCCCCTGCCCCTGTGGAGGGACAATAGACAGACTC
CATGGCTGCTGTCCCTTCGAGGGGAGCTGGTGGCTTCTCTTGAAGACGCCA
GCCTGATGGGACTGTATGTGGACATGAATGCCACCACTGTCACCGTCCAAA
GCCCGAGACAAGGCCTTCTTCAGAGGTGGGAGGTGCTGAACACCTCTGCT
GAGCTCCTGCCACTATGGCTGGTGAGCGGTCACCATGCCTATTCTTTAGAA
GCTGCTTGCCCACCGGTGTCATTCCAGCCAGAGTCGGAGGTCTTAGTTCAC
ATCCCCAAGCAGAGACTGGGTCTAGTCAAAAGAGGTTCCTACATTGAGGA
AACCCTGAGCCTCAGATTCCTCCGAGTCCACCAGTCCAACATCTTTATGGT
GACTGAGAACAAGGACTTTGTGGTGGTCAGCATTCCGGCGGCCGGGGTGC
TCCAGGTCCAGCGATGCCAAGAAGTCGGAGGAACCCCGGGAACACAAGC
TTTCTATAGGGTAGACCTGAGCCTGGAATTTGCCGAGATGGCTGCCCCGGT
CCTCTGGACAGTGGAGAGCTTCTTCCAGTGTGTGGGTTCAGGAACAGAGT
CGCCTGCCTCAACTGCTGCACTGAGGACCACTCCCTCCCCACCATCCCCAG
GACCAGAGACCCCTCCAGCGGGAGTGCCACCTGCTGCTTCCTCCCAGGTG
TGGGCTGCAGGACCAGCTGCCCAGGAATGGCTTTCTCGGGACCTCCTGCA
CCGGCCTTCCGACGCACTGGCCAAAAAGGGGCTTGGACCATTCCTGCAAA
CAGCCAAACCGGCGAGAAGAGGCCAGACATCTGCCTCCATTCTCCCCAGA
GTGGTGCAAGCTCAGCGAGGTCCCCAGCCTCCCCCAGGGGAAGCAGGGAT
CCCTGGACACCCCACACCTCCAGCCACGCTCCCCTCGGAGCCTGTAGAGG
GTGTCCAGGCTAGTCCCTGGCGGCCACGTCCAGTCTTGCCAACGCACCCG
GCTCTGACCCTGCCCGTGTCCTCAGATGCCTCCTCTCCTTCACCGCCAGCC
CCGAGGCCTGAACGACCTGAATCACTTCTGGTCTCAGGACCATCTGTCACC
CTGACTGAAGGTCTAGGAACTGTGAGGCCTGAACAGGACCCCGCCAAGTC
TCCAGGAAGTCCCCTCCTGCTGAGAGGCTTGTCAAGCGGGGATGTGGCTG
CACCTGAGCCCATCATGGGGGAGCCCGGCCAAGCCAGTGAGGAGTTCCAG
CCATTGGCGAGGCCCTGGCGGGCCACACTGGCTGCAGAGGAGCTGGTTTC
TCACCGTTCTCCCGGAGAGCCCCAGGAAACGTGCTCTGGAACGGAGGTGG
AGAGGCCACGCCAGACAGGGCCTGGTCTCCCCAGGGAGGGGGCCAGGGG
GCACATGGACCTTTCATCCTCAGAACCAAGCCAGGACATAGAGGGGCCGG
GACTCTCCATCCTGCCAGCGAGGGATGCCACATTCTCCACCCCAAGTGTGA
GGCAGCCAGACCCCAGTGCCTGGCTGAGTTCAGGACCTGAACTCACCGGG
ATGCCCAGGGTGAGGCTGGCAGCGCCCCTGGCAGTTCTTCCTATGGAACCT
CTGCCACCAGAACCTGTTCGCCCAGCAGCTCTTCTGACACCCGAAGCCTC
ATCTGTGGGAGGGCCAGACCAGGCCCGATACCTGGAGTCAGCCCCTGGCT
GGCCTGTGGGCCAGGAGGAGTGGGGGGTTGCACACACGTCCAGCCCTCCA
TCCACGCAAACCCTGAGCCTGTGGGCTCCCACAGGAGTGTTGCTACCCAG
CCTGGTGGAGCTTGAATACCCCTTCCAGGCTGGCCGGGGGGCCTCACTCCA
GCAGGAGCTGACAGAGCCCACCTTGGCCCTCAGTGCTGAAAGCCACAGGC
CTCCTGAGCTTCAAGACAGTGTGGAGGGGCTTTCTGAGAGGCCCTCACGC
TGA.
Isoform 1 has a protein sequence of SEQ ID NO: 21:
MWGSPALAWAVWLACVQPTVFPWSLSFRSDTDKPSSAAEVLTEASSCWTDT
VECFSDYMTLWIPRSHVEGLRRWLARTLHLPGTWRSPDHLDSSLAKCGYFLH
LASDGDFLFRVQYSACFVQKEKANYRLEIRIFQKGVMGLERSDRYIMKCPML
RSRLGQESVHCGPMFIQVSRPLPLWRDNRQTPWLLSLRGELVASLEDASLMGL
YVDMNATTVTVQSPROGLLQRWEVLNTSAELLPLWLVSGHHAYSLEAACPPV
SFQPESEVLVHIPKQRLGLVKRGSYIEETLSLRFLRVHQSNIFMVTENKDFVVV
SIPAAGVLQVQRCQEVGGTPGTQAFYRVDLSLEFAEMAAPVLWTVESFFQCV
GSGTESPASTAALRTTPSPPSPGPETPPAGVPPAASSQVWAAGPAAQEWLSRDL
LHRPSDALAKKGLGPFLQTAKPARRGQTSASILPRVVQAQRGPQPPPGEAGIP
GHPTPPATLPSEPVEGVQASPWRPRPVLPTHPALTLPVSSDASSPSPPAPRPERP
ESLLVSGPSVTLTEGLGTVRPEQDPAKSPGSPLLLRGLSSGDVAAPEPIMGEPG
QASEEFQPLARPWRATLAAEELVSHRSPGEPQETCSGTEVERPRQTGPGLPRE
GARGHMDLSSSEPSQDIEGPGLSILPARDATFSTPSVRQPDPSAWLSSGPELTG
MPRVRLAAPLAVLPMEPLPPEPVRPAALLTPEASSVGGPDQARYLESAPGWPV
GQEEWGVAHTSSPPSTQTLSLWAPTGVLLPSLVELEYPFQAGRGASLQQELTE
PTLALSAESHRPPELQDSVEGLSERPSR (NP_001164225).
Isoform 2 of C1ORF127 has an mRNA sequence of SEQ ID NO: 22:
GGATACACCTTGAATGAAGAACCATCGGGCTTTCCTGTTGGTATATGCCTTT
CCAGACTTTTAAAAATTACTGCTATTTTTATCCCTTCCAGGCACCTGGAGGT
CCCCTGACCACCTGGATTCTTCTCTTGCTAAATGTGGCTACTTCCTGCATCT
GGCTTCAGACGGTGACTTCCTTTTTCGAGTTCAATACTCGGCCTGCTTTGTG
CAGAAAGAGAAAGCAAATTACAGGCTGGAAATCAGAATATTTCAGAAAGG
GGTGATGGGGTTGGAGCGGAGTGATCGCTACATAATGAAGTGTCCGATGCT
AAGGTCAAGGCTGGGCCAGGAAAGCGTCCACTGTGGGCCCATGTTCATCC
AGGTCTCCCGGCCCCTGCCCCTGTGGAGGGACAATAGACAGACTCCATGG
CTGCTGTCCCTTCGAGGGGAGCTGGTGGCTTCTCTTGAAGACGCCAGCCTG
ATGGGACTGTATGTGGACATGAATGCCACCACTGTCACCGTCCAAAGCCCG
AGACAAGGCCTTCTTCAGAGGTGGGAGGTGCTGAACACCTCTGCTGAGCT
CCTGCCACTATGGCTGGTGAGCGGTCACCATGCCTATTCTTTAGAAGCTGCT
TGCCCACCGGTGTCATTCCAGCCAGAGTCGGAGGTCTTAGTTCACATCCCC
AAGCAGAGACTGGGTCTAGTCAAAAGAGGTTCCTACATTGAGGAAACCCT
GAGCCTCAGATTCCTCCGAGTCCACCAGTCCAACATCTTTATGGTGACTGA
GAACAAGGACTTTGTGGTGGTCAGCATTCCGGCGGCCGGGGTGCTCCAGG
TCCAGCGATGCCAAGAAGTCGGAGGAACCCCGGGAACACAAGCTTTCTAT
AGGGTAGACCTGAGCCTGGAATTTGCCGAGATGGCTGCCCCGGTCCTCTGG
ACAGTGGAGAGCTTCTTCCAGTGTGTGGGTTCAGGAACAGAGTCGCCTGC
CTCAACTGCTGCACTGAGGACCACTCCCTCCCCACCATCCCCAGGACCAG
AGACCCCTCCAGCGGGAGTGCCACCTGCTGCTTCCTCCCAGGTGTGGGCT
GCAGGACCAGCTGCCCAGGAATGGCTTTCTCGGGACCTCCTGCACCGGCC
TTCCGACGCACTGGCCAAAAAGGGGCTTGGACCATTCCTGCAAACAGCCA
AACCGGCGAGAAGAGGCCAGACATCTGCCTCCATTCTCCCCAGAGTGGTG
CAAGCTCAGCGAGGTCCCCAGCCTCCCCCAGGGGAAGCAGGGATCCCTGG
ACACCCCACACCTCCAGCCACGCTCCCCTCGGAGCCTGTAGAGGGTGTCC
AGGCTAGTCCCTGGCGGCCACGTCCAGTCTTGCCAACGCACCCGGCTCTG
ACCCTGCCCGTGTCCTCAGATGCCTCCTCTCCTTCACCGCCAGCCCCGAGG
CCTGAACGACCTGAATCACTTCTGGTCTCAGGACCATCTGTCACCCTGACT
GAAGGTCTAGGAACTGTGAGGCCTGAACAGGACCCCGCCAAGTCTCCAGG
AAGTCCCCTCCTGCTGAGAGGCTTGTCAAGCGGGGATGTGGCTGCACCTG
AGCCCATCATGGGGGAGCCCGGCCAAGCCAGTGAGGAGTTCCAGCCATTG
GCGAGGCCCTGGCGGGCCACACTGGCTGCAGAGGAGCTGGTTTCTCACCG
TTCTCCCGGAGAGCCCCAGGAAACGTGCTCTGGAACGGAGGTGGAGAGG
CCACGCCAGACAGGGCCTGGTCTCCCCAGGGAGGGGGCCAGGGGGCACA
TGGACCTTTCATCCTCAGAACCAAGCCAGGACATAGAGGGGCCGGGACTC
TCCATCCTGCCAGCGAGGGATGCCACATTCTCCACCCCAAGTGTGAGGCAG
CCAGACCCCAGTGCCTGGCTGAGTTCAGGACCTGAACTCACCGGGATGCC
CAGGGTGAGGCTGGCAGCGCCCCTGGCAGTTCTTCCTATGGAACCTCTGCC
ACCAGAACCTGTTCGCCCAGCAGCTCTTCTGACACCCGAAGCCTCATCTGT
GGGAGGGCCAGACCAGGCCCGATACCTGGAGTCAGCCCCTGGCTGGCCTG
TGGGCCAGGAGGAGTGGGGGGTTGCACACACGTCCAGCCCTCCATCCACG
CAAACCCTGAGCCTGTGGGCTCCCACAGGAGTGTTGCTACCCAGCCTGGT
GGAGCTTGAATACCCCTTCCAGGCTGGCCGGGGGGCCTCACTCCAGCAGG
AGCTGACAGAGCCCACCTTGGCCCTCAGTGCTGAAAGCCACAGGCCTCCT
GAGCTTCAAGACAGTGTGGAGGGGCTTTCTGAGAGGCCCTCACGCTGAGC
CTCCATGGAGACTCCAGGGTGGACCCCCAGACGCCCACCTCCAGGCAGAC
CCATCACTGGAAGTGGGATTTGAAATGCAGCCAAGAAGAGGAGACCGTCG
GCAACGCCCGCCCGGAACAAGAAATAAATAAATAAATAATCA
(NM_001366227).
Isoform 2 has a coding sequence of SEQ ID NO: 1:
ATGGGGTTGGAGCGGAGTGATCGCTACATAATGAAGTGTCCGATGCTAAGG
TCAAGGCTGGGTCAGGAAAGCGTCCACTGTGGGCCCATGTTCATCCAGGT
CTCCCGGCCCCTGCCCCTGTGGAGGGACAATAGACAGACTCCATGGCTGCT
GTCCCTTCGAGGGGAGCTGGTGGCTTCTCTTGAAGACGCCAGCCTGATGG
GACTGTATGTGGACATGAATGCCACCACTGTCACCGTCCAAAGCCCGAGAC
AAGGCCTTCTTCAGAGGTGGGAGGTGCTGAACACCTCTGCTGAGCTCCTG
CCACTATGGCTGGTGAGCGGTCACCATGCCTATTCTTTAGAAGCTGCTTGCC
CACCGGTGTCATTCCAGCCAGAGTCGGAGGTCTTAGTTCACATCCCCAAGC
AGAGACTGGGTCTAGTCAAAAGAGGTTCCTACATTGAGGAAACCCTGAGC
CTCAGATTCCTCCGAGTCCACCAGTCCAACATCTTTATGGTGACTGAGAAC
AAGGACTTTGTGGTGGTCAGCATTCCGGCGGCCGGGGTGCTCCAGGTCCA
GCGATGCCAAGAAGTCGGAGGAACCCCGGGAACACAAGCTTTCTATAGGG
TAGACCTGAGCCTGGAATTTGCCGAGATGGCTGCCCCGGTCCTCTGGACAG
TGGAGAGCTTCTTCCAGTGTGTGGGTTCAGGAACAGAGTCGCCTGCCTCA
ACTGCTGCACTGAGGACCACTCCCTCCCCACCATCCCCAGGACCAGAGAC
CCCTCCAGCGGGAGTGCCACCTGCTGCTTCCTCCCAGGTGTGGGCTGCAG
GACCAGCTGCCCAGGAATGGCTTTCTCGGGACCTCCTGCACCGGCCTTCCG
ACGCACTGGCCAAAAAGGGGCTTGGACCATTCCTGCAAACAGCCAAACCG
GCGAGAAGAGGCCAGACATCTGCCTCCATTCTCCCCAGAGTGGTGCAAGC
TCAGCGAGGTCCCCAGCCTCCCCCAGGGGAAGCAGGGATCCCTGGACACC
CCACACCTCCAGCCACGCTCCCCTCGGAGCCTGTAGAGGGTGTCCAGGCT
AGTCCCTGGCGGCCACGTCCAGTCTTGCCAACGCACCCGGCTCTGACCCT
GCCCGTGTCCTCAGATGCCTCCTCTCCTTCACCGCCAGCCCCGAGGCCTGA
ACGACCTGAATCACTTCTGGTCTCAGGACCATCTGTCACCCTGACTGAAGG
TCTAGGAACTGTGAGGCCTGAACAGGACCCCGCCAAGTCTCCAGGAAGTC
CCCTCCTGCTGAGAGGCTTGTCAAGCGGGGATGTGGCTGCACCTGAGCCC
ATCATGGGGGAGCCCGGCCAAGCCAGTGAGGAGTTCCAGCCATTGGCGAG
GCCCTGGCGGGCCACACTGGCTGCAGAGGAGCTGGTTTCTCACCGTTCTC
CCGGAGAGCCCCAGGAAACGTGCTCTGGAACGGAGGTGGAGAGGCCACG
CCAGACAGGGCCTGGTCTCCCCAGGGAGGGGGCCAGGGGGCACATGGAC
CTTTCATCCTCAGAACCAAGCCAGGACATAGAGGGGCCGGGACTCTCCATC
CTGCCAGCGAGGGATGCCACATTCTCCACCCCAAGTGTGAGGCAGCCAGA
CCCCAGTGCCTGGCTGAGTTCAGGACCTGAACTCACCGGGATGCCCAGGG
TGAGGCTGGCAGCGCCCCTGGCAGTTCTTCCTATGGAACCTCTGCCACCAG
AACCTGTTCGCCCAGCAGCTCTTCTGACACCCGAAGCCTCATCTGTGGGAG
GGCCAGACCAGGCCCGATACCTGGAGTCAGCCCCTGGCTGGCCTGTGGGC
CAGGAGGAGTGGGGGGTTGCACACACGTCCAGCCCTCCATCCACGCAAAC
CCTGAGCCTGTGGGCTCCCACAGGAGTGTTGCTACCCAGCCTGGTGGAGC
TTGAATACCCCTTCCAGGCTGGCCGGGGGGCCTCACTCCAGCAGGAGCTG
ACAGAGCCCACCTTGGCCCTCAGTGCTGAAAGCCACAGGCCTCCTGAGCT
TCAAGACAGTGTGGAGGGGCTTTCTGAGAGGCCCTCACGC.
Isoform 2 has a protein sequence of SEQ ID NO: 2:
MGLERSDRYIMKCPMLRSRLGQESVHCGPMFIQVSRPLPLWRDNRQTPWLLS
LRGELVASLEDASLMGLYVDMNATTVTVQSPRQGLLQRWEVLNTSAELLPLW
LVSGHHAYSLEAACPPVSFQPESEVLVHIPKQRLGLVKRGSYIEETLSLRFLRV
HQSNIFMVTENKDFVVVSIPAAGVLQVQRCQEVGGTPGTQAFYRVDLSLEFA
EMAAPVLWTVESFFQCVGSGTESPASTAALRTTPSPPSPGPETPPAGVPPAASS
QVWAAGPAAQEWLSRDLLHRPSDALAKKGLGPFLQTAKPARRGQTSASILPR
VVQAQRGPQPPPGEAGIPGHPTPPATLPSEPVEGVQASPWRPRPVLPTHPALTL
PVSSDASSPSPPAPRPERPESLLVSGPSVTLTEGLGTVRPEQDPAKSPGSPLLLR
GLSSGDVAAPEPIMGEPGQASEEFQPLARPWRATLAAEELVSHRSPGEPQETCS
GTEVERPRQTGPGLPREGARGHMDLSSSEPSQDIEGPGLSILPARDATFSTPSV
RQPDPSAWLSSGPELTGMPRVRLAAPLAVLPMEPLPPEPVRPAALLTPEASSVG
GPDQARYLESAPGWPVGQEEWGVAHTSSPPSTQTLSLWAPTGVLLPSLVELEY
PFQAGRGASLQQELTEPTLALSAESHRPPELQDSVEGLSERPSR
(NP_001353156).

In some embodiments, an agent has a nucleotide sequence having at least about 80%, 85%, 90%, 91%, 92%, 93%, 94%, 95%, 96%, 97%, 98%, or 99% identity to SEQ ID NO: 1. In some embodiments, an agent has a nucleotide sequence having at least about 80%, 85%, 90%, 91%, 92%, 93%, 94%, 95%, 96%, 97%, 98%, or 99% identity to SEQ ID NO: 20.

In some embodiments, C1ORF127 gene product has an amino acid sequence having at least about 80%, 85%, 90%, 91%, 92%, 93%, 94%, 95%, 96%, 97%, 98%, or 99% identity to SEQ ID NO: 2. In some embodiments, C1ORF127 gene product has an amino acid sequence having at least about 80%, 85%, 90%, 91%, 92%, 93%, 94%, 95%, 96%, 97%, 98%, or 99% identity to SEQ ID NO: 21.

In some embodiments, the agent comprises a conserved domain of the C1ORF127 gene product or a functional portion or functional variant thereof. In some embodiments, the conserved domain has the amino acid sequence of SEQ ID NO: 3:

KCPMLRSRLGQESVHCGPMFIQVSRPLPLWRDNRQTPWLLSLRGELVAS
LEDASLMGLYVDMNATTVTVQSPRQGLLQRWEVLNTSAELLPLWLVSGH
HAYSLEAACPPVSFQPESEVLVHIPKQRLGLVKRGSYIEETLSLRFLRV
HQSNIFMVTENKDFVVVSIPAAGVLQVQRCQEVGGTPGTQAFYRVDLSL
EFAEMAAPVLWTVESFFQC.

In some embodiments, the agent comprises a portion of the conserved domain corresponding to SEQ ID NO: 4 or a functional portion or functional variant thereof:

GSYIEETLSLRFLRVHQSNIFMVTENKDFVVVSIPAAGVLQVQRCQEVG
GTPGTQAFYRVDLSLEFAEMAAPVLWTVESFFQC.

In some embodiments, the agent comprises an approximately 57 kDa protein produced by the cleavage of the C1ORF127 gene product or a functional portion or functional variant thereof. In some embodiments, the agent is a fusion protein, e.g., an Fc-fusion protein, comprising the 57 kDa protein. The Fc-fusion protein may be an IgGFc-fusion protein. In some embodiments, the approximately 57 kDa protein has the amino acid sequence of SEQ ID NO: 5:

GSYIEETLSLRFLRVHQSNIFMVTENKDFVVVSIPAAGVLQVQRCQEVG
GTPGTQAFYRVDLSLEFAEMAAPVLWTVESFFQCVGSGTESPASTAALR
TTPSPPSPGPETPPAGVPPAASSQVWAAGPAAQEWLSRDLLHRPSDALA
KKGLGPFLQTAKPARRGQTSASILPRVVQAQRGPQPPPGEAGIPGHPTP
PATLPSEPVEGVQASPWRPRPVLPTHPALTLPVSSDASSPSPPAPRPER
PESLLVSGPSVTLTEGLGTVRPEQDPAKSPGSPLLLRGLSSGDVAAPEP
IMGEPGQASEEFQPLARPWRATLAAEELVSHRSPGEPQETCSGTEVERP
RQTGPGLPREGARGHMDLSSSEPSQDIEGPGLSILPARDATFSTPSVRQ
PDPSAWLSSGPELTGMPRVRLAAPLAVLPMEPLPPEPVRPAALLTPEAS
SVGGPDQARYLESAPGWPVGQEEWGVAHTSSPPSTQTLSLWAPTGVLLP
SLVELEYPFQAGRGASLQQELTEPTLALSAESHRPPELQDSVEGLSERP
SR.

In some embodiments, the agent comprises an approximately 55 kDa or 57 kDa protein produced by cleavage of the C1ORF127 gene product or a functional portion or functional variant thereof.

In some embodiments, the agent comprises an approximately 25 or 26 kDa protein produced by cleavage of the C1ORF127 gene product or a functional portion or functional variant thereof. In some embodiments, the agent is a fusion protein, e.g., a Fc-fusion protein, comprising the 26 kDa protein. In some embodiments, the 26 kDa protein is an N-terminal fragment of C1ORF127 fused to the Fc portion of IgG1. In some embodiments, the approximately 26 kDa protein has the amino acid sequence of SEQ ID NO: 6:

KCPMLRSRLGQESVHCGPMFIQVSRPLPLWRDNRQTPWLLSLRGELVAS
LEDASLMGLYVDMNATTVTVQSPRQGLLQRWEVLNTSAELLPLWLVSGH
HAYSLEAACPPVSFQPESEVLVHIPKQRLGLVKRGSYIEETLSLRFLRV
HQSNIFMVTENKDFVVVSIPAAGVLQVQRCQEVGGTPGTQAFYRVDLSL
EFAEMAAPVLWTVESFFQCVGSGTESPASTA.

In some embodiments, an agent comprises a fusion protein (e.g., an approximately 26 kDa C1ORF127 fragment-Fc fusion protein) having the amino acid sequence of SEQ ID NO: 7:

MDAMKRGLCCVLLLCGAVFVSPDYKDDDDKKCPMLRSRLGQESVHCGPM
FIQVSRPLPLWRDNRQTPWLLSLRGELVASLEDASLMGLYVDMNATTVT
VQSPRQGLLQRWEVLNTSAELLPLWLVSGHHAYSLEAACPPVSFQPESE
VLVHIPKQRLGLVKRGSYIEETLSLRFLRVHQSNIFMVTENKDFVVVSI
PAAGVLQVQRCQEVGGTPGTQAFYRVDLSLEFAEMAAPVLWTVESFFQC
VGSGTESPASTAGGGGSEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPGK.

In some embodiments, the agent comprises an approximately 15 kDa protein produced by the cleavage of the C1ORF127 gene product or a functional portion or functional variant thereof. In some embodiments, the agent is an Fc-fusion protein comprising the 15 kDa protein. In some aspects, the Fc-fusion is an IgGFc-fusion protein. The 15 kDa protein may be an N-terminus fragment of C1ORF127. In some embodiments, the approximately 15 kDa protein has the amino acid sequence of SEQ ID NO: 8: YIMKCPMLRSRLGQESVHCGPMFIQVSRPLPLWRDNRQTPWLLSLRGELVASL EDASLMGLYVDMNATTVTVQSPRQGLLQRWEVLNTSAELLPLWLVSGHHAY SLEAACPPVSFQPESEVLVHIPKQRLGLVKR. In some embodiments, the approximately 15 kDa protein has the amino acid sequence of SEQ ID NO: 9: QTPWLLSLRGELVASLEDASLMGLYVDMNATTVTVQSPROGLLQRWEVLNTS AELLPLWLVSGHHAYSLEAACPPVSFQPESEVLVHIPKQRLGLVKRGSYIEETL SLRFLRVHQSNIFMVTENKDFVVVSIPAAGVLQVQR. In some embodiments, the approximately 15 kDa protein is a protein post-translationally modified to form the 15 kDa protein.

In some embodiments, the agent comprises an approximately 5 to 5.5 kDa protein or peptide fragment of the C1ORF127 gene product or a functional portion or functional variant thereof. The 5 kDa protein may be a N-terminal fragment of C10RF127. In some aspects, the approximately 5 to 5.5 kDa protein comprises or consists of 42 to 46 amino acids. In some aspects, the 5 kDa protein has a 45 or 42 amino acid sequence. In some embodiments, the agent comprises a linear peptide (e.g., linear synthetic peptide or linear lipidated synthetic peptide) or cyclic peptide (e.g., disulfide bridged peptide or cyclic lipidated peptide) comprising the approximately 5 kDa protein. The 5 kDa protein may exhibit glucose lowering activity. In some embodiments, the agent is a fusion protein (e.g., an Fc-fusion protein) comprising the 5 kDa protein. The Fc-fusion may be an IgGFc-fusion protein. In some embodiments, the approximately 5 kDa protein has the amino acid sequence of SEQ ID NO: 10: YIMKCPMLRSRLGQESVHCGPMFIQVSRPLPLWRDNRQTPWLLSL (45 amino acids). In some embodiments, the approximately 5 kDa protein has the amino acid sequence of SEQ ID NO: 11: KCPMLRSRLGQESVHCGPMFIQVSRPLPLWRDNRQTPWLLSL (42 amino acids). In some embodiments, the approximately 5.5 kDa protein has the amino acid sequence of SEQ ID NO: 25: YIMKCPMLRSRLGQESVHCGPMFIQVSRPLPLWRDNRQTPWLLSLR (46 amino acids). In some embodiments, the approximately 5 kDa protein has the amino acid sequence of SEQ ID NO: 26: KCPMLRSRLGQESVHCGPMFIQVSRPLPLWRDNRQTPWLLSLR (43 amino acids). In one embodiment, the agent comprises a cyclic peptide comprising SEQ ID NO: 10. In one embodiment, the agent comprises a linear peptide comprising SEQ ID NO: 10. In one embodiment, the agent comprises a cyclic peptide comprising SEQ ID NO: 11. In one embodiment, the agent comprises a linear peptide comprising SEQ ID NO: 11. In one embodiment, the agent comprises a cyclic peptide comprising SEQ ID NO: 25. In one embodiment, the agent comprises a linear peptide comprising SEQ ID NO: 25. In one embodiment, the agent comprises a cyclic peptide comprising SEQ ID NO: 26. In one embodiment, the agent comprises a linear peptide comprising SEQ ID NO: 26. In one embodiment, the agent comprises a recombinant IgGFc-fusion comprising SEQ ID NO: 11. In one embodiment, the agent comprises a recombinant IgGFc-fusion comprising SEQ ID NO: 10. In one embodiment, the agent comprises a recombinant IgGFc-fusion comprising SEQ ID NO: 25. In one embodiment, the agent comprises a recombinant IgGFc-fusion comprising SEQ ID NO: 26.

In one embodiment, an agent comprises a fusion protein (e.g., an approximately 5 kDa C1ORF127 fragment-Fc fusion protein) having an amino acid sequence of SEQ ID NO: 12:

KCPMLRSRLGQESVHCGPMFIQVSRPLPLWRDNRQTPWLLSLEPKSCDK
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQ
QGNVFSCSVMHEALHNHYTQKSLSLSPGK.

In one embodiment, an agent comprises a fusion protein (e.g., an approximately 5 kDa C1ORF127 fragment-Fc fusion protein) having an amino acid sequence of SEQ ID NO: 23:

YIMKCPMLRSRLGQESVHCGPMFIQVSRPLPLWRDNRQTPWLLSLEPKS
CDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKS
RWQQGNVFSCSVMHEALHNHYTQKSLSLSPGK.

In some embodiments, the agent comprises an approximately 4.4 kDa protein produced by the cleavage of the C1ORF127 gene product or a functional portion or functional variant thereof. In some embodiments, the agent is a fusion protein (e.g., an Fc-fusion protein) comprising the 4.4 kDa protein. In some embodiments, the 4.4 kDa protein is a N-terminus fragment of C1ORF127, e.g., synthesized as a linear or cyclic peptide. In some embodiments, the approximately 4.4 kDa protein has the amino acid sequence of SEQ ID NO: 13: YIMKCPMLRSRLGQESVHCGPMFIQVSRPLPLWRDNR. In some embodiments, an isoform of the approximately 4.4 kDa protein has the amino acid sequence of SEQ ID NO: 14: MKCPMLRSRLGQESVHCGPMFIQVSRPLPLWRDNR.

In some embodiments, the agent is a polypeptide comprising a functional fragment of C1ORF127 (e.g., native or naturally occurring C1ORF127) having approximately 10 to 46, 10 to 45, 12 to 40, 15 to 35, 18 to 30, or 20 to 25 amino acids. In some aspects, the agent comprises a functional fragment of C1ORF127 having 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46 amino acids. In some embodiments, the agent is a fusion protein, e.g., an Fc-fusion protein, comprising a functional fragment of C1ORF127 having approximately 10 to 35, 15 to 30, or 20 to 25 amino acids. In some embodiments, the agent is a linear peptide comprising a functional fragment of C1ORF127 having approximately 10 to 35, 15 to 30, or 20 to 25 amino acids. In some embodiments, the agent is a cyclic peptide comprising a functional fragment of C1ORF127 having approximately 10 to 35, 15 to 30, or 20 to 25 amino acids.

In one embodiment, the agent comprises a functional fragment of C1ORF127 having an amino acid sequence listed in Table 1:

| Sequence | SEQ ID NO: |
|---|---|
| QESVHCGPMFIQVSRPLPLWRDNRQTPWLLSLR | SEQ ID NO: 27 |
| QESVHCGPMFIQVSRPLPLWRDNRQTPWLL | SEQ ID NO: 28 |
| QESVHCGPMFIQVSRPLPLWRDNRQTP | SEQ ID NO: 29 |
| QESVHCGPMFIQVSRPLPLWRDNR | SEQ ID NO: 30 |
| QESVHCGPMFIQVSRPLPLWRDN | SEQ ID NO: 31 |
| CPMLRSRLGQESVHCGPMFIQVSRPLPLWRDNR | SEQ ID NO: 32 |
| CPMLRSRLGQESVHCGPMFIQVSRPL | SEQ ID NO: 33 |
| CPMLRSRLGQESVHCGPMFIQVSR | SEQ ID NO: 34 |
| CPMLRSRLGQESVHCGPMFIQV | SEQ ID NO: 35 |
| ESVHCGPMFIQVSRPLPLWRDNRQTPWLL | SEQ ID NO: 36 |
| ESVHCGPMFIQVSRPLPLWRDNRQTP | SEQ ID NO: 37 |
| ESVHCGPMFIQVSRPLPLWRDNR | SEQ ID NO: 38 |
| CGPMFIQVSRPLPLWRDNRQTPWLLSLR | SEQ ID NO: 39 |
| CGPMFIQVSRPLPLWRDNRQTPWLL | SEQ ID NO: 40 |
| CGPMFIQVSRPLPLWRDNRQTPW | SEQ ID NO: 41 |
| SVHCGPMFIQVSRPLPLWRDNRQTPWL | SEQ ID NO: 42 |
| SVHCGPMFIQVSRPLPLWRDNRQTP | SEQ ID NO: 43 |
| SVHCGPMFIQVSRPLPLWRDNR | SEQ ID NO: 44 |

In some embodiments, a fusion protein comprises a signal sequence, a tag (e.g., a flag tag), and/or an Fc (e.g., an IgG Fc). In some embodiments, the signal sequence has the amino acid sequence of SEQ ID NO: 15: MDAMKRGLCCVLLLCGAVFVSP. In some embodiments, the flag tag has the amino acid sequence of SEQ ID NO: 16: DYKDDDDK. In some embodiments, an Fc region has the amino acid sequence of SEQ ID NO: 17: EPKSCDKTHTCPPCPAPELLGGPSVFLFPPKPKDTLMISRTPEVTCVVVDVSHE DPEVKFNWYVDGVEVHNAKTKPREEQYNSTYRVVSVLTVLHQDWLNGKEY KCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCS VMHEALHNHYTQKSLSLSPGK. In some embodiments, an Fc region has the amino acid sequence of SEQ ID NO: 18:

GGGGSEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFL
YSKLTVDKSRWQQGNVFSCSVMHEALHNHYTQKSLSLSPGK.

In some embodiments, a C1ORF127 gene product comprises an amino acid sequence that has at least about 80%, 85%, 90%, 91%, 92%, 93%, 94%, 95%, 96%, 97%, 98%, or 99% identity to one or more of SEQ ID NOs: 2-6, SEQ ID NOs: 8-11, SEQ ID NO: 13, SEQ ID NO: 14, SEQ ID NO: 21, or SEQ ID NOs: 25-44. In some embodiments, a C1ORF127 fusion protein comprises an amino acid sequence that has at least about 80%, 85%, 90%, 91%, 92%, 93%, 94%, 95%, 96%, 97%, 98%, or 99% identity to SEQ ID NO: 7. SEQ ID NO: 12 or SEQ ID NO: 23. In some embodiments, a C1ORF127 linear or cyclic peptide comprises an amino acid sequence that has at least about 80%, 85%, 90%, 91%, 92%, 93%, 94%, 95%, 96%, 97%, 98%, or 99% identity to SEQ ID NO: 10, SEQ ID NO: 11, SEQ ID NO: 25, or SEQ ID NO: 26.

In some embodiments, the agent comprises a functional portion (i.e., functional fragment) of a C1ORF127 gene product. In some embodiments, the agent comprises a functional fragment of C1ORF127 gene product corresponding to SEQ ID NOs: 3-6, SEQ ID NOs: 8-11, SEQ ID NO: 13, SEQ ID NO: 14, or SEQ ID NOS: 25-44 or a polypeptide sequence having at least about 80%, 85%, 90%, 91%, 92%, 93%, 94%, 95%, 96%, 97%, 98%, or 99% identity to SEQ ID NOs: 3-6, SEQ ID NOs: 8-11, SEQ ID NO: 13, SEQ ID NO: 14, or SEQ ID NOs: 25-44.

In certain embodiments, the agent comprises a functional fragment of C1ORF127 gene product corresponding to SEQ ID NO: 10, or a polypeptide sequence having at least about 80%, 85%, 90%, 91%, 92%, 93%, 94%, 95%, 96%, 97%, 98%, or 99% identity to SEQ ID NO: 10. In certain embodiments, the agent comprises a functional fragment of C1ORF127 gene product corresponding to SEQ ID NO: 11, or a polypeptide sequence having at least about 80%, 85%, 90%, 91%, 92%, 93%, 94%, 95%, 96%, 97%, 98%, or 99% identity to SEQ ID NO: 11. In certain embodiments, the agent comprises a functional fragment of C1ORF127 gene product corresponding to SEQ ID NO: 25, or a polypeptide sequence having at least about 80%, 85%, 90%, 91%, 92%, 93%, 94%, 95%, 96%, 97%, 98%, or 99% identity to SEQ ID NO: 25. In certain embodiments, the agent comprises a functional fragment of C1ORF127 gene product corresponding to SEQ ID NO: 26, or a polypeptide sequence having at least about 80%, 85%, 90%, 91%, 92%, 93%, 94%, 95%, 96%, 97%, 98%, or 99% identity to SEQ ID NO: 26. In certain embodiments, the agent comprises a functional fragment of C1ORF127 gene product corresponding to SEQ ID NO: 26, or a polypeptide sequence having at least about 80%, 85%, 90%, 91%, 92%, 93%, 94%, 95%, 96%, 97%, 98%, or 99% identity to SEQ ID NO: 26. In certain embodiments, the agent comprises a functional fragment of C1ORF127 gene product corresponding to SEQ ID NO: 27, or a polypeptide sequence having at least about 80%, 85%, 90%, 91%, 92%, 93%, 94%, 95%, 96%, 97%, 98%, or 99% identity to SEQ ID NO: 27. In certain embodiments, the agent comprises a functional fragment of C1ORF127 gene product corresponding to SEQ ID NO: 28, or a polypeptide sequence having at least about 80%, 85%, 90%, 91%, 92%, 93%, 94%, 95%, 96%, 97%, 98%, or 99% identity to SEQ ID NO: 28. In certain embodiments, the agent comprises a functional fragment of C1ORF127 gene product corresponding to SEQ ID NO: 29, or a polypeptide sequence having at least about 80%, 85%, 90%, 91%, 92%, 93%, 94%, 95%, 96%, 97%, 98%, or 99% identity to SEQ ID NO: 29. In certain embodiments, the agent comprises a functional fragment of C1ORF127 gene product corresponding to SEQ ID NO: 30, or a polypeptide sequence having at least about 80%, 85%, 90%, 91%, 92%, 93%, 94%, 95%, 96%, 97%, 98%, or 99% identity to SEQ ID NO: 30. In certain embodiments, the agent comprises a functional fragment of C1ORF127 gene product corresponding to SEQ ID NO: 32, or a polypeptide sequence having at least about 80%, 85%, 90%, 91%, 92%, 93%, 94%, 95%, 96%, 97%, 98%, or 99% identity to SEQ ID NO: 32. In certain embodiments, the agent comprises a functional fragment of C1ORF127 gene product corresponding to SEQ ID NO: 33, or a polypeptide sequence having at least about 80%, 85%, 90%, 91%, 92%, 93%, 94%, 95%, 96%, 97%, 98%, or 99% identity to SEQ ID NO: 33. In certain embodiments, the agent comprises a functional fragment of C1ORF127 gene product corresponding to SEQ ID NO: 34, or a polypeptide sequence having at least about 80%, 85%, 90%, 91%, 92%, 93%, 94%, 95%, 96%, 97%, 98%, or 99% identity to SEQ ID NO: 34. In certain embodiments, the agent comprises a functional fragment of C1ORF127 gene product corresponding to SEQ ID NO: 35, or a polypeptide sequence having at least about 80%, 85%, 90%, 91%, 92%, 93%, 94%, 95%, 96%, 97%, 98%, or 99% identity to SEQ ID NO: 35. In certain embodiments, the agent comprises a functional fragment of C1ORF127 gene product corresponding to SEQ ID NO: 36, or a polypeptide sequence having at least about 80%, 85%, 90%, 91%, 92%, 93%, 94%, 95%, 96%, 97%, 98%, or 99% identity to SEQ ID NO: 36. In certain embodiments, the agent comprises a functional fragment of C1ORF127 gene product corresponding to SEQ ID NO: 37, or a polypeptide sequence having at least about 80%, 85%, 90%, 91%, 92%, 93%, 94%, 95%, 96%, 97%, 98%, or 99% identity to SEQ ID NO: 37. In certain embodiments, the agent comprises a functional fragment of C1ORF127 gene product corresponding to SEQ ID NO: 38, or a polypeptide sequence having at least about 80%, 85%, 90%, 91%, 92%, 93%, 94° C., 95%, 96%, 97%, 98%, or 99% identity to SEQ ID NO: 38. In certain embodiments, the agent comprises a functional fragment of C1ORF127 gene product corresponding to SEQ ID NO: 39, or a polypeptide sequence having at least about 80%, 85%, 90%, 91%, 92%, 93%, 94%, 95%, 96%, 97%, 98%, or 99% identity to SEQ ID NO: 39. In certain embodiments, the agent comprises a functional fragment of C1ORF127 gene product corresponding to SEQ ID NO: 40, or a polypeptide sequence having at least about 80%, 85%, 90%, 91%, 92%, 93%, 94%, 95%, 96%, 97%, 98%, or 99% identity to SEQ ID NO: 40. In certain embodiments, the agent comprises a functional fragment of C1ORF127 gene product corresponding to SEQ ID NO: 41, or a polypeptide sequence having at least about 80%, 85%, 90%, 91%, 92%, 93%, 94%, 95%, 96%, 97%, 98%, or 99% identity to SEQ ID NO: 41. In certain embodiments, the agent comprises a functional fragment of C1ORF127 gene product corresponding to SEQ ID NO: 42, or a polypeptide sequence having at least about 80%, 85%, 90%, 91%, 92%, 93%, 94%, 95%, 96%, 97%, 98%, or 99% identity to SEQ ID NO: 42. In certain embodiments, the agent comprises a functional fragment of C1ORF127 gene product corresponding to SEQ ID NO: 43, or a polypeptide sequence having at least about 80%, 85%, 90%, 91%, 92%, 93%, 94%, 95%, 96%, 97%, 98%, or 99% identity to SEQ ID NO: 43. In certain embodiments, the agent comprises a functional fragment of C1ORF127 gene product corresponding to SEQ ID NO: 44, or a polypeptide sequence having at least about 80%, 85%, 90%, 91%, 92%, 93%, 94%, 95%, 96%, 97%, 98%, or 99% identity to SEQ ID NO: 44.

In some embodiments, the agent comprises a nucleotide sequence that codes for a C1ORF127 gene product of SEQ ID NOs: 2-6, SEQ ID NOs: 8-11, SEQ ID NO: 13, SEQ ID NO: 14, SEQ ID NO: 21, or SEQ ID NOs: 25-44 or a functional portion or functional variant thereof. In one embodiment, the agent comprises a nucleotide sequence that codes for a C1ORF127 fusion protein of SEQ ID NO: 7. In one embodiment, the agent comprises a nucleotide sequence that codes for a C1ORF127 fusion protein of SEQ ID NO: 12. In one embodiment, the agent comprises a nucleotide sequence that codes for a C1ORF127 fusion protein of SEQ ID NO: 23. In one embodiment, the agent comprises a nucleotide sequence that codes for a C1ORF127 linear or cyclic peptide comprising SEQ ID NO: 10. In one embodiment, the agent comprises a nucleotide sequence that codes for a C1ORF127 linear or cyclic peptide comprising SEQ ID NO: 11. In one embodiment, the agent comprises a nucleotide sequence that codes for a C1ORF127 linear or cyclic peptide comprising SEQ ID NO: 25. In one embodiment, the agent comprises a nucleotide sequence that codes for a C1ORF127 linear or cyclic peptide comprising SEQ ID NO: 26. In some embodiments, the agent comprises a nucleotide sequence that codes for a polypeptide that has at least about 80%, 85%, 90%, 91%, 92%, 93%, 94%, 95%, 96%, 97%, 98%, or 99% identity to SEQ ID NOs: 2-6, SEQ ID NOs: 8-11, SEQ ID NO: 13, SEQ ID NO: 14, SEQ ID NO: 21, or SEQ ID NOs: 25-44 or a functional portion or functional variant thereof.

In some embodiments, the agent comprises a C1ORF127 gene product (e.g., C1ORF127 protein). In some embodiments, the C1ORF127 gene product is a fusion protein, e.g., an IgGFc-fusion protein. In some embodiments, the agent is a fragment of the C1ORF127 gene product. In some embodiments, the agent is a variant of the C1ORF127 gene product. In some embodiments, the fragment of the C1ORF127 gene product is a N-terminus fragment. In some embodiments, the fragment or variant of the C1ORF127 gene product is about 57 kDa in size. In some embodiments, the fragment or the variant of the C1ORF127 gene product is about 25 or 26 kDa in size. In some embodiments, the fragment or the variant of the C1ORF127 gene product is about 15 kDa in size. In some embodiments, the fragment or the variant of the C1ORF127 gene product is about 5 kDa in size. In some embodiments, the fragment or the variant of the C1ORF127 gene product is about 4.4 kDa in size. In some embodiments, the fragment or the variant of the C1ORF127 gene product is about 10 to 50 amino acids in length, e.g., is about 15 to 45, 20 to 40, 25 to 35 or 20, 21, 22, 23, 24, 25, 26, 27, 28, 29, 30, 31, 32, 33, 34, 35, 36, 37, 38, 39, 40, 41, 42, 43, 44, or 45 amino acids in length. In some embodiments, C1ORF127 or a fragment or variant thereof is fused to an Fc. In some embodiments, C1ORF127 or a fragment or variant thereof is fused to an IgG (e.g., IgG1 or lgG4). The IgG may be modified to become less immunogenic. In some embodiments, C1ORF127 or a fragment or variant thereof is fused to a Fc portion of IgG1. In some embodiments, C1ORF127 or a fragment or variant thereof is formed into a linear or cyclic peptide. For example, cysteines may be used to facilitate cyclization of the fragment.

In some embodiments, the agent comprises a C1ORF127 57 kDa fragment-fusion protein, e.g., a 57 kDa Fc-fusion protein. In some embodiments, the agent comprises a C1ORF127 26 kDa fragment-fusion protein, e.g., a 26 kDa Fc-fusion protein. In some embodiments, the agent comprises a C1ORF127 15 kDa fragment-fusion protein, e.g., a 15 kDa Fc-fusion protein. In some embodiments, the agent comprises a C1ORF127 5 kDa fragment-fusion protein, e.g., a 5 kDa Fc-fusion protein. In some embodiments, the agent comprises a C1ORF127 4.4 kDa fragment-fusion protein, e.g., a 4.4 kDa Fc-fusion protein.

In some embodiments, the agent comprises a linear (e.g., linear synthetic peptide or linear lipidated synthetic peptide) or cyclic peptide (e.g., cyclic lipidated peptide). In some embodiments, the fragment of the C1ORF127 gene product is formed into a linear or cyclic peptide. In some embodiments, the fragment of the C1ORF127 gene product is formed into a cyclic-disulfide bonded peptide. For example, the cysteines at positions 5 and 19 of SEQ ID NO: 10 may be used to facilitate formation of the cyclic peptide.

In some embodiments, the agent is a variant (e.g., a functional variant) of a C1ORF127 gene product. For example, the agent may be a variant of C1ORF127 or a functional fragment thereof, such as a variant of SEQ ID NOs: 2-6, SEQ ID NOs: 8-11, SEQ ID NO: 13, SEQ ID NO: 14, SEQ ID NO: 21, or SEQ ID NOs: 25-44. In some embodiments, the variant is a conservative substitution variant. Variants can be obtained by mutations of native nucleotide sequences, for example. A “variant.” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encodes a variant protein or fragment thereof that retains the relevant biological activity relative to the reference protein, e.g., can facilitate the clearance of glucose at least 50% as well as C1ORF127 or a functional fragment thereof. As to amino acid sequences, one of skill will recognize that individual substitutions, deletions or additions to a nucleic acid, peptide, polypeptide, or protein sequence which alters a single amino acid or a small percentage, (e.g., 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wild type C1ORF127 or a functional fragment thereof, e.g. 110%, 125%, 150%, 175%, 200%, 500%, 1000% or more.

One method of identifying amino acid residues which can be substituted is to conduct an alignment analysis. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence or a nucleic acid encoding one of those amino acid sequences. The degree of homology (percent identity) between a native and a variant sequence can be determined, for example, by comparing the two sequences using freely available computer programs commonly employed for this purpose on the World Wide Web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available at blast.ncbi.nlm.nih.gov), with default parameters set.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired apoptotic activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K): 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. In some embodiments, a C1ORF127 polypeptide or functional fragment thereof as described herein can be chemically synthesized and mutations can be incorporated as part of the chemical synthesis process.

The peptides of the present invention can be synthesized by using well known methods including recombinant methods and chemical synthesis. Recombinant methods of producing a peptide through the introduction of a vector including nucleic acid encoding the peptide into a suitable host cell is well known in the art, such as is described in Sambrook et al., Molecular Cloning: A Laboratory Manual, 2d Ed, Vols 1 to 8, Cold Spring Harbor, NY (1989); M. W. Pennington and B. M. Dunn, Methods in Molecular Biology: Peptide Synthesis Protocols, Vol 35, Hurnana Press, Totawa, NJ (1994), contents of both of which are herein incorporated by reference. Peptides can also be chemically synthesized using methods well known in the art. See for example, Merrifield et al., J. Am. Chern. Soc. 85:2149 (1964); Bodanszky, M., Principles of Peptide Synthesis, Springer-Verlag, New York, NY (1984); Kirnrnerlin, T. and Seebach, D. J. Pept. Res. 65:229-260 (2005); Nilsson et al., Annu. Rev. Biophys. Biornol. Struct. (2005) 34:91-118; W. C. Chan and P. D. White (Eds.) Frnoc Solid Phase Peptide Synthesis: A Practical Approach, Oxford University Press, Cary, NC (2000); N. L. Benoiton, Chemistry of Peptide Synthesis, CRC Press, Boca Raton, FL (2005); J. Jones, Amino Acid and Peptide Synthesis, 2nd Ed, Oxford University Press, Cary, NC (2002); and P. Lloyd-Williams, F. Albericio, and E. Giralt, Chemical Approaches to the synthesis of pep tides and proteins, CRC Press, Boca Raton, FL (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

In some embodiments, the agent is a C1ORF127 gene product having at least one different post-translational modification than a native C1ORF127 gene product or sequence. Such modifications include, but are not limited to, acetylation, carboxylation, glycosylation (e.g., O-linked oligosaccharides, N-linked oligosaccharides, etc.), phosphorylation, lipidation, and acylation. In some embodiments, the agent comprises a C1ORF127 gene product having at least one substituted, deleted, or added amino acid than a native C1ORF127 sequence. In some embodiments, the C1ORF127 gene product has 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more substituted, deleted, or added amino acids than a native C1ORF127 sequence. In some embodiments, the C1ORF127 gene product has 1, 2, 3, 4, or 5 less cysteines than native C1ORF127 sequence.

In some embodiments, the C1ORF127 gene product is differently phosphorylated than naturally occurring C1ORF127 gene product or sequence. In some embodiments, the C1ORF127 gene product has one less phosphorylation than naturally occurring C1ORF127 sequence. In some embodiments, the C1ORF127 gene product has one more phosphorylation than naturally occurring C1ORF127 gene product or sequence.

In some embodiments, the C1ORF127 gene product is differently glycosylated than naturally occurring C1ORF127 gene product or sequence. In some embodiments, the C1ORF127 gene product has one less glycosylation than naturally occurring C1ORF127 gene product. In some embodiments, the C1ORF127 gene product has two less glycosylations than naturally occurring C1ORF127 gene product. In some embodiments, the C1ORF127 gene product has one more glycosylation than naturally occurring C1ORF127 gene product. In some embodiments, the C1ORF127 gene product has two more glycosylations than naturally occurring C1ORF127 gene product.

In some embodiments, one or more modifications to the C1ORF127 gene product occur at E15 and/or D35 with reference to SE ID NO: 26.

In some embodiments, the C1ORF127 gene product is a dimer, trimer, or multimer. In some embodiments, the C1ORF127 gene product is a homodimer, homotrimer, or homomultimer. In some embodiments, the C1ORF127 gene product exists in a different multimerization state than naturally occurring C1ORF127 gene product.

In some embodiments, the agent comprises a C1ORF127 gene product comprising a furin cleavage site. In some embodiments, the agent comprises a C1ORF127 gene product without a PC2 cleavage site. In some embodiments, the agent comprises a C1ORF127 gene product with a furin cleavage site and without a PC2 cleavage site.

In some embodiments, the agent is a variant of a C1ORF127 gene product having one, two, three or four of the following fixed amino acids: G20, S27, W41, and L42 (with reference to SEQ ID NO: 25). In some embodiments, the agent is a variant of a C1ORF127 gene product having one or more (including all) of the following fixed amino acids: G20, S27, W41, L42 and L43 (with reference to SEQ ID NO: 25). In some embodiments, the agent is a variant of a C1ORF127 gene product having one or more (including all) of the following fixed amino acids: G20, S27, L30, R34 W41 and L42 (with reference to SEQ ID NO: 25). In some embodiments, the agent is a variant of a C1ORF127 gene product having one or more (including all) of the following fixed amino acids: G20, V26, S27, L30, W41, L42 and R46 with reference to SEQ ID NO: 25. In some embodiments, the agent is a variant of a C1ORF127 gene product having one or more (including all) of the following fixed amino acids: C5, P6, C19, G20, V26, S27, R28, L30, W41, L42, L43, L45 and R46 with reference to SEQ ID NO: 25. In certain embodiments when the variant has the fixed amino acid positions referenced above, it may also have at least 80%, at least 85%, at least 90%, at least 91%, at least 92%, at least 93%, at least 94%, at least 95%, at least 96%, at least 97%, at least 98%, or at least 99% amino acid identity to a corresponding fragment of C1ORF127 (e.g., to SEQ ID NO: 25), and retain one or more functions of C1ORF127 (e.g., retain the ability to clear glucose from the blood). The variant of a C1ORF127 gene product may have an amino acid sequence of, e.g., 20 to 46, 20-40, etc., amino acids and retain at least one function or activity of C1ORF127, e.g., the ability to clear glucose effectively.

In one embodiment, a variant of a C1ORF127 gene product has an amino acid sequence of XXXXCPXXXXXXXXXXXXCGXXXXXVSRXLXXXXXXXXXXWLLXLR (SEQ ID NO: 45). In some embodiments, a variant of a C1ORF127 gene product has an amino acid sequence of SEQ ID NO: 45, has at least 80%, at least 85%, at least 90%, at least 91%, at least 92%, at least 93%, at least 94%, at least 95%, at least 96%, at least 97%, at least 98%, or at least 99% amino acid identity to a corresponding fragment of C1ORF127 (e.g., to SEQ ID NO: 25, to SEQ ID NO: 38, etc.), and retains one or more functions of C1ORF127 (e.g., retains the ability to clear glucose from the blood).

In some embodiments, the agent is a dual agent. For example, a C1ORF127 gene product or functional fragment or variant thereof may be fused to a second peptide. In some aspects, a C1ORF127 gene product or functional fragment or variant thereof may be fused to GLP-1 or GIP receptor agonist or other peptide. In other embodiments the C1ORF127 gene product or functional fragment or variant thereof can be co-administered with a second peptide, either in the same or separate preparations, and concurrently or sequentially.

In some embodiments, the agent comprises a C1ORF127 gene product having a different composition, activity or activity level than native (naturally occurring) C1ORF127 gene product. In some aspects, the agent exhibits at least one of: improved blood glucose clearance activity/glucose lowering activity, enhanced glucose uptake, enhanced or and/or enabled glucose disposal, improved glycemia, reduction in glycemia, reduction of hepatic glucose production, reduction in aged associated weight gain, and/or reduction in cholesterol levels. In some embodiments, the agent directs or transports glucose into adipose tissue, e.g., white adipose tissue. The agent may facilitate glucose burning in the adipose tissue, e.g., the glucose may be converted to lactate. In some embodiments, the agent directs or transports glucose into adipose tissue without promoting fat storage. In some embodiments, the agent mitigates weight gain (e.g., age related weight gain) and/or elevated cholesterol levels. In some embodiments, the agent promotes glucose oxidation. In some embodiments, the agent does not increase insulin secretion and/or does not cause lipogenesis.

In some embodiments, the agent comprising a C1ORF127 gene product has a blood glucose clearance activity that is about 1.1-fold, 1.2-fold, 1.3-fold, 1.4-fold, 1.5-fold, 1.6-fold, 1.7-fold, 1.8-fold, 2.0-fold, 2.5-fold, 3.0-fold, or more than a native C1ORF127 gene product. In some embodiments, the agent comprising a C1ORF127 gene product has a blood glucose clearance activity that is about 1%, 2.5%, 5%, 7.5%, 10%, 20%, 30%, 40%, 50% or less than a native C1ORF127 gene product.

In some embodiments, the agent is a cell expressing a C1ORF127 gene product, e.g., a C1ORF127 gene product having an amino acid sequence that has at least about 80%, 85%, 90%, 91%, 92%, 93%, 94%, 95%, 96%, 97%, 98%, 99%, or 100% identity to SEQ ID NOs: 2-6, SEQ ID NOs: 8-11, SEQ ID NO: 13, SEQ ID NO: 14, SEQ ID NO: 21, or SEQ ID NOs: 25-44. In some embodiments, the agent is a cell expressing a C1ORF127 fusion protein. In some embodiments, the agent is a cell expressing a C1ORF127 fusion protein, e.g., a C1ORF127 fusion protein having an amino acid sequence that has at least about 80%, 85%, 90%, 91%, 92%, 93%, 94%, 95%, 96%, 97%, 98%, 99%, or 100% identity to SEQ ID NO: 7, SEQ ID NO: 12, or SEQ ID NO: 23. In some embodiments, the cell is an islet cell. In some embodiments, the cell is an enteroendocrine cell. In some embodiments, the cell is a beta cell. In some embodiments, the cell is a pancreatic delta cell. In some embodiments, the cell is a skeletal muscle cell. In some embodiments, the cell is autologous to the subject requiring treatment. In some embodiments, the cell is stem cell derived. In some embodiments, the stem cell derived cell is a stem cell derived beta cell. Methods of deriving beta cells are taught in the art. See, e.g., WO 2015/002724 published Jan. 8, 2015, herein incorporated by reference in its entirety. In some embodiments, the cell is encased in a microcapsule or semi-permeable membrane.

Aspects of the disclosure involve microcapsules comprising isolated populations of cells described herein, e.g., cells expressing a C1ORF127 gene product or functional variant or functional fragment thereof. Microcapsules are well known in the art. Suitable examples of microcapsules are described in the literature (e.g., Jahansouz et al., “Evolution of β-Cell Replacement Therapy in Diabetes Mellitus: Islet Cell Transplantation” Journal of Transplantation 2011; Volume 2011, Article ID 247959; Orive et al., “Application of cell encapsulation for controlled delivery of biological therapeutics”, Advanced Drug Delivery Reviews (2013), http://dx.doi.org/10.1016/j.addr.2013.07.009; Hernandez et al., “Microcapsules and microcarriers for in situ cell delivery”, Advanced Drug Delivery Reviews 2010; 62:711-730; Murua et al., “Cell microencapsulation technology: Towards clinical application”, Journal of Controlled Release 2008; 132:76-83; and Zanin et al., “The development of encapsulated cell technologies as therapies for neurological and sensory diseases”. Journal of Controlled Release 2012; 160:3-13). Microcapsules can be formulated in a variety of ways. Exemplary microcapsules comprise an alginate core surrounded by a polycation layer covered by an outer alginate membrane. The polycation membrane forms a semipermeable membrane, which imparts stability and biocompatibility. Examples of polycations include, without limitation, poly-L-lysine, poly-L-ornithine, chitosan, lactose modified chitosan, and photopolymerized biomaterials. In some embodiments, the alginate core is modified, for example, to produce a scaffold comprising an alginate core having covalently conjugated oligopeptides with an RGD sequence (arginine, glycine, aspartic acid). In some embodiments, the alginate core is modified, for example, to produce a covalently reinforced microcapsule having a chemoenzymatically engineered alginate of enhanced stability. In some embodiments, the alginate core is modified, for example, to produce membrane-mimetic films assembled by in-situ polymerization of acrylate functionalized phospholipids. In some embodiments, microcapsules are composed of enzymatically modified alginates using epimerases. In some embodiments, microcapsules comprise covalent links between adjacent layers of the microcapsule membrane. In some embodiment, the microcapsule comprises a subsieve-size capsule comprising alginate coupled with phenol moieties. In some embodiments, the microcapsule comprises a scaffold comprising alginate-agarose. In some embodiments, the cell is modified with PEG before being encapsulated within alginate. In some embodiments, the isolated populations of cells are encapsulated in photoreactive liposomes and alginate. It should be appreciated that the alginate employed in the microcapsules can be replaced with other suitable biomaterials, including, without limitation, PEG, chitosan, PES hollow fibers, collagen, hyaluronic acid, dextran with RGD, EHD and PEGDA, PMBV and PVA, PGSAS, agarose, agarose with gelatin, PLGA, and multilayer embodiments of these.

### Methods of Treatment or Prevention

Some aspects of the disclosure are related to compositions and methods for the prevention, or in some aspects the treatment, of one or more metabolic diseases or disorders, obesity, weight gain and/or hepatosteatosis. In some embodiments, the disclosure is related to compositions and methods for treating or preventing a disorder associated with elevated blood glucose levels in a subject. For example, the compositions and/or agents may lower blood glucose levels. In some embodiments, the disclosure is related to compositions and methods for treating or preventing obesity in a subject. For example, the compositions or agents may burn fat and/or inhibit weight gain in a subject.

The compositions and methods disclosed herein are useful for the prevention, or in certain instances the treatment, of obesity, weight gain, hepatosteatosis and/or one or more metabolic diseases. As used herein, the term “metabolic disease” generally refers to disorders affecting a subject in which errors of metabolism, imbalances in metabolism, or suboptimal metabolism occur and include, for example, impaired glucose tolerance, insulin resistance, diabetes mellitus and/or hepatosteatosis.

In certain aspects, the compositions and methods disclosed herein may be used to treat one or more metabolic diseases selected from the group consisting of Type I diabetes, Type II diabetes, gestational diabetes, insulin resistance, metabolic syndrome, obesity, impaired glucose tolerance, impaired fasting glucose, and hepatosteatosis. In certain aspects, the metabolic disease is diabetes. In certain aspects, the metabolic disease is hepatosteatosis. In certain aspects, the metabolic disease is metabolic dysfunction-associated steatohepatitis.

In some embodiments, the agent comprises a small molecule, a protein, or a nucleic acid. In particular aspects, desirable agents (e.g., compounds) comprise a functional fragment of native C1ORF127 gene product. Suitable compounds/agents include, but are not limited to, chemical compounds and mixtures of chemical compounds, e.g., small organic or inorganic molecules; saccharides; oligosaccharides; polysaccharides; biological macromolecules, e.g., peptides, proteins, and peptide analogs and derivatives; peptidomimetics; nucleic acids; nucleic acid analogs and derivatives; extracts made from biological materials such as bacteria, plants, fungi, or animal cells or tissues; naturally occurring or synthetic compositions; peptides; aptamers; and antibodies, or fragments thereof.

A compound/agent can be a nucleic acid RNA or DNA and can be either single or double stranded. Example nucleic acid compounds include, but are not limited to, a nucleic acid encoding a protein activator or inhibitor (e.g., transcriptional activators or inhibitors), oligonucleotides, nucleic acid analogues (e.g., peptide-nucleic acid (PNA), pseudo-complementary PNA (pc-PNA), locked nucleic acid (LNA) etc.), antisense molecules, ribozymes, small inhibitory or activating nucleic acid sequences (e.g., RNAi, shRNAi, siRNA, micro RNAi (mRNAi), antisense oligonucleotides etc.).

A protein and/or peptide agent can be any protein that modulates gene expression or protein activity. Non-limiting examples include mutated proteins; therapeutic proteins and truncated proteins, e.g., wherein the protein is normally absent or expressed at lower levels in the target cell. Proteins can also be selected from genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. A compound or agent that increases expression of a gene or increases the level or activity of a protein encoded by a gene is also known as an activator or activating compound. A compound or agent that decreases expression of a gene or decreases the level or activity of a protein encoded by a gene is also known as an inhibitor or inhibiting compound.

In some embodiments, the agent comprises a small molecule, a protein, or a nucleic acid. In some embodiments, the agent comprises C1ORF127 or a functional portion, fragment or variant thereof as described herein. In some embodiments, an agent comprises a C1ORF127 gene product having an amino acid sequence selected from SEQ ID NOs: 2-6, SEQ ID NOs: 8-11, SEQ ID NO: 13, SEQ ID NO: 14, SEQ ID NO: 21, or SEQ ID NOs: 25-44. In some embodiments, an agent has a nucleotide sequence having at least about 80%, 85%, 90%, 91%, 92%, 93%, 94%, 95%, 96%, 97%, 98%, or 99% identity to SEQ ID NO: 1 or SEQ ID NO: 22. In some embodiments, an agent comprises a C1ORF127 gene product having an amino acid sequence that has at least about 80%, 85%, 90%, 91%, 92%, 93%, 94%, 95%, 96%, 97%, 98%, or 99% identity to SEQ ID NOs: 2-6, SEQ ID NOs: 8-11, SEQ ID NO: 13, SEQ ID NO: 14, SEQ ID NO: 21, or SEQ ID NOs: 25-44. In some embodiments, an agent comprises a C1ORF127 fusion protein having an amino acid sequence that has at least about 80%, 85%, 90%, 91%, 92%, 93%, 94%, 95%, 96%, 97%, 98%, or 99% identity to SEQ ID NO: 7, SEQ ID NO: 12 or SEQ ID NO: 23. In some embodiments, an agent comprises a C1ORF127 linear or cyclic peptide comprising an amino acid sequence that has at least about 80%, 85%, 90%, 91%, 92%, 93%, 94%, 95%, 96%, 97%, 98%, or 99% identity to SEQ ID NO: 10, SEQ ID NO: 11, or SEQ ID NO: 26.

In some embodiments, the agent comprises a nucleotide sequence that codes for a C1ORF127 gene product of SEQ ID NOs: 2-6, SEQ ID NOs: 8-11, SEQ ID NO: 13, SEQ ID NO: 14, SEQ ID NO: 21, or SEQ ID NOs: 25-44 or a functional portion or functional variant thereof. In some embodiments, the agent comprises a fusion protein comprising a nucleotide sequence that codes for a C1ORF127 gene product of SEQ ID NOs: 2-6, SEQ ID NOs: 8-11, SEQ ID NO: 13, SEQ ID NO: 14, SEQ ID NO: 21, or SEQ ID NOs: 25-44 or a functional portion or functional variant thereof. In one embodiment, the agent comprises a nucleotide sequence that codes for a C1ORF127 fusion protein of SEQ ID NO: 7. In one embodiment, the agent comprises a nucleotide sequence that codes for a C1ORF127 fusion protein of SEQ ID NO: 12. In one embodiment, the agent comprises a nucleotide sequence that codes for a C1ORF127 fusion protein of SEQ ID NO: 23. In one embodiment, the agent comprises a nucleotide sequence that codes for a C1ORF127 cyclic peptide comprising SEQ ID NO: 10. In one embodiment, the agent comprises a nucleotide sequence that codes for a C1ORF127 cyclic peptide comprising SEQ ID NO: 11. In some embodiments, the agent comprises a nucleotide sequence that codes for a polypeptide that has at least about 80%, 85%, 90%, 91%, 92%, 93%, 94%, 95%, 96%, 97%, 98%, or 99% identity to SEQ ID NOs: 2-6, SEQ ID NOs: 8-11, SEQ ID NO: 13, SEQ ID NO: 14, SEQ ID NO: 21, or SEQ ID NOs: 25-44 or a functional portion or functional variant thereof.

In some embodiments, the agent comprises a C1ORF127 gene product (e.g., C1ORF127 protein). In some embodiments, the C1ORF127 gene product is a fusion protein, e.g., an IgGFc-fusion protein. In some embodiments, the C1ORF127 gene product is a fragment of the C1ORF127 gene product. In some embodiments, the fragment of the C1ORF127 gene product is a N-terminus fragment. In some embodiments, the fragment of the C1ORF127 gene product is about 57 kDa in size. In some embodiments, the fragment of the C1ORF127 gene product is about 25 or 26 kDa in size. In some embodiments, the fragment of the C1ORF127 gene product is about 15 kDa in size. In some embodiments, the fragment of the C1ORF127 gene product is about 5 kDa in size. In some embodiments, the fragment of the C1ORF127 gene product is about 4.4 kDa in size. In some embodiments, the fragment of the C1ORF127 gene product is about 10 to 50 amino acids in length, e.g., is about 15 to 45, 20 to 40, 25 to 35 or 20, 21, 22, 23, 24, 25, 26, 27, 28, 29, 30, 31, 32, 33, 34, 35, 36, 37, 38, 39, 40, 41, 42, 43, 44, or 45 amino acids in length. In some embodiments, the fragment of the C1ORF127 is fused to an Fc. In some embodiments, the fragment of the C1ORF127 gene product is fused to an IgG (e.g., IgG1 or IgG4). In some embodiments, the fragment of the C1ORF127 gene product is fused to a Fc portion of IgG1. In some embodiments, the fragment of the C1ORF127 gene product is a linear or cyclic peptide. For example, cysteines may be used to facilitate cyclization of the fragment. In some embodiments, the agent comprises a C1ORF127 57 kDa fusion protein, e.g., a 57 kDa Fc-fusion protein. In some embodiments, the agent comprises a C1ORF127 26 kDa fusion protein, e.g., a 26 kDa Fc-fusion protein. In some embodiments, the agent comprises a C1ORF127 15 kDa fusion protein, e.g., a 15 kDa Fc-fusion protein. In some embodiments, the agent comprises a C1ORF127 5 kDa fusion protein, e.g., a 5 kDa Fc-fusion protein. In some embodiments, the agent comprises a C1ORF127 4.4 kDa fusion protein, e.g., a 4.4 kDa Fc-fusion protein.

In some embodiments, the agent comprises a linear (e.g., linear synthetic peptide or linear lipidated synthetic peptide) or cyclic peptide. In some embodiments, the fragment of the C1ORF127 gene product is formed into a cyclic peptide. In some embodiments, the fragment of the C1ORF127 gene product is formed into a cyclic-disulfide bonded peptide. For example, the cysteines at positions 5 and 19 of SEQ ID NO: 10 may be used to facilitate formation of the cyclic peptide.

In some embodiments, the agent comprises a C1ORF127 gene product having a different composition, activity or activity level than native C1ORF127 gene product. In some aspects, the agent exhibits at least one of: improve blood glucose clearance activity/glucose lowering activity, improved glycemia, reduction in glycemia, reduction of hepatic glucose production, reduction in aged associated weight gain, and reduction in cholesterol levels. In some embodiments, the agent comprising a C1ORF127 gene product has a blood glucose clearance activity that is about 1.1-fold, 1.2-fold, 1.3-fold, 1.4-fold, 1.5-fold, 1.6-fold, 1.7-fold, 1.8-fold, 2.0-fold, 2.5-fold, 3.0-fold, or more than a native C1ORF127 gene product. In some embodiments, the agent comprising a C1ORF127 gene product has a blood glucose clearance activity that is about 1%, 2.5%, 5%, 7.5%, 10%, 20%, 30%, 40%, 50% or less than a native C1ORF127 gene product.

In some embodiments, the agent comprises a functional portion (i.e., functional fragment) of a C1ORF127 gene product. In some embodiments, the agent comprises a functional fragment of C1ORF127 gene product corresponding to SEQ ID NOs: 3-6, SEQ ID NOs: 8-11, SEQ ID NO: 13, SEQ ID NO: 14, or SEQ ID NOs: 25-44 or a polypeptide sequence having at least about 80%, 85%, 90%, 91%, 92%, 93%, 94%, 95%, 96%, 97%, 98%, or 99% identity to SEQ ID NOs: 3-6, SEQ ID NOs: 8-11, SEQ ID NO: 13, SEQ ID NO: 14, or SEQ ID NOs: 25-44. In certain embodiments, the agent comprises a functional fragment of C1ORF127 gene product corresponding to SEQ ID NO: 10, or a polypeptide sequence having at least about 80%, 85%, 90%, 91%, 92%, 93%, 94%, 95%, 96%, 97%, 98%, or 99% identity to SEQ ID NO: 10. In certain embodiments, the agent comprises a functional fragment of C1ORF127 gene product corresponding to SEQ ID NO: 11, or a polypeptide sequence having at least about 80%, 85%, 90%, 91%, 92%, 93%, 94%, 95%, 96%, 97%, 98%, or 99% identity to SEQ ID NO: 11. In certain embodiments, the agent comprises a functional fragment of C1ORF127 gene product corresponding to SEQ ID NO: 25, or a polypeptide sequence having at least about 80%, 85%, 90%, 91%, 92%, 93%, 94%, 95%, 96%, 97%, 98%, or 99% identity to SEQ ID NO: 25. In certain embodiments, the agent comprises a functional fragment of C1ORF127 gene product corresponding to SEQ ID NO: 26, or a polypeptide sequence having at least about 80%, 85%, 90%, 91%, 92%, 93%, 94%, 95%, 96%, 97%, 98%, or 99% identity to SEQ ID NO: 26. In certain embodiments, the agent comprises a functional fragment of C1ORF127 gene product corresponding to SEQ ID NO: 26, or a polypeptide sequence having at least about 80%, 85%, 90%, 91%, 92%, 93%, 94%, 95%, 96%, 97%, 98%, or 99% identity to SEQ ID NO: 26. In certain embodiments, the agent comprises a functional fragment of C1ORF127 gene product corresponding to SEQ ID NO: 27, or a polypeptide sequence having at least about 80%, 85%, 90%, 91%, 92%, 93%, 94%, 95%, 96%, 97%, 98%, or 99% identity to SEQ ID NO: 27. In certain embodiments, the agent comprises a functional fragment of C1ORF127 gene product corresponding to SEQ ID NO: 28, or a polypeptide sequence having at least about 80%, 85%, 90%, 91%, 92%, 93%, 94%, 95%, 96%, 97%, 98%, or 99% identity to SEQ ID NO: 28. In certain embodiments, the agent comprises a functional fragment of C1ORF127 gene product corresponding to SEQ ID NO: 29, or a polypeptide sequence having at least about 80%, 85%, 90%, 91%, 92%, 93%, 94%, 95%, 96%, 97%, 98%, or 99% identity to SEQ ID NO: 29. In certain embodiments, the agent comprises a functional fragment of C1ORF127 gene product corresponding to SEQ ID NO: 30, or a polypeptide sequence having at least about 80%, 85%, 90%, 91%, 92%, 93%, 94%, 95%, 96%, 97%, 98%, or 99% identity to SEQ ID NO: 30. In certain embodiments, the agent comprises a functional fragment of C1ORF127 gene product corresponding to SEQ ID NO: 32, or a polypeptide sequence having at least about 80%, 85%, 90%, 91%, 92%, 93%, 94%, 95%, 96%, 97%, 98%, or 99% identity to SEQ ID NO: 32. In certain embodiments, the agent comprises a functional fragment of C1ORF127 gene product corresponding to SEQ ID NO: 33, or a polypeptide sequence having at least about 80%, 85%, 90%, 91%, 92%, 93%, 94%, 95%, 96%, 97%, 98%, or 99% identity to SEQ ID NO: 33. In certain embodiments, the agent comprises a functional fragment of C1ORF127 gene product corresponding to SEQ ID NO: 34, or a polypeptide sequence having at least about 80%, 85%, 90%, 91%, 92%, 93%, 94%, 95%, 96%, 97%, 98%, or 99% identity to SEQ ID NO: 34. In certain embodiments, the agent comprises a functional fragment of C1ORF127 gene product corresponding to SEQ ID NO: 35, or a polypeptide sequence having at least about 80%, 85%, 90%, 91%, 92%, 93%, 94%, 95%, 96%, 97%, 98%, or 99% identity to SEQ ID NO: 35. In certain embodiments, the agent comprises a functional fragment of C1ORF127 gene product corresponding to SEQ ID NO: 36, or a polypeptide sequence having at least about 80%, 85%, 90%, 91%, 92%, 93%, 94%, 95%, 96%, 97%, 98%, or 99% identity to SEQ ID NO: 36. In certain embodiments, the agent comprises a functional fragment of C1ORF127 gene product corresponding to SEQ ID NO: 37, or a polypeptide sequence having at least about 80%, 85%, 90%, 91%, 92%, 93%, 94%, 95%, 96%, 97%, 98%, or 99% identity to SEQ ID NO: 37. In certain embodiments, the agent comprises a functional fragment of C1ORF127 gene product corresponding to SEQ ID NO: 38, or a polypeptide sequence having at least about 80%, 85%, 90%, 91%, 92%, 93%, 94%, 95%, 96%, 97%, 98%, or 99% identity to SEQ ID NO: 38. In certain embodiments, the agent comprises a functional fragment of C1ORF127 gene product corresponding to SEQ ID NO: 39, or a polypeptide sequence having at least about 80%, 85%, 90%, 91%, 92%, 93%, 94° C., 95%, 96%, 97%, 98%, or 99% identity to SEQ ID NO: 39. In certain embodiments, the agent comprises a functional fragment of C1ORF127 gene product corresponding to SEQ ID NO: 40, or a polypeptide sequence having at least about 80%, 85%, 90%, 91%, 92%, 93%, 94%, 95%, 96%, 97%, 98%, or 99% identity to SEQ ID NO: 40. In certain embodiments, the agent comprises a functional fragment of C1ORF127 gene product corresponding to SEQ ID NO: 41, or a polypeptide sequence having at least about 80%, 85%, 90%, 91%, 92%, 93%, 94%, 95%, 96%, 97%, 98%, or 99% identity to SEQ ID NO: 41. In certain embodiments, the agent comprises a functional fragment of C1ORF127 gene product corresponding to SEQ ID NO: 42, or a polypeptide sequence having at least about 80%, 85%, 90%, 91%, 92%, 93%, 94%, 95%, 96%, 97%, 98%, or 99% identity to SEQ ID NO: 42. In certain embodiments, the agent comprises a functional fragment of C1ORF127 gene product corresponding to SEQ ID NO: 43, or a polypeptide sequence having at least about 80%, 85%, 90%, 91%, 92%, 93%, 94%, 95%, 96%, 97%, 98%, or 99% identity to SEQ ID NO: 43. In certain embodiments, the agent comprises a functional fragment of C1ORF127 gene product corresponding to SEQ ID NO: 44, or a polypeptide sequence having at least about 80%, 85%, 90%, 91%, 92%, 93%, 94%, 95%, 96%, 97%, 98%, or 99% identity to SEQ ID NO: 44.

A C1ORF127 gene product, fragment, or functional portion thereof described herein may be administered to a subject via any method known to those of skill in the art. For example, the C1ORF127 gene product may be administered via a cell or islet, a viral vector (e.g., an adeno associated viral vector), an exosome, or a lipid vesicle.

In some embodiments, the agent is a cell or islet expressing a C1ORF127 gene product, e.g., a C1ORF127 gene product having an amino acid sequence that has at least about 80%, 85%, 90%, 91%, 92%, 93%, 94%, 95%, 96%, 97%, 98%, 99%, or 100% identity to SEQ ID NOs: 3-6, SEQ ID NOs: 8-11, SEQ ID NO: 13, SEQ ID NO: 14, or SEQ ID NOs: 25-44. In some embodiments, the cell is an islet cell. In some embodiments, the cell is an enteroendocrine cell. In some embodiments, the cell is a beta-cell. In some embodiments, the cell is a pancreatic delta cell. In some embodiments, the cell is a gamma cell, e.g., an adult gamma cell. In some embodiments, the cell is a skeletal muscle cell or a gut enteroendocrine cell. In some embodiments, the cell is autologous to the subject requiring treatment. In some embodiments, the cell is stem cell derived. In some embodiments, the stem cell derived cell is a stem cell derived beta cell. In some embodiments, the cell is encased in a microcapsule or semi-permeable membrane.

In some embodiments, the agent is a cell, e.g., a population of cells, such as a population of stem cell-derived cells expressing a C1ORF127 gene product. The population of cells can be administered to a subject. In some embodiments, the subject who is administered a population of cells, e.g., stem cell-derived cells, is the same subject from whom a pluripotent stem cell used to differentiate into a stem cell-derived cell was obtained (e.g., for autologous cell therapy). In some embodiments, the subject is a different subject.

An agent comprising a population of cells (e.g., stem cell-derived cells, such as SC-β cells) expressing a C1ORF127 gene product can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22 (6): 563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

For administration to a subject, an agent can be administered to a subject, for example in pharmaceutically acceptable compositions. These pharmaceutically acceptable compositions comprise a therapeutically effective amount of a population of the agent as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24:199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound or agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters. polycarbonates and/or polyanhydrides; (22) bulking agents, such as polypeptides and amino acids (23) serum component, such as serum albumin, HDL and LDL; (24) C2-C12 alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to an agent means that amount an agent of the present invention which is effective for producing some desired therapeutic effect in at least a sub-population of cells in an animal at a reasonable benefit/risk ratio applicable to any medical treatment. For example, an amount of an agent administered to a subject that is sufficient to produce a statistically significant, measurable change in at least one symptom of a metabolic disorder, such as Type 1. Type 1.5 or Type 2 diabetes, such as glycosylated hemoglobin level, fasting blood glucose level, hypoinsulinemia, etc. Alternatively, a measurable change may be seen in weight and/or other markers of obesity. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

Therapeutic compositions containing at least one agent can be conventionally administered in a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The dosage ranges for the agent depends upon the potency and are amounts large enough to produce the desired effect e.g., improve blood glucose clearance. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. Typically, the dosage can range from about 0.001 mg/kg body weight to about 0.5 mg/kg body weight. In one embodiment, the dose range is from about 4 μg/kg body weight to about 30 μg/kg body weight.

Administration of the doses recited above can be repeated. In some embodiments, the doses are given once a day, or multiple times a day, for example, but not limited to, three times a day. In some embodiments, the doses recited above are administered daily for weeks or months. The duration of treatment depends upon the subject's clinical progress and responsiveness to therapy.

Precise amounts of active ingredient required to be administered depend on the judgment of the practitioner and are particular to each individual. However, suitable dosage ranges for systemic application are disclosed herein and depend on the route of administration. Suitable regimes for administration are also variable but are typified by an initial administration followed by repeated doses at one or more intervals by a subsequent administration. Alternatively, continuous intravenous infusion sufficient to maintain concentrations in the blood in the ranges specified for in vivo therapies are contemplated. In some embodiments, the dosage range is sufficient to maintain concentrations in the blood in the range found in the blood of a population of normal, healthy human subjects.

In some embodiments, the agent is administered in combination with an additional therapeutic agent. The additional therapeutic agent may be an anti-diabetic agent or an anti-obesity agent. The additional therapeutic is not limited and may be any therapeutic described herein. The additional therapeutic may be administered together with the agent or separately. In some embodiments, the additional therapeutic is in a single dosage form. In some embodiments, the additional therapeutic is in a separate dosage form.

In some embodiments, the methods disclosed herein further comprises administration to the subject of one or more agents known in the art for treatment of diabetes, obesity, and/or for having other anti-hyperglycemic activities, for example, inhibitors of dipeptidyl peptidase 4 (DPP-4) (e.g., Alogliptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide), GLPR1 agonists, insulin and insulin analogs (e.g., short acting or long acting, Insulin lispro, Insulin aspart, Insluin glulisine, Insulin glargine, Insulin detemir, Exubera and NPH insulin), alpha-glucosidase inhibitors (e.g., Acarbose, Miglitol and Voglibose), amylin analogs (e.g. Pramlintide), Sodium-dependent glucose cotransporter T2 (SGLT T2) inhibitors (e.g., Dapgliflozin, Remogliflozin and Sergliflozin), CB1 receptor blockers, apelin agonists, mitochondrial uncouplers, leptin sensitizers, myostatin agonists, sulfonylureas, thiazolidinediones, and others (e.g. Benfluorex and Tolrestat).

An agent described herein can be administrated to the subject at the same time or at different times as the administration of a second pharmaceutically active agent or composition comprising the same. When administrated at different times, the agent described herein and/or a second pharmaceutically active agent for administration to a subject can be administered within 5 minutes, 10 minutes, 20 minutes, 60 minutes, 2 hours, 3 hours, 4, hours, 8 hours, 12 hours, 24 hours of administration of the other. When an agent described herein and a second pharmaceutically active agent are administered in different pharmaceutical compositions, routes of administration can be different. In some embodiments, a subject is administered an agent described herein. In other embodiments, a subject is administered a second pharmaceutically active agent. In another embodiment, a subject is administered a composition comprising an agent described herein mixed with a second pharmaceutically active agent. In another embodiment, a subject is administered an agent described herein and a second pharmaceutically active agent, where administration is substantially at the same time, or subsequent to each other.

In some embodiments, administration of the agent described herein improves blood glucose clearance. In some embodiments, administration of the agent returns blood glucose levels from a pathological level to a non-pathological level (e.g., from a hyperglycemic state to a normal state). In some embodiments, administration of the agent reduces blood glucose levels by about 5%, 10%, 15%, 20%, 25%, 30%, 35%, 40%, 45%, 50%, 55%, 60%, 65%, 70%, 75%, or more. In some embodiments, administration of the agent does not cause hypoglycemia in the subject (e.g., a blood sugar of less than about 70 mg/dL, a blood sugar of less than about 60 mg/dL, or to a blood sugar level wherein the subject does not exhibit signs of hypoglycemia). In some embodiments, the blood glucose clearance property of the agent is independent of insulin activity.

The compositions and methods disclosed herein are useful for the prevention, or in certain instances the treatment, of obesity, weight gain, hepatosteatosis and/or one or more metabolic diseases. As used herein, the term “metabolic disease” generally refers to disorders affecting a subject in which errors of metabolism, imbalances in metabolism, or suboptimal metabolism occur and include, for example, impaired glucose tolerance, insulin resistance, diabetes mellitus and/or hepatosteatosis. In certain embodiments, the metabolic disease relates to or develops as a result of a condition. For example, obesity may contribute to the development or progression of diabetes, rendering an obese subject at an increased risk for the development of the metabolic disease.

In certain aspects, the compositions and methods disclosed herein may be used to treat one or more metabolic diseases selected from the group consisting of Type I diabetes, Type II diabetes, gestational diabetes, insulin resistance, metabolic syndrome, obesity, impaired glucose tolerance, impaired fasting glucose, and hepatosteatosis. In certain aspects, the metabolic disease is diabetes. In certain aspects, the metabolic disease is hepatosteatosis.

In some embodiments, the compositions and methods disclosed herein are useful for reducing or otherwise mitigating the risk that a subject will develop a metabolic disease, or in certain instances are useful for slowing or preventing the metabolic consequences associated with obesity. For example, such compositions and methods may be administered to a subject that is at risk for developing a metabolic disease (e.g., a subject with a family history of diabetes) and thereby reduce or mitigate the risk that such subject will develop the metabolic disease. Similarly, in certain aspects such compositions and methods may be administered to a subject (e.g., a subject with a family history of metabolic disease) to slow or prevent the development of obesity in such subject and thereby reduce or mitigate the risk that such subject will develop the metabolic disease. In some embodiments, the subject's risk of developing the metabolic disease is reduced or reversed by about at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 33%, at least 35%, at least 41%, at least 44%, at least 50%, at least 60%, at least 70%, at least 75%, at least 80%, at least 90%, at least 95%, at least 97%, at least 99%, or at least 100%.

It should be understood that the present inventions are not limited to reducing, preventing, inhibiting or mitigating the risk of one or more metabolic diseases. Rather, also contemplated are methods of treating one or more metabolic diseases using the compositions and methods that are disclosed herein. For example, an effective amount of the compositions disclosed herein may be administered to an obese subject to treat the subject's obesity (e.g., to cause a reduction in the subject's weight), and thereby reduce the risk that the subject will experience further deterioration in their metabolic disease. In certain embodiments, the methods and compositions disclosed herein are coupled with one or more non-pharmacological interventions. For example, the compositions disclosed herein may be administered in combination with one or more lifestyle modifications (e.g., diet, exercise and/or smoking cessation therapy).

In some embodiments, the agent improves blood glucose clearance when administered to a subject. In some embodiments, the agent does not cause hypoglycemia when administered to a subject.

In some embodiments, the agent increases the rate of glucose turnover when administered to the subject. In some embodiments, administration of the agent increases glucose turnover by about 5%, 10%, 20%, 30%, 40%, 50%, 75%, 100%, or more. In some embodiments, administration of the agent increases glucose turnover about 1.1-fold, 1.2-fold, 1.3-fold, 1.4-fold, 1.5-fold, 1.6-fold, 2-fold, 2.5-fold, 3-fold, 3.5-fold, 5-fold, 10-fold, 50-fold, or more.

In some embodiments, the agent increases glycolysis when administered to the subject. In some embodiments, the agent increases the rate of glycolysis when administered to the subject. In some embodiments, administration of the agent increases glycolysis by about 5%, 10%, 20%, 30%, 40%, 50%, 75%, 100%, or more. In some embodiments, administration of the agent increases glycolysis about 1.1-fold. 1.2-fold, 1.3-fold, 1.4-fold, 1.5-fold, 1.6-fold, 2-fold, 2.5-fold, 3-fold, 3.5-fold, 5-fold, 10-fold, 50-fold, or more.

In some embodiments, the agent increases the rate of glycogen synthesis when administered to the subject. In some embodiments, the agent increases the rate of glycogen synthesis when administered to the subject. In some embodiments, administration of the agent increases glycogen synthesis by about 5%, 10%, 20%, 30%, 40%, 50%, 75%, 100%, or more. In some embodiments, administration of the agent increases glycogen synthesis about 1.1-fold, 1.2-fold, 1.3-fold, 1.4-fold, 1.5-fold, 1.6-fold, 2-fold, 2.5-fold, 3-fold, 3.5-fold, 5-fold, 10-fold, 50-fold, or more. In some embodiments, the agent has glucagon-like activity when administered to the subject.

As used herein a disorder associated with elevated blood glucose levels is any disorder wherein the subject has elevated blood glucose levels. In some embodiments, the disorder is a metabolic disorder, such as, diabetes (e.g., Type I diabetes or Type II diabetes), metabolic syndrome, glucose intolerance, or obesity.

A subject can be one who has been previously diagnosed with or identified as suffering from or having a condition in need of treatment (e.g., a metabolic disease) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. Rather, a subject can include one who exhibits one or more risk factors for a condition or one or more complications related to a condition. A “subject in need” of treatment for a particular condition can be a subject having that condition, diagnosed as having that condition, or at increased risk of developing that condition relative to a given reference population.

Mammals other than humans can be advantageously used as subjects that represent animal models of metabolic disorders, such as Type 1 diabetes, Type 2 Diabetes Mellitus, or pre-diabetic conditions. A subject can be one who has been previously diagnosed with or identified as suffering from or having diabetes (e.g., Type 1 or Type 2), one or more complications related to diabetes, or a pre-diabetic condition, and optionally, but need not have already undergone treatment for the diabetes, the one or more complications related to diabetes, or the pre-diabetic condition. A subject can also be one who is not suffering from diabetes or a pre-diabetic condition. A subject can also be one who has been diagnosed with or identified as suffering from diabetes, one or more complications related to diabetes, or a pre-diabetic condition, but who show improvements in known diabetes risk factors as a result of receiving one or more treatments for diabetes, one or more complications related to diabetes, or the pre-diabetic condition. Alternatively, a subject can also be one who has not been previously diagnosed as having diabetes. one or more complications related to diabetes, or a pre-diabetic condition. For example, a subject can be one who exhibits one or more risk factors for diabetes, complications related to diabetes, or a pre-diabetic condition, or a subject who does not exhibit diabetes risk factors, or a subject who is asymptomatic for diabetes, one or more diabetes-related complications, or a pre-diabetic condition. A subject can also be one who is suffering from or at risk of developing diabetes or a pre-diabetic condition. A subject can also be one who has been diagnosed with or identified as having one or more complications related to diabetes or a pre-diabetic condition as defined herein, or alternatively, a subject can be one who has not been previously diagnosed with or identified as having one or more complications related to diabetes or a pre-diabetic condition.

In some embodiments, the subject has diabetes (e.g., Type I diabetes or Type II diabetes), metabolic syndrome, metabolic disease (e.g., metabolic dysfunction-associated steatohepatitis), glucose intolerance, hyperlipidemia, or obesity. In some embodiments, the subject has diabetes. In some embodiments, the subject's genome comprises a mutant or variant form of C1ORF127. In some embodiments, the variant is one of the following: 1_11014118_C_T, 1_11015165_A_G, 1_11008102_G_A, 1_11009679_G_A, 1_11007881_G_T. 1_11008778_T_A,C, 1_11008799_G_C. 1_11008417_G_A, 1_11008127_C_T. 1_11009703_C_T, 1_11008594_A_T, 1_11007997_C_T. 1_11024271_G_T,A, 1_11008685_T_C, 1_11009716_C_G, 1_11009844_G_A, 1_11008417_G_A, 1_11036248_G_A, 1_11024271_G_T,A, 1_11009844_G_A, 1_11008799_G_C, 1_11008778_T_A,C, 1_11008685_T_C, 1_11014118_C_T, 1_11009716_C_G, 1_11008102_G_A, 1_11007895_G_T, 1_11008127_C_T, 1_11009703_C_T, 1_11008594_A_T, 1_11009679_G_A, 1_11014127_C_T, 1_11008844_C_G,T, 1_11015165_A_G, 1_11009703_C_T, 1_11009679_G_A, 1_11008594_A_T, 1_11008102_G_A, 1_11009844_G_A, 1_11007895_G_T, 1_11008127_C_T, 1_11008417_G_A, 1_11007724_C_T, 1_11024271_G_T,A, 1_11008778_T_A,C, 1_11009716_C_G, 1_11015165_A_G, 1_11014118_C_T, 1_11008799_G_C, 1_11007881_G_T, 1_11008685_T_C, 1_11007997_C_T, 1_11015165_A_G, 1_11008102_G_A, 1_11008685_T_C, 1_11008799_G_C, 1_11008127_C_T, 1_11009679_G_A, 1_11014118_C_T, 1_11009703_C_T, 1_11007724_C_T, 1_11008594_A_T, 1_11009716_C_G, 1_11007895_G_T, 1_11007881_G_T, 1_11008778_T_A,C, 1_11024271_G_T,A, or 1_11007997_C_T. In some embodiments, the variant is one of the following identified in Table 1.

TABLE 1
C1ORF127 Variants

| VariantID | Alt | Phenotype | Position | Reference dbSNP | Consequ\|ence |
|---|---|---|---|---|---|
| 1:17311190:A:G | G | Height | 17311190 A | rs9435734 | upstream_gene_variant |
| 1:72765116:G:A | A | BMI | 72765116 G | rs2568958 | upstream_gene_variant |
| 1:25760920:C:T | T | LDL | 25760920 C | rs11802413 | intron_variant |
| 1:25808948:T:C | C | CHOL | 25808948 T | rs61775190 | intron_variant |
| 1:11862778:A:G | G | SBP | 11862778 A | rs17367504 | intron_variant |
| 1:11862278:A:G | G | DBP | 11862778 A | rs17367504 | intron_variant |
| 1:16505320:A:G | G | ALT | 16505320 A | rs1497406 | intergenic_variant |
| 1:10825577:C:T | T | Varicose Veins | 10825577 C | rs11121615 | intron_variant |
| 1:27021913:G:C | C | IGF1 | 27021913 G | rs114165349 | upstream_gene_variant |
| 1:16505320:A:G | G | AST | 16505320 A | rs1497406 | intergenic_varinat |
| 1:11887382:A:G | G | Hypertension | 11887382 A | rs56190030 | intron_variant |
| 1:11862778:A:G | G | PulsePress | 11862778 A | rs17367504 | intron_variant |
| 1:16513926:A:G | G | Alb | 16513926 A | rs924204 | intergenic_variant |
| 1:15815922:G:A | A | eGFRcrea | 15815922 G | rs9659443 | intron_variant |
| 1:23699340:G:A | A | Creatinine | 23699340 G | rs2749153 | upstream_gene_variant |
| 1:9336748:C:T | T | WHRadjBMI | 9336748 C | rs72641835 | upstream_gene_variant |
| 1:15855123:G:C | C | EGFRcreaNoDiabetes | 15855123 G | rs7546668 | intron_variant |
| 1:12794144:C:T | T | Optic_cup_area | 12794144 C | rs181939629 | intergenic_variant |
| 1:7884525:G:T | T | SleepChronotype | 7884525 G | rs61773390 | intron_variant |
| 1:15909480:G:C | C | CystatinC | 15909480 G | rs10803394 | intron_variant |
| 1:16510894:C:T | T | nonHDL | 16510894 C | rs36086195 | intergenic_variant |
| 1:16510894:C:T | T | ApoB | 16510894 C | rs36086195 | intergenic_variant |
| 1:11179484:C:T | T | Weight | 11179484 C | rs56803472 | intron_variant |
| 1:935222:C:A | A | HDL | 935222 C | rs2298214 | missense_variant |
| 1:7888438:C:T | T | ChronotypeSingle | 7888438 C | rs17374439 | intron_variant |
| 1:9336678:A:G | G | WHR | 9336678 A | rs113563611 | upstream_gene_variant |
| 1:9438175:C:T | T | AbAA | 9438175 C | rs12408895 | intergenic_variant |
| 1:11253817:C:T | T | POAG | 11253817 C | rs147660927 | missense_variant |
| 1:6679848:C:T | T | T2D | 6679848 C | rs2072948 | intron_variant |
| 1:1686962:C:T | T | TG | 1686962 C | rs2076327 | intron_variant |
| 1:16371510:G:A | A | BUN | 16371510 G | rs7365304 | intron_variant |

| VariantID | Nearest minor Allele | Group |
|---|---|---|
| 1:17311190:A:G | [“MFAP2”] G | Anthropometric - Height |
| 1:72765116:G:A | [“NEGR1”] G | Anthropometric - Body mass |
| | | index (BMI) |
| 1:25760920:C:T | [“TMEM57”] T | Lipids - LDL cholesterol |
| 1:25808948:T:C | [“TMEM57”] T | Lipids - Total cholesterol |
| 1:11862778:A:G | [“MTHFR”] G | Cardiovascular - Systolic blood |
| | | pressure |
| 1:11862278:A:G | [“MTHFR”] G | Cardiovascular - Diastolic blood |
| | | pressure |
| 1:16505320:A:G | [“EPHA2”] G | Hepatic - Alamine transanuasse |
| | | (ALT) |
| 1:10825577:C:T | [“CASZ1”] T | Cardiovascular - Varicose veins |
| 1:27021913:G:C | [“ARID1A”] C | Insulin-like growth factor (IGF-1) |
| 1:16505320:A:G | [“EPHA2”] G | Hepatic - Aspartate |
| | | aminotransferase (AST) |
| 1:11887382:A:G | [“MTHFR”] G | Cardiovascular - hypertension |
| 1:11862778:A:G | [“MTHFR”] G | Cardiovascular - pulse pressure |
| 1:16513926:A:G | [“ARHGEF19”] G | Hepatic - serum albumin |
| 1:15815922:G:A | [“CELA2B”] A | Renal - eGFRcreat (serum |
| | | creatinine) |
| 1:23699340:G:A | [“C1orf213”] G | Renal - Serum creatinine |
| 1:9336748:C:T | [“SPSB1”] T | Anthropometric - Waist-hip ratio |
| | | adj BMI |
| 1:15855123:G:C | [“DNAJC16”] C | Renal - eGERcreat in individuals |
| | | without diabetes |
| 1:12794144:C:T | [“C1orf158”] T | |
| 1:7884525:G:T | [“UTS2”] T | Sleep and circadian - chronotype |
| | | (morningness) |
| 1:15909480:G:C | [“AGMAT”] C | Renal - Cystatin C |
| 1:16510894:C:T | [“ARHGEF19”] T | Lipids - Non-HDL cholesterol |
| 1:16510894:C:T | [“ARHGEF19”] T | Lipids - Serum ApoB |
| 1:11179484:C:T | [“MTOR”] T | Anthropometric -- Weight |
| 1:935222:C:A | [“HES4”] A | Lipids - HDL cholesterol |
| 1:7888438:C:T | [“UTS2”] T | Sleep and circadian - chronotype |
| | | (single question) |
| 1:9336678:A:G | [“SPSB1”] G | Anthropometric - waist-hip ratio |
| 1:9438175:C:T | [“SPSB1”] T | Cardiovascular - Abdominal |
| | | aortic aneurysm |
| 1:11253817:C:T | [“ANGPTL7”] T | |
| 1:6679848:C:T | [“THAP3”] T | Glycemic - Type 2 diabetes |
| | | (T2D) |
| 1:1686962:C:T | [“NADK”] T | Lipids - Triglycerides |
| 1:16371510:G:A | [“CLCNKB”] A | Renal - Blood urea nitrogen |

In some embodiments, the C1ORF127 variant is associated with diabetes and high BMI. In some embodiments, the C1ORF127 variant associated with diabetes and high BMI is 1_11033415_G_A. In some embodiments, the C1ORF127 variant is associated with elevated fasting glucose levels. In some embodiments, the C1ORF127 variant associated with elevated fasting glucose levels is 1_11014118_C_T. In some embodiments, the variant is associated with type 1 diabetes. In some embodiments, the variant is associated with type 2 diabetes. In some embodiments, the variant is associated with high BMI.

C1ORF127 may be implicated in one or more of the following:

| Gene | Phenotype | Group |
|---|---|---|
| C1ORF127 | ALT | Hepatic |
| C1ORF127 | Varicose veins | Cardiovascular |
| C1ORF127 | Alb | Hepatic |
| C1ORF127 | CystatinC | Renal |
| C1ORF127 | AST | Hepatic |
| C1ORF127 | Weight | Anthropometric |
| C1ORF127 | T2D | Glycemic |
| C1ORF127 | HDL | Lipids |
| C1ORF127 | Height | Anthropometric |
| C1ORF127 | CRF | Ocular |
| C1ORF127 | POAG | Ocular |
| C1ORF127 | VATadjBMIHeight | Anthropometric |
| C1ORF127 | Urate | Renal |
| C1ORF127 | BMI | Anthropometric |

The methods described herein may lead to a reduction in the severity or the alleviation of one or more symptoms of the disorder. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), blood pressure at or above 140/90 mm/Hg; abnormal blood fat levels, such as high-density lipoproteins (HDL) less than or equal to 35 mg/dL, or triglycerides greater than or equal to 250 mg/dL (mg/dL=milligrams per deciliter of blood), presence of ketones in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

In some embodiments, the methods disclosed herein delay the onset of a metabolic disorder, such as diabetes. Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

### Compositions and Kits

Described herein are compositions which comprise an agent described herein (e.g., Atollin or a fragment thereof). In some embodiments, the composition also includes a second agent (e.g., a second therapeutic agent) described herein. Also described herein are kits. Another aspect of the present invention relates to kits for practicing methods disclosed herein and for making the agents disclosed herein.

In some embodiment, the agent(s) in the kit can be provided in a watertight or gas tight container which in some embodiments is substantially free of other components of the kit. The agent(s) can be provided in any form, e.g., liquid, dried or lyophilized form. It is preferred that an agent(s) described herein be substantially pure and/or sterile. When an agent(s) described herein is provided in a liquid solution, the liquid solution preferably is an aqueous solution, with a sterile aqueous solution being preferred. When an agent(s) described herein is provided as a dried form, reconstitution generally is by the addition of a suitable solvent. The solvent, e.g., sterile water or buffer, can optionally be provided in the kit.

In some embodiments, the kit further optionally comprises information material. The informational material can be descriptive, instructional, marketing or other material that relates to the methods described herein and/or the use of an agent(s) described herein for the methods described herein.

The informational material of the kits is not limited in its instruction or informative material. In one embodiment, the informational material can include information about production of the agent, molecular weight of the agent, concentration, date of expiration, batch or production site information, and so forth. In one embodiment, the informational material relates to methods for administering the agent. Additionally, the informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about an agent described herein and/or its use in the methods described herein. Of course, the informational material can also be provided in any combination of formats.

In one embodiment, the informational material can include instructions to administer an agent(s) (e.g., Atollin or a fragment thereof) as described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer an agent(s) described herein to a suitable subject, e.g., a human, e.g., a human having or at risk for a disorder described herein or to a cell in vitro.

In addition to an agent(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional therapeutic agent, e.g., for treating a condition or disorder described herein. Alternatively, the other ingredients can be included in the kit, but in different compositions or containers than an agent described herein. In such embodiments, the kit can include instructions for admixing an agent(s) described herein and the other ingredients, or for using a compound(s) described herein together with the other ingredients, e.g., instructions on combining the two agents prior to administration.

The kit can include one or more containers for the composition containing at least one agent as described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The kit can also include a component for the detection of a marker for Atollin, e.g., for a marker described herein, e.g., a reagent for the detection of Atollin. Or in some embodiments, the kit can also comprise reagents for the detection of negative markers of Atollin for the purposes of negative selection of Atollin. The reagents can be, for example, an antibody against the marker or primers for a RT-PCR or PCR reaction, e.g., a semi-quantitative or quantitative RT-PCR or PCR reaction. Such markers can be used to evaluate whether Atollin has been produced. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

### Biomarkers and Assays

The disclosure further contemplates the use of C1ORF127 as a biomarker for a metabolic disease or condition. In some aspects, the disclosure contemplates the use of C1ORF127 as a biomarker for a disease or condition associated with elevated blood glucose levels. In some aspects, the disclosure contemplates the use of C1ORF127 as a biomarker for a disease or condition associated with elevated blood glucose levels where the subject is insulin resistant. In some aspects, C1ORF127 may act as a biomarker for the presence of a disease or condition. In other aspects, C1ORF127 may act as a biomarker for monitoring the progression of a disease or condition. In some aspects, C1ORF127 secretion acts as a biomarker for a metabolic disease or condition or a disease or condition associated with elevated blood glucose levels.

In some aspects, assays are used to identify a metabolic disease or condition in a subject, e.g., using C1ORF127 protein levels or secretion values as a biomarker. In some aspects, assays are used to identify a disease or condition associated with elevated blood glucose levels in a subject, e.g., using C1ORF127 protein levels or secretion values as a biomarker.

In some embodiments, a disease or condition is associated with elevated levels of C1ORF127 secretion. In some embodiments the disease or condition is a metabolic disease (e.g., Type I diabetes, Type II diabetes, gestational diabetes, insulin resistance, metabolic syndrome, obesity, impaired glucose tolerance, impaired fasting glucose, hepatosteatosis and metabolic dysfunction-associated steatohepatitis). In some embodiments, the subject is resistant to insulin. In some embodiments the disease or condition is associated with or occurs as a result of elevated blood glucose levels.

In some embodiments, a disease or condition is associated with decreased protein levels of C1ORF127. In some embodiments the disease or condition is a metabolic disease (e.g., Type I diabetes, Type II diabetes, gestational diabetes, insulin resistance, metabolic syndrome, obesity, impaired glucose tolerance, impaired fasting glucose, hepatosteatosis and metabolic dysfunction-associated steatohepatitis). In some embodiments the disease or condition is associated with or occurs as a result of elevated blood glucose levels.

In some aspects, a method for detecting a metabolic disease or condition comprises obtaining a sample from a subject and assessing the sample to determine if it exhibits increased secretion levels of C1ORF127. In some embodiments, the secretion of C1ORF127 is measured using any method known to those of skill in the art. Increased secretion levels of C1ORF127 may be an indication of elevated blood glucose levels as a result of a disease or disorder. In some aspects, the progression of a disease or condition associated with elevated blood glucose levels is assessed by analyzing multiple samples from a subject over an extended period of time to monitor the secretion levels of C1ORF127 (e.g., in response to a treatment protocol).

In some aspects, a method for detecting a metabolic disease or condition comprises obtaining a sample from a subject and assessing the sample to determine if it exhibits decreased levels of C1ORF127 protein. In some embodiments, the protein levels of C1ORF127 is measured using any method known to those of skill in the art. Decreased protein levels of C1ORF127 may be an indication of elevated blood glucose levels as a result of a disease or disorder. In some aspects, the progression of a disease or condition associated with elevated blood glucose levels is assessed by analyzing multiple samples from a subject over an extended period of time to monitor the protein levels of C1ORF127 (e.g., in response to a treatment protocol).

In some aspects a method for detecting a metabolic disease (e.g., Type I diabetes. Type II diabetes, gestational diabetes, insulin resistance, metabolic syndrome, obesity, impaired glucose tolerance, impaired fasting glucose, hepatosteatosis and metabolic dysfunction-associated steatohepatitis) in a subject comprises obtaining a sample (e.g., a biofluid sample) from the subject suffering, and determining if the sample contains increased secretion levels of C1ORF127. Increased secretion levels of C1ORF127 may be an indication of elevated blood glucose levels as a result of a metabolic disease or disorder, e.g., levels of C1ORF127 secretion are increased in response to the increase in blood glucose in a subject. In some embodiments, the subject is resistant to insulin.

In some aspects a method for detecting a metabolic disease (e.g., Type I diabetes, Type II diabetes, gestational diabetes, insulin resistance, metabolic syndrome, obesity, impaired glucose tolerance, impaired fasting glucose, hepatosteatosis and metabolic dysfunction-associated steatohepatitis) in a subject comprises obtaining a sample (e.g., a biofluid sample) from the subject suffering, and determining if the sample contains decreased protein levels of C1ORF127. Decreased protein levels of C1ORF127 may be an indication of elevated blood glucose levels as a result of a metabolic disease or disorder, e.g., levels of C1ORF127 protein are decreased and thus blood glucose is increased in a subject.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

### EXAMPLES

### Example 1: MRNA Sequencing of Translocon Associated Transcripts Identifies, Atollin, a Novel Endocrine Activity of Pancreatic Beta-Cells

Most studies of secreted proteins rely on algorithms to predict signal peptides. However, these miss many secreted proteins that lack canonical signals (Diehn et al., 2000; Emanuelsson et al., 2007; Käll et al., 2004; Meinken et al., 2015, Petersen et al., 2011; Jan el at., 2014). Recent efforts have used biochemical isolation of ER-associated ribosomes to characterize secreted factor mRNAs in non-mammalian and cancer models (Jan et al., 2014; Fazl et al., 2019; Reid et al., 2014; Reid and Nicchitta, 2012). Translating ribosome affinity purification with GFP-tagged ribosomes has also been applied to profile actively translated mRNA pools in neuronal tissues and plants (Doyle 2008, Heiman 2008; Reynoso 2015). Evidence that mRNAs partition to ER-localized ribosomes motivated using an ER translocon label instead to identify secreted factors independent of their topological signals for trafficking through the secretory pathway (Reid and Nicchitta 2012; Hoffman A et al JBC from Nicchitta's group).

Here the inventors describe the development and application of an endoplasmic reticulum mRNA sequencing technique (ER-seq) that enriches for secreted and transmembrane transcripts in insulin expressing stem cell derived pancreatic beta cells and their progenitors. The method uses transgenic human embryonic stem cell lines that constitutively mark the ER-translocon complex using a Sec61 beta-GFP fusion protein and a subcellular fractionation and immunoprecipitation steps to identify transcripts encoding secreted proteins. Among the mRNAs identified, the inventors discovered C1ORF127 (also referred to herein as “Atollin”). Atollin is highly expressed in mouse and human beta cells and is under the control of beta cell transcription factors. Mice overexpressing Atollin showed glucose-dependent reductions in blood glucose without hypoglycemia, and lowering glucose when insulin action is blocked through insulin receptors or after beta cell ablation. Long-term overexpression models demonstrate an increase in peripheral Insulin sensitivity and reduced age-related weight gain and circulating cholesterol levels. Together, this suggests that Atollin is an important regulator of glucose homeostasis and metabolism operating through novel insulin-independent mechanisms. The ability of Atollin to promote glucose utilization has significant therapeutic potential.

### Results

Combining Transgenic hPSC with a Biochemical Fractionation Method for the Isolation of Ribosome-Translocon Complexes

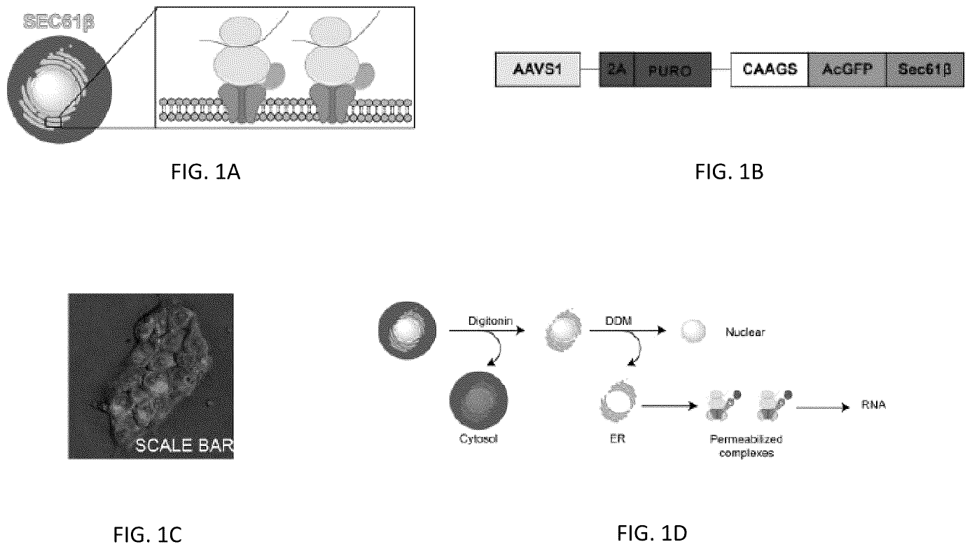

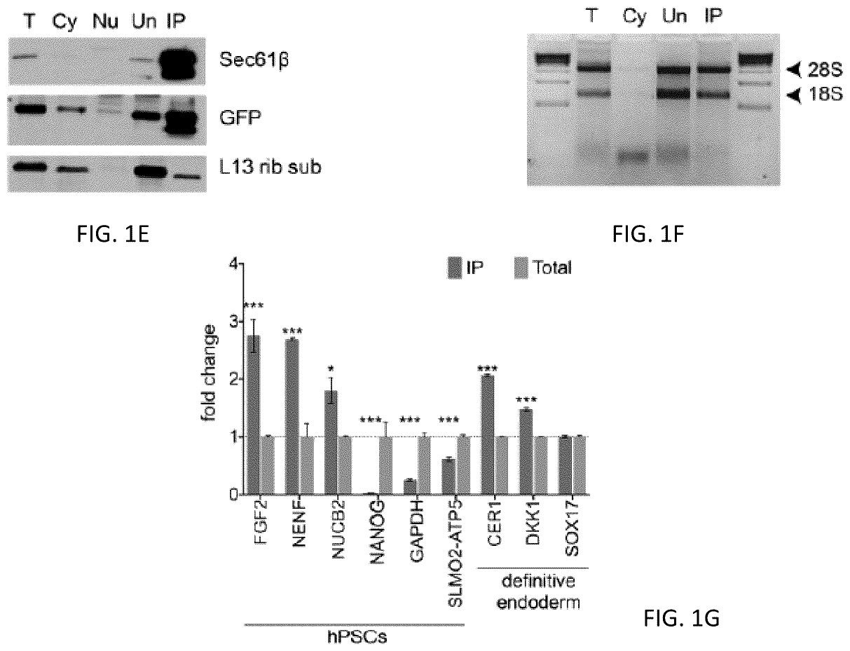

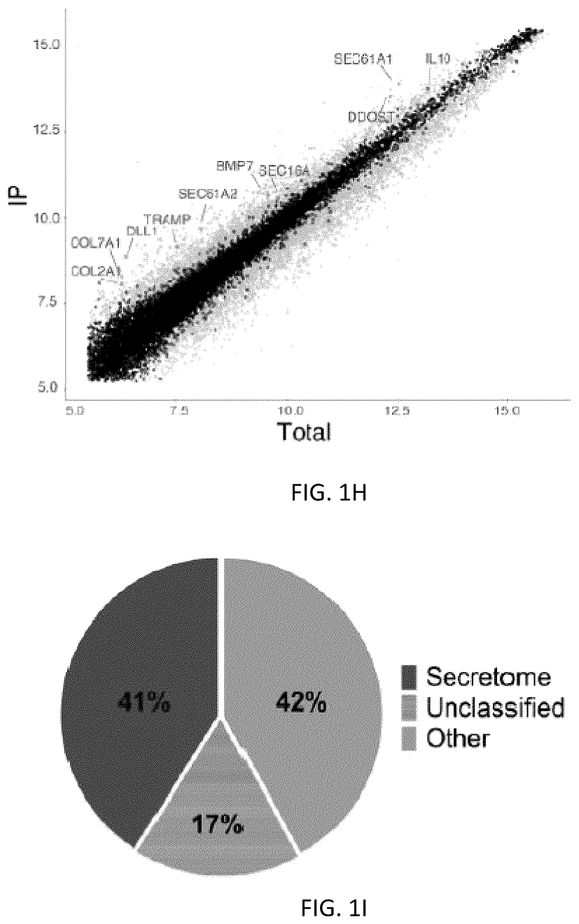

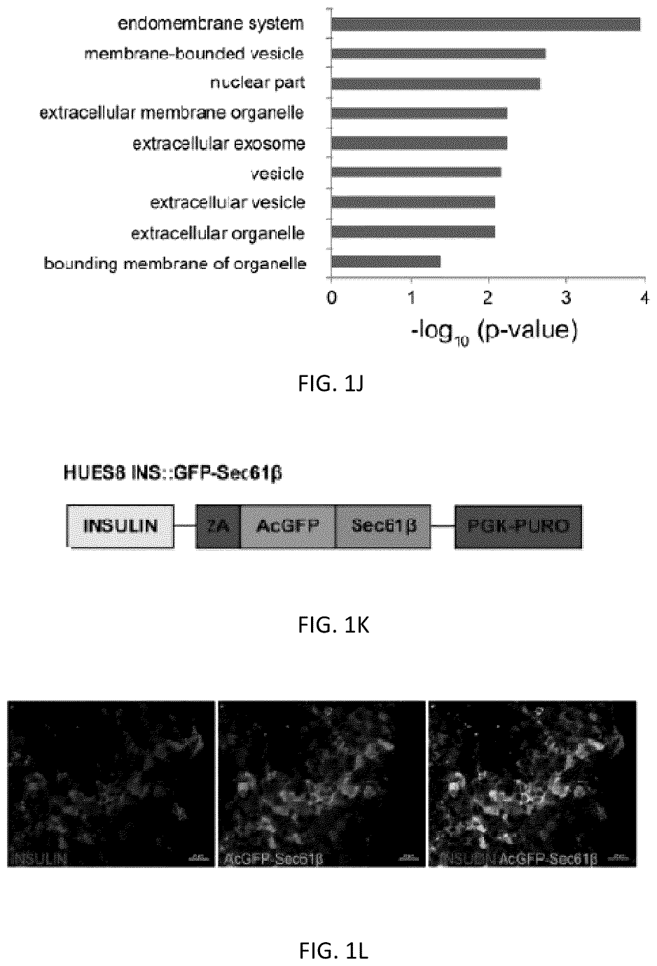

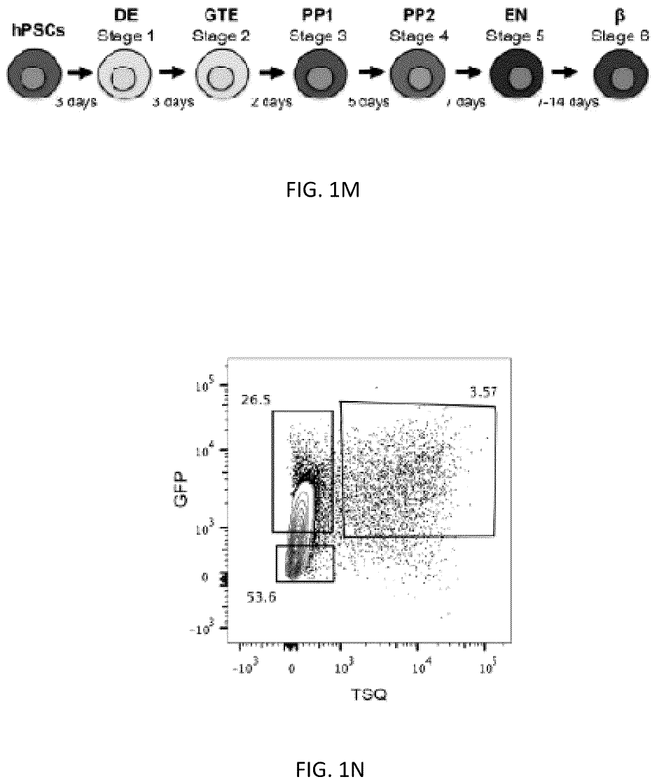

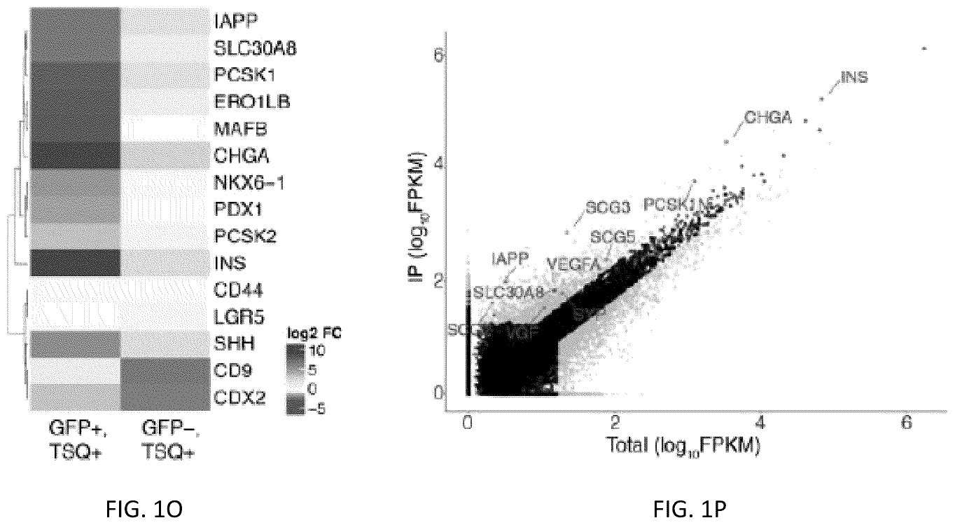

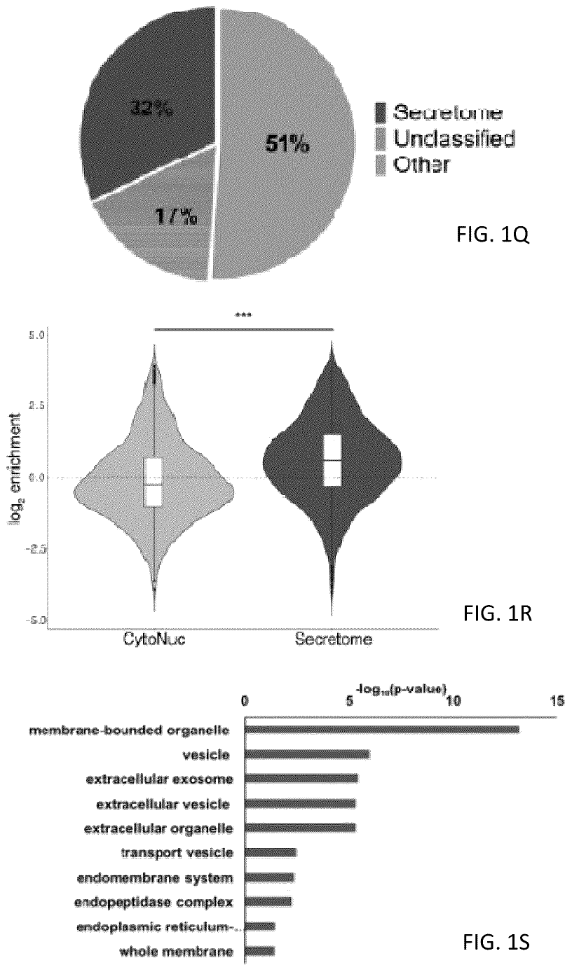

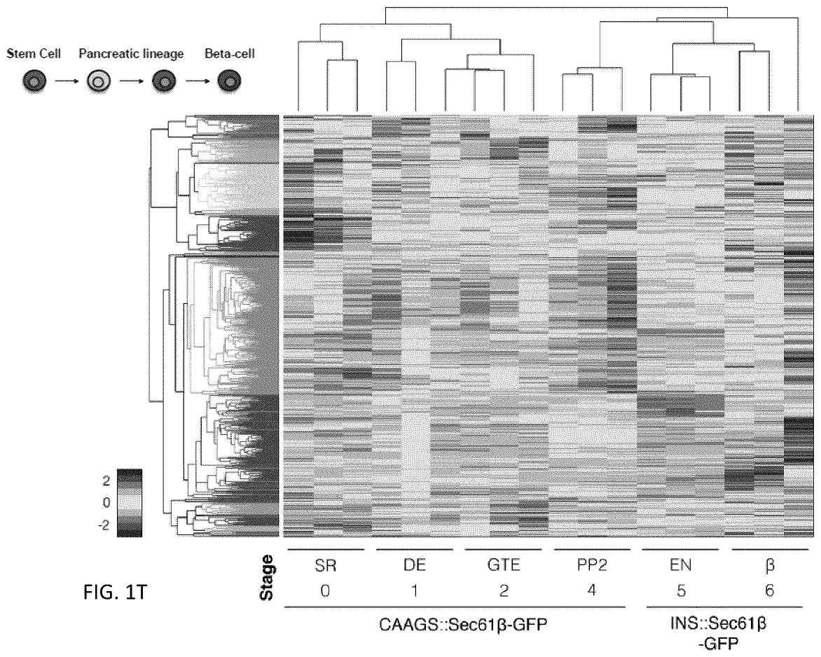

As the biogenesis of secreted factors, including hormones, occurs in ER-localized ribosome/translocon complexes, the inventors reasoned that the isolation of translocon-associated mRNAs should enrich for mRNAs encoding secreted factors (Jan et al., 2014). To isolate ribosome/translocon complexes, the inventors generated a human pluripotent stem cell (hPSC) line that expresses a subunit of the translocon complex, Sec61β, fused to Aequorea coerulescens GFP (AcGFP), a monomeric variant of GFP (FIGS. 1A-1C). To achieve ubiquitous and constitutive expression in hPCSs and differentiated progeny, TALEN-mediated genome editing was used to knock-in the AcGFP-SEC61β fusion protein into the AAVS1 locus along with the CAGGS promoter (CAGGS::AcGFP-SEC61B; FIG. 1B). In hPSCs and SC-β cells, the expression of the transgene was perinuclear as expected for an ER-localized protein (FIG. 1C).

To isolate ER-localized ribosome/translocon complexes, a stepwise subcellular fractionation protocol was developed that can selectively permeabilize ER components in hPSCs, stem cell-derived tissues, and HepG2 cells (FIG. 1D).

After ER permeabilization, the ER fraction was subjected to immunoprecipitation with anti-GFP nanobodies coupled to magnetic beads to isolate ribosome/translocon complexes and associated mRNAs (FIG. 1D). The inventors applied this protocol to hPCSs and detected a significant enrichment in Sec61β and AcGFP protein expression in the immunopurified (IP) fraction relative to unfractionated cell extracts, as assayed by western blot (FIG. 1E). Importantly, the ribosomal protein subunit L13a and ribosomal RNA subunits 28S and 18S were also co-purified (FIGS. 1E-1F). The immunopurified ER fraction was subjected to mass spectrometry and peptides of the translocon subunit SEC61a, translocon-associated protein disulfide isomerase (PDI) and multiple ribosomal protein subunits were detected. Therefore, the biochemical protocol allows for an effective enrichment of ribosome/translocon complexes and associated mRNAs.

ER-Seq Robustly Enriches for mRNAs of Secreted Factors in hPSCs

To determine whether this approach effectively enriches for mRNAs that encode secreted factors and membrane proteins, the inventors first performed qPCR analysis of candidate secreted factors on translocon-associated mRNAs purified from hPSCs and SC-definitive endoderm progenitors. In hPSCs, the inventors detected a significant 1.5 to 3-fold enrichment in IP mRNAs of secreted factors including Fibroblast growth-factor 2 (FGF2, 2.75-fold increase), Neudesin (NENF. 2.69-fold increase) and Nucleobindin2 (NUCB2, 1.8-fold increase) in hPSCs relative to total unfractionated RNA. In SC-derived definitive endoderm cells, the inventors observed a 2.1-fold increase of Cerberus1 (CER1) and a 1.5-fold increase of Dickkopf WNT Signaling Pathway Inhibitor 1 (DKK1) mRNAs in IP fractions relative to total unfractionated RNA (FIG. 1G). Concomitantly, a depletion greater than 4-fold of mRNAs coding for cytosolic and nuclear factors including GAPDH (3.9-fold decrease), NANOG (37.1-fold decrease) and SLMO2-ATP5 (1.6-fold decrease) (FIG. 1G) was observed. These results suggest that ER-seq effectively enriches mRNAs coding for secreted factors.

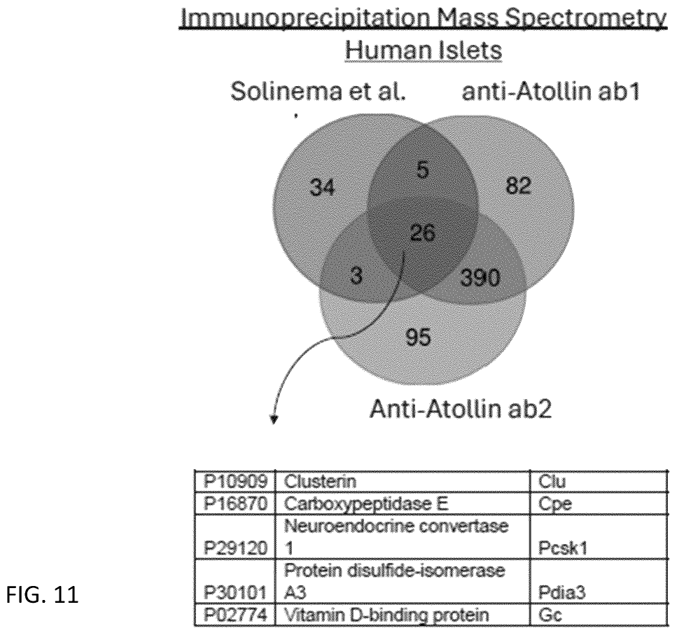

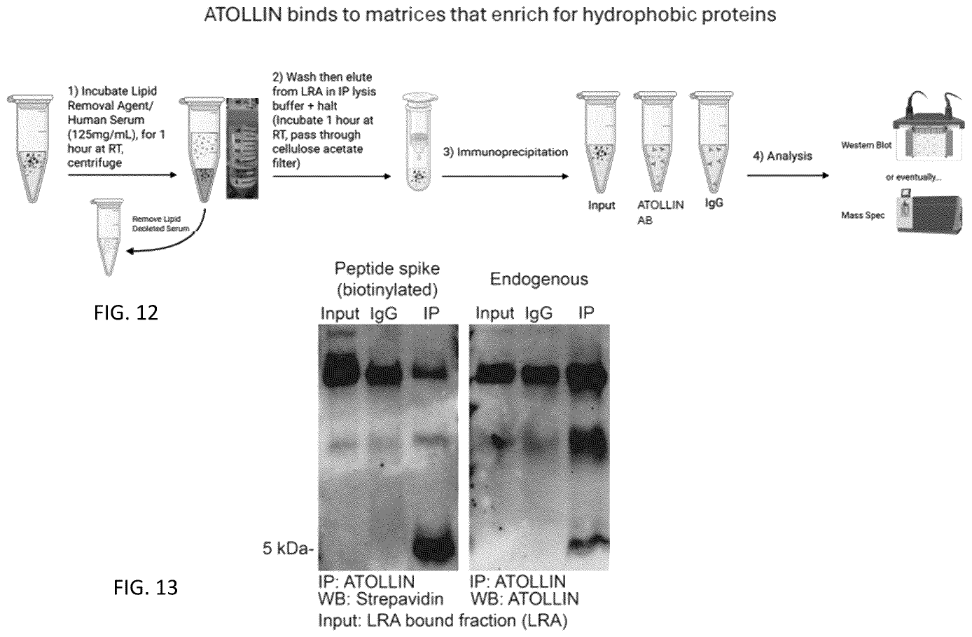

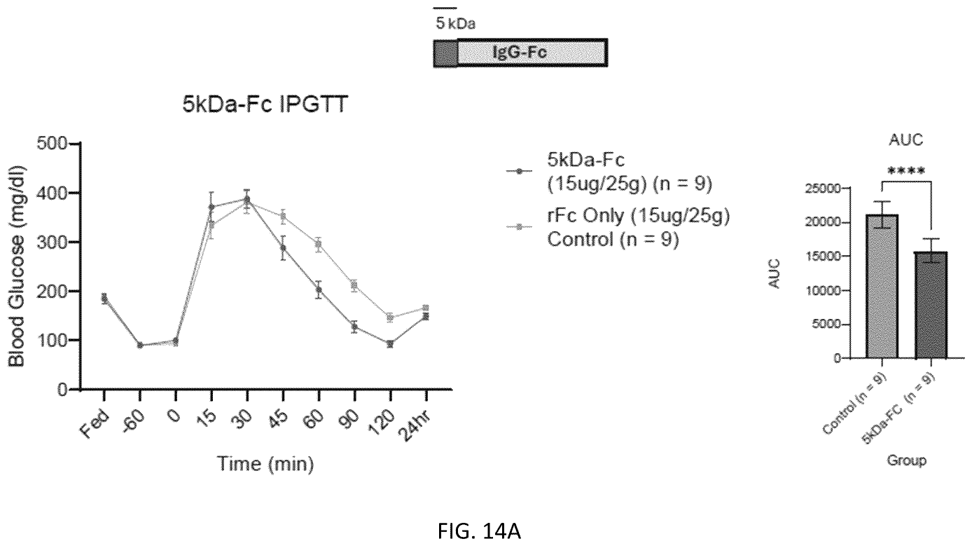

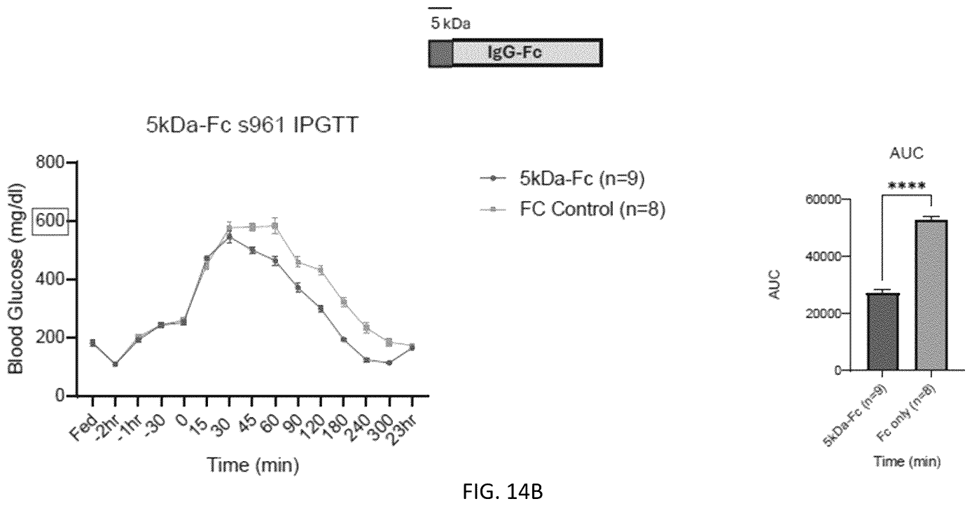

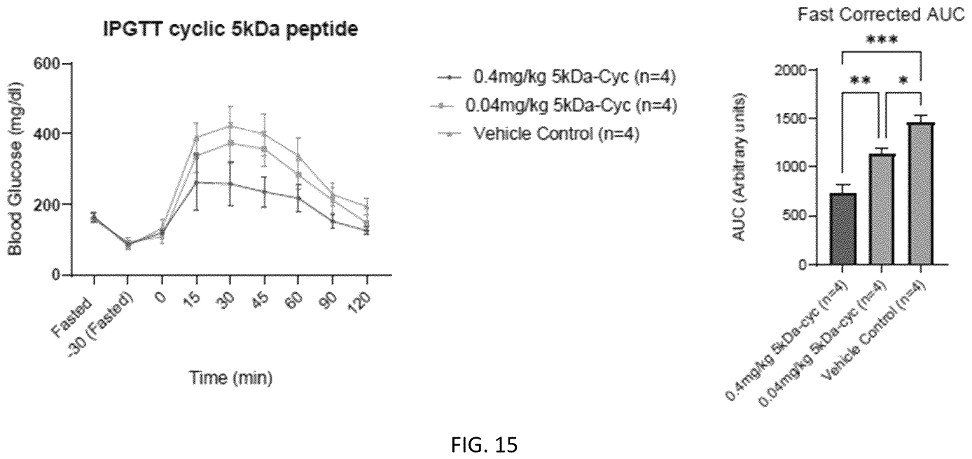

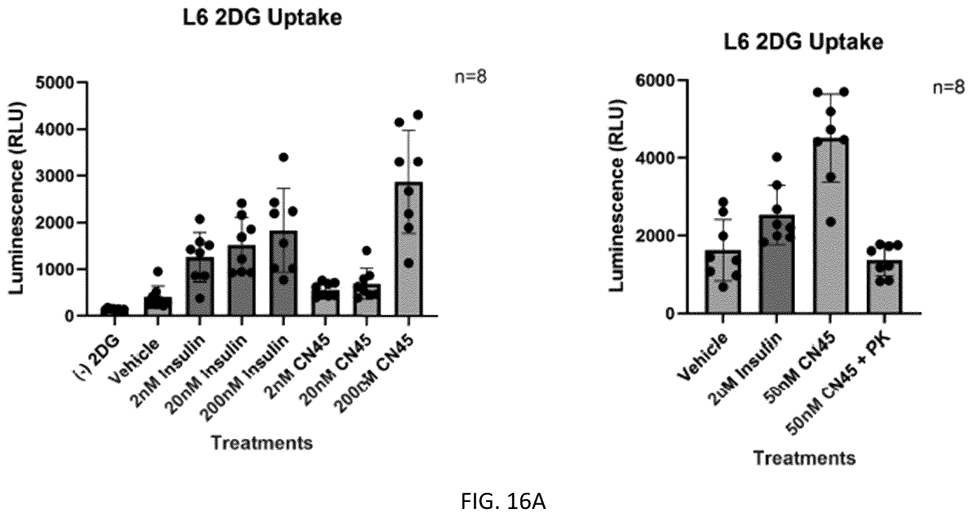

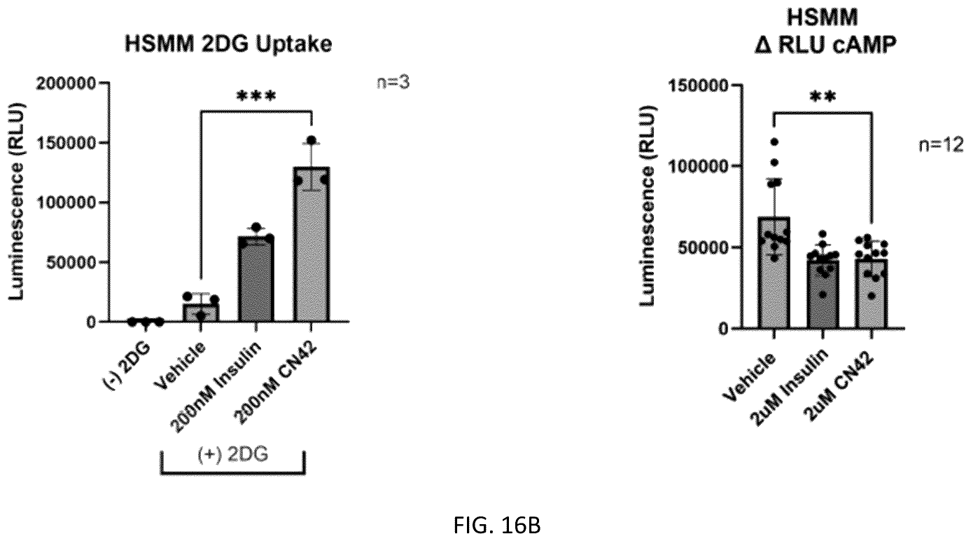

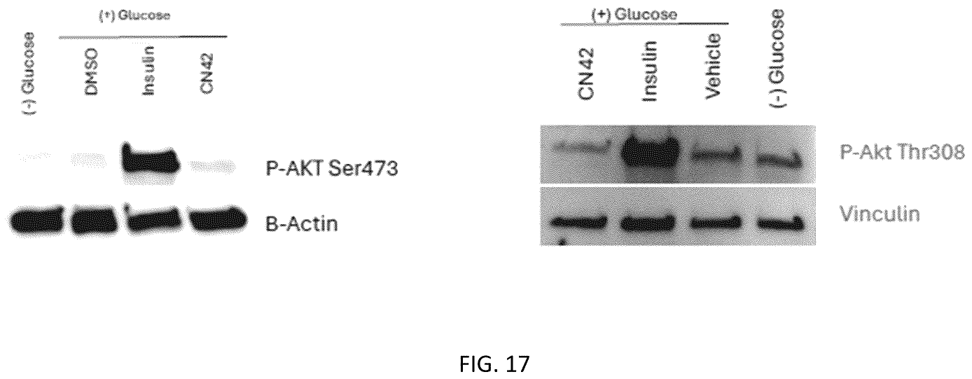

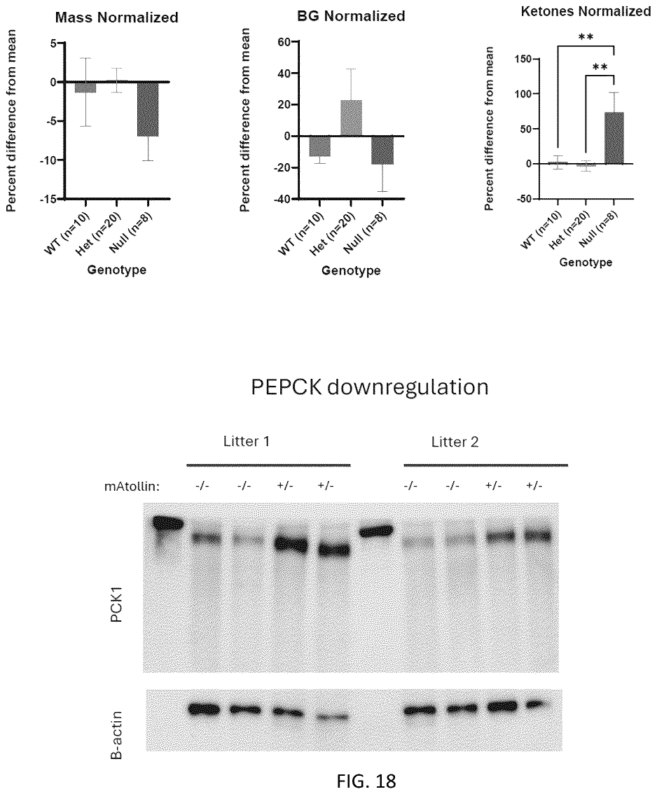

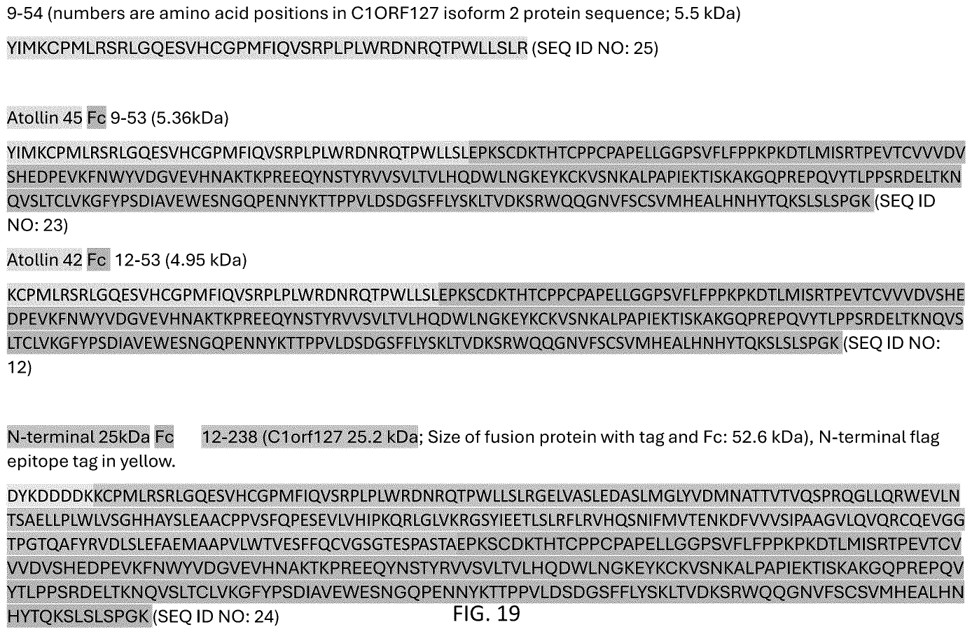

To estimate the transcriptome-wide enrichment of secreted factors in IP fractions relative to unfractionated mRNAs, the inventors performed microarray analysis on translocon-associated mRNAs purified from hPSCs. Compared to mRNAs collected from total unfractionated cell extracts, mRNAs encoding for the ER factors including Sec61α and DDOST as well as secreted factors such as BMP7, DLL1, COL2A1, COL7A1, among others, displayed a 1.5-fold or greater increase in expression in IP relative to unfractionated RNA (FIG. 1H). Out of 3174 genes that are enriched (fold change>2) in the IP fraction relative to unfractionated mRNA, the inventors detected 989 genes (41.1%) that are predicted to be secreted factors based on canonical topogenic signal prediction (FIG. 11). Based on gene ontology analysis, there is a significant (P-value<0.05) enrichment of genes that are part of the endomembrane system, vesicles and extracellular components among genes enriched in the IP fraction (FIG. 1J). Importantly, the inventors also detected 650 IP-enriched unannotated genes with no predicted canonical localization signal prediction. 10-15% of all genes expressed in most cell types are predicted to be secreted (Uhlén et al., 2015) and the computational analysis suggests this fractionation method effectively enriches for mRNAs of secreted factors expressed in hPSCs.

### β Cell ER-Seq

The inventors next used the ER-seq protocol to characterize the secretory landscape in SC-beta cells. To express the AcGFP-labelled translocon complexes specifically in Insulin-expressing B cells, the CRISPR-Cas9 system was used to knock-in the AcGFP-SEC61β fusion transgene into the last exon of the endogenous insulin gene in hPSCs (INS: AcGFP-SEC61β; FIG. 1K). An in vitro directed differentiation protocol was used (Pagliuca et al., 2014) to generate SC-β cells from hPSCs using the INS: AcGFP-SEC61β cell line (FIG. 1M). The fidelity of the transgenic line for Insulin expression was verified in three ways. First, Insulin and GFP colocalization experiments were performed in differentiated SC-β cells (FIG. 1L). The inventors demonstrated by indirect immunofluorescence staining a high degree of concordance between Insulin and GFP protein expression in SC-β cells at stage 6 of directed differentiation (FIG. 1L, right panel). Since the differentiation into the SC-β lineage also results in the formation of polyhormonal cells that co-express Insulin and Glucagon (Pagliuca et al., 2014; Veres et al., 2019), the inventors next sought to prove that bonafide SC-β cells could be identified by their ability to bind Zinc chelating dyes, such as TSQ (Davis et al., 2019). Insulin is packed in granules that contain high levels of Zinc, thus TSQ was used to stain and sort the transgenic SC-β cells. (FIG. 1N, shows flow cytometry analysis of SC-β cells stained for TSQ and expressing AcGFP). The inventors speculated that the GFP+/TSQ-population were either immature β or polyhormonal cells. Lastly, Nanostring gene expression analysis was performed using a custom set of about 100 genes expressed during β cell differentiation and it was demonstrated that sorted transgenic TSQ+, AcGFP+SC-β cells display increased expression (fold change >2) of β cell specific factors compared to TSQ-non-endocrine cells (FIG. 1O). This set of genes included Insulin (INS), NKX6-1, PDX1, amylin (IAPP), among others (FIG. 1O). The results suggest that the transgenic SC-β cells can make bonafide β cells.

Thus, this transgenic cell line allowed for the isolation of ribosome/translocon complexes from β cells in a heterogeneous mixture of cell types (without the need to sort the cells) and perform RNA-sequencing on translocon-associated mRNAs. The inventors applied the protocol to SC-β cells and, by RNA-sequencing of translocon-associated mRNAs, identified a significant enrichment (fold change>2) of hormones such as insulin and amylin (IAPP), the angiogenic factor VEGFA, and VGF, as well as other genes involved in insulin secretion such as chromogranin A (CHGA), secretogranins (SCG2, SCG3, SCG5), and synapthophysin (SYP, FIG. 1P). 2,732 genes were identified that are enriched in the IP fraction relative to total unfractionated RNA (fold change>2). 874 of this set of genes (32%) are predicted to be secreted factors based on computational topogenic signal prediction (Kall et al., 2005) (FIG. 1Q). 17% of the genes did not have a predicted localization pattern and were not annotated as nuclear, cytoplasmic, secreted or membrane localized. Among factors predicted to be part of the secretome of the cell, the inventors detected a robust enrichment of the mRNAs in the IP fraction relative to total RNA (FIG. 1R, average fold change=1.65) while genes that are annotated as nuclear and/or cytoplasmic were depleted in the IP fraction (FIG. 1R). Accordingly, gene ontology analysis of IP-enriched genes suggests an enrichment in factors that are part of the endomembrane system, ER and extracellular part of the cells (FIG. 1S). The computational analysis, in both the constitutive and the lineage-specific cell lines, suggests an effective enrichment and quantification of mRNAs encoding for secreted factors by ER-seq.

Stage-Specific Expression Patterns of Translocon-Associated mRNAs

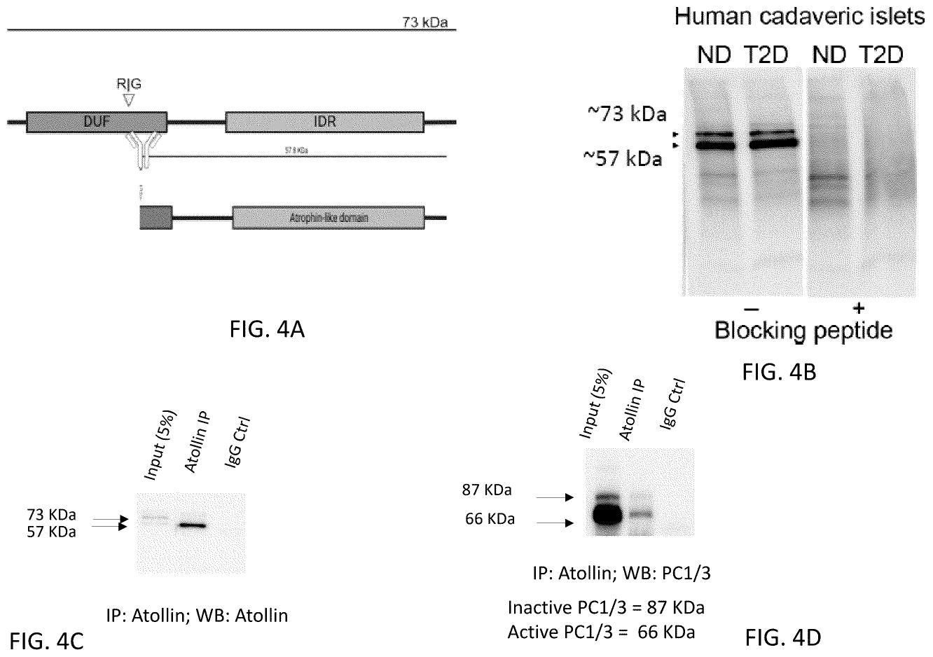

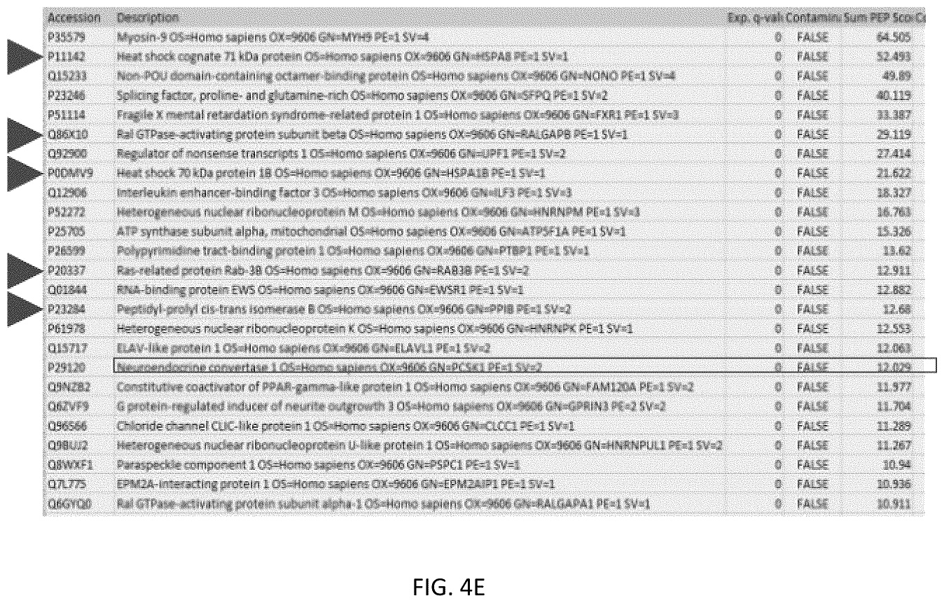

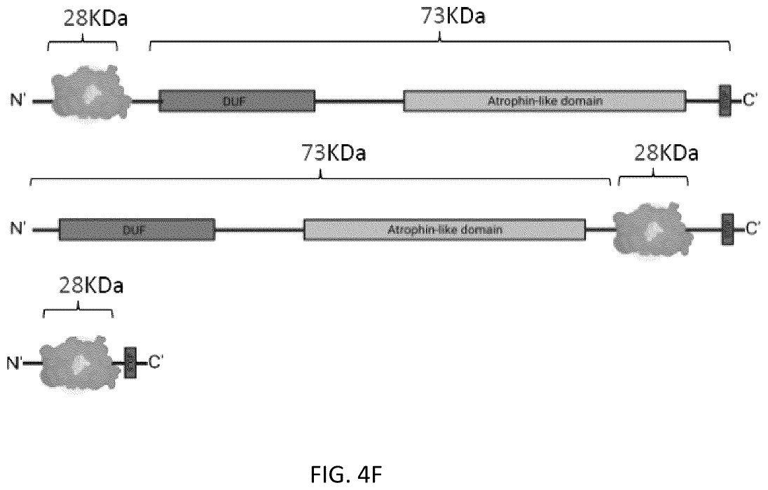

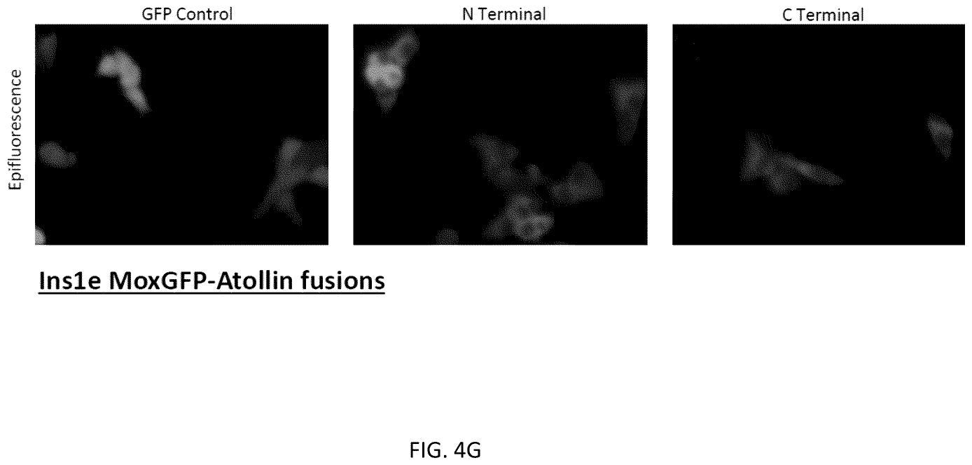

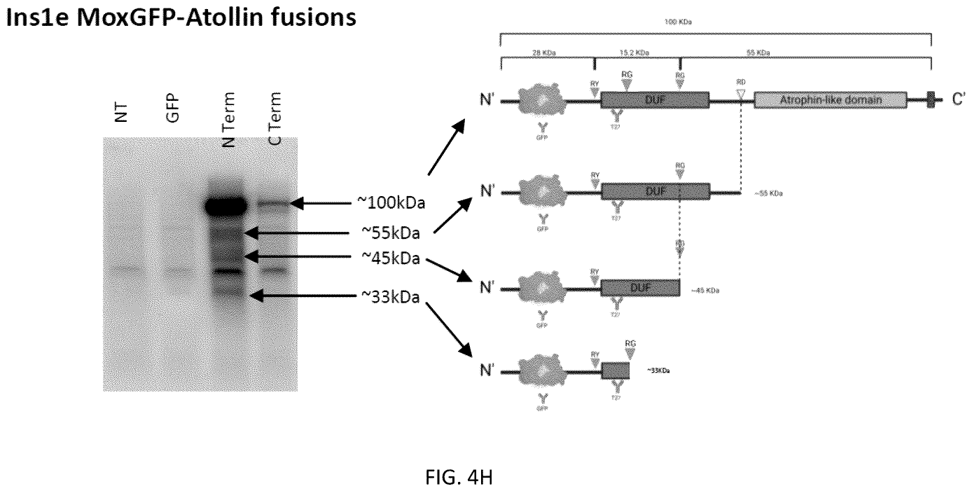

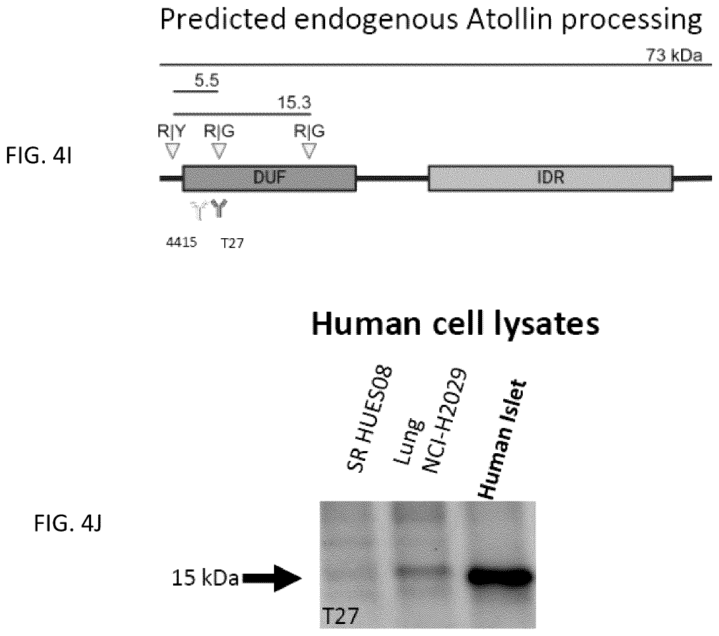

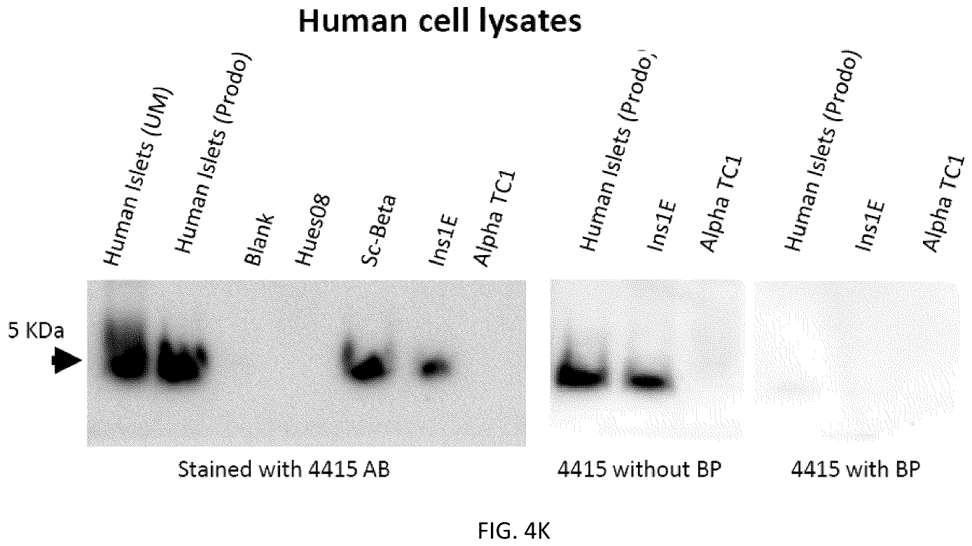

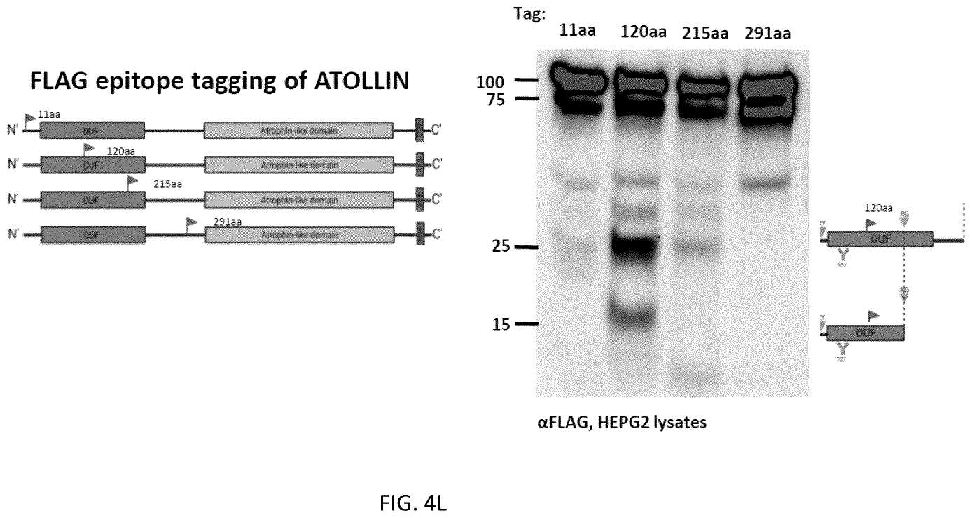

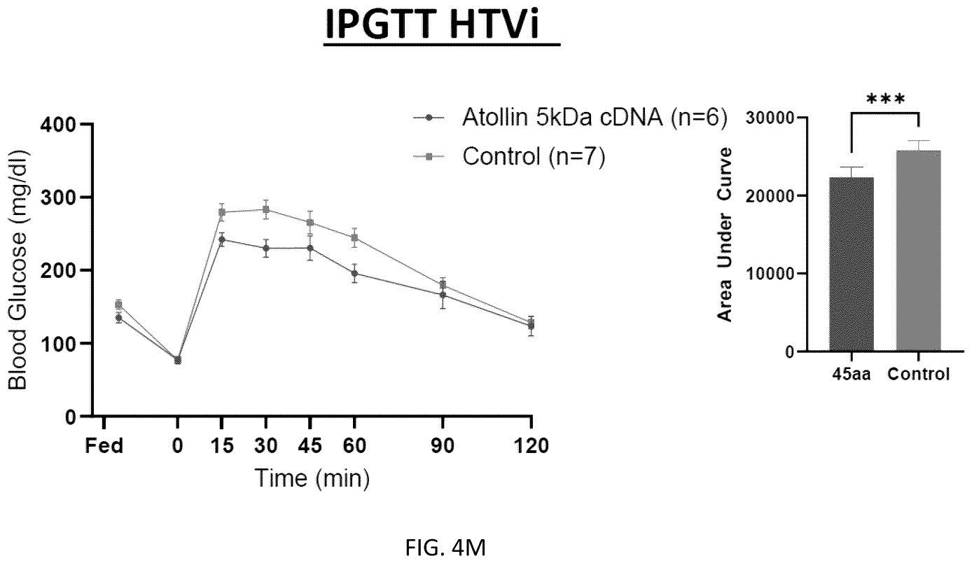

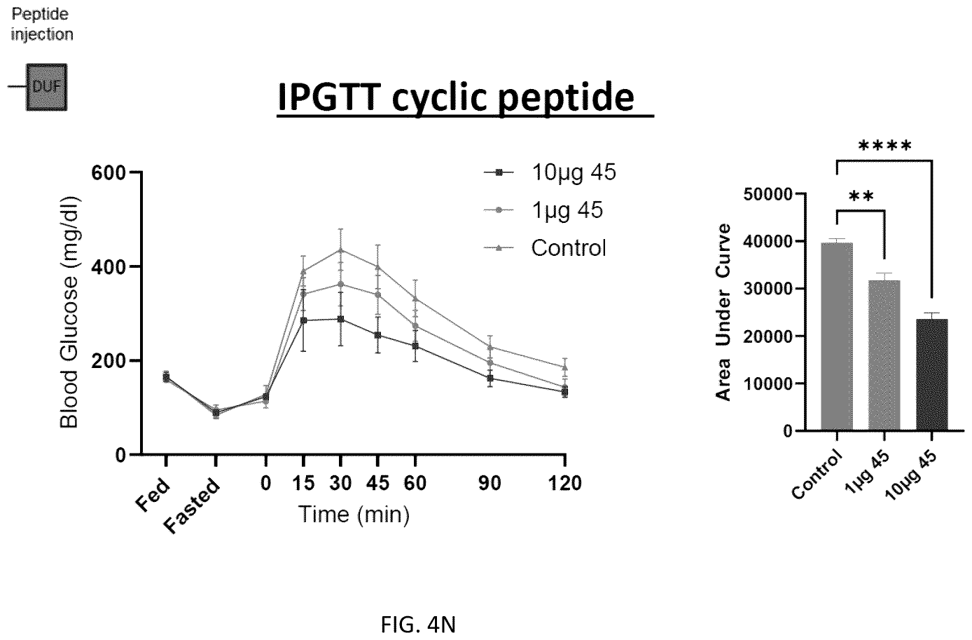

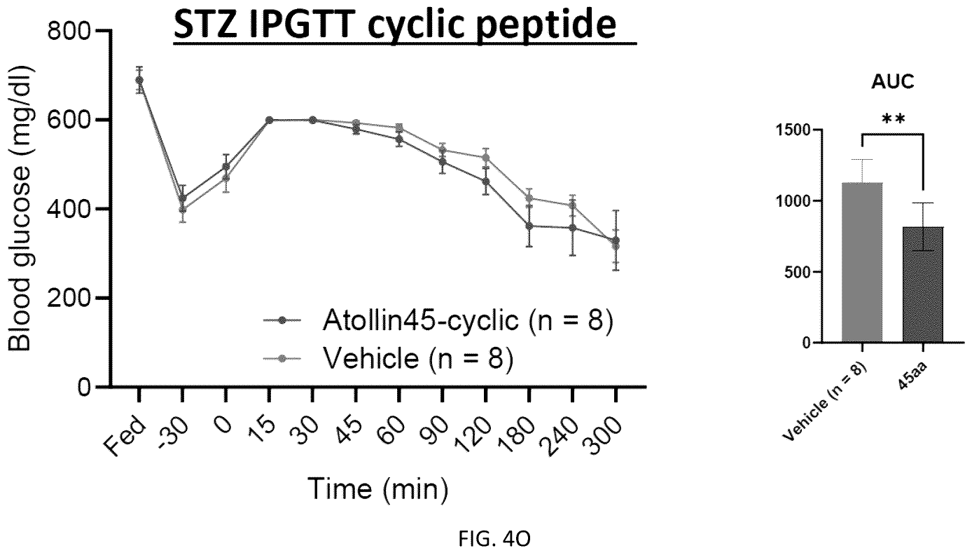

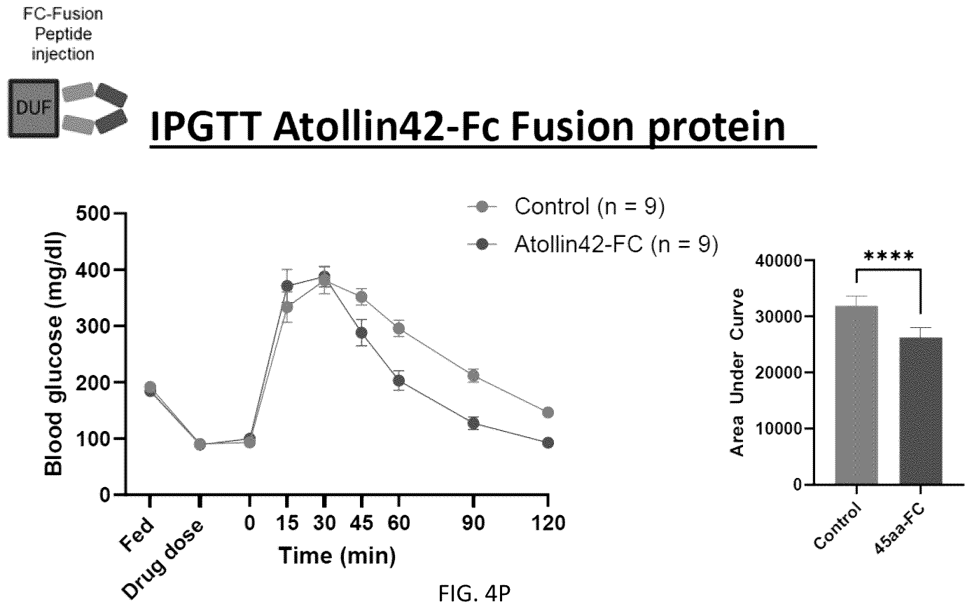

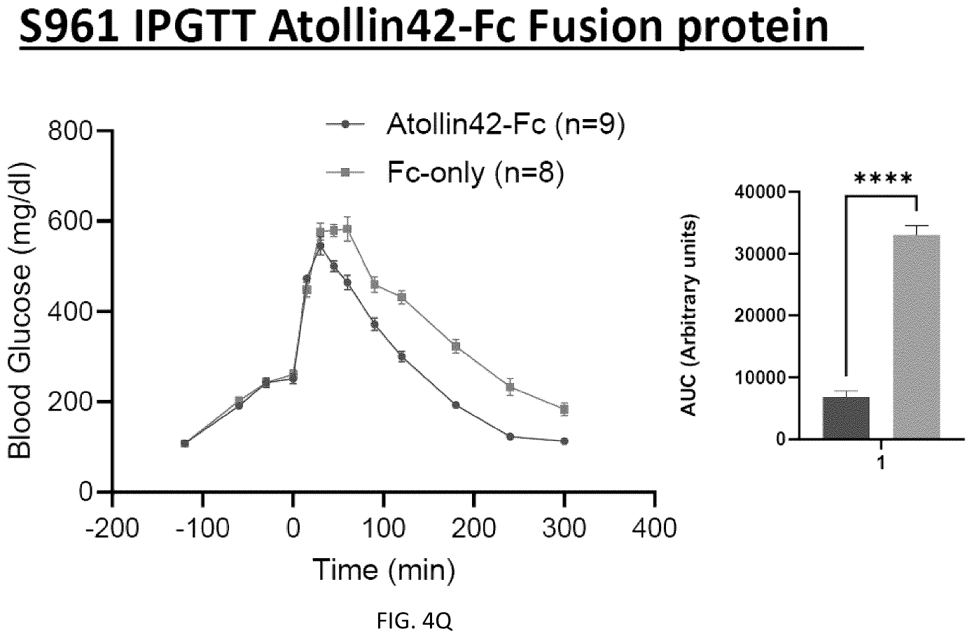

The analysis of translocon-associated mRNAs in hPSC and SC-β cells revealed an enrichment of a substantial number of genes that may represent novel secreted factors expressed in SC-β cells. To identify translocon-associated genes that are preferentially expressed in β cells, the inventors applied the ER-seq protocol to cells at multiple stages of the in vitro differentiation process towards the β cell lineage. During the initial stages of differentiation when cell populations are more homogeneous, the inventors relied on the constitutive expression of AcGFP-SEC61β in the CAGGS: AcGFP-SEC61β cell line to isolate translocon-associated mRNAs in hPSCs and their differentiated progeny (FIG. 1T). During the last stages of differentiation, cell heterogeneity is more prominent, hence, the INS: AcGFP-SEC61β cell line was used to isolate translocon-associated mRNAs in insulin-expressing SC-β cells. Translocon-associated mRNAs were sequenced at all stages of differentiation and stage-specific gene expression signatures were defined (FIG. 1T). 601 genes that are differentially expressed (false discovery rate <0.05) across all stages of differentiation were identified. The inventors resolved a gene expression signature that was specific to SC-β cells that included 139 genes, 44 of which are predicted secreted factors (FIG. 4B). An ingenuity pathway analysis (IPA) was performed on differentially expressed genes to identify secreted upstream regulators of genetic pathways that are differentially regulated during in vitro SC-cell differentiation. The inventors identified candidate upstream regulators that displayed stage-specific patterns of pathway activity. These included regulators known to be important for stem cell maintenance and differentiation such as FGF2 signaling in self-renewing hPSCs (Diecke et al., 2008) and WNT signaling in gut tube endoderm and pancreatic progenitors (Sharon et al., 2019). The analysis identified multiple upstream signaling mediators that may play important roles during in vitro human β cell differentiation.

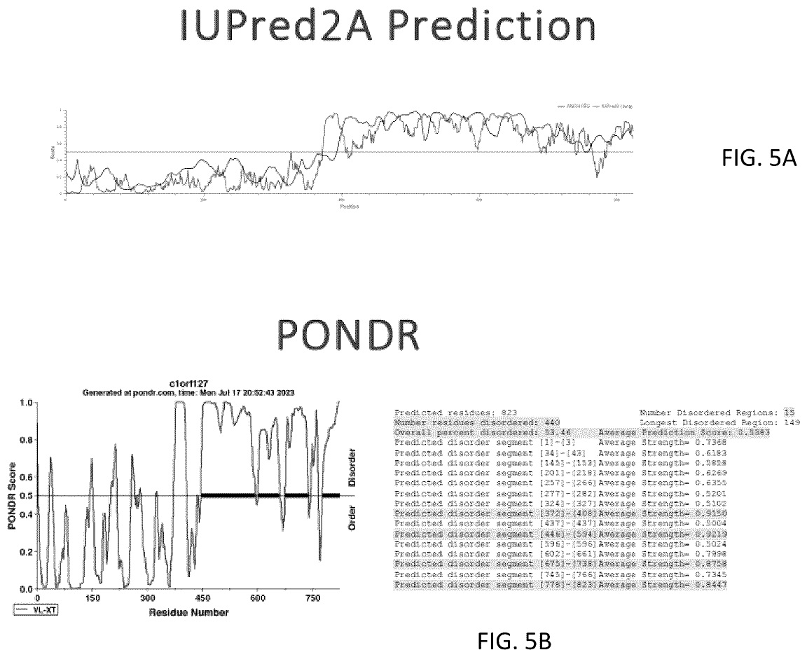

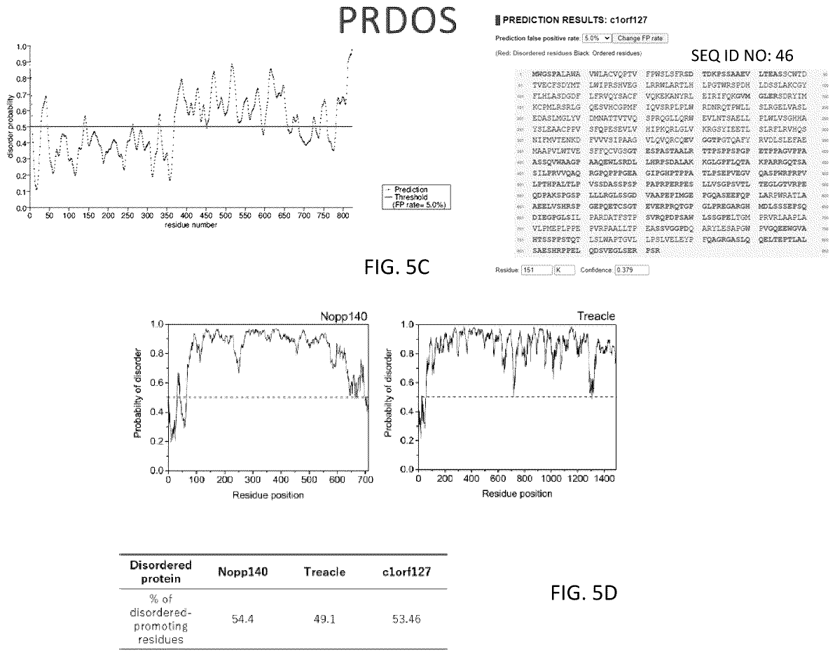

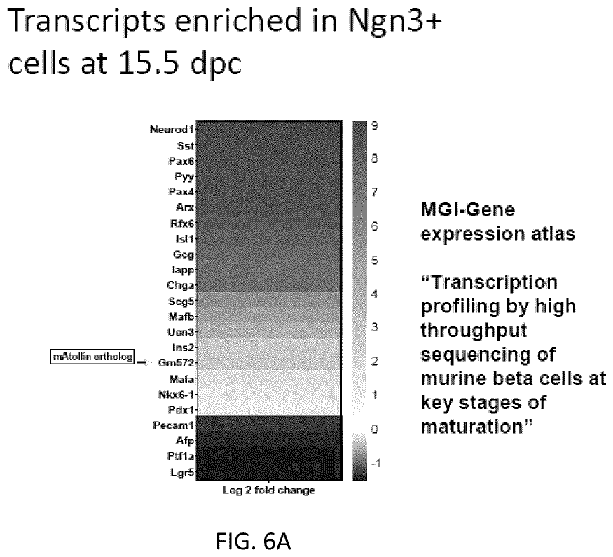

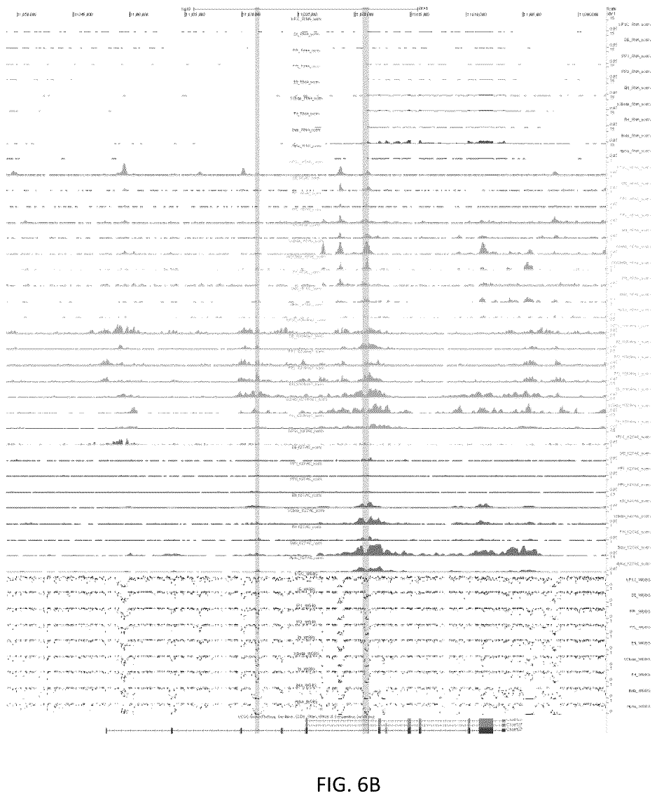

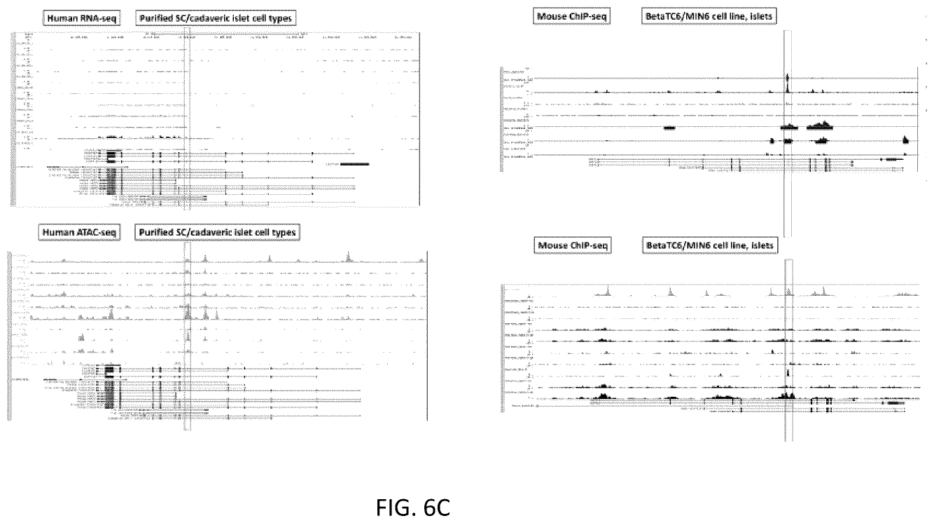

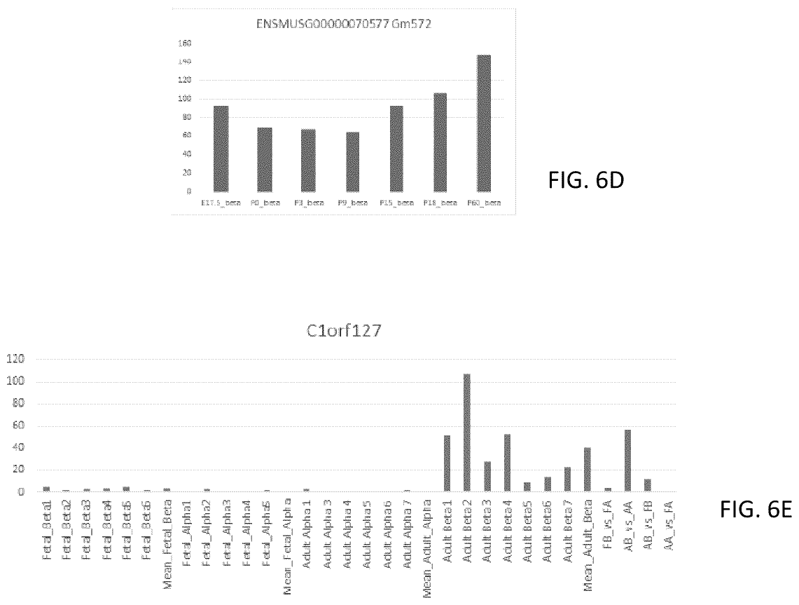

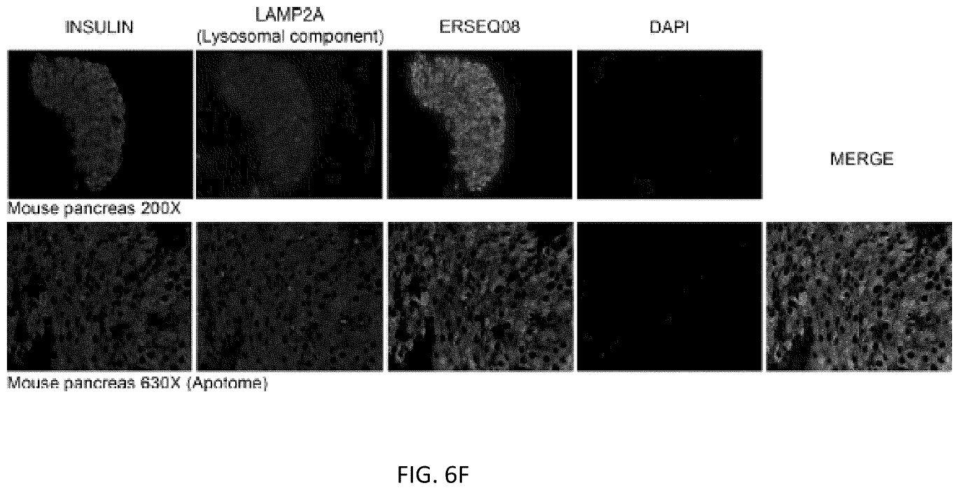

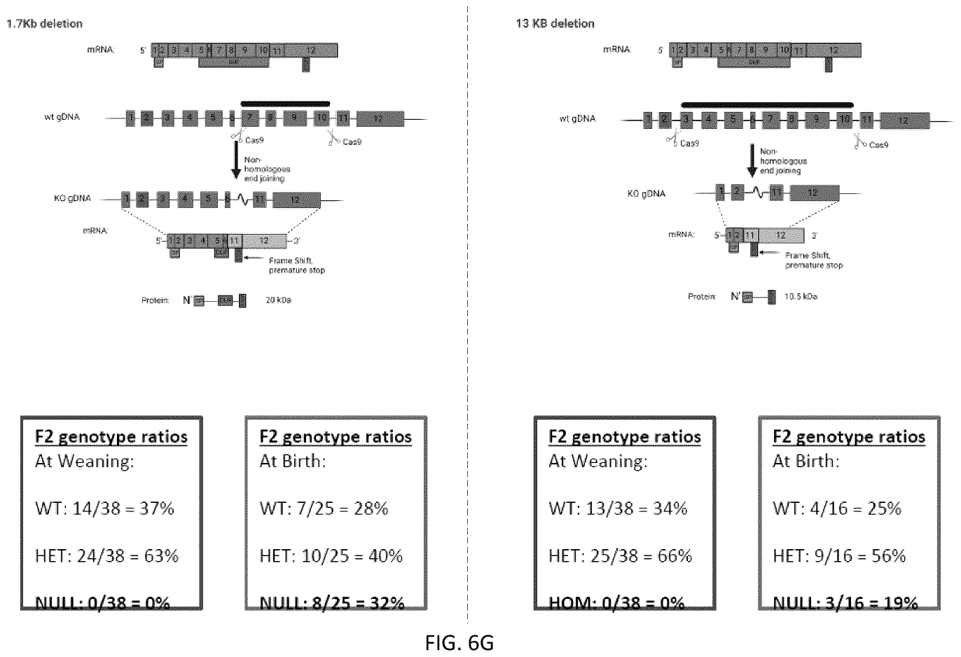

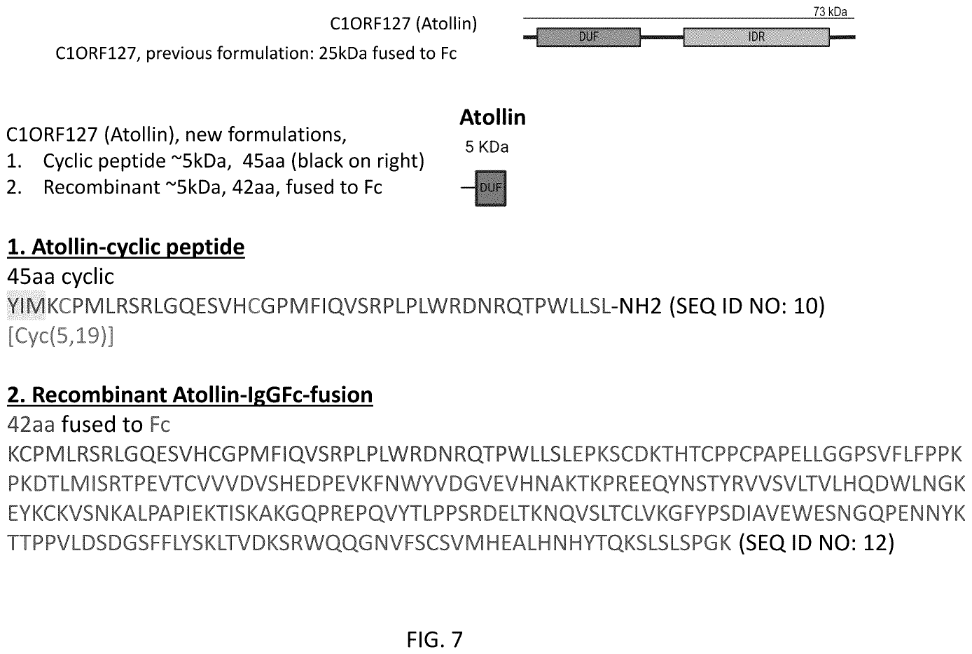

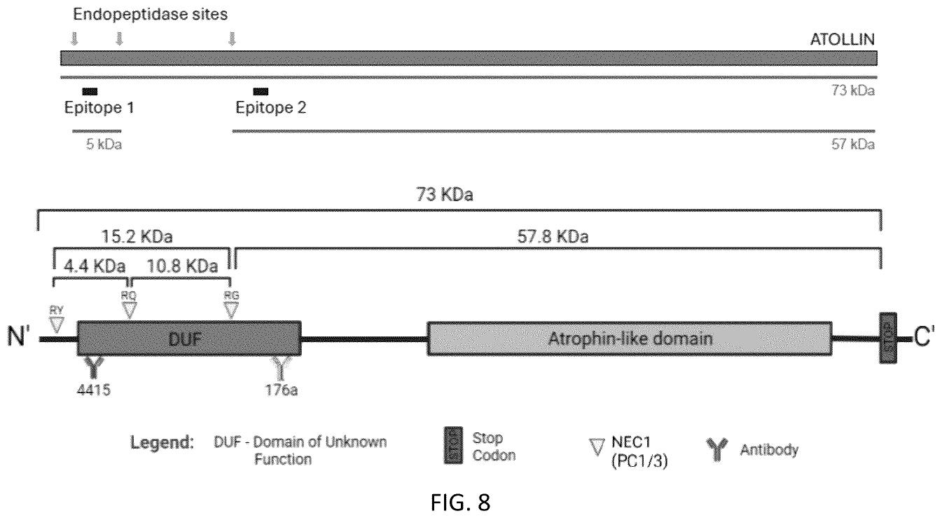

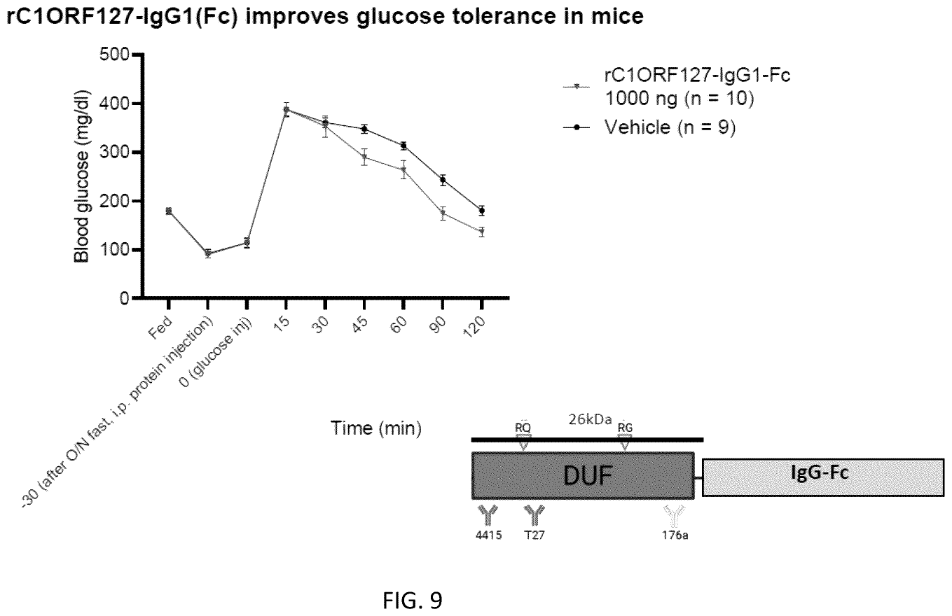

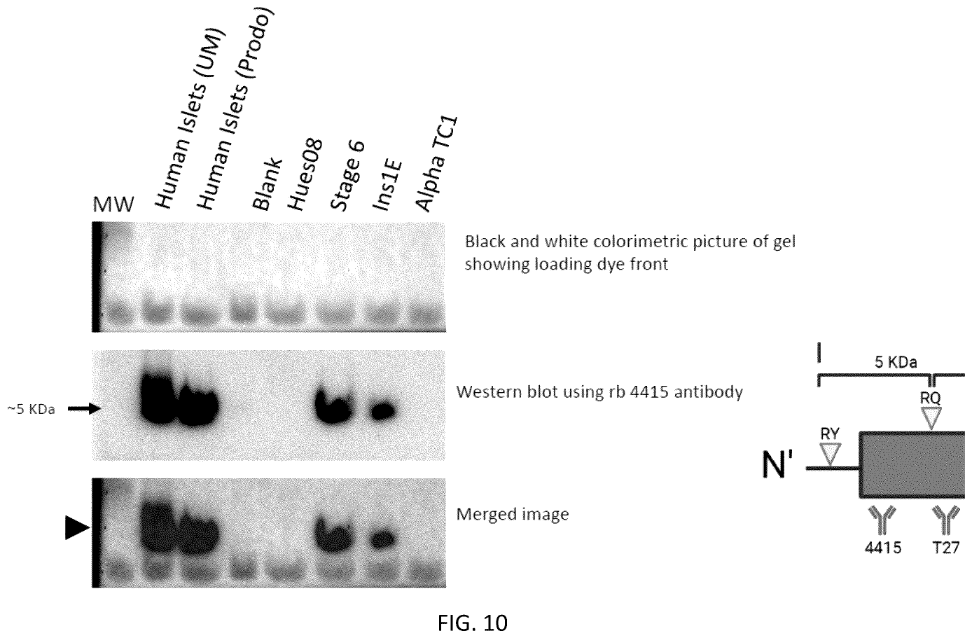

At later stages of differentiation, the inventors found enrichment for genes involved in insulin secretion and preferentially expressed in endocrine cells in SC-β cells compared to cells at earlier stages of differentiation (FIG. 1T). Interestingly, 11 unannotated genes were identified that also displayed an expression pattern specific to SC-cells. Gene ontology analysis of SC-β cell-enriched genes showed a significant enrichment of factors involved in insulin secretion, glucose homeostasis, extracellular space, secretory granules, as well as other categories that correlate with secretion and membrane-targeting processes (FIGS. 5C-5D).

### Glucose Handling Screen of Novel β Cell ER-Seq Hits

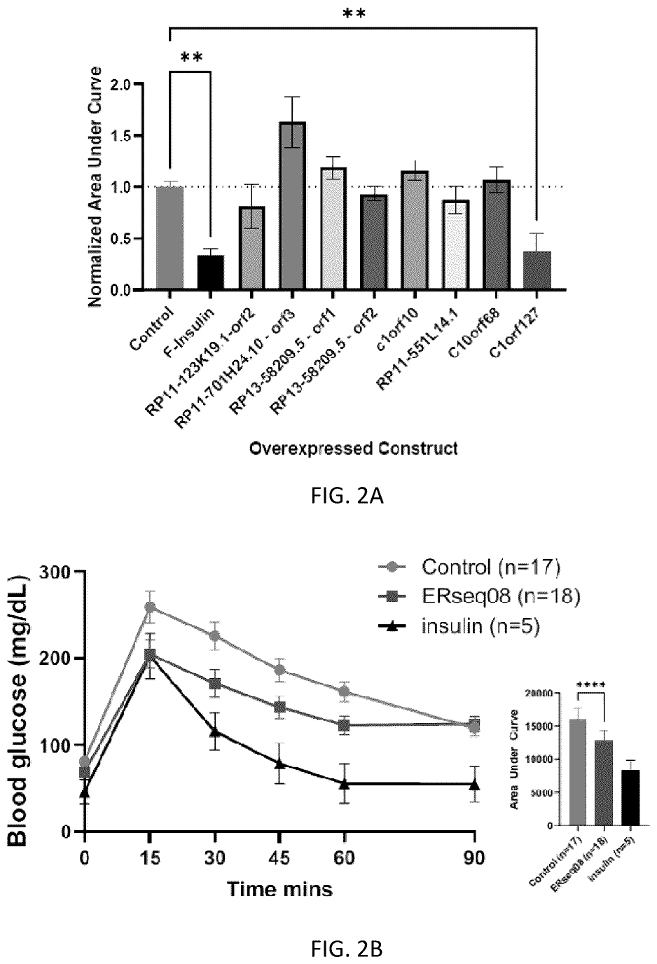

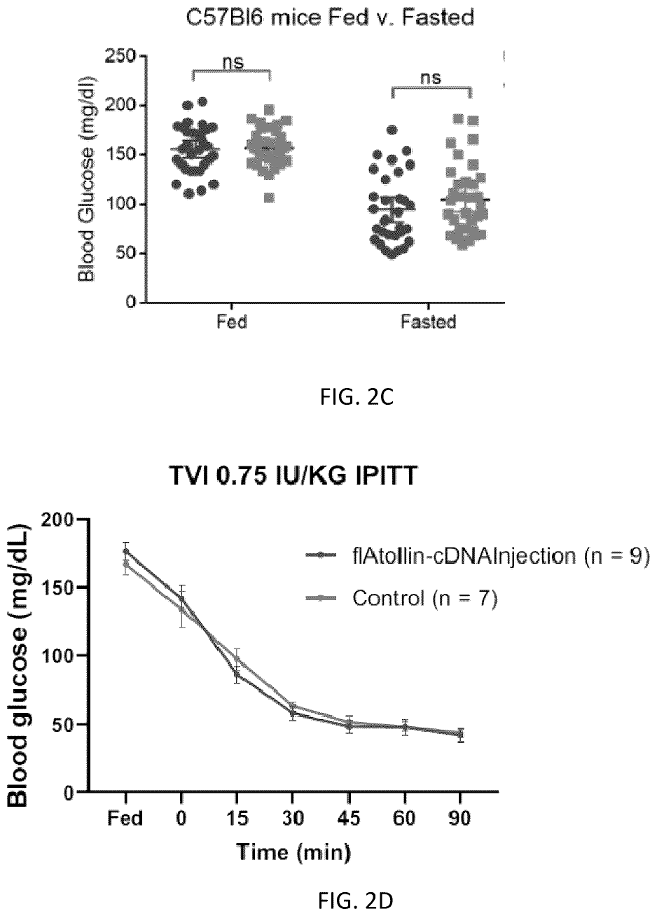

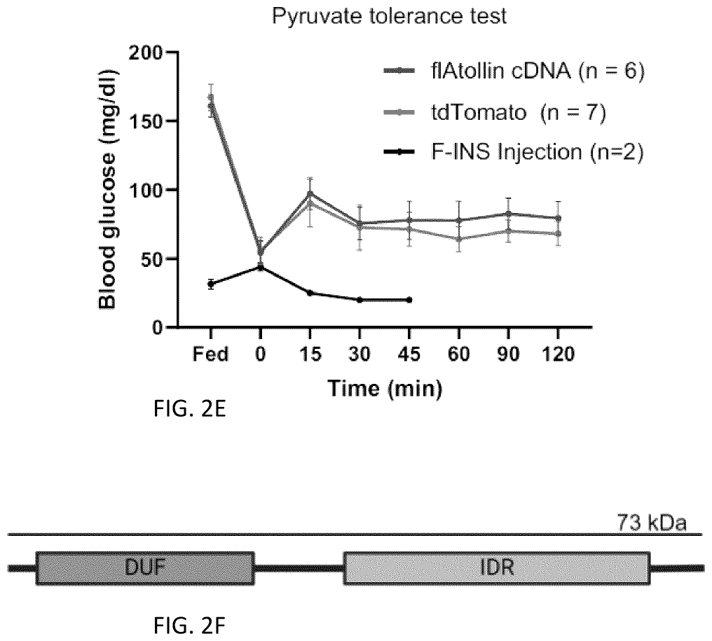

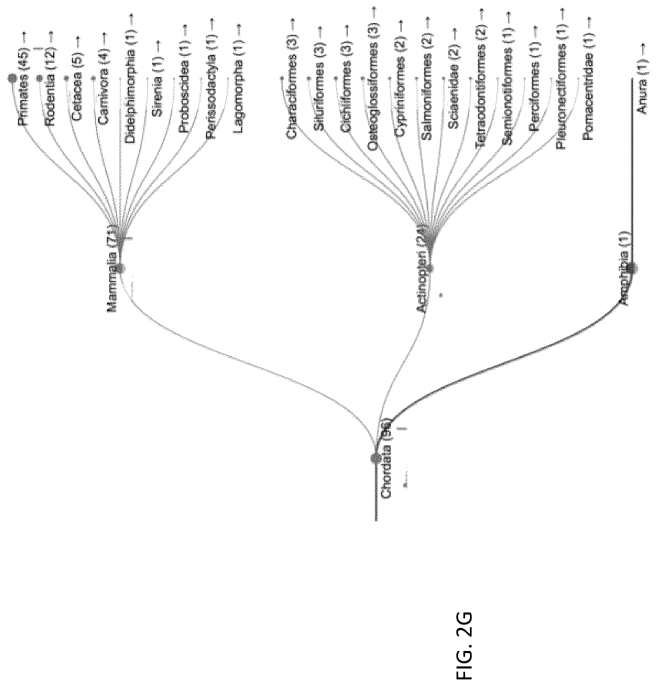

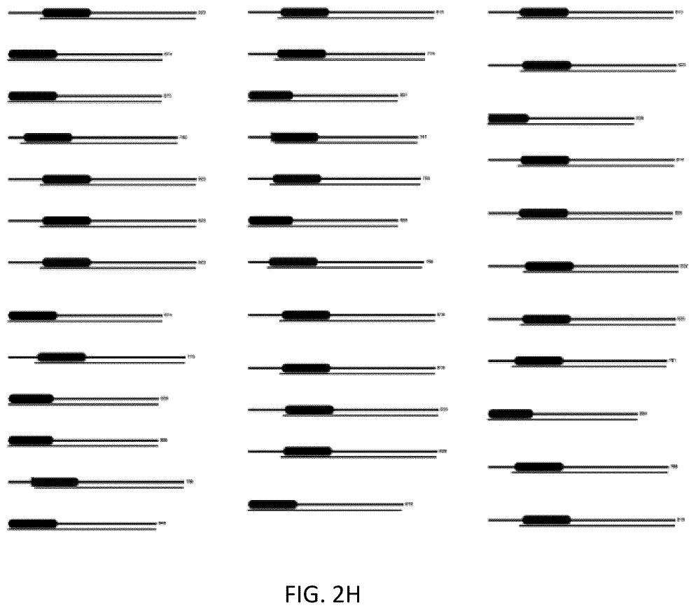

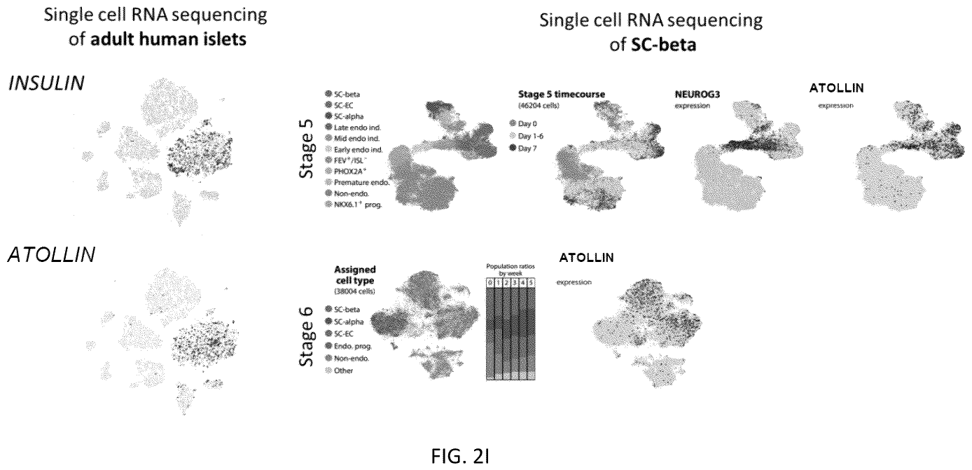

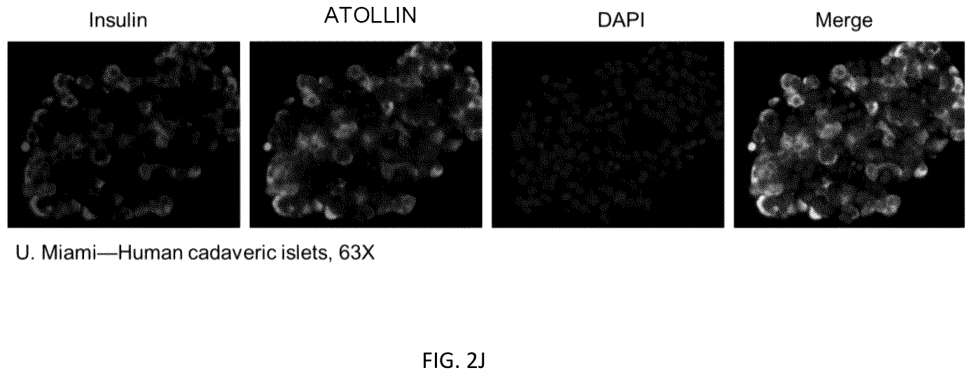

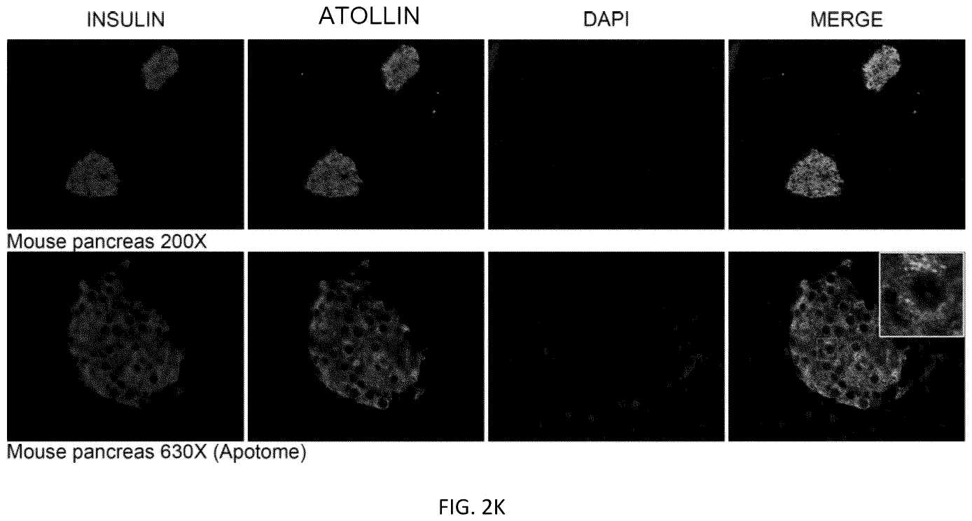

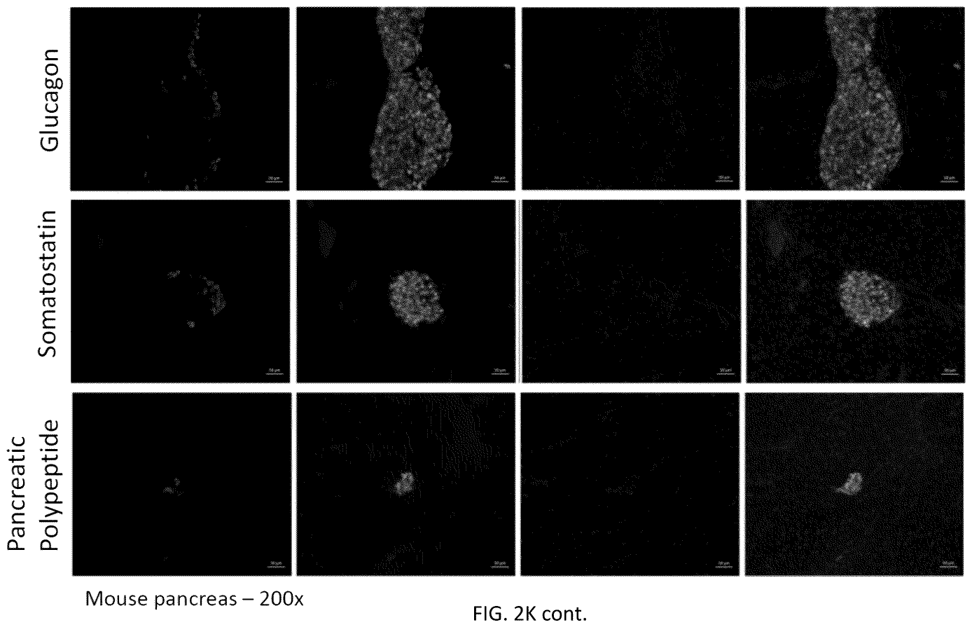

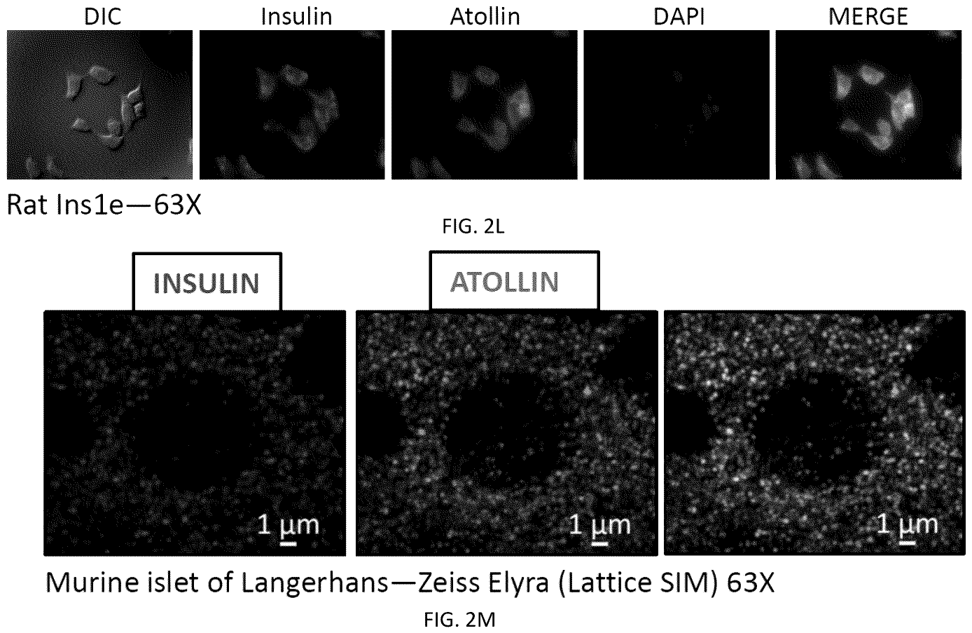

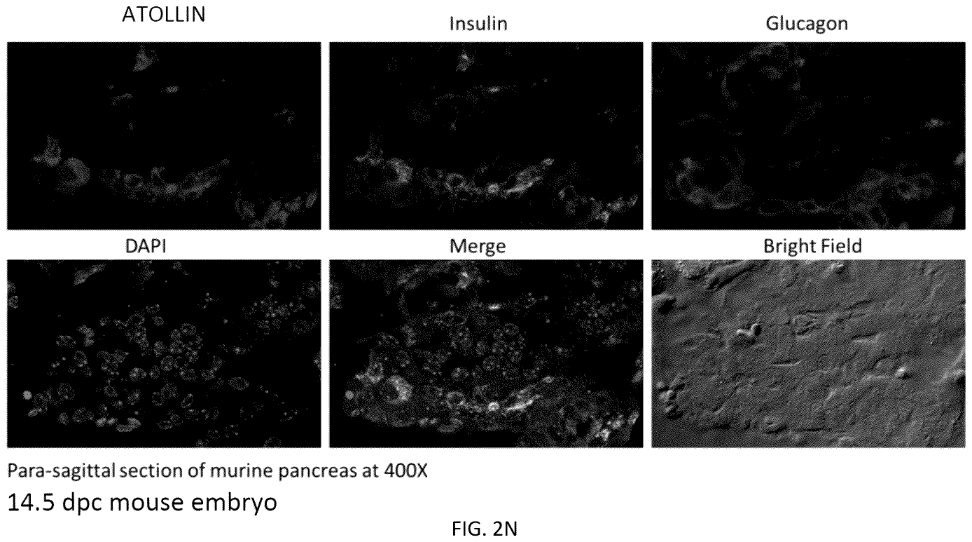

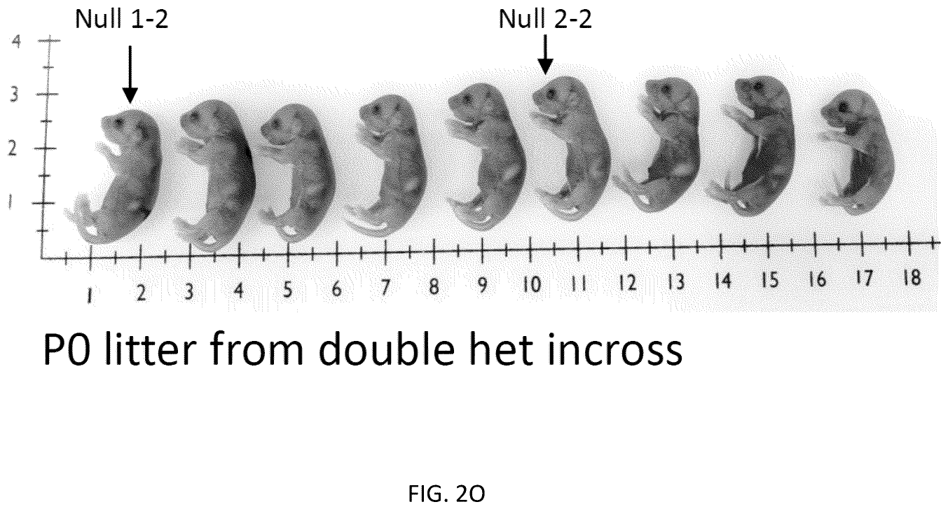

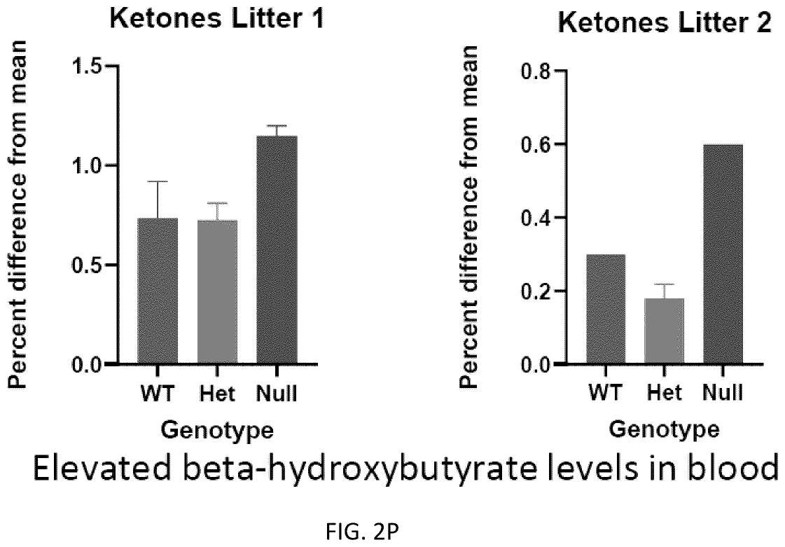

Since beta-cells are master regulators of glycemia, it was hypothesized that the 11 unannotated transcripts that were found enriched in beta cells may have roles in controlling glucose utilization. To test the ability of these genes to modulate glucose tolerance in mice, the inventors delivered via hydrodynamics-based liver transfection plasmids that would allow the constitutive expression of each gene and a Furin-cleavable Insulin as a positive control (Gerace et al). On the fourth day after plasmid delivery, and after an overnight fast, an intraperitoneal glucose tolerance test was performed on the mice to assess the glucose lowering activity of each gene. One human gene, an undescribed transcript which has been named Atollin, lowered blood glucose without causing hypoglycemia (FIGS. 2A-2C). This immediately piqued the inventors' interest as iatrogenic hypoglycemia is a major setback of Insulin therapy and for many other standard of care drugs in diabetes (Cryer review). The improvements in glycemia were not associated with increased tolerance to Insulin or pyruvate treatment (FIGS. 2D-2E). The lack of fasting hypoglycemia, the absence of synergism with Insulin and the normal response to convert pyruvate into glucose made the inventors wonder if tissues other than liver could be responsible for the improvements in glucose handling. Thus, Atollin became the lead candidate for further analysis.

Atollin mRNA Expression Correlates

The isoform of Atollin that was identified in the screen is predicted to code for a protein of 684 amino acids with a molecular weight of 73 kDa (FIG. 2F). It is located on Human Chromosome 1 at 1p36.22, and it is a highly conserved protein that only exists in vertebrates, with a notable absence in Reptilia (FIG. 2G). Atollin has been associated with roles in primary ciliary dyskinesia and may participate in left-right axis formation (Alsamri M T et al, 2021, Szenker-Ravi et al, 2022). In has no intra-species homologues. The predicted sequence of the isoform lacks canonical signals for secretion or membrane insertion but has an extremely well conserved N-terminal region of 215 amino acids from the start methionine (black bars in FIG. 2H). Region 2-198 is predicted to encode an all-Beta structure containing domain (DUF4556; Interpro: IPR049521). The C-terminal of the protein is extremely disordered and rich in Proline, and, computationally, it has been determined to be an intrinsically disordered region (IDR).

Atollin expression was confirmed in SC-beta cells and cadaveric human Islets in the scRNA sequencing databases (Veres et al, FIG. 2I). Neurogenin3 is a master regulator, and one of the earliest markers, of the endocrine lineage. As judged by the synexpression with Neurogenin3 in the psuedotime trajectory, in SC-beta cells, Atollin is expressed from the inception of the endocrine lineage. Analysis of published datasets revealed that the mouse ortholog of Uncharacterized protein C1ORF127, Gm572, heretofore referred to as mAtollin, is also coexpressed with Neurogenin3 at embryonic day 15. In SC-beta cells, its expression is later restricted to beta and enterochromaffin cells. In cadaveric human islets, Atollin is expressed in the same cluster as beta cells with modest representation in delta (somatostatin expressing) and gamma (pancreatic polypeptide expressing) cells.

Since, Atollin is expressed at a critical stage in the development of the endocrine lineage, the inventors next sought to determine if its chromatin was dynamically regulated during development, accessible to transcription, and regulated by beta-cell transcription factors. Analysis of the gene regulatory network, chromatin accessibility, chromatin occupancy, and bulk RNAseq data at key stages in the maturation of SC-endocrine progenitors into SC-beta cells demonstrated that the genetic interval encompassing Atollin is dynamically regulated during development and converges in a mature stable signature (Alvarez-Dominguez et al). Next, the inventors turned their attention to murine transcription factor binding CHIP-Seq datasets, and were able to find that NeuroD, PDX1 and NKX6.1 can bind to conserved and actively transcribed regions in the mAtollin genetic interval in murine insulinoma cell lines and islets. Moreover, in mice and humans, the expression of Atollin is maturity associated. In mice, its expression increases during the postnatal period and in humans higher expression was found in adult beta cells relative to fetal tissue. Other notable tissues with expression in publicly available datasets are in humans-skeletal muscle and cerebellum; in mice-gut enteroendocrine cells.

### Cellular and Tissue Level Characterization of Atollin Protein

To confirm the spatiotemporal mRNA expression of Atollin at the protein level, the inventors developed a series of rabbit polyclonal antibodies to Atollin. Several of these antibodies were used in immunofluorescence staining of adult human cadaveric islets, adult mouse islets, and insulinoma cell lines to demonstrate that Atollin is co-expressed in beta-cells with Insulin (FIGS. 2J-2L). Immunofluorescence staining of human cadaveric islets and mouse islets evealed a high degree of stain concordance with Insulin containing vesicles (FIGS. 2J-2L). Using the mouse, the inventors looked more closely at the expression of Atollin in other hormone lineages and found that in adult mice Atollin is exclusively expressed in beta-cells (FIG. 2K). To rule out expression in other vesicular compartments not directly associated with hormone secretion immunofluorescence staining was performed using antibodies that mark endosomes, lysosomes, or autophagosomes in a rat Insulinoma cell line, Ins1e. As judged by the lack of co-expression of Atollin with Rab5, Lamp2, and p62, it was concluded that Atollin is found with Insulin in secretory vesicles. To confirm this observation, the inventors took advantage of super resolution microscopy, which can resolve structures in the nm range, and the colocalization of Atollin with Insulin in secretory vesicles was confirmed (FIG. 2M). These images provided the inspiration for the name Atollin.

The transcriptomic data associating Atollin with early cell fate specification events in the pancreatic endocrine lineage and the adult expression in beta cells prompted the inventors to look more closely at Atollin expression in mouse embryos at 14.5 dpc (FIG. 2N), a time where pancreatic endocrine progenitors are actively being specified. Immunofluorescence microscopy was used to confirm that mAtollin and Insulin are co-expressed in developing mouse pancreas and to confirm that it is not present in glucagon producing alpha cells.

mAtollin Loss-of-Function

Intrigued by the expression of Atollin in beta-cells and knowing that it had a strong glucose lowering activity the inventors decided to generate loss-of-function alleles to mAtollin. To this end CRISPR/Cas9 technology was utilized and two deletion alleles were generated using a dual guide zygote injection strategy. The deletion strategy resulted in two alleles, eliminating 13.5 kb and 1.4 kb of genomic sequence in the mAtollin locus. Both alleles were predicted to produce truncated transcripts that result in premature translation stops. Remarkably, when heterozygous animals were bred to generate null embryos, the inventors failed to recover null animals at weaning for both alleles. Intrigued by this finding, the analysis shifted to the first days after birth and the inventors recovered live null offspring at birth which died within the first 36 hours.

To investigate the perinatal lethality, the inventors examined pups with the 13.5 kb deletion. Pups with the 13.5 kb deletion underwent normal parturition, appeared to breath well, and were able to feed, as evidenced by the presence of milk spots in their stomach (FIG. 2O). They responded well to touch stimuli and were able to move. Since it was expected that the null allele would have metabolic phenotypes associated with glucose utilization, the inventors next measured blood glucose and beta-hydroxybutyrate levels. There was no apparent effect on glycemia but, surprisingly, the animals had elevated levels of ketones, as judged by the elevated beta-hydroxybutyrate levels in blood (FIG. 2P). The perinatal lethality and increase in ketones phenocopies the phenotype first described for the Insulin receptor null allele (Acilli et al).

With no apparent gross morphological deficits present the inventors next looked internally at the liver and pancreas

### Atollin Improves Glycemia in Diabetic Models

The inventors have demonstrated that Atollin is expressed in beta-cells and, like Insulin, can lower glycemia. The null phenotype indicates that it plays important roles in controlling metabolic fuel utilization. Thus, the inventors wanted to test if Atollin could lower blood glucose levels in models of Type 1 and Type 2 diabetes. Type 1 diabetes is characterized by patent hyperglycemia, resulting from the autoimmune destruction of the beta-cells. Type 2 diabetes is characterized by post-prandial hyperglycemia and hyperlipidemia and results from peripheral insensitivity to Insulin.

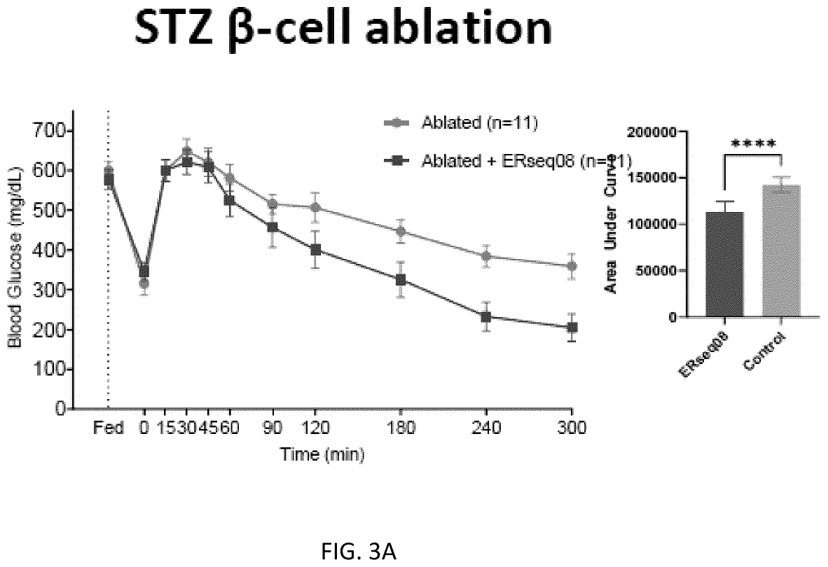

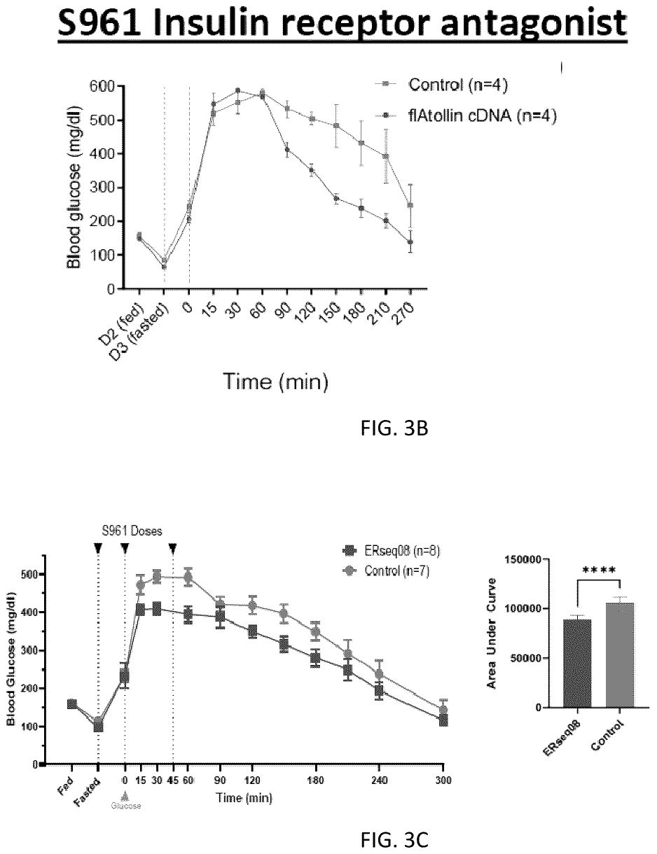

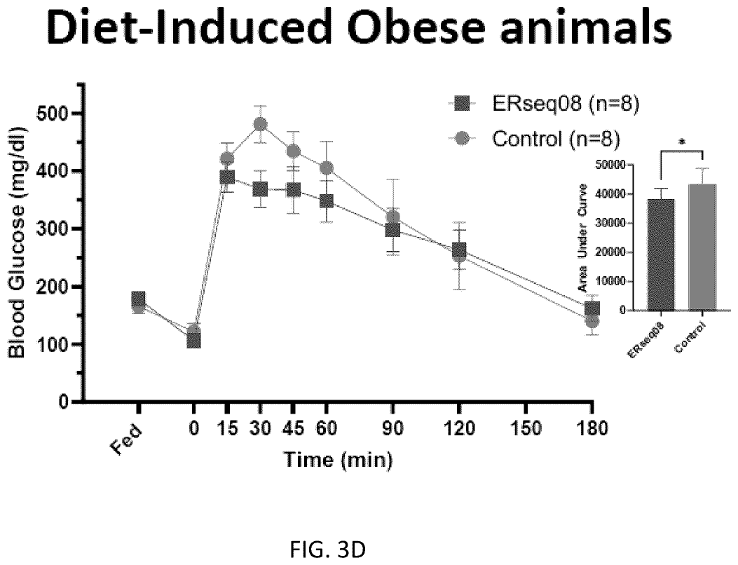

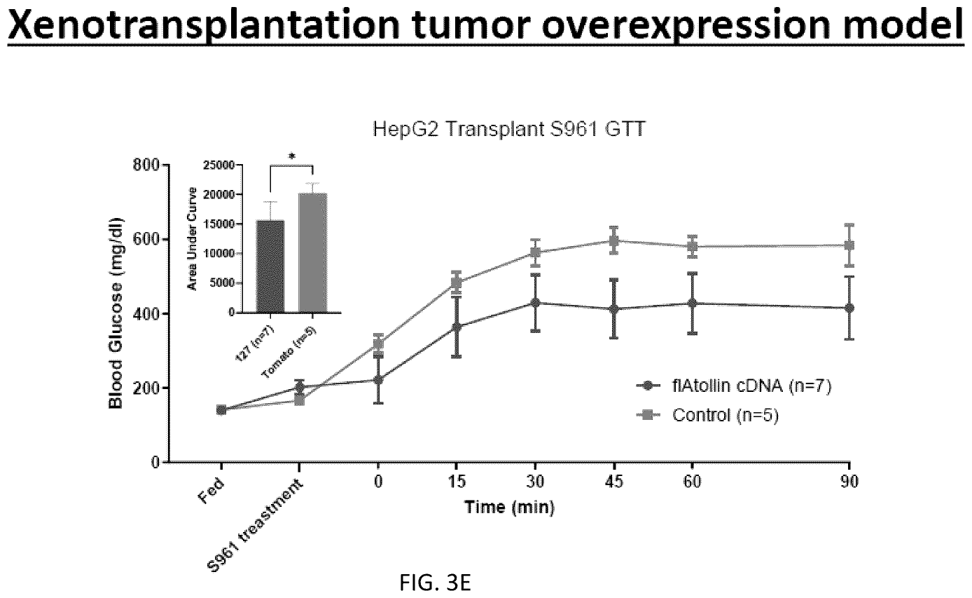

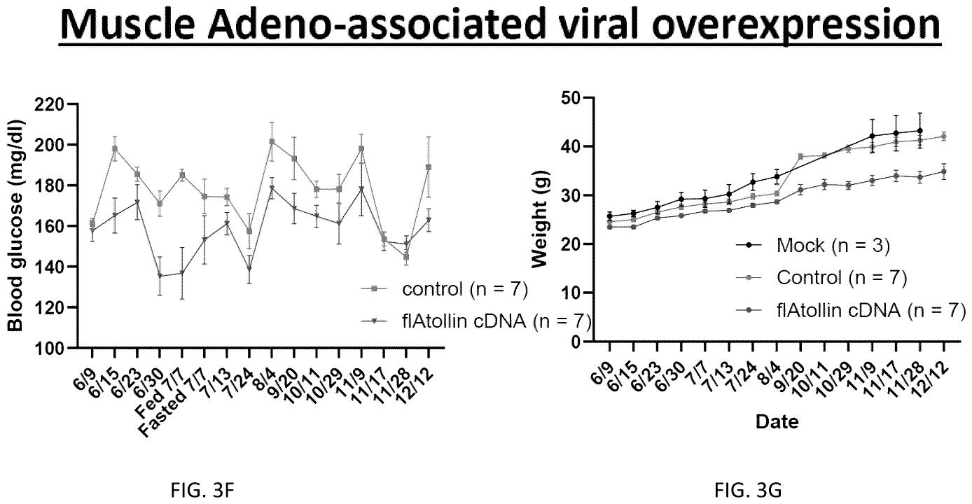

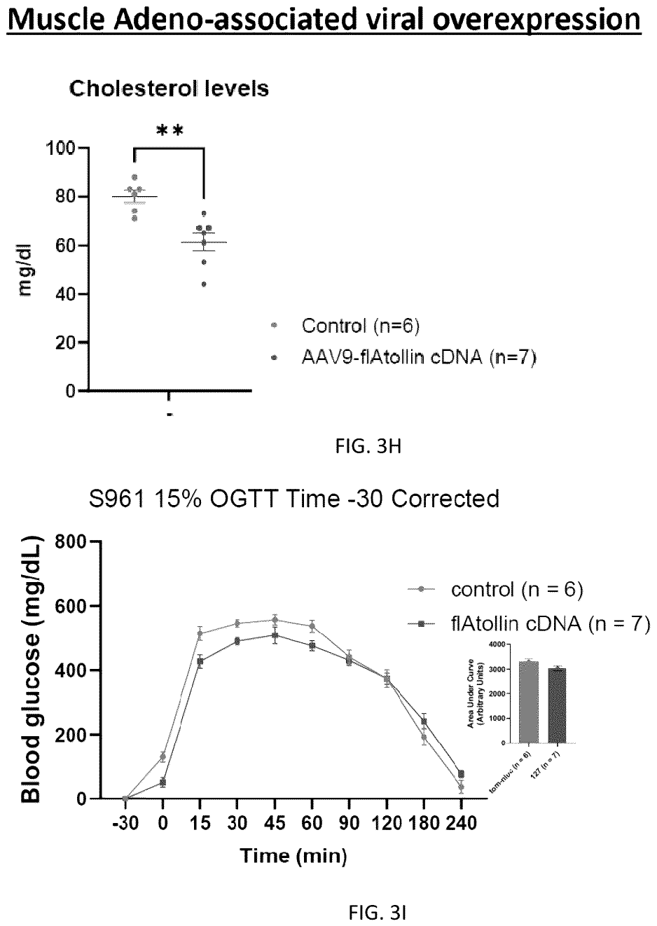

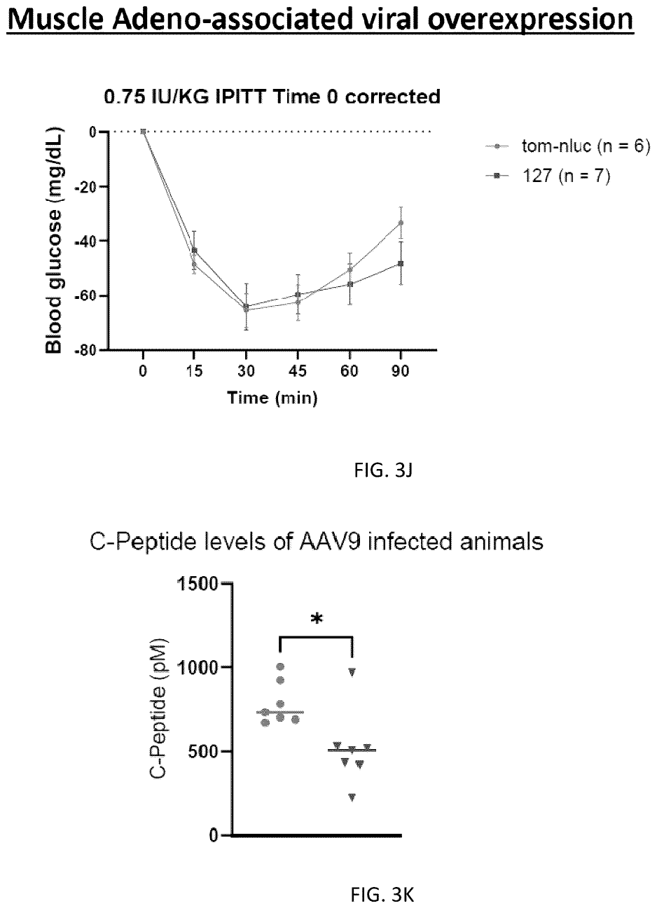

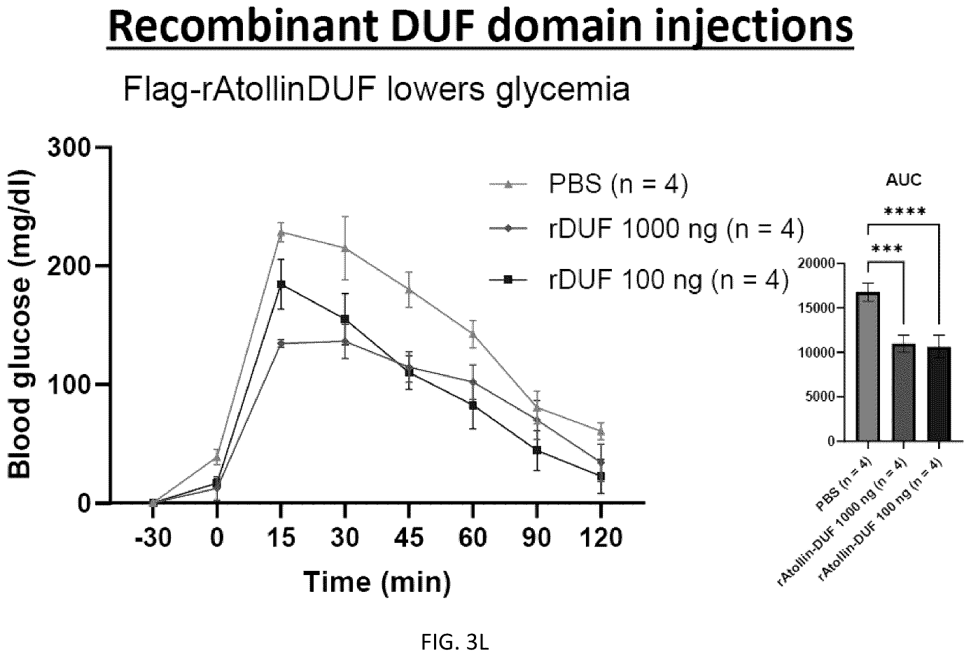

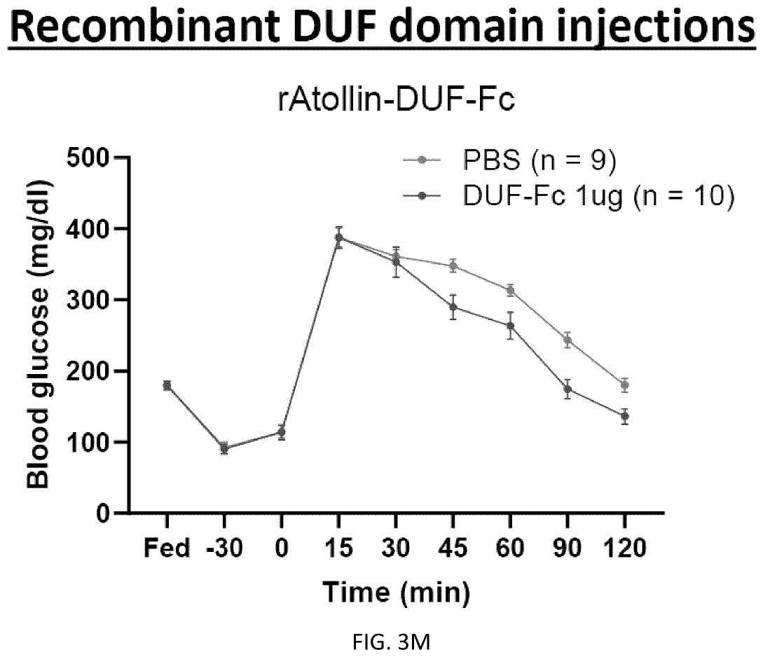

To model Type 1 diabetes, C57B16NJ mice were treated with the beta-cell antibiotic toxin, Streptozotocin (STZ), using a multiday low-dose injection regimen. After patent diabetes had taken hold, mice were transduced via hydrodynamic tail vein liver transfection with plasmid constructs encoding the full-length cDNA of Atollin or a control red fluorescent protein (tandem dimer Tomato). Four days after transfection the inventors performed an intraperitoneal glucose tolerance test after an overnight fast. Overexpression of Atollin in livers of STZ-induced diabetic animals resulted in two notable phenotypes: (1) Atollin did not cause fasting hypoglycemia (FIG. 3A, Time 0); and (2) Atollin was able to dramatically improve the glucose tolerance of STZ-treated animals. Interestingly, Atollin overexpressing animals had a peculiar response towards the end of the assay where their glycemia was lower than the fasted values (FIG. 3A) pushing them into the normoglycemic range. Thus, Atollin overexpression can overcome chronic inhibition of insulin signaling resulting from beta-cell ablation.

The improved glycemia seen in the STZ model was so remarkable that the inventors wanted to confirm the findings using a different method, an acute hyperglycemia model. For this, Insulin signaling was inhibited by treating animals with a competitive peptide antagonist of the Insulin receptor, S961. S961 causes acute hyperglycemia and hyperinsulinemia in mice. The inventors overexpressed Atollin's cDNA or a control red fluorescent protein using hydrodynamics-based tail vein transfection. Four days after the transfection, an intraperitoneal glucose tolerance test was performed after an overnight fast. S961 administration caused acute hyperglycemia in mice, demonstrating the efficacy of the S961 regimen at inhibiting insulin signaling and preventing glucose uptake (FIG. 3B, Time 0). Mice injected with the cDNA of Atollin cleared glucose from the circulation faster than controls (FIG. 3B). The 2 g glucose/kg of body weight that was used in this experiment precluded the investigation of the early kinetics of Atollin's action (the S961 injection regimen induced hyperglycemia to a degree larger than what could be measured with the glucometers). Thus, to resolve the early effects of Atollin, the inventors decided to repeat the experiment in a new cohort by reducing the glucose bolus to 1.5 g glucose/kg of body weight. To guarantee blockade of the Insulin signaling during the glucose tolerance test S961 was injected 45 minutes after delivering the glucose bolus. It was found that animals overexpressing the cDNA for Atollin have a rapid (as early as 15 min) and marked improvement in glucose tolerance relative to control animals overexpressing a red-fluorescent protein (FIG. 3C). The increased glucose tolerance mediated by Atollin in the absence of beta-cells or after blockade of the Insulin receptor suggests that Atollin may be able to remove glucose from the circulation independent of insulin action.

Animals rendered obese by feeding a high fat diet regimen mimic the metabolic phenotypes seen in Type 2 Diabetes, which is usually associated with obesity in humans. Thus, the inventors wanted to see if Atollin was able to improve glycemia in obese animals. Obese animals were injected with the cDNA of Atollin or that that of a control red-fluorescent protein as described. On the fourth day after transfection and after an overnight fast the inventors performed an intraperitoneal glucose tolerance test. Atollin driven improvements were also seen in this model as indicated by the increase in glucose tolerance seen in Atollin overexpressing animals relative to controls (FIG. 3D). Thus, Atollin can improve glycemia in mouse models of Type 1 and Type 2 diabetes.

### Atollin is an Endocrine Modulator of Glucose Homeostasis and Metabolism

To test if Atollin functions as a secreted protein independent of the hydrodynamics-based transfection, and to explore long-term effects of this activity, two long term gain-of-function approaches were employed. The first, a tumor xenotransplantation experiment, and the second, an adeno-associated virus overexpression model.

For the tumor xenotransplantation model, control or Atollin overexpressing cells were transplanted into mice and their glucose metabolism was assayed. For this experiment, a tumor xenograft transplant model was used where HEPG2 hepatocellular carcinoma cells stably overexpressing Atollin or control constructs were injected into the kidney capsule of immunocompromised SCID-Beige mice. After four months when tumors had formed, mice with tumors overexpressing Atollin did not have differences in ad-libitum of fasted glycemia as compared to controls (FIG. 3E, −240 time-after O/N fast). Since the activity seemed to be glucose dependent, the animals were treated with the insulin receptor inhibitor S961. When injected with glucose, this treatment resulted in long-lasting high blood glucose levels in control mice (FIG. 3E). Together, this suggests that Atollin can work as an endocrine activity to control glucose utilization.

For the adeno-associated virus overexpression system (AAV), the inventors generated constructs to overexpress the full-length cDNA of human Atollin (from the ER-seq screen and as described for the hydrodynamic-based delivery) and a control red-fluorescent protein. Since the inventors were looking to overexpress the activity in tissues other than liver, the construct was designed to be expressed under the control of tMCK, a muscle specific promoter (muscle-specific creatine kinase). AAV9 viral particles were delivered systemically through the retro-orbital plexus at seven weeks after birth and the experiment was terminated when the animals reached eight months of age. As early as the second week of treatment, it was noticed that the Atollin overexpressing animals had reduced ad-libitum glycemia (FIG. 3F). This phenotype was maintained throughout the study period. Initially, there was no difference in weight gain among groups but starting around three months after injection, Atollin overexpressing mice had reduced age associated weight gain relative to controls (FIG. 3G). Four months after injection, an S961 oral glucose tolerance test was performed following an overnight fast and glucose tolerance levels were compared between Atollin overexpressing and control animals (FIG. 3I). Animals overexpressing Atollin overcame S961 mediated, insulin receptor, inhibition and had an improved glucose tolerance phenotype. Albeit having lower glycemia, the inventors were surprised to find that at eight-weeks post-infection, levels of C-peptide, a by-product of insulin production, were lower in Atollin overexpressing animals relative to controls (FIG. 3K), consistent with Atollin having a strong Insulin sensitizing effect. Glucagon was not differentially regulated amongst groups. Next, an Insulin tolerance test was performed in both virally transfected groups and it was concluded that Insulin tolerance was similar among the two groups (FIG. 3J) a notable phenotype in the hydrodynamics-based liver transfections (FIG. 2D). Total blood plasma was collected from ad-libitum fed mice at the end of experimentation and submitted for blood chemistry analysis. Insulin and Glucagon ELISAS were also performed on this plasma. The terminal blood chemistry analysis revealed a marked reduction in total cholesterol accompanied by an increase in bile acids (FIG. 3H) while other parameters remained unchanged amongst groups.

While improvements were seen in glucose tolerance after S961 treatment in both the tumor and the viral cohort, neither cohort showed glucose improvement in regular tolerance tests. One explanation for these observations is that Atollin works as a proprotein and thus, would have greatest activity when processed endogenously in beta-cells. Nevertheless, the results from both the tumor and the viral model suggest that Atollin works as a secreted protein to lower blood glucose levels and may operate through Insulin-independent mechanisms.

### Atollin's Glucose Lowering Activity Resides in the DUF Domain

Molecular conservation analysis showed that the domain of unknown function (DUF) is the most evolutionary conserved part of the Atollin protein. To test if the glucose lowering activity was in this region, the inventors decided to move away from cDNA overexpression, and engineered constructs to produce a recombinant protein fragment encompassing the first 215 aa of Atollin, where the DUF domain is located, and this was expressed and purified from HEK293 cells using an N-terminal FLAG-tag (Flag-rAtollin-DUF). Mice were fasted overnight, and an oral glucose tolerance test was performed. 30 minutes before the injection of glucose the recombinant DUF fragment was injected into the intraperitoneal cavity. As seen in FIG. 3L, animals injected with Flag-rAtollin-DUF can clear glucose from the circulation faster than controls. To increase the perdurance of the glucose lowering phenotype, C-terminal IgG-Fc (Fc) fusions to the rAtollin-DUF (rAtollin-DUF-Fc) were engineered. Using a similar recombinant protein production and injection paradigm, the inventors were able to show that rAtollin-DUF-Fc is also able to improve glucose tolerance (FIG. 3M). Together this suggests that the glucose lowering activity of Atollin resides in the DUF, and validates the results described earlier with the cDNA overexpression via hydrodynamics-based transfection. Moreover, the activity of the Fc-fusion suggests that it may operate through a receptor.

### Atollin is a Proprotein, Processed by Prohormone Convertases

Since Atollin is produced in beta cells and can work as an endocrine modulator of metabolism, the inventors wanted to explore the possibility that Atollin proprotein may harbor a smaller active fragment. To characterize its processing, protein extracts from primary human and mouse islets, and insulinoma cells lines were analyzed by western blot (FIG. 4A). Analysis of protein extracts from cadaveric human islets revealed the presence of two prominent bands of ˜73 kDa and ˜57 kDa. The 73 kDa band corresponds to the predicted molecular weight of the isoform of Atollin identified in the ERseq screen. The 57 kD band may correspond to a processing event at LVKR|G (SEQ ID NO: 52), a canonical endopeptidase, proprotein convertase-like, recognition site (depicted as R|G in diagram). This site is remarkably like that for Insulin between the C-peptide and the A-chain: LQKRG (SEQ ID NO: 53). This immunoblot was performed with an antibody located C-terminal to the R|G site in the conserved domain of unknown function (FIG. 4A). In Insulin, this site has recently been proposed to be cleaved by proprotein convertase 1/3 in human cells (Kieffer paper and ref therein). Therefore, the inventors wanted to investigate if Atollin interacts with endopeptidases at the molecular level. For this the inventors turned to both a candidate and a discovery approach. For the candidate approach, immunoprecipitations were performed with the same antibody using protein extracts from cadaveric human islets and the lysates were tested against antibodies to Atollin and PC1/3 by western blot. As seen in FIG. 4B, the antibody was able to pulldown the bait (Atollin). Using this strategy, it was found that Atollin immunoprecipitated PC1/3, suggesting that Atollin and PC1/3 interact at the molecular level in human islets. For the discovery approach, protein immunoprecipitation lysates were prepared from human cadaveric islets using the same antibody used in the pulldowns and another antibody designed against the DUF domain N-terminal and proximal to the R|G site and they were submitted for protein identification via liquid chromatography coupled mass spectrometry analysis. This analysis confirmed that Atollin is in a molecular complex with PC1/3, and also revealed that Atollin is closely associated with many proteins found in Insulin containing vesicles that have been implicated to participate in Insulin trafficking through the co-translational protein translocation pathway (FIG. 3D).

To define these and other processing events that may not be detected by the two antibodies used above, the inventors turned their attention to analyzing the expression and processing of fusion proteins between Atollin and moxGFP (an ER stable, enhanced GFP monomer) in stably transfected insulinoma cell lines. Both N-terminal and C-terminal fusion constructs to Atollin (moxGFP-Atollin and Atollin-moxGFP respectively) were generated and then transfected into Ins1e cells using Lentiviral vectors. Lines only expressing moxGFP were used as a control (FIGS. 4F-4G). Fluorescent micrographs of the moxGFP/Atollin fusions show a subcellular distribution pattern like that of a secreted protein (cytoplasmic, vesicular-like, and absent from the nucleus, FIG. 4G). Next, protein lysates were prepared from these cells and western blot analysis was performed with antibodies against Atollin. The blots were stained with T27, an antibody in the DUF domain that recognizes the human protein made by the rat Ins1e cells. Staining with this antibody revealed the existence of several bands which may correspond to endogenous processing events (FIG. 4H). The band at around 45 kDa corresponds to the predicted molecular weight of the cleavage at R|G when measured from the N-terminus R|Y site (moxGFP is 28 kDa plus 15.3 kDa from Atollin's N-terminus). The T27 antibody can detect an endogenous band of ˜15 kDa in tissue extracts from cadaveric human islets (FIGS. 4I-4J). The presence of other bands suggested other potential processing events. However, the band that piqued the most interest was a band running at ˜33 kDa (FIG. 4H). This band would correspond to a product of moxGFP (28 kDa) fused to a ˜5 kDa N-terminal fragment of Atollin. This region is the same length as the fragment predicted to exist from R|Y near the start methionine to the first conserved endopeptidase cleavage site found in Atollin R|G (FIG. 4I). Fortuitously, the inventors had made a rabbit polyclonal antibody to this region: 4415. Using this antibody, the inventors performed western blot analysis from cell lysates of primary tissues or cell lines containing Insulin-expressing beta cells and compared them to negative control lysates from cells lines that do not have beta cells. As described in FIG. 4K, a band of ˜5 kDa was found in lysates from either Human cadaveric islets, or Human Stem cell derived beta cells, or rat insulinoma Ins1e cells, but not in Human self-renewing stem cell progenitors nor in a glucagon expressing Alpha cell line (TC1-9). This ˜5 kDa protein was also found in islet preps from rodents.

In addition to the moxGFP-Atollin fusion strategy described above, the inventors undertook a second protein tagging approach to try and elucidate other cleavage events that may be working in the liver to generate active fragments, as the liver lacks prohormone convertase activity. For this Flag epitope tagging was utilized and a series of in-frame Flag tags were engineered at four locations throughout the Atollin protein (FIG. 4L). The inventors focused on the N-terminus of Atollin, as the glucose lowering activity was found to be in this region (FIGS. 3M-3N). HepG2 cells were transiently transfected with these constructs, and protein extracts were analyzed by western blots with anti-Flag antibodies. All tags picked up the full-length protein and a potential C-terminal processing event. However, the most promising bands were seen when the Flag tag was placed in the middle of the DUF domain at 120 aa from the start Methionine. In this lysate strong bands were detected at ˜17 kDa and ˜25 kDa. The former may correspond to Furin-processing events at RSD|R (SEQ ID NO: 54) and RFL|R (SEQ ID NO: 55) predicted to generate a band of 17 kDa (inclusive of FLAG tag). The latter may start at the same RSD|R (SEQ ID NO: 54) site and would end on an uncharacterized site in between the DUF domain and the IDR. These processing events would release the DUF domain (or a portion of it) and can explain the glucose lowering phenotype seen in the hydrodynamics tail vein injections.

Together this suggests that Atollin is a proprotein that is endogenously processed into smaller fragments by prohormone convertase ⅓ in pancreas and can be facultatively processed by Furin in liver upon overexpression. Next, the inventors looked to see if any of these fragments have glucose lowering activity.

### The Glucose Lowering Activity of Atollin Resides in its N-Terminus

As an initial attempt to assess the activity of both the ˜5 kDa and the ˜15 kDa fragments, the inventors decided to go back to the Hydrodynamics-based tail vein transfection liver overexpression system. The cDNA for both proteins was cloned into the CAGGS expression vector and the signal sequence of Insulin was used for secretion. As seen in FIG. 4M, the 5 kDa fragment had the same glucose dependent glucose lowering activity as full-length Atollin and the DUF domain (FIG. 3L). The 15 kDa fragment did not demonstrate any glucose lowering activity. The 5 kDa fragment when expressed from liver as cDNA, is not sufficient to improve glycemia in the S961 model.

It was decided to confirm the glucose lowering activity of the 5 kDa fragment by generating synthetic peptides and recombinant-IGgFc fusions to this region of Atollin. Both a linear and a cyclic, disulfide bridged, peptide to the following 45 aa of Atollin: YIMKCPMLRSRLGQESVHCGPMFIQVSRPLPLWRDNRQTPWLLSL (SEQ ID NO: 10) were generated. Amidated at the C-terminus. The bold positions indicate the cysteines used for cyclization. Since an interaction with Carboxypeptidases was demonstrated in the co-immunoprecipitation work (FIG. 4E), and the protein is expressed in Insulin containing vesicles (FIG. 2), it was decided to remove the N-terminal R at the conserved endopeptidase site SLR|G. As controls, a shorter 37 aa peptide fragment ending at a putative Furin-site (Underlined), was used in two forms: linear and cyclic; and a 98 aa fragment starting at the Furin-site and ending at LVK and sequences in. These peptides were injected 30 minutes before performing a glucose tolerance test and glucose tolerance was compared relative to vehicle injected controls. As can be appreciated in FIG. 4M, the 45aa acid fragment was sufficient to improve glycemia. The cyclic peptide had increased glucose lowering activity in a dose-dependent manner (FIG. 4N). None of the other peptides tested had glucose lowering activity. The cyclic peptide had a modest but significant improvement in glycemia in the absence of beta cells in the STZ model (FIG. 4O).

It was puzzling to see that the 5 kDa fragment had glucose lowering activity as both cDNA and synthetic peptide but lacked activity in the S961, and had modest activity in the STZ models, respectively. It was reasoned that because of its small size the 5 kDa form could be rapidly cleared. Thus, it was attempted to extend the perdurance of the activity by generating recombinant fragment of Atollin42 fused to IGgFc. The inventors made both N- and C-terminal IGgFc fusions to this region: KCPMLRSRLGQESVHCGPMFIQVSRPLPLWRDNRQTPWLLSL (SEQ ID NO: 11) (Fc-Atollin42 and Atollin42-Fc respectively). The first three amino acids described in the cyclic peptide sequence (−YIM) were removed to append a flag tag to the N-terminus for purification of the DUF recombinant protein. To assess the activity of the two Fc fusions, recombinant protein was injected 30 minutes before performing an intraperitoneal tolerance test in animals that were fasted overnight. Animals injected with Atollin42-Fc had improved glucose tolerance relative to controls injected with Fc-only recombinant protein (FIG. 4P). The Fc-Atollin42 fusion did not lower glucose levels. The activity of Atollin42-Fc lasted until the next morning after the animals were returned to ad libitum feeding.

Next, the inventors tested if the 42 amino acid fragment of Atollin was sufficient to overcome acute Insulin receptor inhibition using the S961 model. Mice were fasted overnight for an intraperitoneal glucose tolerance test. S961 was given intraperitoneally at two time points: 2-hours and 30 minutes before the glucose bolus. One-hour before glucose administration, Atollin42-Fc was injected intraperitoneally. In the presence of glucose, S961 caused rapid and sustained hyperglycemia in control animals (FIG. 4Q). Animals injected with the recombinant protein had improved glucose tolerance relative to Fc-only injected controls. Thus, this 42 amino acid fragment harbors Atollin's endocrine activity.

### Example 2: Atollin, a Beta-Cell Hormone Enabling Insulin-Independent Glucose Control

Insulin secretion from pancreatic beta cells is classically viewed as the central mechanism maintaining vertebrate glucose homeostasis, but the full hormonal repertoire of beta cells and their systemic roles remain incompletely defined. Here, the inventors show that Atollin, a previously unrecognized beta cell-derived hormone present in human serum, acts as a potent, insulin-independent regulator of glucose metabolism. Using Endoplasmic Reticulum Sequencing (ER-seq) in stem cell-derived beta cells, the inventors discovered that C1ORF127 encodes Atollin, which is highly enriched in human and mouse beta cells. Acute administration of recombinant Atollin robustly lowers blood glucose and restores glucose tolerance in wildtype, obese, and insulin-deficient mice, even under pharmacological blockade of the insulin or GLP-1 receptor, indicating that its glucose-lowering action does not require insulin secretion or signaling. Mechanistically, Atollin suppresses hepatic glucose production and redirects glucose flux into glycolysis within white adipose tissue, resulting in rapid, hypoglycemia-free normalization of glycemia. Genetic loss of Atollin is perinatally lethal, resulting in severe metabolic derangement, hepatic lipid accumulation, and suppression of hepatic gluconeogenic gene programs. These findings expand the recognized repertoire of beta cell hormones and position Atollin as a promising target for overcoming insulin resistance and advancing therapies for diabetes and metabolic disease.

### Results

Glucose homeostasis is fundamental to life, with plasma glucose levels tightly controlled by precise coordination of uptake, storage, and endogenous production across feeding, fasting, and stress. This balance is exerted largely via a handful of pancreatic and adrenal hormones, including insulin, glucagon, and glucocorticoids, that together orchestrate peripheral glucose disposal, glycogen storage, and hepatic glucose production in response to metabolic cues. Disruption of this regulatory network underlies diabetes mellitus, in which uncontrolled hepatic glucose output and impaired glucose utilization drive chronic hyperglycemia and its complications (Saltiel and Kahn, 2001; Lin and Accilli, 2011; Petersen . . . Shulman, 2017; Roden and Shulman 2019, James . . . Birnbaum, 2021; DeFronzo et al., 2015)

Classically, insulin secretion from pancreatic beta cells is viewed as the primary hormonal signal lowering postprandial glucose, simultaneously suppressing hepatic glucose production and stimulating glucose uptake in peripheral tissues. Conversely, glucagon and glucocorticoids counteract hypoglycemia by mobilizing hepatic glucose stores and promoting gluconeogenesis during fasting or stress. However, the full complement of beta cell-secreted hormones and their roles in systemic metabolic regulation remain incompletely defined. Notably, as far back as 1923, Best and Scott observed that pancreatic extracts harbored not only insulin but also an insoluble “fatty” fraction with distinct glucose-lowering properties, suggesting the existence of additional secreted regulators. (Stanford and Goodyear, 2014; Moore . . . Cherrington, 2012; Radziuk and Pye, 2001).

Intriguingly, insulin-independent routes for glucose disposal have long been recognized: glucose uptake into skeletal muscle can occur via exercise- or contraction-dependent pathways, and in pivotal studies, knocking out the insulin receptor in fat or muscle produced only a mild metabolic phenotype. This suggests that other beta cell-derived hormones may help buffer glycemic excursions and adapt whole-body metabolism beyond the canonical insulin-glucagon axis. This is especially relevant given the limitations of current diabetes therapies, which focus on increasing insulin or its action but are associated with risks of hypoglycemia, weight gain, and progression to hepatic metabolic disease. (Murphy . . . Febbraio, 2020; Severisen and Pedersen, 2020).

To address these open questions, the inventors developed Endoplasmic Reticulum Sequencing (ER-seq), a targeted RNA profiling method to isolate transcripts encoding secreted and membrane proteins from endoplasmic reticulum-bound ribosomes in human stem cell derived beta cells. Among various candidates, the inventors identified C1ORF127, a poorly characterized gene highly enriched in human and mouse beta cells. Here the inventors show that C1ORF127 encodes Atollin, a hormone present in human circulation that acutely and robustly lowers blood glucose across wildtype, obese, and insulin-deficient mice, even under insulin or GLP-1 receptor blockade. Strikingly, Atollin acts independently of insulin secretion, powerfully suppresses hepatic glucose output, and diverts glucose flux into a glycolytic “burning” shunt in white adipose tissue, without promoting fat storage. Loss of Atollin is perinatally lethal, resulting in metabolic collapse. These results establish Atollin as a key player in a fundamentally new endocrine axis in glucose regulation, with broad implications for diabetes, insulin resistance, and metabolic disease.

ERseq Identifies Proteins Secreted During Human Stem Cell Differentiation into Pancreatic Islets

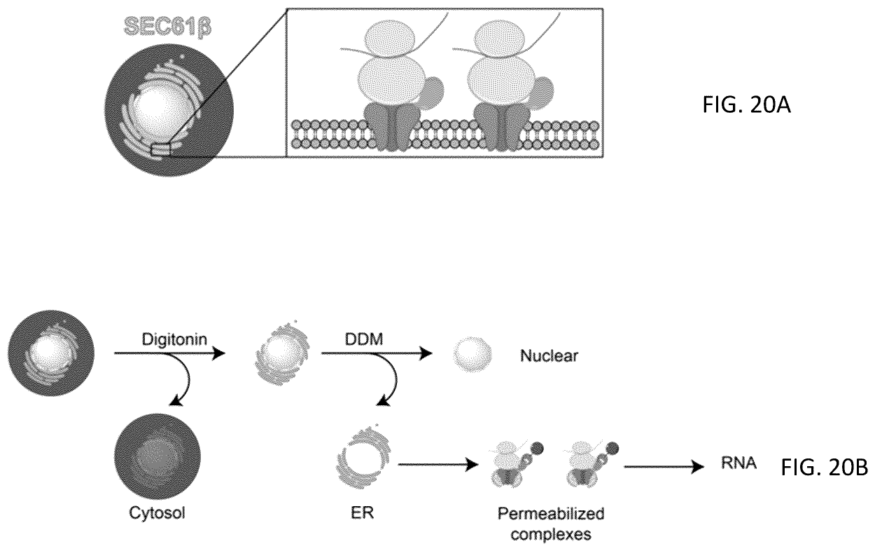

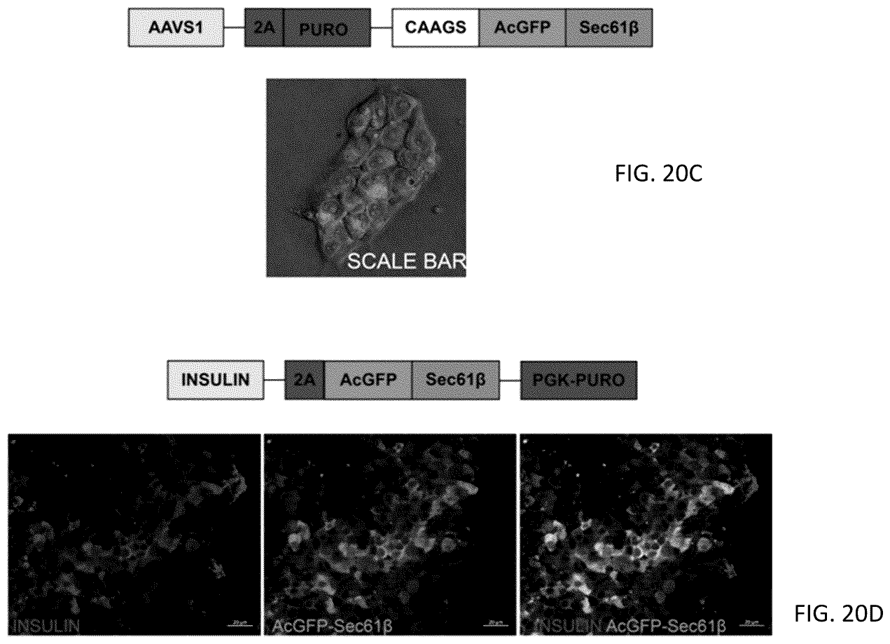

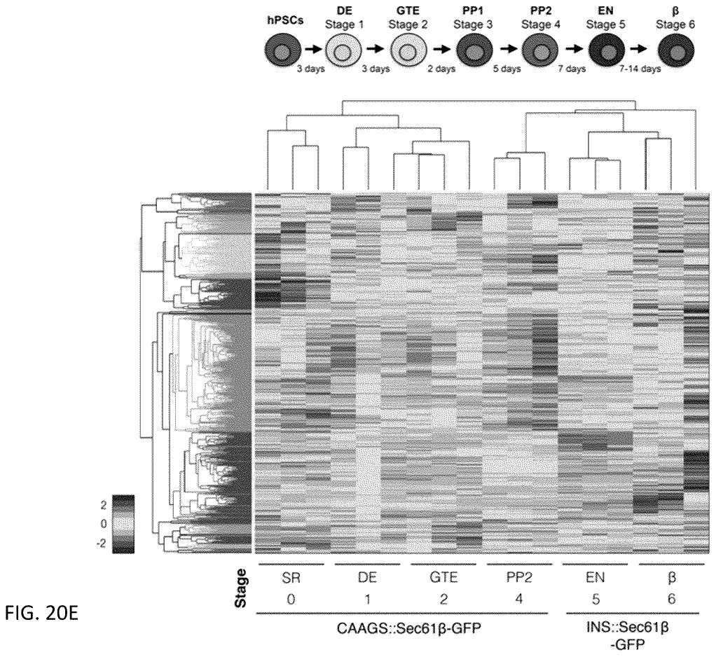

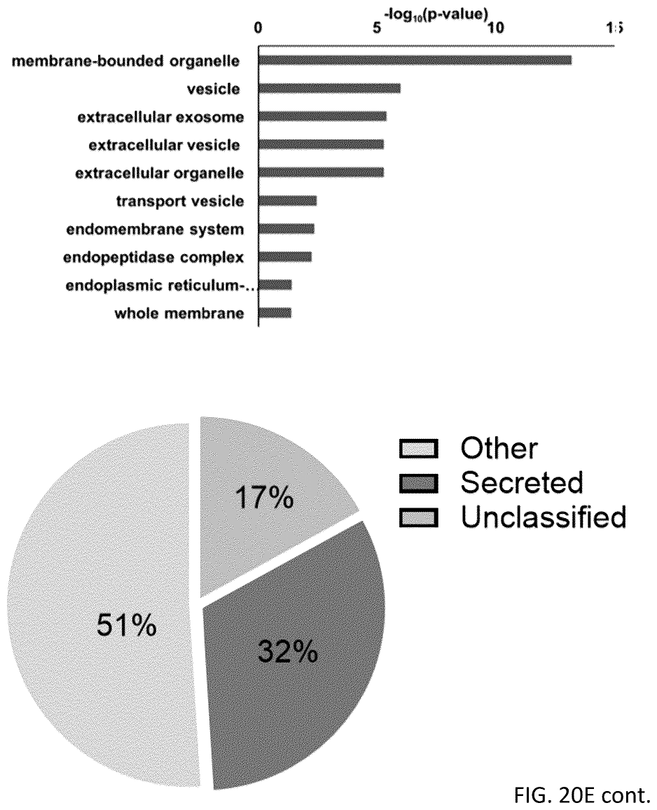

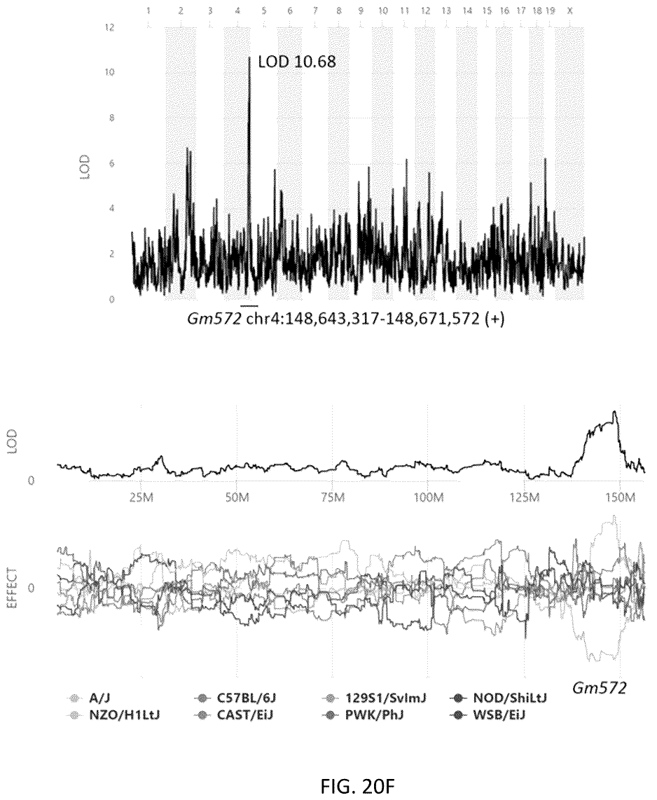

The inventors sought to develop a method that could unbiasedly profile the cell's secretome. Secreted and membrane proteins, including insulin, are translated by ribosomes associated with the endoplasmic reticulum (ER), where they undergo processing and export. Traditional signal peptide-based prediction methods miss many non-canonical or cryptic secreted factors. By tagging the ER translocon with GFP (via Sec61B), we were able to directly label and immunopurify ER-bound ribosomes and their associated mRNAs, thereby enriching for the spectrum of transcripts encoding secreted and membrane proteins. This approach enabled unbiased discovery of cell-type specific secretomes, independent of predicted topological signals, and proved effective for identifying secreted and transmembrane proteins in pluripotent embryonic (ES) cells (FIGS. 20A-20B).

Confident in the ability of the ER-Seq technique to identify the secretome in ES-cells the inventors targeted the reporter into the endogenous insulin locus, the inventors ensured expression was limited strictly to beta cells upon differentiation (FIG. 20D). Upon generation of stem cell-derived beta (SC-β) cells, immunoprecipitation of ER membranes using anti-GFP beads enabled high-purity isolation of ER-bound ribosome complexes, markedly enriching for mRNAs encoding secreted or membrane-bound proteins. RNA sequencing of the ERseq-derived fraction revealed a focused set of transcripts, with a strong enrichment for expected secreted products (INS, IAPP) and minimal contamination by cytoplasmic or nuclear RNAs (FIG. 20E). An unbiased analysis identified a panel of understudied or unannotated transcripts showing robust and specific ER enrichment in beta cells (Supplementary Methods).

### A Genome-Wide Association Study (GWAS) Links C1ORF127 to Metabolic Dysfunction.

To further prioritize the analysis, the inventors leveraged previous research examining genetic loci associated with obesity and diabetes. Specifically, the inventors cross-referenced the mouse orthologs of the SC-beta ERseq human gene hits with data from a study that utilized the Diversity Outbred (DO) mouse strain fed a Western diet. Expression quantitative trait loci (eQTL) analysis revealed that increased expression of Gm572 at this locus is associated with resistance to diabetes and obesity, whereas decreased expression correlates with impaired glucose tolerance, severe obesity, and diabetes. Notably, the Gm572 locus contains the mouse ortholog of C1ORF127, making it the primary candidate gene from the SC-beta ERseq panel implicated in metabolic disease risk (FIG. 20F).

### C1ORF127 is Expressed in Beta Cells

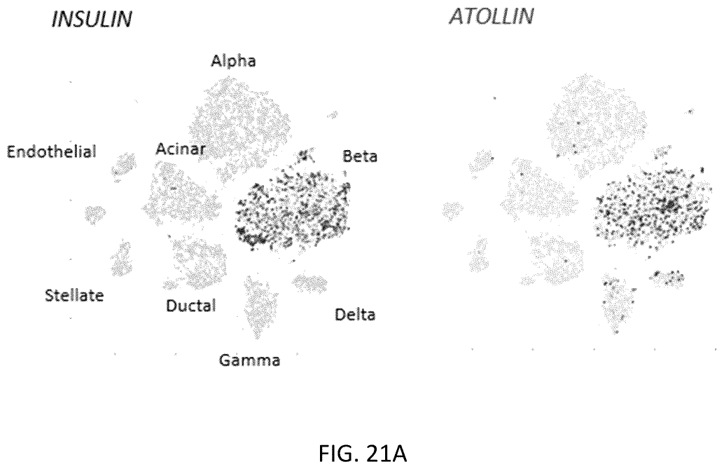

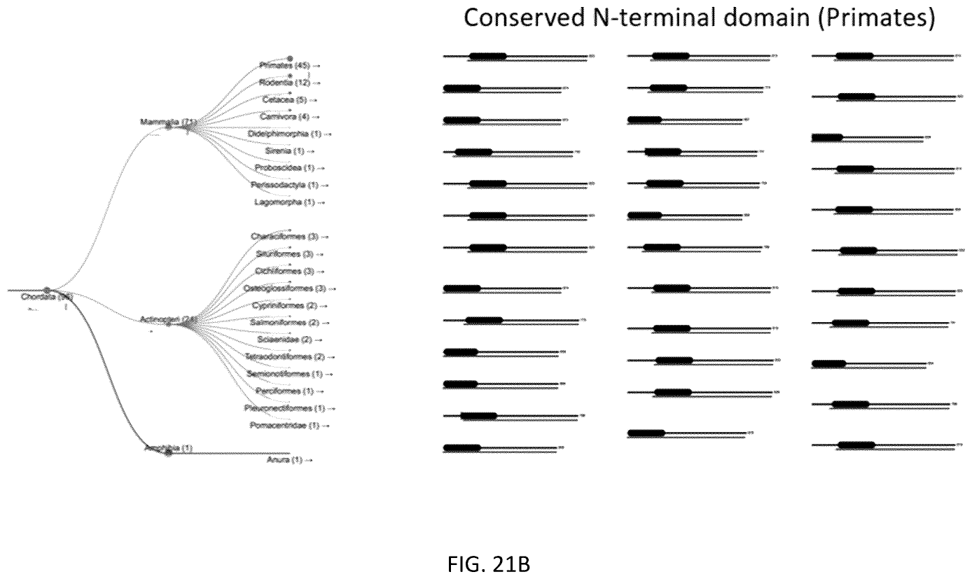

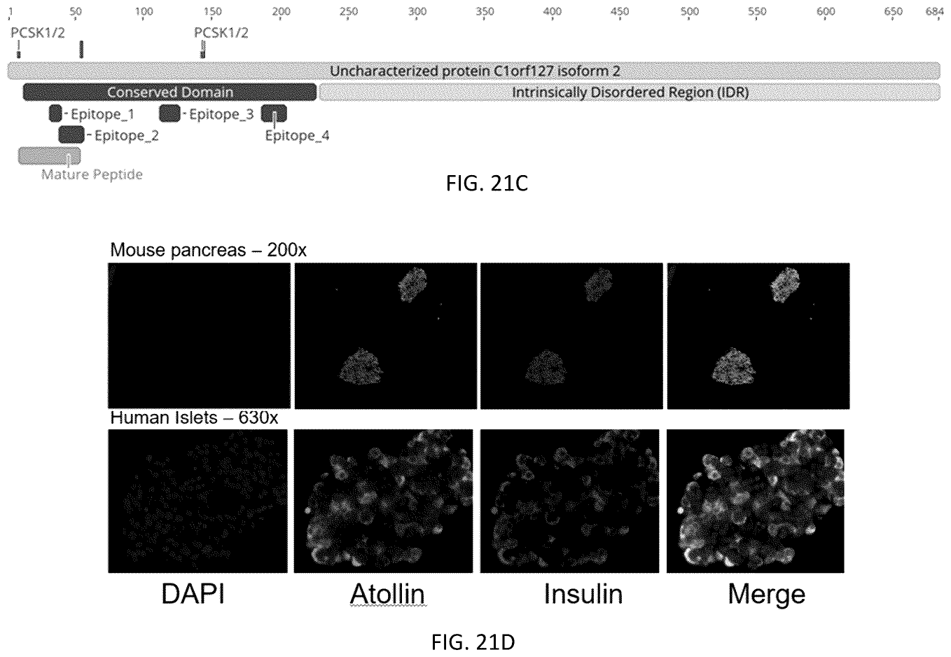

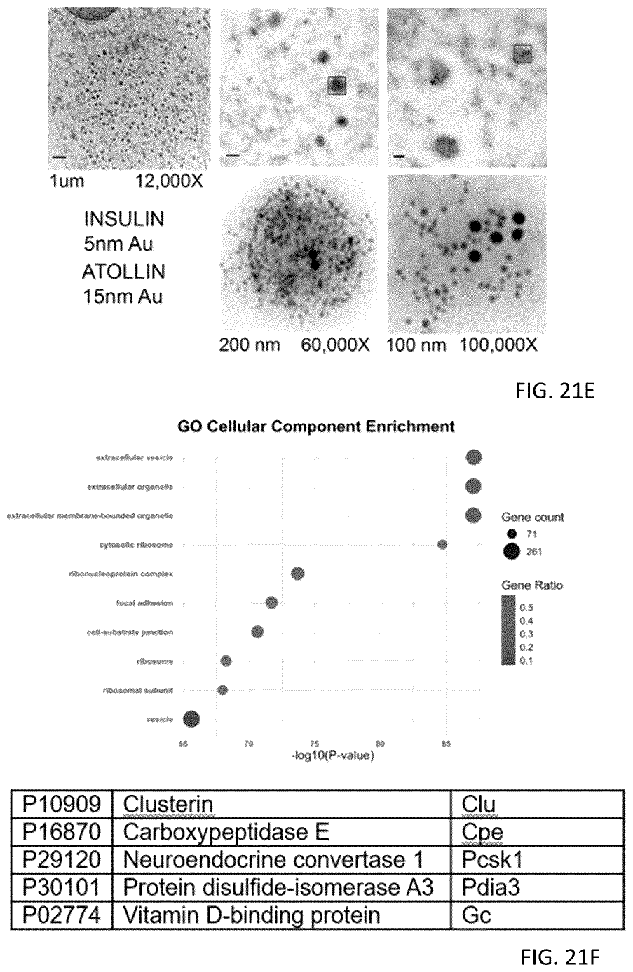

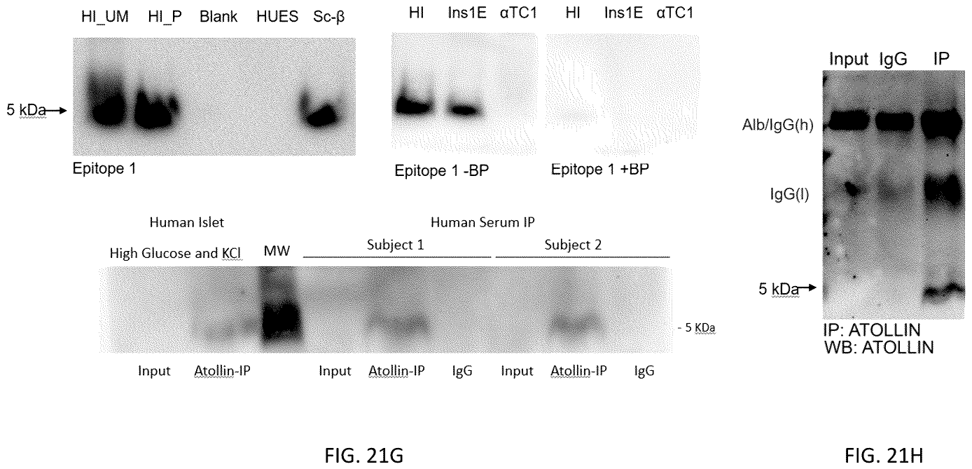

To define the cell-type specificity of C1ORF127, the inventors analyzed single-cell RNA-seq datasets from human islets as well as SC-β and primary beta cells (Veres et al, FIG. 21A). C1ORF127 expression was restricted to beta cells from the inception of the endocrine lineage, with negligible levels in alpha, delta, or pancreatic polypeptide cells (FIG. 21A). Additionally, the expression of C1ORF127 is maturity associated. C1ORF127 is only found in vertebrates and conservation analysis reveals the existence of an evolutionarily conserved domain towards the N-terminus (FIGS. 21B-21C). The predominant isoform of C1ORF127 found in islets is depicted in FIG. 21C. Other notable tissues with expression in publicly available datasets (Human Protein Atlas, GTex, and the like) are gut enteroendocrine cells and cerebellum. Immunofluorescence studies confirmed that C1ORF127 is present in vesicular pools within insulin-expressing cell populations in both human and mouse pancreas (FIG. 21D), as well as in insulinoma cell lines (supplementary text). Double immunogold transmission electron microscopy of human cadaveric islet sections demonstrated that both INSULIN and C1ORF127 are localized within dense core granules (FIG. 21E).

To investigate potential interactions between C1ORF127 and components of the secretory pathway, the inventors prepared protein lysates from human cadaveric islets and performed immunoprecipitations using anti-C1ORF127 antibodies. The resulting pulldowns were analyzed by liquid chromatography-mass spectrometry for protein identification. Cellular compartment ontology analysis showed that C1ORF127 is associated with numerous proteins found in insulin-containing vesicles (FIG. 21F) and suggested that it forms a molecular complex with Pcsk1 (PC1/3), a prohormone convertase involved in the proteolytic processing of insulin and other hormones. This interaction was further validated by western blot analysis of independent co-immunoprecipitation experiments.

### Atollin, a Proprotein Encoded by C1ORF127

Since C1ORF127 is in dense core granules and interacts at the molecular level with endopeptidases, the inventors next performed computational analysis to investigate whether C1ORF127 could be proteolytically processed like insulin (Dodson and Steiner, 1998) and other hormones. Computational analysis of cleavage sites in the conserved N-terminal region identified potential sites for PCSK1 and PCSK2 (FIG. 21C and neuropred, procleave, prosperous outputs). With this in mind, the inventors designed an anti-C1ORF127 antibody to recognize a predicted product of ˜5 kDa (46 aa) nestled between these sites. Western blot analysis of protein extracts from islets and beta cell lines but no other cell types (FIG. 21G) reveal the existence of this cleavage product. The inventors also find this ˜5 kDa protein in islet preps from rodents. Suspecting that C1ORF127 could be a pro-protein, the inventors next examined human serum for the cleavage product. Based on the 1923 study by Best and Scott, it was hypothesized that the hydrophobic nature of a processed fragment of C1ORF127 in circulation would allow it to bind to resins designed to capture hydrophobic proteins (Lipid removal agent, LRA). The inventors developed an enrichment method using such a resin and were able to detect an approximately 5 kDa fragment of C1ORF127 in human serum (FIG. 21H). Additionally, the inventors found the 5 kDa is secreted from human cadaveric isltes upon deporlarization stimuli (FIG. 2G, bottom)

The inventors were unable to prospectively identify the immunoprecipitated Atollin fragment, thus, a semiquantitative LC-MS/MS approach was developed to directly detect Atollin in human serum. Serum samples underwent the same lipid resin enrichment as in the pulldowns, but the bound fraction was eluted with Cleland's reagent (dithiothreitol, DTT) before high-resolution LC-MS/MS analysis. Taken together, these findings indicate that C1ORF127 undergoes endogenous processing in a manner similar to other islet proproteins. In alignment with the cartographic nomenclature associated with the Islets of Langerhans—and drawing on the resemblance between the subcellular distribution of C1ORF127 observed in micrographs and the ring-shaped structure of coral atolls—the inventors henceforth refer to the N-terminal, 5 kDa, processed fragment of C1ORF127 as Atollin.

### Atollin Lowers Blood Glucose in Mice Independent of Insulin Action

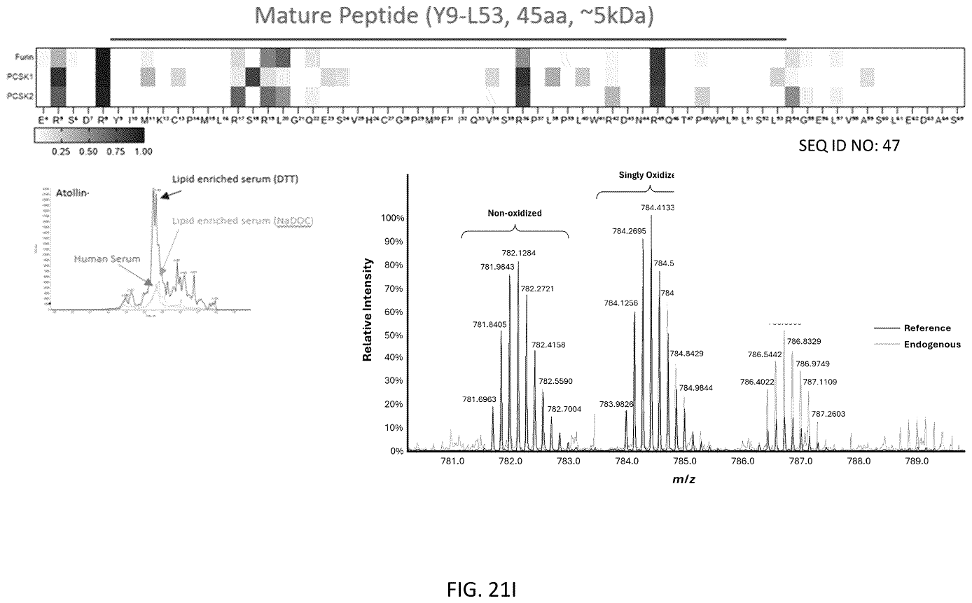

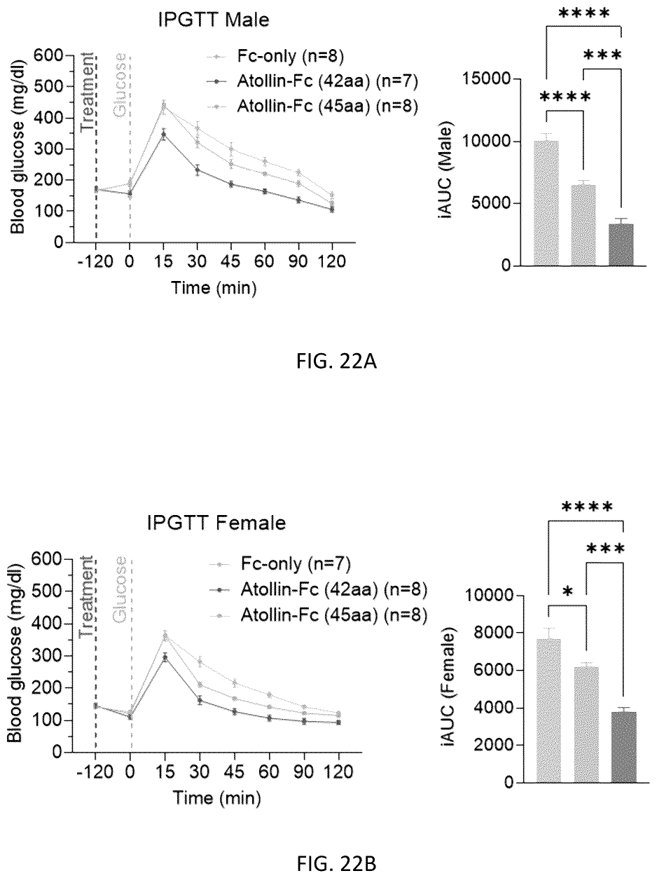

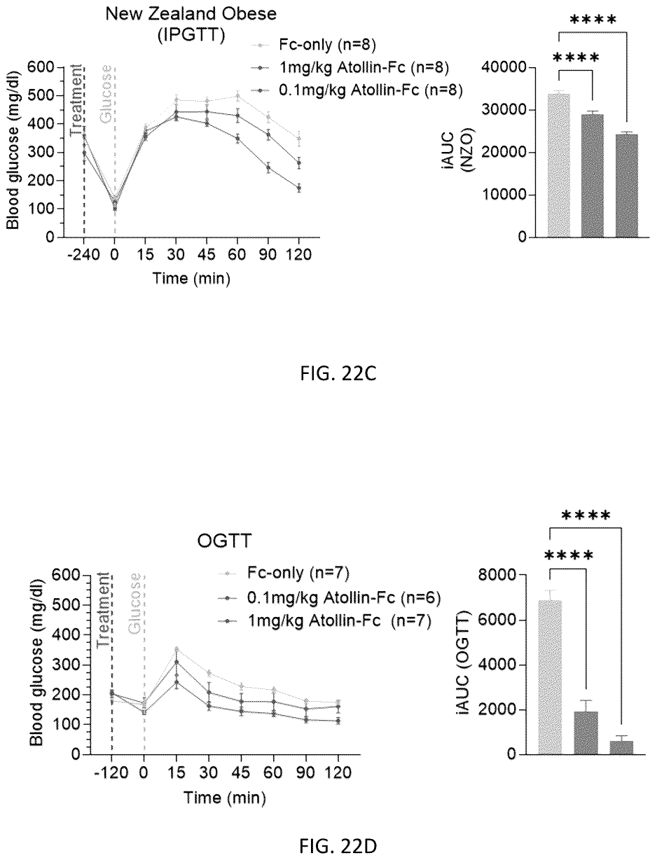

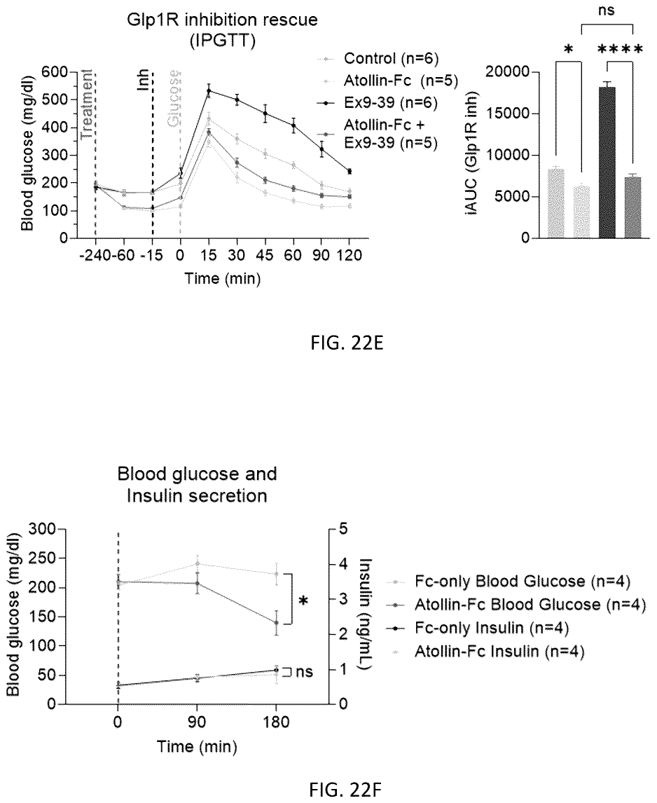

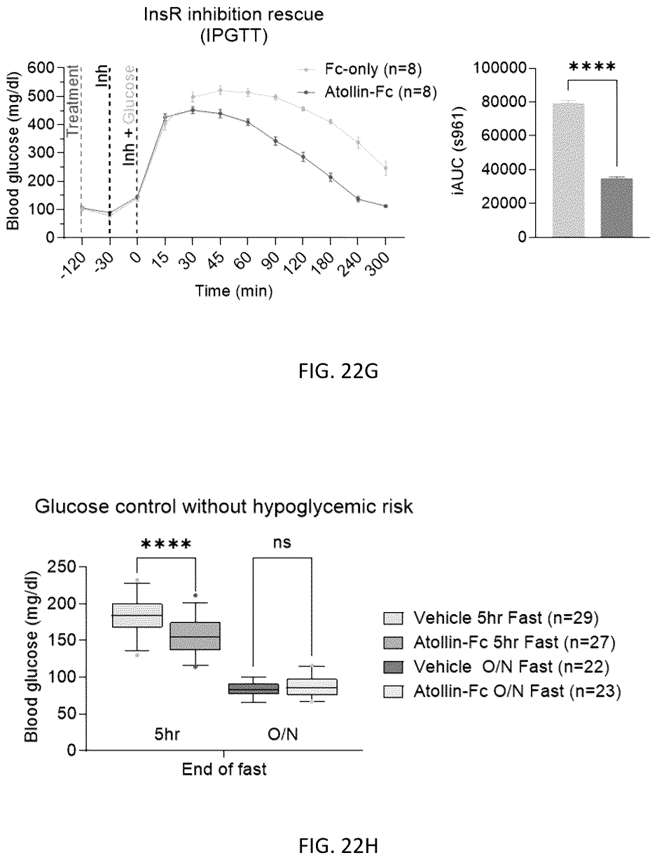

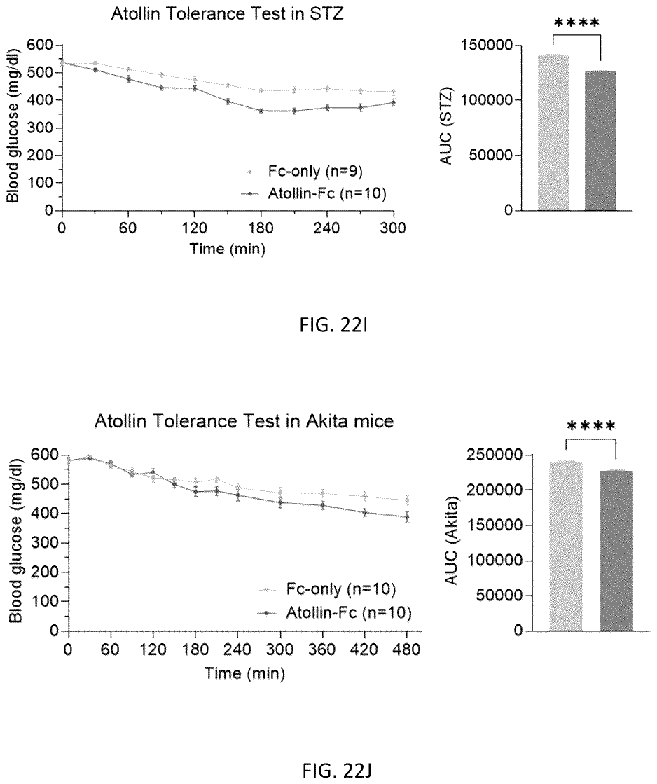

Given that pancreatic beta-cells serve as key regulators of blood glucose levels, the inventors next tested if Atollin could improve glucose tolerance. Aware that small peptides are rapidly degraded and cleared from circulation and to increase the perdurance in circulation, the inventors engineered recombinant fusion proteins combining Atollin with the Fc region of human IgG1 (Atollin-Fc). An Fc-only recombinant protein was also produced as a control. The inventors tested two versions of Atollin encoding for 45 or 42 aa. The 45 aa mimics the predicted Carboxypeptidase E polishing of the C-terminal R in insulin. The 42 aa version is an N-terminal truncation. This was made to avoid potential dipeptidase cleavage liabilities. To assess the potential of these Atollin fragments to enhance glycemic control, the inventors conducted intraperitoneal (i.p.) glucose tolerance tests (IPGTT) in normoglycemic wild type (C57B16NJ) male and female mice and determined that the 42aa Atollin fragment was better at lowering blood glucose (FIGS. 22A-22B). The inventors continue the analysis with this fragment heretofore referred to as Atollin-Fc. Next the inventors wanted to see if injection of Atollin could improve glycemia in the obese and insulin resistant NZO strain. Injection of Atollin-Fc significantly improved glucose tolerance in NZO mice relative to Fc-only controls in a dose responsive manner (FIG. 22C).

Atollin could be lowering blood glucose by promoting insulin secretion, like the effect of Glucagon-like peptide-1 (Glp1) or Glp1Receptor agonists (Glp1RA) or by improving peripheral glucose uptake. To test if Atollin could work as an incretin, the inventors injected wild type mice with Atollin-Fc and performed an oral glucose tolerance test (FIG. 22D). Atollin-Fc was able to improve glycemia in a dose-responsive manner and was more potent at lowering blood glucose than when glucose was delivered i.p., suggesting a classical incretin effect. The incretin-like effect in the OGTT made us wonder if Atollin could be working through the Glp1R pathway. To test whether Atollin's effects depend on this pathway, the inventors used Ex9-39, a drug that blocks the GLP1R and prevents it from stimulating insulin secretion. When the inventors gave mice Ex9-39 before a glucose tolerance test blood sugar levels rose much higher than normal (FIG. 22E), demonstrating the known efficacy of Ex9-39 to blunt insulin secretion. Surprisingly, Atollin-Fc effectively reduced Ex9-39-induced hyperglycemia, indicating that its action does not depend on GLP1R-mediated pathways and suggesting that Atollin-Fc is unlikely to function as an insulin secretagogue. To test this hypothesis further, the inventors injected wildtype mice with Atollin-Fc and controls and monitored both blood glucose and insulin levels in circulation for three hours. As seen in FIG. 22F, Atollin-Fc lowers glycemia without a concomitant increase in circulating insulin.

Having demonstrated that Atollin can enhance glucose tolerance independent of the Glp1R and that it does not promote insulin secretion, the inventors wondered if Atollin could bypass insulin action to regulate blood glucose. To test this, the inventors performed glucose tolerance tests in mice treated with S961, a potent competitive peptide antagonist of the insulin and IGF1 receptors (Schauffer L). As shown in FIG. 22G, the administration of S961 induced acute hyperglycemia, confirming the effectiveness of the S961 regimen in blocking insulin signaling and impairing glucose uptake. Remarkably, Atollin-Fc was able to improve glucose tolerance despite potent inhibition of the insulin receptor. This suggests that Atollin can promote glucose uptake independent of insulin action.

A surprising observation from the glucose tolerance tests was that, at all concentrations tested, the inventors never observed hypoglycemia, even when animals were treated with Atollin after an overnight fast, when tissues are most sensitive to insulin action (FIG. 22H).

Having demonstrated that Atollin can bypass pharmacological inhibition of the InsR and Glp1R the inventors next turned their attention to testing Atollin action in beta-cell ablation and severely insulinopenic models of diabetes, namely Streptozotocin (STZ) mediated beta-cell ablation and Akita mice. Akita mice harbor a point mutation in the Ins2 gene that render the mice diabetic. STZ or Akita diabetic mice were treated acutely with Atollin-Fc or controls and their blood glucose levels were measured over time. As seen in FIGS. 22H-221, Atollin-Fc can improve glycemia in both models, suggesting that Atollin can bypass insulin action in vivo.

### Atollin is a Strong Suppressor of Hepatic Glucose Production

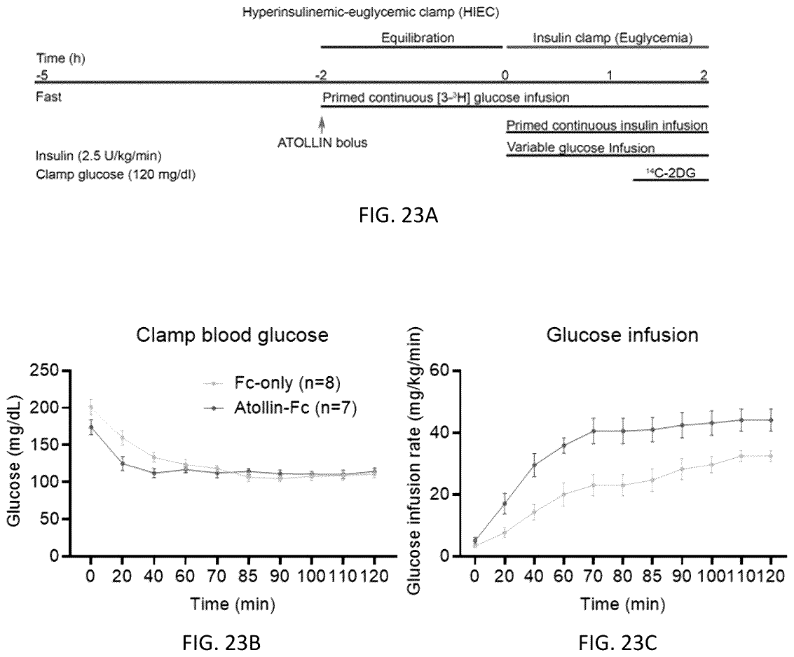

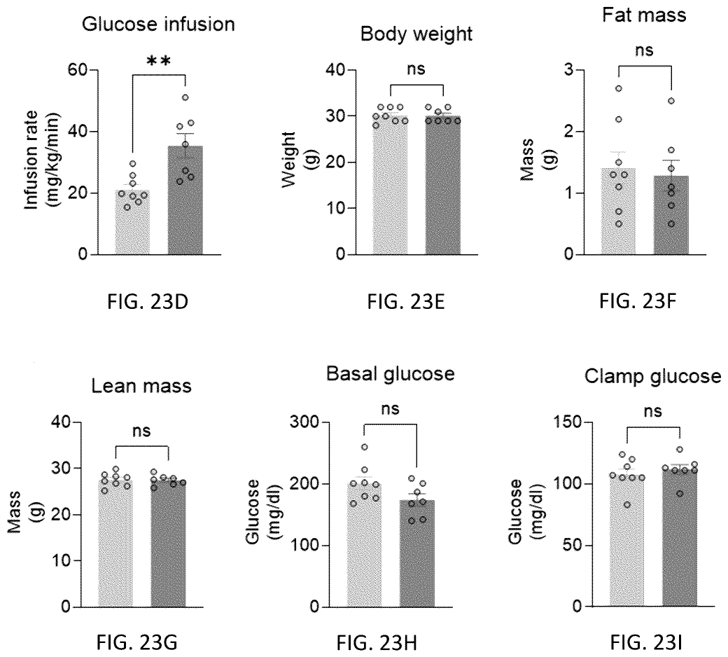

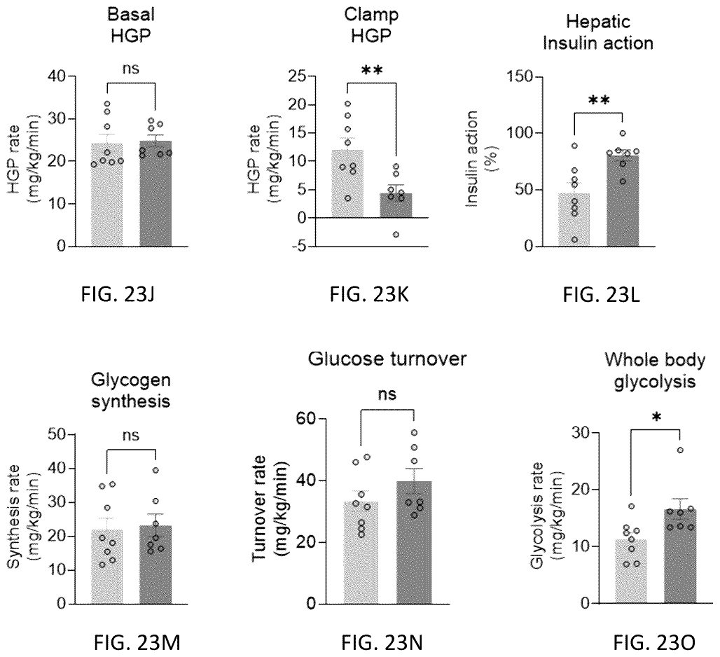

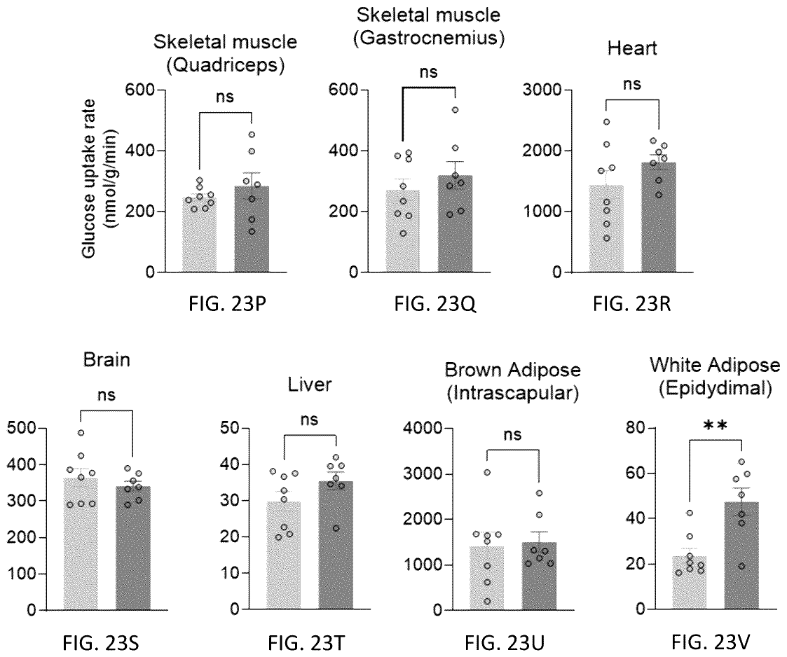

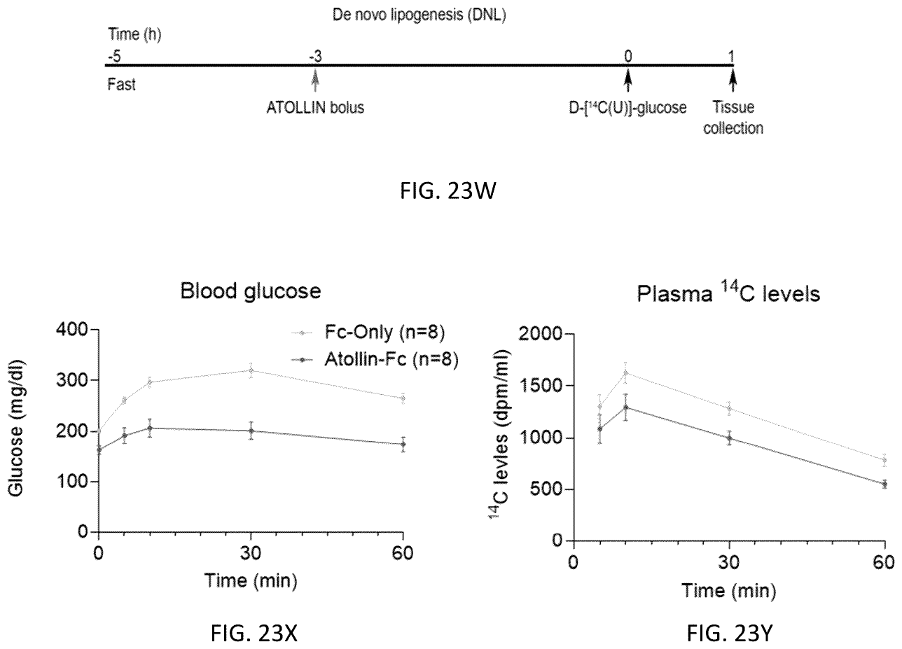

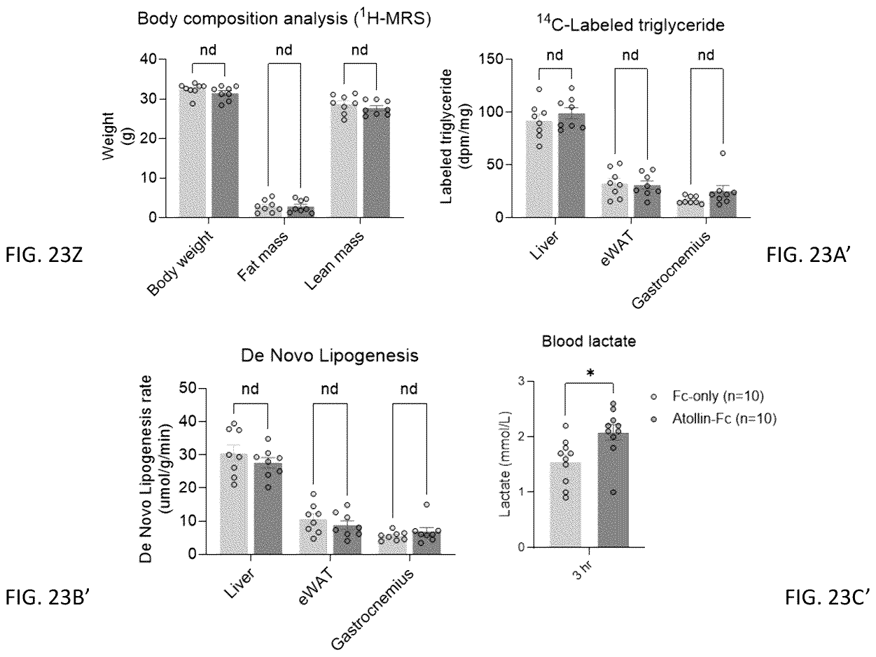

Mechanistically, the inventors thought Atollin could lower blood glucose by suppressing hepatic glucose production, by increasing peripheral glucose uptake, or by a combination of both. To distinguish between these mechanisms, the inventors performed hyperinsulinemic-euglycemic clamp studies (HIEC) with radiolabeled glucose tracers in four-month-old C57B16J mice, an age where the strain exhibits emerging insulin resistance. Mice were fasted for five hours and Atollin-Fc or control was administered i.p. three hours into the fast (FIG. 23A). The effect of Atollin-Fc to lower blood glucose was seen immediately during the insulin clamp period (FIG. 23B) and resulted in an increase in glucose infusion rate that lasted throughout the end of the experiment (FIGS. 23C-23D). No difference in body composition, basal glucose or clamp glucose levels was noted (FIGS. 23E-23I). As expected, basal hepatic glucose production was unremarkable (FIG. 23J). On the other hand, Atollin injection was able to significantly suppress hepatic glucose production during the clamp (FIG. 23K, ˜64% decrease) resulting in a notable increase in hepatic insulin action (FIG. 23L, ˜33% increase). Atollin treatment did not increase glycogen synthesis or glucose turnover rates (FIGS. 23M-23N) but had a notable effect in promoting whole body glycolysis. Together this indicates that Atollin has a prominent central effect in liver as a potent suppressor of hepatic glucose production and a secondary peripheral effect that promotes glucose oxidation.

### Atollin Shunts Glucose to White Adipose Tissue

To assess tissue-specific glucose uptake, the inventors injected radiolabeled 2-deoxyglucose 75 minutes into the clamp period during the HIEC. Once taken up by cells, 2-deoxyglucose is phosphorylated to 2-deoxyglucose-6-phosphate, which cannot be further metabolized or exported, causing it to accumulate intracellularly. Thus, scintillation counts from tissue extracts are proportional to the cumulative glucose uptake by each tissue over the measurement period (Sokoloff L, 1977). Strikingly, Atollin-Fc treatment caused a robust, two-fold increase in glucose uptake exclusively in epididymal white adipose tissue (eWAT; FIGS. 23P-23V), with no significant effect observed in skeletal or cardiac muscle, brain, liver, or intrascapular brown adipose tissue (iBAT). Thus, Atollin shunts circulating glucose preferentially toward white adipose depots, establishing selective substrate redistribution as a key mechanism of its action.

Atollin does not Promote De Novo Lipogenesis and Burns Glucose into Lactate

Given the increased whole-body glycolysis observed during the clamp, the inventors wondered whether Atollin directs excess glucose into energy-burning pathways in adipose tissue, rather than storing it as fat. Typically, insulin lowers lipolysis in white adipose tissue (WAT) and stimulates de novo lipogenesis (DNL), the conversion of glucose into new fatty acids (Santora and Kahn).

To determine whether Atollin promotes fat storage or instead encourages glucose breakdown, the inventors administered radiolabeled glucose after a bolus of Atollin-Fc (FIG. 23W). Consistent with the clamp findings, Atollin-Fc rapidly lowered both blood glucose and plasma ¹⁴C-glucose levels, indicating enhanced glucose clearance (FIGS. 23X-23Y). Importantly, body composition assessed by ‘H-MRS showed no changes among groups (FIG. 23Z), and there was no increase in labeled triglycerides in WAT (FIGS. 23A’-23B′). Thus, Atollin does not appear to drive de novo lipogenesis. The inventors conclude that white fat does not use the extra glucose to make or store more lipid.

If glucose is not stored, is it instead burned? To test whether Atollin stimulates glucose catabolism, the inventors measured blood lactate at the time of maximal glucose disposal in Akita mice. The inventors found a clear, transient rise in circulating lactate levels just before the peak of glucose clearance (FIG. 23C′ vs. FIG. 22J), indicating that Atollin promotes the conversion of glucose to lactate in WAT, a metabolic switch from anabolic storage to catabolic breakdown.

### Atollin is an Essential Gene Required for Normal Oxidative Metabolism.

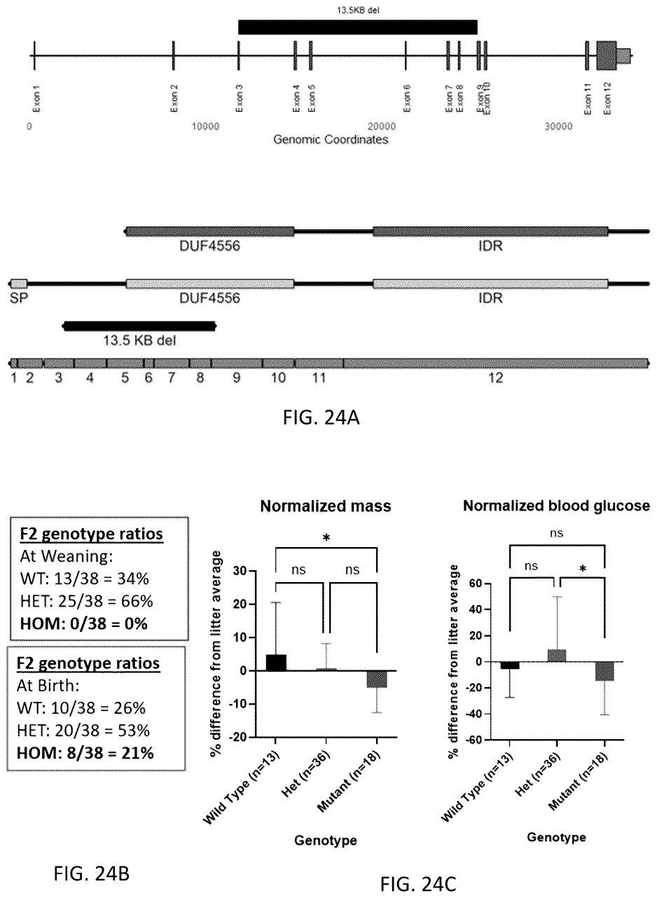

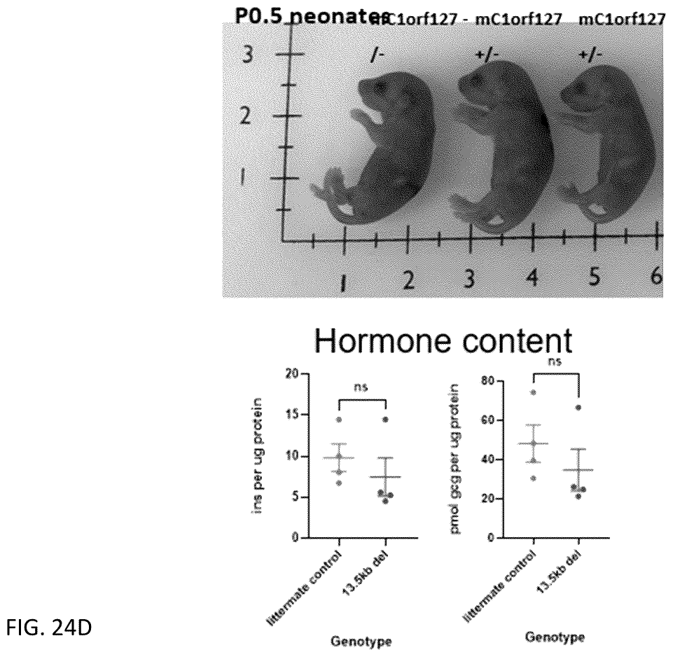

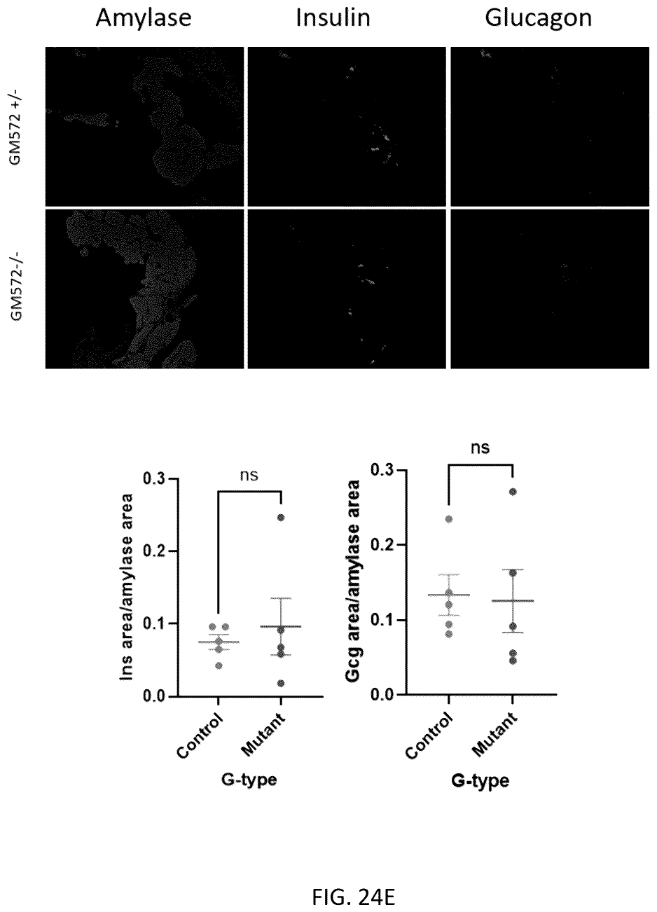

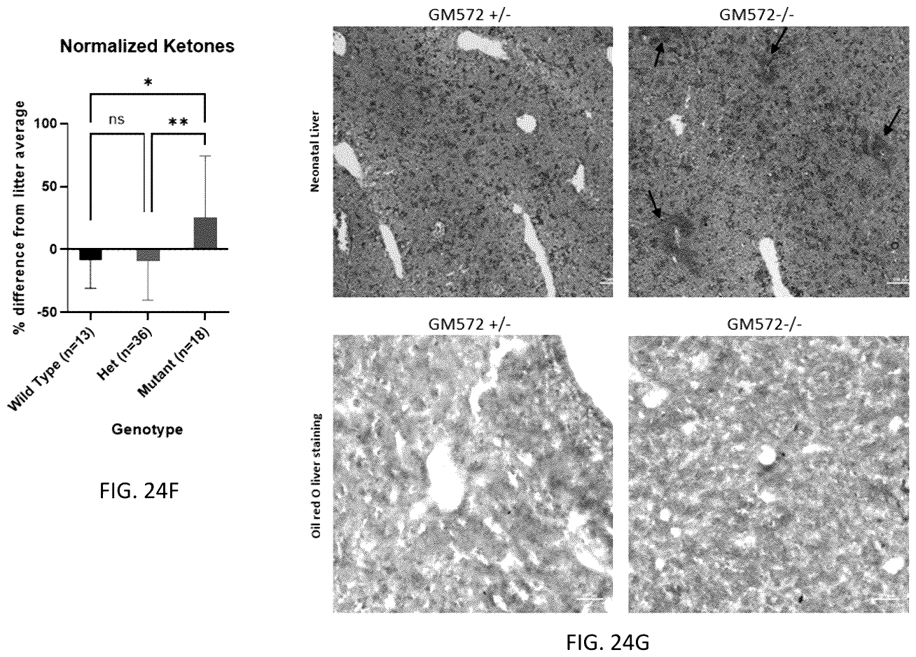

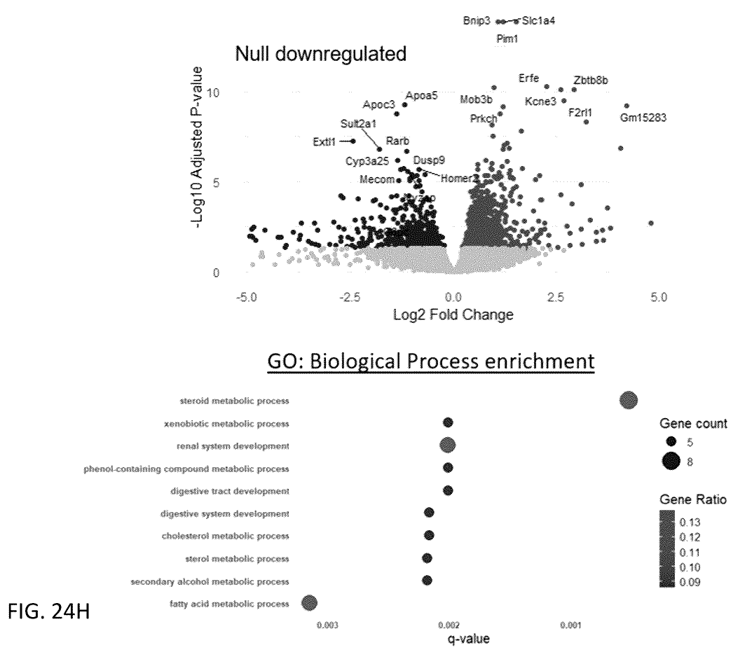

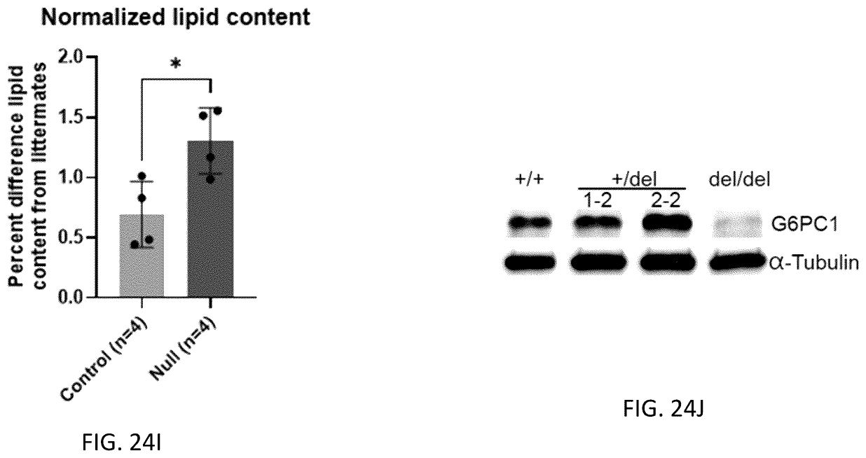

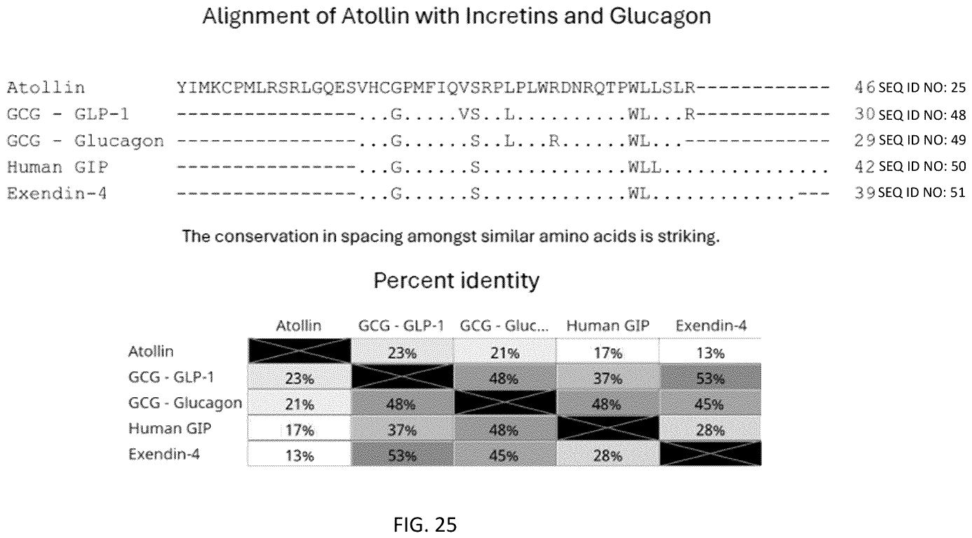

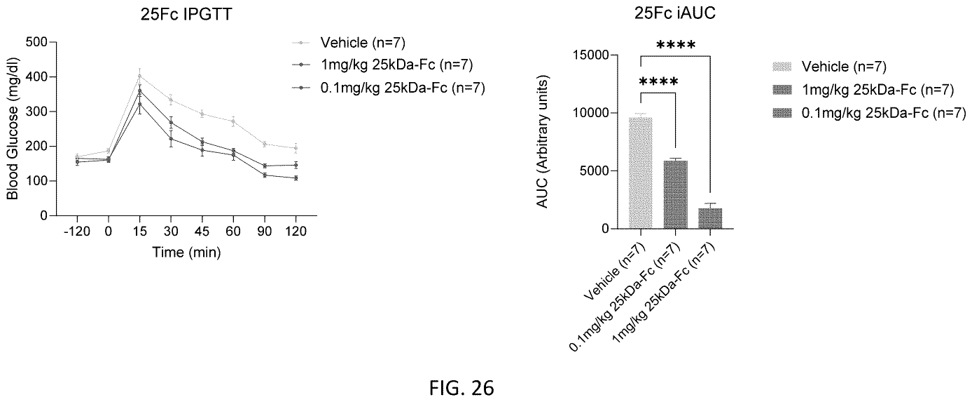

Given Atollin's potent ability to enhance glucose tolerance, and the strong association between reduced Gm572 levels and diabetes susceptibility in mouse GWAS, the inventors next questioned whether Gm572 is essential for organismal metabolism. To investigate this, the inventors generated CRISPR/Cas9-mediated deletions of Gm572 in mice (FIG. 24A), creating two independent deletion alleles. Mutant pups were born normally at mendelian ratios, exhibited regular breathing, and were able to feed, as demonstrated by visible milk spots in their stomachs (FIGS. 24B-24D). They were responsive to touch and capable of movement. Body mass and blood glucose levels were unremarkable. However, both alleles resulted in perinatal lethality within 36 hours of birth.

The inventors measured total insulin and glucagon content in PO mutant and control pancreata and observed no significant differences in hormone levels or cell numbers (FIG. 24E). Histological analysis also revealed no abnormalities in pancreatic structure. Although mutant pups maintained normal glycemia, they displayed significantly elevated blood ketones, as indicated by increased beta-hydroxybutyrate levels (FIG. 24F). This phenotype is reminiscent of insulin receptor knockout mice and suggests a critical role for Gm572 in regulating oxidative metabolism (Acilli et al.).

As diabetic ketoacidosis is linked to elevated hepatic fatty acid oxidation and increased circulating ketones (such as beta-hydroxybutyrate, refs), the inventors assessed neutral lipid accumulation in the livers of mutant mice. Consistent with this phenotype, Oil Red O staining revealed an accumulation of neutral lipids in mutants compared to controls (FIG. 24G). To further validate these findings, the inventors performed bulk RNA-seq analysis on livers from PO mutant and control animals. This analysis revealed strong upregulation of genes involved in sterol synthesis and suppression of key genes involved in limiting triglyceride synthesis (e.g., Apoc3, Apoa5). Notably, the inventors also observed a marked reduction in G6pc transcript abundance. Gopc is a rate-limiting enzyme in hepatic gluconeogenesis, which is expected to restrict hepatic glucose output. This downregulation was confirmed at the protein level by immunoblot using a G6pc-specific antibody, which revealed a potent decrease of G6pc in mutant liver extracts (FIG. 24J). Together these findings demonstrate that Gm572 is essential for maintaining the transcriptional and metabolic programs that support hepatic gluconeogenesis and overall metabolic homeostasis in mice.

### Discussion

Here the inventors identified Atollin as a previously unrecognized beta cell-derived hormone that operates as a potent regulator of glucose metabolism and enables insulin-independent blood glucose control. This work broadens the physiological landscape of islet hormones beyond the century-old paradigm that places insulin at the center of postprandial glucose homeostasis. Through the development of Endoplasmic Reticulum Sequencing (ER-seq), the inventors uncovered C1ORF127 as a candidate prohormone, showing specific enrichment in beta cells, and demonstrate that its processed peptide, Atollin, functions as a highly effective and unique endocrine regulator in vivo.

Classically, insulin's central role as the principal beta cell effector is to suppress hepatic glucose production and stimulate peripheral glucose uptake, particularly in muscle and fat. Therapeutic strategies for diabetes and other metabolic disorders have thus been predominantly focused on either replacing or sensitizing the actions of insulin. However, the results reveal that the endocrine output of beta cells comprises more than just insulin, and that Atollin can robustly normalize glycemia even when insulin signaling is blocked or absent. The ability of Atollin to lower blood glucose in wildtype, obese, and severely insulin-deficient mice, including in the context of pharmacological insulin receptor or GLP-1 receptor antagonism, positions it as a fundamentally new axis in glycemic regulation. These findings not only challenge longstanding assumptions about beta cell biology but also provide mechanistic insight into how glucose control can be achieved in the absence of insulin.

Mechanistically, Atollin exerts its glucose-lowering effect through a dual strategy that is distinct from that of insulin. Hyperinsulinemic-euglycemic clamp studies established that Atollin is a potent suppressor of hepatic glucose production, a key determinant of fasting and postprandial glycemia. Unlike insulin, which simultaneously promotes de novo lipogenesis and glycogen storage, Atollin does not stimulate anabolic pathways or fat accumulation. Instead, tracer analyses reveal that Atollin preferentially increases glucose uptake into white adipose tissue, yet this diverted glucose is not stored as triglyceride. Rather, Atollin promotes a rapid glycolytic conversion of glucose to lactate, effectively “burning” glucose in adipose rather than storing it. This selective catabolic routing of excess glucose represents an unexpected mode of substrate disposal, sidestepping the risks associated with hypoglycemia or excessive fat deposition that commonly complicate insulin-based therapies.

These results extend the physiological repertoire of beta cell function and reveal Atollin as a key mediator of metabolic flexibility. In the context of insulin resistance, beta cell stress, or outright insulin deficiency, an endocrine effector that can safely lower glycemia without driving hypoglycemia or promoting ectopic lipid storage provides a powerful advantage. The finding that Atollin does not provoke hypoglycemia at the tested doses or during fasting, distinguishes it from conventional therapeutics and underscores its therapeutic potential. This unique pharmacology may reflect tight tissue-selectivity and a preference for redirecting glucose flux into rapid oxidation and ultimately disposal as lactate, providing a protective bypass during metabolic overload or insulin failure.

The genetic analyses further demonstrate that Atollin is an essential hormone for perinatal metabolic homeostasis. Mice lacking the C1ORF127 ortholog, Gm572, succumb within 36 hours after birth, displaying profound ketoacidosis and hepatic lipid accumulation, despite otherwise normal insulin and glucagon content and pancreatic morphology. The early lethality and severe dysmetabolism observed in mutant neonates implicate Atollin as an indispensable buffer for oxidative metabolism during a critical developmental window, and highlight the existence of non-insulin, beta cell-mediated checkpoints in systemic energy balance. The comparable perinatal mortality seen in insulin receptor knockout mice draws a striking parallel, but the preservation of insulin levels in Atollin-deficient animals underscores the non-redundant and complementary nature of Atollin's function. Importantly, Atollin is not expressed in the liver, indicating these metabolic defects reflect disrupted endocrine signaling during a critical window of development. Thus, Atollin loss impairs the establishment of hepatic gluconeogenesis, revealing a developmentally essential beta cell-liver communication pathway not compensated by insulin alone.

The selective tissue targeting and metabolic route favored by Atollin also raise intriguing questions about the molecular mechanisms underlying its action. Although the biochemical studies support a classical prohormone processing pathway involving pro-hormone convertases, the identity of Atollin's receptor and downstream signaling networks remain unknown. The restricted pattern of Atollin expression, largely confined to beta cells and potentially select enteroendocrine populations, suggests tight regulation at the level of both synthesis and secretion. The presence of Atollin as a circulating hormone in healthy human serum hints at evolutionarily conserved roles but also raises the possibility that altered Atollin biology may contribute to the pathogenesis or heterogeneity of human diabetes and metabolic disease.

From a translational perspective, these findings have far-reaching implications. The ability of Atollin to bypass insulin and GLP-1 receptor signaling, while avoiding hypoglycemia and excess adiposity, suggests potential for therapeutic development as an adjunct or alternative to existing diabetes treatments. In forms of diabetes characterized by insulin resistance or beta cell failure, where intensifying or replacing insulin is fraught with risk, Atollin or Atollin analogs could provide a novel, mechanistically distinct option for normalizing glycemia and improving metabolic outcomes. The demonstration of serum Atollin in humans, together with genetic links to metabolic traits in both humans and mice, support relevance to human disease and highlight the need for studies examining Atollin in larger animal models and patients.

In summary, the work expands the functional repertoire of beta cells and broadens the conceptual framework of glycemic control. Atollin emerges as a critical endocrine regulator that can normalize blood glucose in the absence of insulin action by suppressing hepatic glucose output and promoting glucose oxidation in adipose tissue. These findings reframe endocrine biology, illuminate hidden complexity in the hormonal control of metabolism, and open new avenues for the treatment of diabetes and metabolic disease.

### Supplemental Material

### Expression Correlates:

The inventors identified an isoform of C1ORF127 that is predicted to encode a 684 amino acid (aa) protein with a molecular weight of 73 kDa. It is located in Human Chromosome 1 at 1p36.22, and it is a highly conserved protein that only exists in vertebrates, with a notable absence in Reptilia. In has no intra-species homologues. The predicted sequence of the isoform lacks canonical signals for secretion or membrane insertion and has an extremely well conserved N-terminal region of 215 aa, containing domain DUF4556 (Interpro: IPR049521). The C-terminal of the protein is extremely disordered and rich in Proline, and, computationally, the inventors have determined it to be an intrinsically disordered region (IDR). There is an annotated larger transcript encoding for a protein of 823 aa with a signal peptide. The inventors performed exon specific quantitative PCR analysis and conclude that the shorter ER-seq identified form is the most abundant isoform in beta cells.

As judged by the co-expression with Neurogenin3 in the psuedotime trajectory, in SC-beta, C1ORF127 is expressed from the inception of the endocrine lineage. Analysis of published datasets revealed that Gm572, is also expressed with Neurogenin3 at embryonic day 15. In SC beta, its expression is later restricted to beta and enterochromaffin cells. In mice, its expression increases during the postnatal period and in humans the inventors find higher expression in adult beta cells relative to fetal tissue. Parallel RT-qPCR analysis across major tissues demonstrated C1ORF127 specificity for beta cells.

Analysis of the gene regulatory network, chromatin accessibility, chromatin occupancy, and bulk RNAseq data at key stages in the maturation of SC-endocrine progenitors into SC-beta demonstrated that the genetic interval encompassing C1ORF127 is dynamically regulated during development and converges in a mature stable signature (Alvarez-Dominguez et al). Mining CHIP-Seq datasets, the inventors were able to find that NeuroD, PDX1 and NKX6.1 can bind to conserved and actively transcribed regions in the Gm572 genetic interval in murine insulinoma cell lines and islets.

### Immunofluorescence:

Using the mouse, the inventors looked more closely at the expression of Gm572 in other hormone lineages and found that in adult mice Gm572 is exclusively expressed in beta-cells judged by the lack of co-expression of Gm572 with Rab5, Lamp2, and p62, the inventors conclude that C1ORF127 is found with Insulin in secretory vesicles. To confirm this observation, the inventors took advantage of super resolution microscopy, which can resolve structures in the nm range, and confirmed the colocalization of Gm572 with Insulin in secretory vesicles.

Development and Validation of ER-Seq for Secretome Profiling in hPSCs and Stem Cell-Derived β Cells

To enable the discovery of cell-type-specific secreted factors, the inventors developed a biochemical fractionation protocol (ER-seq) to isolate mRNAs associated with endoplasmic reticulum (ER)-bound ribosome-translocon complexes, the key sites of secretory protein synthesis. The inventors first engineered a human pluripotent stem cell (hPSC) line expressing an ER translocon subunit, Sec61β, fused to monomeric AcGFP. Using TALEN-mediated genome editing, AcGFP-SEC61β was inserted into the AAVS1 locus under the control of a ubiquitous CAGGS promoter (CAGGS::AcGFP-SEC61B), enabling constitutive expression in hPSCs and their differentiated progeny. Confocal microscopy in both hPSCs and stem cell-derived β (SC-β) cells confirmed perinuclear localization of the fusion protein, consistent with ER residency.

Subcellular fractionation was performed by sequential digitonin permeabilization (releasing cytoplasmic components) followed by extraction with n-dodecyl-β-D-maltoside (DDM) to solubilize ER membranes and luminal contents. This yielded an ER-enriched fraction in hPSCs, stem cell-derived tissues, and HepG2 cells. Immunoprecipitation of this fraction using anti-GFP magnetic beads isolated ribosome/translocon complexes and associated mRNAs. Western blots confirmed significant enrichment of Sec61β and AcGFP in the immunoprecipitated (IP) fraction versus whole cell lysate. Ribosomal protein L13a, and the RNA subunits 28S and 18S, were co-purified, and mass spectrometry further validated the presence of translocon subunits (SEC61α), translocon-associated protein disulfide isomerase (PDI), and multiple ribosomal proteins.

To assess enrichment for secreted factor transcripts, the inventors performed qPCR for candidate secreted genes on translocon-associated mRNAs in hPSCs and stem cell-derived definitive endoderm progenitors. Secreted factors such as FGF2, NENF, NUCB2 (in hPSCs) and CER1, DKK1 (in endoderm) showed 1.5-3-fold enrichment in IP versus total RNA, while cytosolic/nuclear transcripts (e.g. GAPDH, NANOG, SLMO2-ATP5) were depleted over 4-fold. Microarray analysis revealed transcriptome-wide enrichment of secreted and membrane protein genes, with 41% of genes enriched >2-fold in the IP fraction predicted to encode secreted proteins Gene ontology analysis showed significant overrepresentation of endomembrane, vesicle, and extracellular compartment genes.

For β cell-specific profiling, the inventors generated an INS::AcGFP-SEC61β hPSC line by CRISPR-Cas9-mediated knock-in at the endogenous INS locus. Differentiation yielded SC-β cells, with Insulin and GFP co-localization, TSQ/Zn2+ flow cytometry for mature β cells, and Nanostring analysis confirming β cell-specific gene expression. ER-seq of SC-β cells identified marked enrichment of insulin and IAPP as well as secretory pathway and unannotated secreted genes. 32% of IP-enriched genes were predicted secreted factors, while nuclear and cytoplasmic genes were depleted; gene ontology confirmed enrichment of secretory pathway genes.

Stage-specific ER-seq during in vitro β cell differentiation established developmental and β cell-specific signatures, and pathway analysis identified regulators known in β cell biology and disease. Notably, upstream regulator profiles in SC-β cells matched those in non-diabetic islets and highlighted candidate pathways dysregulated in disease.

### Immunoprecipitation Mass Spectrometry Analysis

Analysis of islet and cell line lysates by western blotting found ˜73 kDa and ˜57 kDa bands in human islets, matching full length and LVKR|G (SEQ ID NO: 52) cleavage. Co-IP showed Atollin interacts with PC1/3 and confirms the mass spec results (FIG. 3I).

### Atollin-moxGFP Fusions

To map processing events, the inventors generated Atollin-moxGFP (an ER stable, enhanced GFP monomer) fusion constructs in INS1E cells. Epifluorescence micrographs of the moxGFP/Atollin fusions show a subcellular distribution pattern like that of a secreted protein (cytoplasmic, vesicular-like, and absent from the nucleus). Western blotting with an anti-C1ORF127 antibody revealed bands suggesting processing, including one at ˜45 kDa matching cleavage at LVKR|G (SEQ ID NO: 52), which is a canonical endopeptidase, proprotein convertase 1/3-like (PC1/3), recognition site (depicted as R|G in diagram). This site is remarkably like that for Insulin between the C-peptide and the A-chain: LQKRG (SEQ ID NO: 53). In Insulin, this site has recently been proposed to be cleaved by proprotein convertase 1/3 in human cells (Kieffer). A ˜33 kDa band matched cleavage producing a ˜5 kDa N-terminal fragment, accounting for the addition of GFP.

### Methods

### Generation of Transgenic Cell Lines

All the experiments were performed using the human embryonic stem cell line HUES8. gRNA sequences for the insulin genomic region were ligated into either eCas9 (Addgene 71814) or LbCpf1 (Addgene 84742) CRISPR plasmids. Homology arms flanking ˜750 bp upstream and downstream of the stop codon in the last non-coding exon of the insulin gene were generated by PCR with primers flanking this region. 5′ and 3′ homology arms were ligated to 2A-Sec61β-AcGFP transgene and a puromycin antibiotic selection marker. For the generation of CAGGS::Sec61β-AcGFP cell line, TALEN constructs were designed to target the safe-harbor AAVS1 locus. 5′ and 3′ homology arms were ligated to 2A-PURO (puromycin resistance gene), a linker and CAGGS promoter driving the expression of the Sec61β-AcGFP transgene. HUES8 cells were dispersed into single cells using TrypLE Express and transfected with targeting vectors using the Nucleofector kit (Invitrogen). 72 hr post-electroporation cells were treated with puromycin at a concentration of 1 μg/mL for 7 days to obtain single colonies. Colonies were picked under a microscope around 18-21 days post electroporation into a 96 well plate and expanded. Genomic DNA (gDNA) from the 96 well plate was extracted using the Zymo Research Quick-DNA 96 Plus Kit and insertion of 5′ and 3′ homology arms was confirmed with PCR. Clones that were confirmed as karyotypically normal by karyotype analysis through Cell Line Genetics were used for directed differentiation towards beta cells.

### Differentiation of SC-β Cells

Human pluripotent stem cells (hPSCs) were maintained in mTeSR1 (Stem Cell Technologies) in 500 mL spinner flaks on a stir plate (Chemglass) set to 70 rpm in a 37° C. incubator, 5% CO2, and 100% humidity. Differentiations into SC-β cells were performed following a protocol described by the group (Pagliuca et al., 2014) as follows: HUES8 cells were seeded at 6×10⁵ cells/mL in mTeSR1 media and 10 μm Y27632 (Sigma-Aldrich). The media was changed 48 h later and the differentiations were started 72 h after the cells were seeded. The media changes were as follows:
Stage 1 definitive endoderm: S1+100 ng/mL ActivinA (R&D Systems)+3 μM Chir99021 (Stemgent) in day 1 andS1+100 ng/mL ActivinA on day 2.
Stage 2 gut tube endoderm: days 4, 6: S2+50 ng/mL KGF (Peprotech).
Stage 3 pancreatic progenitor 1: days 7, 8: S3+50 ng/ml KGF+0.25 μM Sant1 (Sigma)+2 μM RA (Sigma)+200 nM LDN193189 (only Day 7) (Sigma)+500 nM PdBU (EMD Millipore).
Stage 4 pancreatic progenitor 2: days 9, 11, 13: S3+50 ng/mL KGF+0.25 μM Sant1+100 nM RA+10 μm Y27632+5 ng/ml Activin A.
Stage 5 endocrine progenitors: Days 14, 16: S5+0.25 μM Sant1+100 nM RA+1 μM XXI (EMD Millipore)+10 μM Alk5i II (Axxora)+1 μM T3 (EMD Millipore)+20 ng/mL Betacellulin (Thermo Fisher Scientific). Days 18, 20: S5+25 nM RA+1 μM XXI+10 μM Alk5i II+1 μM T3+20 ng/mL Betacellulin.
Stage 6 β cells: S3 media change every other day. In the final stage, cells were analyzed between 7 and 21 after stage 6 differentiation was started.

### Biochemical Fractionation Protocol

Around 100×10⁶ cells were collected from differentiation spinner flasks. To stall ribosomes, 100 μg/mL cycloheximide (CHX) was added and incubated for 10 mins. All the following steps of the fractionation were performed with solutions containing 100 μg/mL cycloheximide (Sigma-Aldrich). Cell clusters were washed in PBS/CHX. 4 mL of Accutase/CHX was added to suspension of cell clusters and incubated for 7 mins RT. Clusters were then dissociated by mechanical dissociation using pipettes, resuspended in PBS/CHX and cells were pelleted with centrifugation for 5 mins at 230 rcf at RT. Pellet was resuspended in 3 mL of cytoplasmic buffer containing 110 mM potassium acetate (Sigma-Aldrich), 25 mM K-HEPES (Sigma-Aldrich), 15 mM magnesium chloride (Sigma-Aldrich), 4 mM calcium chloride (Sigma-Aldrich), 0.015% digitonin, 1.0 mM dithiothreitol (Sigma-Aldrich), 100 μg/ml CHX, 1× complete protease inhibitor cocktail (Millipore) and 500U/ml RNasin ribonuclease inhibitors (Promega) and incubated for 20 mins at 4° C. Cell suspension was centrifuged for 5 mins at 845 ref at 4° C. and supernatant (cytoplasmic fraction) and stored at −80° C. for subsequent analysis. The pellet was resuspended in 1 mL ER permeabilization hypotonic buffer containing 20 mM HEPES, 1.5 mM MgCl₂, 0.42M NaCl, 0.2 mM EDTA, 25% glycerol, 2% n-Dodecyl β-D-maltoside (DDM), 1.0 mM DTT. 100 μg/ml CHX and 1× cOmplete protease inhibitor cocktail (Millipore) and 500U/ml RNasin ribonuclease inhibitors (Promega) and 50 uL magnetic agarose binding control beads (Chromotek) and incubated for 1 hour at 4° C. rotating head-to-tail. GFP-MA-TRAP beads (Chromotek) were blocked in 1% BSA-PBS solution for at least 30 mins on ice. Cell extract was homogenized using a Wheaton Glass Homogenizer by slowly moving pestle up and down 15 times. The extract was centrifuged for 5 min at 850 rcf at 4° C. Tube with blocked GFP-MA-TRAP beads was inserted into a DynaMag-2 magnetic stand (Thermo Fisher Scientific) and the solution was discarded. Supernatant was then added to GFP-MA-TRAP bead slurry and incubated for 30 mins at 4° C. with head to tail rotation. Samples were inserted into magnetic stand to collect the unbound fraction and beads were subsequently washed with 500 μL ER Permeabilization buffer twice. After the washes, 500 μl of Trizol was added to beads and stored at −80° C.

### RNA Extraction, Library Preparation and Sequencing

Subcellular fractions stored in TRIzol reagent were combined with 0.2 mL chloroform per 1 mL TRIZol reagent used and incubated for 2-3 mins followed by centrifugation for 15 mins at 12,000 rcf at 4° C. The aqueous phase containing the RNA was combined with 0.5 mL isopropanol per 1 mL TRIZol reagent used. After incubating for 10 mins on ice, RNA was precipitated for 10 minutes at 12,000×g at 4° C. Pellet was washed in 1 mL 75% ethanol and air dried for 5-10 mins. RNA was stored at −80° C. in RNA storage solution (Invitrogen) for subsequent analysis.

Precipitated RNA was subjected to in vitro RNA amplification with reverse transcription following a published protocol (Hashimshony et al., 2012). Reverse-transcription primers were designed with an anchored polyT, the 5′ Illumina adaptor of Illumina small RNA kit and a T7 promoter. The MessageAmp II RNA kit (Ambion) was used with the modified reverse-transcription primer (Hashimshony et al., 2012). The reaction was performed with 50 ng of RNA and 25 ng/uL amplification primers following MessageAmp II RNA kit's protocol. cDNA cleanup was performed with AMPure XP beads (Beckman coulter) by magnetic bead isolation, cleanup with 80% EtOH followed by in vitro transcription for 13 hours following MessageAmp II RNA kit's protocol. RNA was fragmented in 200 mM Tris-acetate [pH 8.1], 500 mM KOAc, 150 mM MgOAc and reaction was stopped by placing on ice and the addition of one-tenth the volume of 0.5M EDTA, followed by RNA cleanup. RNA quality and yield were assayed using a Bioanalyzer (Agilent). Illumina's directional RNA sequencing protocol was used and only the 3′ Illumina adaptor was ligated. A total of 12 cycles of PCR with elongation time of 30s was performed. Libraries were sequenced using the NextSeq 500 platform (Illumina) according to standard protocols. Paired-end sequencing was performed, reading at least 15 bases for read 1, and 50 bases for read 2, and Illumina barcodes.

### Transcriptomic Analysis

Reads were trimmed for universal ilumina adapters and polyA sequences with Cutadapt v1.8.1 and assessed for quality control using Fastqc v0.11.5. Reads were aligned to the human reference genome (hg38) with Tophat v2.1.1 using default parameters. Downstream transcript quantification and differential gene expression analysis was performed with Cufflinks v2.2.1 (Trapnell et al., 2013). Differentially expressed genes were defined as those with adjusted p-values below 0.05 using the cuffdiff algorithm.

### Gene Ontology Analysis

Differentially expressed genes (adjusted p-value<0.05) were used for gene ontology analysis using WebGestalt GSAT (Wang et al., 2017) and ingenuity pathway analysis (IPA, QIAGEN Inc., available at qiagenbioinformatics.com/products/ingenuity-pathway-analysis). Gene ontology terms and enriched pathways with P-value<0.05 were considered as enriched in differentially expressed genes.

### Prediction of Secreted Factors

Translated protein sequences from candidate transcripts were submitted to the Phobius algorithm to predict signal peptide using a hidden Markov model (Kall et al., 2005). Transcripts with predicted signal peptide but no predicted transmembrane topology were classified as part of the secretome of the cell. Ingenuity pathway analysis (IPA, QIAGEN Inc., available at qiagenbioinformatics.com/products/ingenuity-pathway-analysis) was also used to classify candidate transcripts as cytoplasmic or nuclear.

### Western Blot Analysis

After removing the aqueous phase for RNA extraction as described above, the phenol-ethanol was resuspended in 1.5 mL isopropanol per 1 mL TRIzol reagent used and incubated for 10 mins. After centrifugation for 10 mins at 12,000 g at 4° C., the pellet was washed in 2 mL of 0.3 M guanidine hydrochloride in 95% ethanol per 1 mL TRIzol reagent used, incubated for 20 min at RT followed by centrifugation for 5 mins at 7500 g at 4° C. This step was repeated twice. In the final, 2 mL of 100% ethanol per 1 mL TRIzol used was added and incubated for 20 mins. After centrifugation for 5 mins at 7500 g at 4° C., pellet was air dried for 5-10 mins and resuspended in 200 μL of 1% SDS buffer. Protein concentration was measured using the BCA Protein Assay kit (Thermo Scientific). 5-10 ug of protein extracts were separated by AnyKD Mini-Protein precast gels (Bio-Rad) and transferred to nitrocellulose membranes (Bio-Rad). Membranes were blocked in 3% BSA+0.1% Tween 20 TBS for 30 mins at RT and then incubated with the following primary antibodies overnight at 4° C.: mouse anti-Sec61B (Santa Cruz, sc-393633), chick anti-AcGFP (Aves, AcGFP1020), rabbit anti-ribosomal protein L13a (Cell signaling, 2765). After washing, the membranes were incubated with HRP-conjugated secondary antibodies for 1 h at RT, and then incubated in chemiluminescent ECL detection reagent (VWR) for signal detection and development.

### Body Composition and Energy Balance

Whole-body fat and lean mass were non-invasively measured using ¹H-Magnetic Resonance Spectroscopy (MRS) (Echo Medical Systems, Houston, TX). Indirect calorimetry and energy balance parameters, including food/water intake, energy expenditure, respiratory exchange ratio, and physical activity, were noninvasively measured for 3 days using metabolic cages with an environmental chamber (TSE-Systems Inc., Chesterfield, Mo.).

### Hyperinsulinemic-Euglycemic Clamp to Assess Insulin Sensitivity and Glucose Metabolism

Survival surgery was performed in anesthetized mice to establish an indwelling catheter in the jugular vein. Mice were allowed to recuperate from surgery for 5˜6 days before the clamp experiments. Following the overnight fast (˜14 hours), a basal period of the experiment began with a continuous infusion of D-[3-³H]glucose (0.05 mCi/min) using microdialysis pumps to assess the basal rates of whole-body glucose turnover and hepatic glucose production (HGP)⁴⁴. A blood sample (20 ml) was taken at the end of the basal period for the measurement of plasma glucose, insulin, and [³H]glucose concentrations (basal parameters). Following the basal period, a standard 2-hour hyperinsulinemic-euglycemic clamp was conducted with a primed and continuous infusion of human insulin (150 mU/kg body weight priming followed by 2.5 mU/kg/min; Novolin R; Novo Nordisk, Denmark) to raise plasma insulin within a physiological range (˜0.8 ng/ml) 44. Blood samples (10 ml) were collected at 10˜20 min intervals for the immediate measurement of plasma glucose, and 20% dextrose (glucose) was infused at variable rates (glucose infusion rates) to maintain euglycemia (˜120 mg/dl). During the clamp, [3-3H]glucose was continuously infused (0.1 mCi/min) to measure whole-body glucose turnover and glucose metabolic fluxes (glycolysis and glycogen plus lipid synthesis) and clamp HGP during the insulin-stimulated state⁴⁴. The percent change from basal HGP to clamp HGP reflects hepatic insulin action as insulin-mediated percent suppression of HGP. To measure insulin-stimulated glucose uptake in individual organs, 2-[1-¹⁴C]deoxy-D-glucose (2-[¹⁴C]DG) was administered as a bolus (10 mCi) at 75 min after the start of the clamp⁴⁴. Blood samples (20 ml) were taken at 80, 85, 90, 100, 110, and 120 min of the clamp for the measurement of plasma [³H]glucose, ³H₂O, and 2-[¹⁴C]DG concentrations. Additional blood samples (20 ml) were taken at 120 min to measure plasma insulin concentrations (clamp parameters). At the end of the clamp, mice were anesthetized, and tissue samples, such as liver, skeletal muscle, and adipose tissue, were rapidly taken for biochemical and molecular analysis⁴⁴.

### Biochemical Analysis and Calculation

Glucose concentrations during the clamp were measured with an Analox GM9 Analyser (Analox Instruments Ltd., Hammersmith, London, UK) using 5 ml of plasma via glucose oxidase methodology. Plasma concentrations of [3-³H]glucose, 2-[¹⁴C]DG, and ³H₂O were determined following the deproteinization of plasma samples as previously described⁴⁴. For the determination of tissue 2-[¹⁴C]DG-6-Phosphate content, tissue samples were homogenized, and the supernatants were subjected to an ion-exchange column to separate 2-[¹⁴C]DG-6-P from 2-[¹⁴C]DG⁴⁴. Rates of basal HGP and insulin-stimulated whole-body glucose turnover were determined as previously described⁴⁴. HGP during an insulin-stimulated state was determined by subtracting the glucose infusion rate from whole-body glucose turnover. Whole-body glycolysis and glycogen plus lipid synthesis were calculated as previously described⁴⁴. Insulin-stimulated glucose uptake in individual organs was measured by determining the tissue content of 2-[¹⁴C]DG-6-phosphate and plasma 2-[¹⁴C]DG profile⁴⁴.

1. An agent comprising a functional portion of a C1ORF127 gene product having the amino acid sequence of KCPMLRSRLGQESVHCGPMFIQVSRPLPLWRDNRQTPWLLSL (SEQ ID NO: 11).

2. The agent of claim 1, wherein the functional portion of a C1ROF127 gene product is in the form of a cyclic or linear peptide.

3. The agent of claim 1, wherein the functional portion of a C1ORF127 gene product is fused to an Fc region of an antibody.

4. The agent of claim 3, wherein the functional portion of a C1ORF127 gene product is fused to an Fc region of an IgG1 antibody.

5. The agent of claim 1, wherein the agent is provided as a fusion protein having the amino acid sequence of SEQ ID NO: 12.

6. The agent of claim 1, wherein the functional portion of the C1ORF127 gene product further comprises an arginine (R) resulting in an amino acid sequence of KCPMLRSRLGQESVHCGPMFIQVSRPLPLWRDNRQTPWLLSLR (SEQ ID NO: 26).

7. The agent of claim 6, wherein the functional portion of a C1ORF127 gene product in the form of a cyclic or linear peptide.

8. The agent of claim 6, wherein the functional portion of a C1ORF127 gene product is fused to an Fc region of an antibody.

9. The agent of claim 1, wherein the functional portion of the C1ORF127 gene product further comprises tyrosine (Y), isoleucine (I), and methionine (M), resulting in an amino acid sequence of YIMKCPMLRSRLGQESVHCGPMFIQVSRPLPLWRDNRQTPWLLSL (SEQ ID NO: 10).

10. The agent of claim 9, wherein the functional portion of a C1ORF127 gene product is in the form of a cyclic or linear peptide.

11. The agent of claim 9, wherein the functional portion of a C1ORF127 gene product is fused to an Fc region of an antibody.

12. (canceled)

13. The agent of claim 11, wherein the functional portion of a C1ORF127 gene product is fused to an Fc region of an IgG1 antibody.

14. The agent of claim 8, wherein the functional portion of a C1ORF127 gene product is fused to an Fc region of an IgG1 antibody.

15. The agent of claim 9, wherein the agent is provided as a fusion protein having the amino acid sequence of SEQ ID NO: 23.

16. (canceled)

17. (canceled)

18. (canceled)

19. (canceled)

20. The agent of claim 1, wherein the agent lowers blood glucose, enhances glucose uptake, enhances glucose disposal, directs glucose into adipose tissue, facilitates glucose burning in adipose tissue, reduces hepatic glucose production, does not cause hypoglycemia, facilitates weight loss, reduces age-related weight gain and/or reduces elevated cholesterol levels upon administration to a subject.

21. The agent of claim 20, wherein the subject has a metabolic disorder.

22. The agent of claim 21, wherein the metabolic disorder is selected from the group consisting of diabetes, metabolic dysfunction-associated steatohepatitis, hepatosteatosis, obesity, and combinations thereof.

23. The agent of claim 1, wherein the agent acts independently of insulin.

24. A pharmaceutical composition comprising the agent of claim 1 and a pharmaceutically acceptable carrier.

25. The pharmaceutical composition of claim 24, further comprising an additional therapeutic agent.

26. The pharmaceutical composition of claim 25, wherein the additional therapeutic agent is an anti-diabetic therapeutic and/or an anti-obesity therapeutic.

27. The pharmaceutical composition of claim 25, wherein the additional therapeutic agent is selected from the group consisting of insulin, GLPR1 agonist, SGLT2 inhibitor, metformin, sulfonylureas, thiazolidinediones, CB1 receptor blocker, Apellin agonist, leptin sensitizer, myostatin agonist, mitochondrial uncoupler, and combinations thereof.

28. A method of treating or preventing a disorder associated with elevated blood glucose levels in a subject, comprising administering to said subject an effective amount of the agent of claim 1.

29. A method of treating or preventing a metabolic disorder in a subject, comprising administering to said subject an effective amount of the agent of claim 1.

30. (canceled)

31. The agent of claim 6, wherein the agent lowers blood glucose, enhances glucose uptake, enhances glucose disposal, directs glucose into adipose tissue, facilitates glucose burning in adipose tissue, reduces hepatic glucose production, does not cause hypoglycemia, facilitates weight loss, reduces age-related weight gain and/or reduces elevated cholesterol levels upon administration to a subject.

32. The agent of claim 6, wherein the agent acts independently of insulin.

33. The agent of claim 9, wherein the agent lowers blood glucose, enhances glucose uptake, enhances glucose disposal, directs glucose into adipose tissue, facilitates glucose burning in adipose tissue, reduces hepatic glucose production, does not cause hypoglycemia, facilitates weight loss, reduces age-related weight gain and/or reduces elevated cholesterol levels upon administration to a subject.

34. The agent of claim 9, wherein the agent acts independently of insulin.

35. A method of treating or preventing a metabolic disorder in a subject, comprising administering to said subject an effective amount of the agent of claim 6.

36. A method of treating or preventing a metabolic disorder in a subject, comprising administering to said subject an effective amount of the agent of claim 9.


SEQUENCE LISTING (55 sequences)

SEQ ID NO: 1 - RNA, 2052 nt, synthetic construct
  source 1..2052: mol_type = other RNA
atggggttggagcggagtgatcgctacataatgaagtgtccgatgctaaggtcaaggctgggtcaggaaagcgtccactgtgggcccatgttcatccaggtctcccggcccctgcccctgtggagggacaatagacagactccatggctgctgtcccttcgaggggagctggtggcttctcttgaagacgccagcctgatgggactgtatgtggacatgaatgccaccactgtcaccgtccaaagcccgagacaaggccttcttcagaggtgggaggtgctgaacacctctgctgagctcctgccactatggctggtgagcggtcaccatgcctattctttagaagctgcttgcccaccggtgtcattccagccagagtcggaggtcttagttcacatccccaagcagagactgggtctagtcaaaagaggttcctacattgaggaaaccctgagcctcagattcctccgagtccaccagtccaacatctttatggtgactgagaacaaggactttgtggtggtcagcattccggcggccggggtgctccaggtccagcgatgccaagaagtcggaggaaccccgggaacacaagctttctatagggtagacctgagcctggaatttgccgagatggctgccccggtcctctggacagtggagagcttcttccagtgtgtgggttcaggaacagagtcgcctgcctcaactgctgcactgaggaccactccctccccaccatccccaggaccagagacccctccagcgggagtgccacctgctgcttcctcccaggtgtgggctgcaggaccagctgcccaggaatggctttctcgggacctcctgcaccggccttccgacgcactggccaaaaaggggcttggaccattcctgcaaacagccaaaccggcgagaagaggccagacatctgcctccattctccccagagtggtgcaagctcagcgaggtccccagcctcccccaggggaagcagggatccctggacaccccacacctccagccacgctcccctcggagcctgtagagggtgtccaggctagtccctggcggccacgtccagtcttgccaacgcacccggctctgaccctgcccgtgtcctcagatgcctcctctccttcaccgccagccccgaggcctgaacgacctgaatcacttctggtctcaggaccatctgtcaccctgactgaaggtctaggaactgtgaggcctgaacaggaccccgccaagtctccaggaagtcccctcctgctgagaggcttgtcaagcggggatgtggctgcacctgagcccatcatgggggagcccggccaagccagtgaggagttccagccattggcgaggccctggcgggccacactggctgcagaggagctggtttctcaccgttctcccggagagccccaggaaacgtgctctggaacggaggtggagaggccacgccagacagggcctggtctccccagggagggggccagggggcacatggacctttcatcctcagaaccaagccaggacatagaggggccgggactctccatcctgccagcgagggatgccacattctccaccccaagtgtgaggcagccagaccccagtgcctggctgagttcaggacctgaactcaccgggatgcccagggtgaggctggcagcgcccctggcagttcttcctatggaacctctgccaccagaacctgttcgcccagcagctcttctgacacccgaagcctcatctgtgggagggccagaccaggcccgatacctggagtcagcccctggctggcctgtgggccaggaggagtggggggttgcacacacgtccagccctccatccacgcaaaccctgagcctgtgggctcccacaggagtgttgctacccagcctggtggagcttgaataccccttccaggctggccggggggcctcactccagcaggagctgacagagcccaccttggccctcagtgctgaaagccacaggcctcctgagcttcaagacagtgtggaggggctttctgagaggccctcacgc

SEQ ID NO: 2 - protein, 684 aa, Homo sapiens
MGLERSDRYIMKCPMLRSRLGQESVHCGPMFIQVSRPLPLWRDNRQTPWLLSLRGELVASLEDASLMGLYVDMNATTVTVQSPRQGLLQRWEVLNTSAELLPLWLVSGHHAYSLEAACPPVSFQPESEVLVHIPKQRLGLVKRGSYIEETLSLRFLRVHQSNIFMVTENKDFVVVSIPAAGVLQVQRCQEVGGTPGTQAFYRVDLSLEFAEMAAPVLWTVESFFQCVGSGTESPASTAALRTTPSPPSPGPETPPAGVPPAASSQVWAAGPAAQEWLSRDLLHRPSDALAKKGLGPFLQTAKPARRGQTSASILPRVVQAQRGPQPPPGEAGIPGHPTPPATLPSEPVEGVQASPWRPRPVLPTHPALTLPVSSDASSPSPPAPRPERPESLLVSGPSVTLTEGLGTVRPEQDPAKSPGSPLLLRGLSSGDVAAPEPIMGEPGQASEEFQPLARPWRATLAAEELVSHRSPGEPQETCSGTEVERPRQTGPGLPREGARGHMDLSSSEPSQDIEGPGLSILPARDATFSTPSVRQPDPSAWLSSGPELTGMPRVRLAAPLAVLPMEPLPPEPVRPAALLTPEASSVGGPDQARYLESAPGWPVGQEEWGVAHTSSPPSTQTLSLWAPTGVLLPSLVELEYPFQAGRGASLQQELTEPTLALSAESHRPPELQDSVEGLSERPSR

SEQ ID NO: 3 - protein, 215 aa, synthetic construct
KCPMLRSRLGQESVHCGPMFIQVSRPLPLWRDNRQTPWLLSLRGELVASLEDASLMGLYVDMNATTVTVQSPRQGLLQRWEVLNTSAELLPLWLVSGHHAYSLEAACPPVSFQPESEVLVHIPKQRLGLVKRGSYIEETLSLRFLRVHQSNIFMVTENKDFVVVSIPAAGVLQVQRCQEVGGTPGTQAFYRVDLSLEFAEMAAPVLWTVESFFQC

SEQ ID NO: 4 - protein, 83 aa, synthetic construct
GSYIEETLSLRFLRVHQSNIFMVTENKDFVVVSIPAAGVLQVQRCQEVGGTPGTQAFYRVDLSLEFAEMAAPVLWTVESFFQC

SEQ ID NO: 5 - protein, 541 aa, synthetic construct
GSYIEETLSLRFLRVHQSNIFMVTENKDFVVVSIPAAGVLQVQRCQEVGGTPGTQAFYRVDLSLEFAEMAAPVLWTVESFFQCVGSGTESPASTAALRTTPSPPSPGPETPPAGVPPAASSQVWAAGPAAQEWLSRDLLHRPSDALAKKGLGPFLQTAKPARRGQTSASILPRVVQAQRGPQPPPGEAGIPGHPTPPATLPSEPVEGVQASPWRPRPVLPTHPALTLPVSSDASSPSPPAPRPERPESLLVSGPSVTLTEGLGTVRPEQDPAKSPGSPLLLRGLSSGDVAAPEPIMGEPGQASEEFQPLARPWRATLAAEELVSHRSPGEPQETCSGTEVERPRQTGPGLPREGARGHMDLSSSEPSQDIEGPGLSILPARDATFSTPSVRQPDPSAWLSSGPELTGMPRVRLAAPLAVLPMEPLPPEPVRPAALLTPEASSVGGPDQARYLESAPGWPVGQEEWGVAHTSSPPSTQTLSLWAPTGVLLPSLVELEYPFQAGRGASLQQELTEPTLALSAESHRPPELQDSVEGLSERPSR

SEQ ID NO: 6 - protein, 227 aa, synthetic construct
KCPMLRSRLGQESVHCGPMFIQVSRPLPLWRDNRQTPWLLSLRGELVASLEDASLMGLYVDMNATTVTVQSPRQGLLQRWEVLNTSAELLPLWLVSGHHAYSLEAACPPVSFQPESEVLVHIPKQRLGLVKRGSYIEETLSLRFLRVHQSNIFMVTENKDFVVVSIPAAGVLQVQRCQEVGGTPGTQAFYRVDLSLEFAEMAAPVLWTVESFFQCVGSGTESPASTA

SEQ ID NO: 7 - protein, 494 aa, synthetic construct
MDAMKRGLCCVLLLCGAVFVSPDYKDDDDKKCPMLRSRLGQESVHCGPMFIQVSRPLPLWRDNRQTPWLLSLRGELVASLEDASLMGLYVDMNATTVTVQSPRQGLLQRWEVLNTSAELLPLWLVSGHHAYSLEAACPPVSFQPESEVLVHIPKQRLGLVKRGSYIEETLSLRFLRVHQSNIFMVTENKDFVVVSIPAAGVLQVQRCQEVGGTPGTQAFYRVDLSLEFAEMAAPVLWTVESFFQCVGSGTESPASTAGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 135 aa, synthetic construct
YIMKCPMLRSRLGQESVHCGPMFIQVSRPLPLWRDNRQTPWLLSLRGELVASLEDASLMGLYVDMNATTVTVQSPRQGLLQRWEVLNTSAELLPLWLVSGHHAYSLEAACPPVSFQPESEVLVHIPKQRLGLVKR

SEQ ID NO: 9 - protein, 142 aa, synthetic construct
QTPWLLSLRGELVASLEDASLMGLYVDMNATTVTVQSPRQGLLQRWEVLNTSAELLPLWLVSGHHAYSLEAACPPVSFQPESEVLVHIPKQRLGLVKRGSYIEETLSLRFLRVHQSNIFMVTENKDFVVVSIPAAGVLQVQR

SEQ ID NO: 10 - protein, 45 aa, synthetic construct
  DISULFID 5..19: note = Disulfide bond
  REGION 5..19: note = Peptide is circular
YIMKCPMLRSRLGQESVHCGPMFIQVSRPLPLWRDNRQTPWLLSL

SEQ ID NO: 11 - protein, 42 aa, synthetic construct
KCPMLRSRLGQESVHCGPMFIQVSRPLPLWRDNRQTPWLLSL

SEQ ID NO: 12 - protein, 274 aa, synthetic construct
KCPMLRSRLGQESVHCGPMFIQVSRPLPLWRDNRQTPWLLSLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 37 aa, synthetic construct
YIMKCPMLRSRLGQESVHCGPMFIQVSRPLPLWRDNR

SEQ ID NO: 14 - protein, 35 aa, synthetic construct
MKCPMLRSRLGQESVHCGPMFIQVSRPLPLWRDNR

SEQ ID NO: 15 - protein, 22 aa, synthetic construct
MDAMKRGLCCVLLLCGAVFVSP

SEQ ID NO: 16 - protein, 8 aa, synthetic construct
DYKDDDDK

SEQ ID NO: 17 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 237 aa, synthetic construct
GGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - RNA, 2798 nt, Homo sapiens
  source 1..2798: mol_type = mRNA
aaattctcccagggcaggagagagctgctgggcctgaggatgtgggggtccccagcacttgcctgggccgtctggcttgcctgtgttcagcccacggtgttcccatggagcctctccttcaggtcagacacggacaagccaagctctgcagcagaagttttaacagaagcatcttcttgttggacagacacagttgagtgcttctcagactacatgaccctgtggatcccaaggagccacgtggaggggctgaggcggtggctggccaggaccttgcacttgccaggcacctggaggtcccctgaccacctggattcttctcttgctaaatgtggctacttcctgcatctggcttcagacggtgacttcctttttcgagttcaatactcggcctgctttgtgcagaaagagaaagcaaattacaggctggaaatcagaatatttcagaaaggggtgatggggttggagcggagtgatcgctacataatgaagtgtccgatgctaaggtcaaggctgggccaggaaagcgtccactgtgggcccatgttcatccaggtctcccggcccctgcccctgtggagggacaatagacagactccatggctgctgtcccttcgaggggagctggtggcttctcttgaagacgccagcctgatgggactgtatgtggacatgaatgccaccactgtcaccgtccaaagcccgagacaaggccttcttcagaggtgggaggtgctgaacacctctgctgagctcctgccactatggctggtgagcggtcaccatgcctattctttagaagctgcttgcccaccggtgtcattccagccagagtcggaggtcttagttcacatccccaagcagagactgggtctagtcaaaagaggttcctacattgaggaaaccctgagcctcagattcctccgagtccaccagtccaacatctttatggtgactgagaacaaggactttgtggtggtcagcattccggcggccggggtgctccaggtccagcgatgccaagaagtcggaggaaccccgggaacacaagctttctatagggtagacctgagcctggaatttgccgagatggctgccccggtcctctggacagtggagagcttcttccagtgtgtgggttcaggaacagagtcgcctgcctcaactgctgcactgaggaccactccctccccaccatccccaggaccagagacccctccagcgggagtgccacctgctgcttcctcccaggtgtgggctgcaggaccagctgcccaggaatggctttctcgggacctcctgcaccggccttccgacgcactggccaaaaaggggcttggaccattcctgcaaacagccaaaccggcgagaagaggccagacatctgcctccattctccccagagtggtgcaagctcagcgaggtccccagcctcccccaggggaagcagggatccctggacaccccacacctccagccacgctcccctcggagcctgtagagggtgtccaggctagtccctggcggccacgtccagtcttgccaacgcacccggctctgaccctgcccgtgtcctcagatgcctcctctccttcaccgccagccccgaggcctgaacgacctgaatcacttctggtctcaggaccatctgtcaccctgactgaaggtctaggaactgtgaggcctgaacaggaccccgccaagtctccaggaagtcccctcctgctgagaggcttgtcaagcggggatgtggctgcacctgagcccatcatgggggagcccggccaagccagtgaggagttccagccattggcgaggccctggcgggccacactggctgcagaggagctggtttctcaccgttctcccggagagccccaggaaacgtgctctggaacggaggtggagaggccacgccagacagggcctggtctccccagggagggggccagggggcacatggacctttcatcctcagaaccaagccaggacatagaggggccgggactctccatcctgccagcgagggatgccacattctccaccccaagtgtgaggcagccagaccccagtgcctggctgagttcaggacctgaactcaccgggatgcccagggtgaggctggcagcgcccctggcagttcttcctatggaacctctgccaccagaacctgttcgcccagcagctcttctgacacccgaagcctcatctgtgggagggccagaccaggcccgatacctggagtcagcccctggctggcctgtgggccaggaggagtggggggttgcacacacgtccagccctccatccacgcaaaccctgagcctgtgggctcccacaggagtgttgctacccagcctggtggagcttgaataccccttccaggctggccggggggcctcactccagcaggagctgacagagcccaccttggccctcagtgctgaaagccacaggcctcctgagcttcaagacagtgtggaggggctttctgagaggccctcacgctgagcctccatggagactccaggcagccggtaccccgctggcttggacagccccagtgaccctcagtgacccagctgtccccaggctccctggcttagatgcccagctgtggctgggtacagtgttctgggccggaagagcactcactcagtgcttccctggaaaggtggacccccagacgcccacctccaggcagacccatcactggaagtgggatttgaaatgcagccaagaagaggagaccgtcggcaacgcccgcccggaacaagaaataaataaataaataatca

SEQ ID NO: 20 - RNA, 2472 nt, Homo sapiens
  source 1..2472: mol_type = mRNA
atgtgggggtccccagcacttgcctgggccgtctggcttgcctgtgttcagcccacggtgttcccatggagcctctccttcaggtcagacacggacaagccaagctctgcagcagaagttttaacagaagcatcttcttgttggacagacacagttgagtgcttctcagactacatgaccctgtggatcccaaggagccacgtggaggggctgaggcggtggctggccaggaccttgcacttgccaggcacctggaggtcccctgaccacctggattcttctcttgctaaatgtggctacttcctgcatctggcttcagacggtgacttcctttttcgagttcaatactcggcctgctttgtgcagaaagagaaagcaaattacaggctggaaatcagaatatttcagaaaggggtgatggggttggagcggagtgatcgctacataatgaagtgtccgatgctaaggtcaaggctgggccaggaaagcgtccactgtgggcccatgttcatccaggtctcccggcccctgcccctgtggagggacaatagacagactccatggctgctgtcccttcgaggggagctggtggcttctcttgaagacgccagcctgatgggactgtatgtggacatgaatgccaccactgtcaccgtccaaagcccgagacaaggccttcttcagaggtgggaggtgctgaacacctctgctgagctcctgccactatggctggtgagcggtcaccatgcctattctttagaagctgcttgcccaccggtgtcattccagccagagtcggaggtcttagttcacatccccaagcagagactgggtctagtcaaaagaggttcctacattgaggaaaccctgagcctcagattcctccgagtccaccagtccaacatctttatggtgactgagaacaaggactttgtggtggtcagcattccggcggccggggtgctccaggtccagcgatgccaagaagtcggaggaaccccgggaacacaagctttctatagggtagacctgagcctggaatttgccgagatggctgccccggtcctctggacagtggagagcttcttccagtgtgtgggttcaggaacagagtcgcctgcctcaactgctgcactgaggaccactccctccccaccatccccaggaccagagacccctccagcgggagtgccacctgctgcttcctcccaggtgtgggctgcaggaccagctgcccaggaatggctttctcgggacctcctgcaccggccttccgacgcactggccaaaaaggggcttggaccattcctgcaaacagccaaaccggcgagaagaggccagacatctgcctccattctccccagagtggtgcaagctcagcgaggtccccagcctcccccaggggaagcagggatccctggacaccccacacctccagccacgctcccctcggagcctgtagagggtgtccaggctagtccctggcggccacgtccagtcttgccaacgcacccggctctgaccctgcccgtgtcctcagatgcctcctctccttcaccgccagccccgaggcctgaacgacctgaatcacttctggtctcaggaccatctgtcaccctgactgaaggtctaggaactgtgaggcctgaacaggaccccgccaagtctccaggaagtcccctcctgctgagaggcttgtcaagcggggatgtggctgcacctgagcccatcatgggggagcccggccaagccagtgaggagttccagccattggcgaggccctggcgggccacactggctgcagaggagctggtttctcaccgttctcccggagagccccaggaaacgtgctctggaacggaggtggagaggccacgccagacagggcctggtctccccagggagggggccagggggcacatggacctttcatcctcagaaccaagccaggacatagaggggccgggactctccatcctgccagcgagggatgccacattctccaccccaagtgtgaggcagccagaccccagtgcctggctgagttcaggacctgaactcaccgggatgcccagggtgaggctggcagcgcccctggcagttcttcctatggaacctctgccaccagaacctgttcgcccagcagctcttctgacacccgaagcctcatctgtgggagggccagaccaggcccgatacctggagtcagcccctggctggcctgtgggccaggaggagtggggggttgcacacacgtccagccctccatccacgcaaaccctgagcctgtgggctcccacaggagtgttgctacccagcctggtggagcttgaataccccttccaggctggccggggggcctcactccagcaggagctgacagagcccaccttggccctcagtgctgaaagccacaggcctcctgagcttcaagacagtgtggaggggctttctgagaggccctcacgctga

SEQ ID NO: 21 - protein, 823 aa, Homo sapiens
MWGSPALAWAVWLACVQPTVFPWSLSFRSDTDKPSSAAEVLTEASSCWTDTVECFSDYMTLWIPRSHVEGLRRWLARTLHLPGTWRSPDHLDSSLAKCGYFLHLASDGDFLFRVQYSACFVQKEKANYRLEIRIFQKGVMGLERSDRYIMKCPMLRSRLGQESVHCGPMFIQVSRPLPLWRDNRQTPWLLSLRGELVASLEDASLMGLYVDMNATTVTVQSPRQGLLQRWEVLNTSAELLPLWLVSGHHAYSLEAACPPVSFQPESEVLVHIPKQRLGLVKRGSYIEETLSLRFLRVHQSNIFMVTENKDFVVVSIPAAGVLQVQRCQEVGGTPGTQAFYRVDLSLEFAEMAAPVLWTVESFFQCVGSGTESPASTAALRTTPSPPSPGPETPPAGVPPAASSQVWAAGPAAQEWLSRDLLHRPSDALAKKGLGPFLQTAKPARRGQTSASILPRVVQAQRGPQPPPGEAGIPGHPTPPATLPSEPVEGVQASPWRPRPVLPTHPALTLPVSSDASSPSPPAPRPERPESLLVSGPSVTLTEGLGTVRPEQDPAKSPGSPLLLRGLSSGDVAAPEPIMGEPGQASEEFQPLARPWRATLAAEELVSHRSPGEPQETCSGTEVERPRQTGPGLPREGARGHMDLSSSEPSQDIEGPGLSILPARDATFSTPSVRQPDPSAWLSSGPELTGMPRVRLAAPLAVLPMEPLPPEPVRPAALLTPEASSVGGPDQARYLESAPGWPVGQEEWGVAHTSSPPSTQTLSLWAPTGVLLPSLVELEYPFQAGRGASLQQELTEPTLALSAESHRPPELQDSVEGLSERPSR

SEQ ID NO: 22 - RNA, 2461 nt, Homo sapiens
  source 1..2461: mol_type = mRNA
ggatacaccttgaatgaagaaccatcgggctttcctgttggtatatgcctttccagacttttaaaaattactgctatttttatcccttccaggcacctggaggtcccctgaccacctggattcttctcttgctaaatgtggctacttcctgcatctggcttcagacggtgacttcctttttcgagttcaatactcggcctgctttgtgcagaaagagaaagcaaattacaggctggaaatcagaatatttcagaaaggggtgatggggttggagcggagtgatcgctacataatgaagtgtccgatgctaaggtcaaggctgggccaggaaagcgtccactgtgggcccatgttcatccaggtctcccggcccctgcccctgtggagggacaatagacagactccatggctgctgtcccttcgaggggagctggtggcttctcttgaagacgccagcctgatgggactgtatgtggacatgaatgccaccactgtcaccgtccaaagcccgagacaaggccttcttcagaggtgggaggtgctgaacacctctgctgagctcctgccactatggctggtgagcggtcaccatgcctattctttagaagctgcttgcccaccggtgtcattccagccagagtcggaggtcttagttcacatccccaagcagagactgggtctagtcaaaagaggttcctacattgaggaaaccctgagcctcagattcctccgagtccaccagtccaacatctttatggtgactgagaacaaggactttgtggtggtcagcattccggcggccggggtgctccaggtccagcgatgccaagaagtcggaggaaccccgggaacacaagctttctatagggtagacctgagcctggaatttgccgagatggctgccccggtcctctggacagtggagagcttcttccagtgtgtgggttcaggaacagagtcgcctgcctcaactgctgcactgaggaccactccctccccaccatccccaggaccagagacccctccagcgggagtgccacctgctgcttcctcccaggtgtgggctgcaggaccagctgcccaggaatggctttctcgggacctcctgcaccggccttccgacgcactggccaaaaaggggcttggaccattcctgcaaacagccaaaccggcgagaagaggccagacatctgcctccattctccccagagtggtgcaagctcagcgaggtccccagcctcccccaggggaagcagggatccctggacaccccacacctccagccacgctcccctcggagcctgtagagggtgtccaggctagtccctggcggccacgtccagtcttgccaacgcacccggctctgaccctgcccgtgtcctcagatgcctcctctccttcaccgccagccccgaggcctgaacgacctgaatcacttctggtctcaggaccatctgtcaccctgactgaaggtctaggaactgtgaggcctgaacaggaccccgccaagtctccaggaagtcccctcctgctgagaggcttgtcaagcggggatgtggctgcacctgagcccatcatgggggagcccggccaagccagtgaggagttccagccattggcgaggccctggcgggccacactggctgcagaggagctggtttctcaccgttctcccggagagccccaggaaacgtgctctggaacggaggtggagaggccacgccagacagggcctggtctccccagggagggggccagggggcacatggacctttcatcctcagaaccaagccaggacatagaggggccgggactctccatcctgccagcgagggatgccacattctccaccccaagtgtgaggcagccagaccccagtgcctggctgagttcaggacctgaactcaccgggatgcccagggtgaggctggcagcgcccctggcagttcttcctatggaacctctgccaccagaacctgttcgcccagcagctcttctgacacccgaagcctcatctgtgggagggccagaccaggcccgatacctggagtcagcccctggctggcctgtgggccaggaggagtggggggttgcacacacgtccagccctccatccacgcaaaccctgagcctgtgggctcccacaggagtgttgctacccagcctggtggagcttgaataccccttccaggctggccggggggcctcactccagcaggagctgacagagcccaccttggccctcagtgctgaaagccacaggcctcctgagcttcaagacagtgtggaggggctttctgagaggccctcacgctgagcctccatggagactccagggtggacccccagacgcccacctccaggcagacccatcactggaagtgggatttgaaatgcagccaagaagaggagaccgtcggcaacgcccgcccggaacaagaaataaataaataaataatca

SEQ ID NO: 23 - protein, 277 aa, synthetic construct
YIMKCPMLRSRLGQESVHCGPMFIQVSRPLPLWRDNRQTPWLLSLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 467 aa, synthetic construct
DYKDDDDKKCPMLRSRLGQESVHCGPMFIQVSRPLPLWRDNRQTPWLLSLRGELVASLEDASLMGLYVDMNATTVTVQSPRQGLLQRWEVLNTSAELLPLWLVSGHHAYSLEAACPPVSFQPESEVLVHIPKQRLGLVKRGSYIEETLSLRFLRVHQSNIFMVTENKDFVVVSIPAAGVLQVQRCQEVGGTPGTQAFYRVDLSLEFAEMAAPVLWTVESFFQCVGSGTESPASTAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 46 aa, synthetic construct
YIMKCPMLRSRLGQESVHCGPMFIQVSRPLPLWRDNRQTPWLLSLR

SEQ ID NO: 26 - protein, 43 aa, synthetic construct
KCPMLRSRLGQESVHCGPMFIQVSRPLPLWRDNRQTPWLLSLR

SEQ ID NO: 27 - protein, 33 aa, synthetic construct
QESVHCGPMFIQVSRPLPLWRDNRQTPWLLSLR

SEQ ID NO: 28 - protein, 30 aa, synthetic construct
QESVHCGPMFIQVSRPLPLWRDNRQTPWLL

SEQ ID NO: 29 - protein, 27 aa, synthetic construct
QESVHCGPMFIQVSRPLPLWRDNRQTP

SEQ ID NO: 30 - protein, 24 aa, synthetic construct
QESVHCGPMFIQVSRPLPLWRDNR

SEQ ID NO: 31 - protein, 23 aa, synthetic construct
QESVHCGPMFIQVSRPLPLWRDN

SEQ ID NO: 32 - protein, 33 aa, synthetic construct
CPMLRSRLGQESVHCGPMFIQVSRPLPLWRDNR

SEQ ID NO: 33 - protein, 26 aa, synthetic construct
CPMLRSRLGQESVHCGPMFIQVSRPL

SEQ ID NO: 34 - protein, 24 aa, synthetic construct
CPMLRSRLGQESVHCGPMFIQVSR

SEQ ID NO: 35 - protein, 22 aa, synthetic construct
CPMLRSRLGQESVHCGPMFIQV

SEQ ID NO: 36 - protein, 29 aa, synthetic construct
ESVHCGPMFIQVSRPLPLWRDNRQTPWLL

SEQ ID NO: 37 - protein, 26 aa, synthetic construct
ESVHCGPMFIQVSRPLPLWRDNRQTP

SEQ ID NO: 38 - protein, 23 aa, synthetic construct
ESVHCGPMFIQVSRPLPLWRDNR

SEQ ID NO: 39 - protein, 28 aa, synthetic construct
CGPMFIQVSRPLPLWRDNRQTPWLLSLR

SEQ ID NO: 40 - protein, 25 aa, synthetic construct
CGPMFIQVSRPLPLWRDNRQTPWLL

SEQ ID NO: 41 - protein, 23 aa, synthetic construct
CGPMFIQVSRPLPLWRDNRQTPW

SEQ ID NO: 42 - protein, 27 aa, synthetic construct
SVHCGPMFIQVSRPLPLWRDNRQTPWL

SEQ ID NO: 43 - protein, 25 aa, synthetic construct
SVHCGPMFIQVSRPLPLWRDNRQTP

SEQ ID NO: 44 - protein, 22 aa, synthetic construct
SVHCGPMFIQVSRPLPLWRDNR

SEQ ID NO: 45 - protein, 46 aa, synthetic construct
  VARIANT 1..4: note = X can be any amino acid
  VARIANT 7..18: note = X can be any amino acid
  VARIANT 21..25: note = X can be any amino acid
  VARIANT 29: note = X can be any amino acid
  VARIANT 31..40: note = X can be any amino acid
  VARIANT 44: note = X can be any amino acid
XXXXCPXXXXXXXXXXXXCGXXXXXVSRXLXXXXXXXXXXWLLXLR

SEQ ID NO: 46 - protein, 823 aa, synthetic construct
MWGSPALAWAVWLACVQPTVFPWSLSFRSDTDKPSSAAEVLTEASSCWTDTVECFSDYMTLWIPRSHVEGLRRWLARTLHLPGTWRSPDHLDSSLAKCGYFLHLASDGDFLERVQYSACFVQKEKANYRLEIRIFQKGVMGLERSDRYIMKCPMLRSRLGQESVHCGPMFIQVSRPLPLWRDNRQTPWLLSLRGELVASLEDASLMGLYVDMNATTVTVQSPRQGLLQRWEVLNTSAELLPLWLVSGHHAYSLEAACPPVSFQPESEVLVHIPKQRLGLVKRGSYIEETLSLRFLRVHQSNIFMVTENKDFVVVSIPAAGVLQVQRCQEVGGTPGTQAFYRVDLSLEFAEMAAPVLWTVESFFQCVGSGTESPASTAALRTTPSPPSPGPETPPAGVPPAASSQVWAAGPAAQEWLSRDLLHRPSDALAKKGLGPFLQTAKPARRGQTSASILPRVVQAQRGPQPPPGEAGIPGHPTPPATLPSEPVEGVQASPWRPRPVLPTHPALTLPVSSDASSPSPPAPRPERPESLLVSGPSVTLTEGLGTVRPEQDPAKSPGSPLLLRGLSSGDVAAPEPIMGEPGQASEEFQPLARPWRATLAAEELVSHRSPGEPQETCSGTEVERPRQTGPGLPREGARGHMDLSSSEPSQDIEGPGLSILPARDATFSTPSVRQPDPSAWLSSGPELTGMPRVRLAAPLAVLPMEPLPPEPVRPAALLTPEASSVGGPDQARYLESAPGWPVGQEEWGVAHTSSPPSTQTLSLWAPTGVLLPSLVELEYPFQAGRGASLQQELTEPTLALSAESHRPPELQDSVEGLSERPSR

SEQ ID NO: 47 - protein, 62 aa, synthetic construct
ERSDRYIMKCPMLRSRLGQESVHCGPMFIQVSRPLPLWRDNRQTPWLLSLRGELVASLEDAS

SEQ ID NO: 48 - protein, 30 aa, synthetic construct
  VARIANT 1..3: note = X can be any amino acid
  VARIANT 5..9: note = X can be any amino acid
  VARIANT 12..13: note = X can be any amino acid
  VARIANT 15..24: note = X can be any amino acid
  VARIANT 27..29: note = X can be any amino acid
XXXGXXXXXVSXXLXXXXXXXXXXWLXXXR

SEQ ID NO: 49 - protein, 29 aa, synthetic construct
  VARIANT 1..3: note = X can be any amino acid
  VARIANT 5..10: note = X can be any amino acid
  VARIANT 12..13: note = X can be any amino acid
  VARIANT 15..17: note = X can be any amino acid
  VARIANT 19..24: note = X can be any amino acid
  VARIANT 27..29: note = X can be any amino acid
XXXGXXXXXXSXXLXXXRXXXXXXWLXXX

SEQ ID NO: 50 - protein, 42 aa, synthetic construct
  VARIANT 1..3: note = X can be any amino acid
  VARIANT 5..10: note = X can be any amino acid
  VARIANT 12..24: note = X can be any amino acid
  VARIANT 28..42: note = X can be any amino acid
XXXGXXXXXXSXXXXXXXXXXXXXWLLXXXXXXXXXXXXXXX

SEQ ID NO: 51 - protein, 39 aa, synthetic construct
  VARIANT 1..3: note = X can be any amino acid
  VARIANT 5..10: note = X can be any amino acid
  VARIANT 12..24: note = X can be any amino acid
  VARIANT 27..39: note = X can be any amino acid
XXXGXXXXXXSXXXXXXXXXXXXXWLXXXXXXXXXXXXX

SEQ ID NO: 52 - protein, 5 aa, synthetic construct
LVKRG

SEQ ID NO: 53 - protein, 5 aa, synthetic construct
LQKRG

SEQ ID NO: 54 - protein, 4 aa, synthetic construct
RSDR

SEQ ID NO: 55 - protein, 4 aa, synthetic construct
RFLR